(19) United States
(12) Patent Application Publication
(10) Pub. No.: US 2025/0376519 A1
(43) Pub. Date: Dec. 11, 2025

(54) ANTIGEN-BINDING MOLECULE SPECIFICALLY BINDING TO DLL3 AND CD3, AND PHARMACEUTICAL USE THEREOF

(71) Applicants: JIANGSU HENGRUI PHARMACEUTICALS CO., LTD., Lianyungang (CN); SHANGHAI HENGRUI PHARMACEUTICAL CO., LTD., Shanghai (CN)

(72) Inventors: Xin YE, Shanghai (CN); Qingqing YAO, Shanghai (CN); Xinsheng JIN, Shanghai (CN); Hua YING, Shanghai (CN); Weikang TAO, Shanghai (CN)

(21) Appl. No.: 18/877,185

(22) PCT Filed: Jun. 21, 2023
(86) PCT No.: PCT/CN2023/101778

§ 371 Date: Dec. 19, 2024

(30) Foreign Application Priority Data
Jun. 23, 2022 (CN) 202210725979.3

(51) Int. Cl. C07K 16/28; A61K 39/00; A61P 35/00

(52) U.S. Cl. CPC C07K 16/28; A61P 35/00; C07K 16/2809; A61K 2039/505; C07K 2317/24; C07K 2317/31; C07K 2317/92

(57) ABSTRACT

The present disclosure relates to an antigen-binding molecule specifically binding to DLL3 and CD3, and pharmaceutical use thereof. The antigen-binding molecule can be used for treating cancers.

### TECHNICAL FIELD

The present disclosure pertains to the technical field of biology. More specifically, the present disclosure relates to a DLL3/CD3 antigen-binding molecule and use thereof.

### BACKGROUND

The statements herein merely provide background information related to the present disclosure and may not necessarily constitute the prior art.

Small cell lung cancer (SCLC) is a relatively malignant type of lung cancer, accounting for 10%-15% of all lung cancer cases. Tumors of small cell lung cancer grow fast and easily metastasize, with a five-year survival rate below 7%. The drugs for treating small cell lung cancer are quite limited and are primarily chemotherapy drugs, such as platinum/etoposide combined chemotherapy. Patients with small cell lung cancer initially respond well to chemotherapy but are very likely to develop resistance and relapse. In recent years, immunotherapies such as PD-L1 antibodies and PD1 antibodies have shown some efficacy in SCLC patients, with an effective rate of about 15%. There are still no specific targeted therapy drugs developed for this disease.

DLL3 is a ligand that inhibits Notch. Under normal conditions, DLL3 is located on the Golgi apparatus, but in cancer cells (such as cells of small cell lung cancer), DLL3 can be expressed on the cell surface and bind to Notch in a cis manner, impeding cell-to-cell binding and the endocytosis of Notch in target cells, thereby inhibiting the Notch signaling pathway and promoting tumor cell growth. DLL3 is primarily expressed in nerve or neuroendocrine tumors, including small cell lung cancer, large cell neuroendocrine cancer, gastrointestinal neuroendocrine tumors, small cell bladder cancer, glioblastoma multiforme, metastatic castration prostate cancer, melanoma, and the like, especially SCLC. More than 80% of SCLC cases have positive expression of DLL3. However, DLL3 is not expressed in normal lung cancer tissue and paracancerous tissue. This differential expression makes DLL3 a very potential therapeutic target for SCLC treatment.

CD3 is a homodimeric or heterodimeric antigen expressed on T cells. Functional CD3 is formed by dimer binding of two of four different chains: ε, ζ, δ, and γ. CD3 dimer arrangements include γ/ε. δ/ε, and ζ/ζ. CD3 binds to the T-cell receptor complex (TCR) and is essential for T-cell activation. Therefore, it has been proposed to use anti-CD3 antibodies that activate T cells for cancer treatment. However, administration of anti-CD3 antibodies may trigger T cell activation and release of related cytokines. Excessive cytokine release leads to severe cytokine release syndrome (CRS), which is a significant challenge in the clinical use of anti-CD3 antibodies.

### SUMMARY

The present disclosure provides an antigen-binding molecule binding specifically to DLL3 and CD3.

In one aspect, the present disclosure provides an antigen-binding molecule comprising at least one antigen-binding moiety binding specifically to DLL3 and at least one antigen-binding moiety binding specifically to CD3; the antigen-binding moiety binding specifically to DLL3 comprises a heavy chain variable region DLL3-VH and a light chain variable region DLL3-VL, and the antigen-binding moiety binding specifically to CD3 comprises a heavy chain variable region CD3-VH and a light chain variable region CD3-VL.

In some embodiments, provided is the antigen-binding molecule according to the foregoing, wherein:
(i) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 12, 56, or 57, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 13, 63, 64, 65, 66, or 67, respectively; or
(ii) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 14, 79, 80, 81, or 82, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 15, respectively; or
(iii) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 16, 91, 92, 93, or 94, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 17, respectively; or
(iv) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 18, 107, 108, 109, 110, 112, 113, 114, or 115, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 19, respectively.

In some embodiments, provided is the antigen-binding molecule according to the foregoing, wherein:
(i) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 12, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 13, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 54, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 64, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 54, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 63, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 56, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 65, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 56, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 66, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 56, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 67, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 57, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 65, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 57, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 66, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 57, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 67, respectively; or
(ii) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 14, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 15, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 81, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 77, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 79, 80, or 82, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 77, respectively; or
(iii) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 16, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 17, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 91, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 87, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 92, 93, or 94, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 87, respectively; or
(iv) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 18, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 19, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 109, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 100, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 107, 108, 110, 112, 113, 114, or 115, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 100, respectively.

In some embodiments, the DLL3-HCDR1, DLL3-HCDR2, DLL3-HCDR3, DLL3-LCDR1, DLL3-LCDR2, and DLL3-LCDR3 are defined according to the Kabat, IMGT, Chothia, AbM, or Contact numbering scheme.

In some embodiments, provided is the antigen-binding molecule according to any one of the foregoing, wherein:
i) the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, 71, 72, or 73, and a DLL3-LCDR3 comprising the amino acid sequence of SEQ ID NO: 25 or 74; or
ii) the DLL3-VH comprises: a DLL3-HCDR1 comprising the amino acid sequence of SEQ ID NO: 26 or 84, a DLL3-HCDR2 comprising the amino acid sequence of SEQ ID NO: 27 or 85, and a DLL3-HCDR3 comprising the amino acid sequence of SEQ ID NO: 28 or 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
iii) the DLL3-VH comprises: a DLL3-HCDR1 comprising the amino acid sequence of SEQ ID NO: 20, a DLL3-HCDR2 comprising the amino acid sequence of SEQ ID NO: 32, 96, 97, 98, or 99, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; or
iv) the DLL3-VH comprises: a DLL3-HCDR1 comprising the amino acid sequence of SEQ ID NO: 20, a DLL3-HCDR2 comprising the amino acid sequence of SEQ ID NO: 37, 117, 118, 119, or 120, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 69 or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25;
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25.

In some embodiments, provided is the antigen-binding molecule according to any one of the foregoing, wherein:
the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 84, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 84, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 28; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; or
the DLL3-VH comprises: a DLL3-HCDR1 comprising the amino acid sequence of SEQ ID NO: 20, a DLL3-HCDR2 comprising the amino acid sequence of SEQ ID NO: 97, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; or
the DLL3-VH comprises: a DLL3-HCDR1 comprising the amino acid sequence of SEQ ID NO: 20, a DLL3-HCDR2 comprising the amino acid sequence of SEQ ID NO: 98, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; or
the DLL3-VH comprises: a DLL3-HCDR1 comprising the amino acid sequence of SEQ ID NO: 20, a DLL3-HCDR2 comprising the amino acid sequence of SEQ ID NO: 99, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40; or
the DLL3-VH comprises: a DLL3-HCDR1 comprising the amino acid sequence of SEQ ID NO: 20, a DLL3-HCDR2 comprising the amino acid sequence of SEQ ID NO: 117, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40; or
the DLL3-VH comprises: a DLL3-HCDR1 comprising the amino acid sequence of SEQ ID NO: 20, a DLL3-HCDR2 comprising the amino acid sequence of SEQ ID NO: 118, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40; or
the DLL3-VH comprises: a DLL3-HCDR1 comprising the amino acid sequence of SEQ ID NO: 20, a DLL3-HCDR2 comprising the amino acid sequence of SEQ ID NO: 120, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25.

In some embodiments, provided is the antigen-binding molecule according to any one of the foregoing, wherein:
the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40.

In some embodiments, in the antigen-binding molecule according to any one of the foregoing, the DLL3-HCDR1, DLL3-HCDR2, DLL3-HCDR3, DLL3-LCDR1, DLL3-LCDR2, and DLL3-LCDR3 are defined according to the Kabat numbering scheme.

In some embodiments, the antigen-binding molecule according to any one of the foregoing binds to human DLL3 at 25° C. with a KD of less than 4×10⁻⁹ M, 3×10⁻⁹ M, 2×10⁻⁹ M, 1×10⁻⁹ M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M, 3×10⁻¹⁰ M, 2×10⁻¹⁰ M, 1×10⁻¹⁰ M, or 9×10⁻¹¹ M, as measured by surface plasmon resonance.

In some embodiments, provided is the antigen-binding molecule according to any one of the foregoing, wherein the DLL3-VH and DLL3-VL are humanized and comprise FR regions of a human antibody.

In some embodiments, provided is the antigen-binding molecule according to any one of the foregoing, wherein:
(i) the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and
the DLL3-VL comprises: a DLL3-LCDR1 comprising the amino acid sequence of SEQ ID NO: 23, a DLL3-LCDR2 comprising the amino acid sequence of SEQ ID NO: 24, 71, 72, or 73, and a DLL3-LCDR3 comprising the amino acid sequence of SEQ ID NO: 25 or 74, and FR regions of the DLL3-VL comprise one or more amino acid substitutions selected from the group consisting of 1N, 36L, 42K, 43S, 44F, 46G, 69A, 71Y, 79Q, and 85D; or (ii) the DLL3-VH comprises: a DLL3-HCDR1 comprising the amino acid sequence of SEQ ID NO: 26 or 84, a DLL3-HCDR2 comprising the amino acid sequence of SEQ ID NO: 27 or 85, and a DLL3-HCDR3 comprising the amino acid sequence of SEQ ID NO: 28 or 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y; or
(iii) the DLL3-VH comprises: a DLL3-HCDR1 comprising the amino acid sequence of SEQ ID NO: 20, a DLL3-HCDR2 comprising the amino acid sequence of SEQ ID NO: 32, 96, 97, 98, or 99, and a DLL3-HCDR3 comprising the amino acid sequence of SEQ ID NO: 33, and FR regions of the DLL3-VH comprise one or more amino acid substitutions selected from the group consisting of 1E, 24T, 44S, 71V, and 73K; and
the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36, and FR regions of the DLL3-VL comprise one or more amino acid substitutions selected from the group consisting of 1N, 9K, 43S, 68A, and 85D; or
(iv) the DLL3-VH comprises: a DLL3-HCDR1 comprising the amino acid sequence of SEQ ID NO: 20, a DLL3-HCDR2 comprising the amino acid sequence of SEQ ID NO: 37, 117, 118, 119, or 120, and a DLL3-HCDR3 comprising the amino acid sequence of SEQ ID NO: 38, and FR regions of the DLL3-VH comprise one or more amino acid substitutions selected from the group consisting of 1E, 3R, 5Q, 7F, 9P, 10V, 12V, 40S, 41H, 44S, 48I, 67A, 69L, 71V, 73K, 75S, and 83T; and
the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40, and FR regions of the DLL3-VL comprise one or more amino acid substitutions selected from the group consisting of 9K, 43S, 60D, 68A, 87H, and 100S.

In some embodiments, the antigen-binding molecule according to any one of the foregoing binds to human DLL3 at 25° C. with a KD of less than 4×10⁻⁹ M, 3×10⁻⁹ M, 2×10⁻⁹M, 1×10⁻⁹M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M, 3×10⁻¹⁰ M, 2×10⁻¹⁰ M, 1×10⁻¹⁰ M, or 9×10⁻¹¹ M, as measured by surface plasmon resonance.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25, and FR regions of the DLL3-VL comprise one or more amino acid substitutions selected from the group consisting of TN, 36L, 42K, 43S, 44F, 46G, 69A, 71Y, 79Q, and 85D.

In some embodiments, provided is the antigen-binding molecule according to any one of the foregoing, wherein:
the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y; or
the DLL3-VH comprises: a DLL3-HCDR1 comprising the amino acid sequence of SEQ ID NO: 84, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y; or
the DLL3-VH comprises: a DLL3-HCDR1 comprising the amino acid sequence of SEQ ID NO: 84, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 28; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33, and FR regions of the DLL3-VH comprise one or more amino acid substitutions selected from the group consisting of 1E, 24T, 44S, 71V, and 73K; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36, and FR regions of the DLL3-VL comprise one or more amino acid substitutions selected from the group consisting of 1N, 9K, 43S, 68A, and 85D.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38, and FR regions of the DLL3-VH comprise one or more amino acid substitutions selected from the group consisting of 1E, 3R, 5Q, 7F, 9P, 10V, 12V, 40S, 41H, 44S, 48I, 67A, 69L, 71V, 73K, 75S, and 83T; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40, and FR regions of the DLL3-VL comprise one or more amino acid substitutions selected from the group consisting of 9K, 43S, 60D, 68A, 87H, and 100S.

In some embodiments, provided is the antigen-binding molecule according to any one of the foregoing, wherein:
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises the amino acid sequence of SEQ ID NO: 50 or a variant thereof. In some embodiments, the variant comprises one or more amino acid substitutions selected from the group consisting of Q1E, Q43K, M48I, V67A, I69L, R71V, T73K, and S76N in FR regions of SEQ ID NO: 50.

In some embodiments, provided is the antigen-binding molecule according to any one of the foregoing, wherein:
the DLL3-VH comprises the amino acid sequence of SEQ ID NO: 51 or a variant thereof. In some embodiments, the variant comprises one or more amino acid substitutions selected from the group consisting of Q1E, 5Q, R38K, V68A, F69L, L71V, T73K, V75S, and S76N in FR regions of SEQ ID NO: 51.

In some embodiments, provided is the antigen-binding molecule according to any one of the foregoing, wherein:
the DLL3-VH comprises the amino acid sequence of SEQ ID NO: 52 or a variant thereof. In some embodiments, the variant comprises one or more amino acid substitutions selected from the group consisting of F27Y, S30T, I69L, R71V, D73K, and T93A in FR regions of SEQ ID NO: 52.

In some embodiments, provided is the antigen-binding molecule according to any one of the foregoing, wherein:
the DLL3-VL comprises the amino acid sequence of SEQ ID NO: 62 or a variant thereof. In some embodiments, the variant comprises one or more amino acid substitutions selected from the group consisting of F36L, A43S, P44F, S46G, T69A, F71Y, and T85D in FR regions of SEQ ID NO: 62.

In some embodiments, provided is the antigen-binding molecule according to any one of the foregoing, wherein:
the DLL3-VL comprises the amino acid sequence of SEQ ID NO: 68 or a variant thereof. In some embodiments, the variant comprises one or more amino acid substitutions selected from the group consisting of Y36L, Q42K, L46G, T69A, F71Y, E79Q, and V85D in FR regions of SEQ ID NO: 68.

In some embodiments, provided is the antigen-binding molecule according to any one of the foregoing, wherein:
the DLL3-VH comprises the amino acid sequence of SEQ ID NO: 78 or a variant thereof. In some embodiments, the variant comprises one or more amino acid substitutions in FR regions of SEQ ID NO: 78.

In some embodiments, provided is the antigen-binding molecule according to any one of the foregoing, wherein:
the DLL3-VL comprises the amino acid sequence of SEQ ID NO: 75 or a variant thereof. In some embodiments, the variant comprises one or more amino acid substitutions selected from the group consisting of K42G, P44V, and F71Y in FR regions of SEQ ID NO: 75.

In some embodiments, provided is the antigen-binding molecule according to any one of the foregoing, wherein:
the DLL3-VH comprises the amino acid sequence of SEQ ID NO: 95 or a variant thereof. In some embodiments, the variant comprises one or more amino acid substitutions selected from the group consisting of Q1E, A24T, R44S, R71V, and T73K in FR regions of SEQ ID NO: 95.

In some embodiments, provided is the antigen-binding molecule according to any one of the foregoing, wherein:
the DLL3-VL comprises the amino acid sequence of SEQ ID NO: 88 or a variant thereof. In some embodiments, the variant comprises one or more amino acid substitutions selected from the group consisting of DIN, D9K, P43S, G68A, and V85D in FR regions of SEQ ID NO: 88.

In some embodiments, provided is the antigen-binding molecule according to any one of the foregoing, wherein:
the DLL3-VH comprises the amino acid sequence of SEQ ID NO: 116 or a variant thereof. In some embodiments, the variant comprises one or more amino acid substitutions selected from the group consisting of Q1E, Q3R, V5Q, S7F, A9P, E10V, K12V, A40S, P41H, R44S, M48I, V67A, I69L, R71V, T73K, A75S, and R83T in FR regions of SEQ ID NO: 116.

In some embodiments, provided is the antigen-binding molecule according to any one of the foregoing, wherein:
the DLL3-VL comprises the amino acid sequence of SEQ ID NO: 100 or a variant thereof. In some embodiments, the variant comprises one or more amino acid substitutions selected from the group consisting of D9K, P43S, G68A, and Y87H in FR regions of SEQ ID NO: 100.

In some embodiments, provided is the antigen-binding molecule according to any one of the foregoing, wherein:
the DLL3-VL comprises the amino acid sequence of SEQ ID NO: 103 or a variant thereof. In some embodiments, the variant comprises one or more amino acid substitutions selected from the group consisting of A43S, S60D, and Q100S in FR regions of SEQ ID NO: 103.

In some embodiments, provided is the antigen-binding molecule according to any one of the foregoing, wherein:
the DLL3-VH comprises the amino acid sequence of SEQ ID NO: 56 or a variant thereof. In some embodiments, the variant comprises one or more amino acid substitutions in FR regions of SEQ ID NO: 56.

In some embodiments, provided is the antigen-binding molecule according to any one of the foregoing, wherein:
the DLL3-VH comprises the amino acid sequence of SEQ ID NO: 57 or a variant thereof. In some embodiments, the variant comprises one or more amino acid substitutions in FR regions of SEQ ID NO: 57.

In some embodiments, provided is the antigen-binding molecule according to any one of the foregoing, wherein:
the DLL3-VL comprises the amino acid sequence of SEQ ID NO: 63 or a variant thereof.

In some embodiments, the variant comprises one or more amino acid substitutions in FR regions of SEQ ID NO: 63; in some embodiments, the amino acid substitutions are one or more amino acid substitutions selected from the group consisting of A43S and P44F.

In some embodiments, provided is the antigen-binding molecule according to any one of the foregoing, wherein:
the DLL3-VL comprises the amino acid sequence of SEQ ID NO: 64 or a variant thereof. In some embodiments, the variant comprises one or more amino acid substitutions in FR regions of SEQ ID NO: 64; in some embodiments, the amino acid substitutions are one or more amino acid substitutions selected from the group consisting of A43S and P44F.

In some embodiments, provided is the antigen-binding molecule according to any one of the foregoing, wherein:
the DLL3-VL comprises the amino acid sequence of SEQ ID NO: 65 or a variant thereof. In some embodiments, the variant comprises one or more amino acid substitutions in FR regions of SEQ ID NO: 65; in some embodiments, the amino acid substitutions are one or more amino acid substitutions selected from the group consisting of A43S and P44F.

In some embodiments, provided is the antigen-binding molecule according to any one of the foregoing, wherein:
the DLL3-VL comprises the amino acid sequence of SEQ ID NO: 66 or a variant thereof. In some embodiments, the variant comprises one or more amino acid substitutions in FR regions of SEQ ID NO: 66; in some embodiments, the amino acid substitutions are one or more amino acid substitutions selected from the group consisting of A43S and P44F.

In some embodiments, provided is the antigen-binding molecule according to any one of the foregoing, wherein:
the DLL3-VL comprises the amino acid sequence of SEQ ID NO: 67 or a variant thereof. In some embodiments, the variant comprises one or more amino acid substitutions in FR regions of SEQ ID NO: 67.

In some embodiments, provided is the antigen-binding molecule according to any one of the foregoing, wherein:
the DLL3-VH comprises the amino acid sequence of SEQ ID NO: 79, 80, 81, or 82, or a variant of SEQ ID NO: 79, 80, 81, or 82. In some embodiments, the variant comprises one or more amino acid substitutions in FR regions of SEQ ID NO: 79, 80, 81, or 82; and
the DLL3-VL comprises the amino acid sequence of SEQ ID NO: 75 or a variant thereof. In some embodiments, the variant comprises one or more amino acid substitutions selected from the group consisting of K42G, P44V, and F71Y in FR regions of SEQ ID NO: 75.

In some embodiments, provided is the antigen-binding molecule according to any one of the foregoing, wherein:
the DLL3-VH comprises the amino acid sequence of SEQ ID NO: 91, 92, 93, or 94, or a variant of SEQ ID NO: 91, 92, 93, or 94. In some embodiments, the variant comprises one or more amino acid substitutions in FR regions of SEQ ID NO: 91, 92, 93, or 94; and
the DLL3-VL comprises the amino acid sequence of SEQ ID NO: 88 or a variant thereof. In some embodiments, the variant comprises one or more amino acid substitutions selected from the group consisting of DIN, D9K, P43S, G68A, and V85D in FR regions of SEQ ID NO: 88.

In some embodiments, provided is the antigen-binding molecule according to any one of the foregoing, wherein:
the DLL3-VH comprises the amino acid sequence of SEQ ID NO: 107, 108, 109, or 110, or a variant of SEQ ID NO: 107, 108, 109, or 110. In some embodiments, the variant comprises one or more amino acid substitutions in FR regions of SEQ ID NO: 107, 108, 109, or 110; in some embodiments, the amino acid substitutions are one or more amino acid substitutions selected from the group consisting of V5Q, S7F, A9P, E10V, K12V, A40S, P41H, M48I, V67A, A75S, and R83T.

In some embodiments, provided is the antigen-binding molecule according to any one of the foregoing, wherein:
the DLL3-VH comprises the amino acid sequence of SEQ ID NO: 112, or a variant of SEQ ID NO: 112. In some embodiments, the variant comprises one or more amino acid substitutions in FR regions of SEQ ID NO: 112; in some embodiments, the amino acid substitutions are one or more amino acid substitutions selected from the group consisting of V5Q, A9P, K12V, A40S, P41H, 4M8I, V67A, and R83T.

In some embodiments, provided is the antigen-binding molecule according to any one of the foregoing, wherein:
the DLL3-VH comprises the amino acid sequence of SEQ ID NO: 113, or a variant of SEQ ID NO: 113. In some embodiments, the variant comprises one or more amino acid substitutions in FR regions of SEQ ID NO: 113; in some embodiments, the amino acid substitutions are one or more amino acid substitutions selected from the group consisting of V5Q, S7F, A9P, E10V, K12V, A40S, P41H, A75S, and R83T.

In some embodiments, provided is the antigen-binding molecule according to any one of the foregoing, wherein:
the DLL3-VH comprises the amino acid sequence of SEQ ID NO: 114, or a variant of SEQ ID NO: 114. In some embodiments, the variant comprises one or more amino acid substitutions in FR regions of SEQ ID NO: 114; in some embodiments, the amino acid substitutions are one or more amino acid substitutions selected from the group consisting of V5Q, S7F, A9P, E10V, K12V, M48I, V67A, A75S, and R83T.

In some embodiments, provided is the antigen-binding molecule according to any one of the foregoing, wherein:
the DLL3-VH comprises the amino acid sequence of SEQ ID NO: 115, or a variant of SEQ ID NO: 115. In some embodiments, the variant comprises one or more amino acid substitutions in FR regions of SEQ ID NO: 115; in some embodiments, the amino acid substitutions are one or more amino acid substitutions selected from the group consisting of V5Q, A9P, K12V, A40S, P41H, M48I, and V67A.

In some embodiments, provided is the antigen-binding molecule according to any one of the foregoing, wherein:
the DLL3-VL comprises the amino acid sequence of SEQ ID NO: 100 or a variant thereof. In some embodiments, the variant comprises one or more amino acid substitutions selected from the group consisting of D9K, P43S, G68A, and Y87H in FR regions of SEQ ID NO: 100.

In some embodiments, provided is the antigen-binding molecule according to any one of the foregoing, wherein:
the DLL3-VL comprises the amino acid sequence of SEQ ID NO: 103 or a variant thereof. In some embodiments, the variant comprises one or more amino acid substitutions selected from the group consisting of A43S, S60D, and Q100S in FR regions of SEQ ID NO: 103.

In some embodiments, provided is the antigen-binding molecule according to any one of the foregoing, wherein:
i) the DLL3-VH comprises the amino acid sequence of SEQ ID NO: 12, 43, 44, 45, 46, 47, 48, 49, 50, 51, 52, 53, 54, 55, 56, or 57, and the DLL3-VL comprises the amino acid sequence of SEQ ID NO: 13, 58, 59, 60, 61, 62, 63, 64, 65, 66, 67, or 68; or
ii) the DLL3-VH comprises the amino acid sequence of SEQ ID NO: 14, 78, 79, 80, 81, or 82, and the DLL3-VL comprises the amino acid sequence of SEQ ID NO: 15, 75, 76, or 77; or
iii) the DLL3-VH comprises the amino acid sequence of SEQ ID NO: 16, 89, 90, 91, 92, 93, 94, or 95, and the DLL3-VL comprises the amino acid sequence of SEQ ID NO: 17, 86, 87, or 88; or
iv) the DLL3-VH comprises the amino acid sequence of SEQ ID NO: 18, 105, 106, 107, 108, 109, 110, 111, 112, 113, 114, 115, or 116, and the DLL3-VL comprises the amino acid sequence of SEQ ID NO: 19, 100, 101, 102, 103, or 104.

In some embodiments, provided is the antigen-binding molecule according to any one of the foregoing, wherein:
the DLL3-VH comprises the amino acid sequence of SEQ ID NO: 43, 44, or 45, and the DLL3-VL comprises the amino acid sequence of SEQ ID NO: 58, 59, or 60; or
the DLL3-VH comprises the amino acid sequence of SEQ ID NO: 47, 48, 49, 53, 54, or 55, and the DLL3-VL comprises the amino acid sequence of SEQ ID NO: 59; or
the DLL3-VH comprises the amino acid sequence of SEQ ID NO: 43, 44, or 45, and the DLL3-VL comprises the amino acid sequence of SEQ ID NO: 60; or
the DLL3-VH comprises the amino acid sequence of SEQ ID NO: 46, 48, 53, 54, or 55, and the DLL3-VL comprises the amino acid sequence of SEQ ID NO: 61; or
the DLL3-VH comprises the amino acid sequence of SEQ ID NO: 54, and the DLL3-VL comprises the amino acid sequence of SEQ ID NO: 63 or 64; or
the DLL3-VH comprises the amino acid sequence of SEQ ID NO: 56 or 57, and the DLL3-VL comprises the amino acid sequence of SEQ ID NO: 65, 66, or 67.

In some embodiments, provided is the antigen-binding molecule according to any one of the foregoing, wherein:
the DLL3-VH comprises the amino acid sequence of SEQ ID NO: 78 or 79, and the DLL3-VL comprises the amino acid sequence of SEQ ID NO: 75, 76, or 77; or
the DLL3-VH comprises the amino acid sequence of SEQ ID NO: 78, 79, 80, 81, or 82, and the DLL3-VL comprises the amino acid sequence of SEQ ID NO: 77.

In some embodiments, provided is the antigen-binding molecule according to any one of the foregoing, wherein:
the DLL3-VH comprises the amino acid sequence of SEQ ID NO: 89, 90, 91, 92, 93, or 94, and the DLL3-VL comprises the amino acid sequence of SEQ ID NO: 86 or 87; or
the DLL3-VH comprises the amino acid sequence of SEQ ID NO: 95, and the DLL3-VL comprises the amino acid sequence of SEQ ID NO: 87.

In some embodiments, provided is the antigen-binding molecule according to any one of the foregoing, wherein:
the DLL3-VH comprises the amino acid sequence of SEQ ID NO: 105, 106, 107, 108, 109, 110, or 111, and the DLL3-VL comprises the amino acid sequence of SEQ ID NO: 100, 101, or 102; or
the DLL3-VH comprises the amino acid sequence of SEQ ID NO: 112, 113, 114, or 115, and the DLL3-VL comprises the amino acid sequence of SEQ ID NO: 100, 103, or 104; or the DLL3-VH comprises the amino acid sequence of SEQ ID NO: 109, and the DLL3-VL comprises the amino acid sequence of SEQ ID NO: 103.

In some embodiments, provided is the antigen-binding molecule according to any one of the foregoing, wherein:
i) the DLL3-VH comprises the amino acid sequence of SEQ ID NO: 54, and the DLL3-VL comprises the amino acid sequence of SEQ ID NO: 63; or
the DLL3-VH comprises the amino acid sequence of SEQ ID NO: 54, and the DLL3-VL comprises the amino acid sequence of SEQ ID NO: 61; or
the DLL3-VH comprises the amino acid sequence of SEQ ID NO: 54, and the DLL3-VL comprises the amino acid sequence of SEQ ID NO: 64; or
the DLL3-VH comprises the amino acid sequence of SEQ ID NO: 56, and the DLL3-VL comprises the amino acid sequence of SEQ ID NO: 65; or
the DLL3-VH comprises the amino acid sequence of SEQ ID NO: 56, and the DLL3-VL comprises the amino acid sequence of SEQ ID NO: 66; or
the DLL3-VH comprises the amino acid sequence of SEQ ID NO: 56, and the DLL3-VL comprises the amino acid sequence of SEQ ID NO: 67; or
the DLL3-VH comprises the amino acid sequence of SEQ ID NO: 57, and the DLL3-VL comprises the amino acid sequence of SEQ ID NO: 65; or
the DLL3-VH comprises the amino acid sequence of SEQ ID NO: 57, and the DLL3-VL comprises the amino acid sequence of SEQ ID NO: 66; or
the DLL3-VH comprises the amino acid sequence of SEQ ID NO: 57, and the DLL3-VL comprises the amino acid sequence of SEQ ID NO: 67; or
ii) the DLL3-VH comprises the amino acid sequence of SEQ ID NO: 81, and the DLL3-VL comprises the amino acid sequence of SEQ ID NO: 77; or
iii) the DLL3-VH comprises the amino acid sequence of SEQ ID NO: 91, and the DLL3-VL comprises the amino acid sequence of SEQ ID NO: 87; or
iv) the DLL3-VH comprises the amino acid sequence of SEQ ID NO: 109, and the DLL3-VL comprises the amino acid sequence of SEQ ID NO: 100.

In some embodiments, provided is the antigen-binding molecule according to any one of the foregoing, wherein:
i) the DLL3-VH comprises the amino acid sequence of SEQ ID NO: 12, and the DLL3-VL comprises the amino acid sequence of SEQ ID NO: 13; or
ii) the DLL3-VH comprises the amino acid sequence of SEQ ID NO: 14, and the DLL3-VL comprises the amino acid sequence of SEQ ID NO: 15; or
iii) the DLL3-VH comprises the amino acid sequence of SEQ ID NO: 16, and the DLL3-VL comprises the amino acid sequence of SEQ ID NO: 17; or
iv) the DLL3-VH comprises the amino acid sequence of SEQ ID NO: 18, and the DLL3-VL comprises the amino acid sequence of SEQ ID NO: 19.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 63.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 61.

In some embodiments, provided is the antigen-binding molecule according to any one of the foregoing, wherein:
the DLL3-VH comprises the amino acid sequence of SEQ ID NO: 81, and the DLL3-VL comprises the amino acid sequence of SEQ ID NO: 77.

In some embodiments, provided is the antigen-binding molecule according to any one of the foregoing, wherein:
the DLL3-VH comprises the amino acid sequence of SEQ ID NO: 91, and the DLL3-VL comprises the amino acid sequence of SEQ ID NO: 87.

In some embodiments, provided is the antigen-binding molecule according to any one of the foregoing, wherein:
the DLL3-VH comprises the amino acid sequence of SEQ ID NO: 109, and the DLL3-VL comprises the amino acid sequence of SEQ ID NO: 100.

In some embodiments, provided is the antigen-binding molecule according to any one of the foregoing, wherein:
the CD3-VH comprises the amino acid sequence of SEQ ID NO: 136, and the CD3-VL comprises the amino acid sequence of SEQ ID NO: 137.

In some embodiments, provided is the antigen-binding molecule according to any one of the foregoing, wherein:
the CD3-VH comprises the amino acid sequence of SEQ ID NO: 138, and the CD3-VL comprises the amino acid sequence of SEQ ID NO: 137.

In some embodiments, provided is the antigen-binding molecule according to any one of the foregoing, wherein the antigen-binding moiety binding specifically to DLL3 or the antigen-binding moiety binding specifically to CD3 comprises a Titin chain and an Obscurin chain capable of forming a dimer.

In some embodiments, provided is the antigen-binding molecule according to any one of the foregoing, wherein the antigen-binding moiety binding specifically to DLL3 comprises a Titin chain and an Obscurin chain capable of forming a dimer.

In some embodiments, provided is the antigen-binding molecule according to any one of the foregoing, wherein the antigen-binding moiety binding specifically to CD3 comprises a Titin chain and an Obscurin chain capable of forming a dimer.

In some embodiments, provided is the antigen-binding molecule according to any one of the foregoing, wherein the Titin chain comprises an amino acid sequence selected from the group consisting of SEQ ID NO: 192 to SEQ ID NO: 210, and the Obscurin chain comprises an amino acid sequence selected from the group consisting of SEQ ID NO: 211 to SEQ ID NO: 251.

In some embodiments, provided is the antigen-binding molecule according to any one of the foregoing, wherein the Titin chain comprises the amino acid sequence of SEQ ID NO: 208.

In some embodiments, provided is the antigen-binding molecule according to any one of the foregoing, wherein the Obscurin chain comprises the amino acid sequence of SEQ ID NO: 246.

In some embodiments, provided is the antigen-binding molecule according to any one of the foregoing, wherein the Titin chain comprises the amino acid sequence of SEQ ID NO: 208, and the Obscurin chain comprises the amino acid sequence of SEQ ID NO: 246.

In some embodiments, provided is the antigen-binding molecule according to any one of the foregoing, wherein the antigen-binding molecule comprises an Fc region.

In some embodiments, provided is the antigen-binding molecule according to any one of the foregoing, wherein the antigen-binding molecule comprises a human IgG Fc region.

In some embodiments, provided is the antigen-binding molecule according to any one of the foregoing, wherein the antigen-binding molecule comprises a human IgG1 Fc region.

In some embodiments, provided is the antigen-binding molecule according to any one of the foregoing, wherein the antigen-binding molecule comprises an Fc region comprising one or more amino acid substitutions capable of reducing binding of the Fc region to an Fcγ receptor.

In some embodiments, provided is the antigen-binding molecule according to any one of the foregoing, wherein the antigen-binding molecule comprises an Fc region, and the Fc region is a human IgG1 Fc region and has amino acids A at positions 234 and 235, with numbering in accordance with the EU index.

In some embodiments, provided is the antigen-binding molecule according to any one of the foregoing, wherein the antigen-binding molecule comprises an Fc region comprising a first subunit Fc1 and a second subunit Fc2 capable of associating with each other, and the Fc1 and Fc2 each independently have one or more amino acid substitutions that reduce homodimerization of the Fc region.

In some embodiments, provided is the antigen-binding molecule according to any one of the foregoing, wherein the antigen-binding molecule comprises an Fc region comprising a first subunit Fc1 and a second subunit Fc2 capable of associating with each other; the Fc1 has a knob structure according to the knob-into-hole technique, and the Fc2 has a hole structure according to the knob-into-hole technique.

In some embodiments, provided is the antigen-binding molecule according to any one of the foregoing, wherein the antigen-binding molecule comprises an Fc region comprising a first subunit Fc1 and a second subunit Fc2 capable of associating with each other; the Fc1 has a knob structure according to the knob-into-hole technique, and the Fc2 has a hole structure according to the knob-into-hole technique; the Fc1 has an amino acid W at position 366, and the Fc2 has an amino acid S at position 366, an amino acid A at position 368, and an amino acid V at position 407, with numbering in accordance with the EU index.

In some embodiments, provided is the antigen-binding molecule according to any one of the foregoing, wherein the antigen-binding molecule comprises an Fc region comprising a first subunit Fc1 and a second subunit Fc2 capable of associating with each other; the Fc1 has a knob structure according to the knob-into-hole technique, and the Fc2 has a hole structure according to the knob-into-hole technique, wherein the Fc1 has an amino acid C at position 354 and an amino acid W at position 366, and the Fc2 has an amino acid C at position 349, an amino acid S at position 366, an amino acid A at position 368, and an amino acid V at position 407, with numbering in accordance with the EU index.

In some embodiments, provided is the antigen-binding molecule according to any one of the foregoing, wherein the antigen-binding molecule comprises an Fc region comprising a first subunit Fc1 and a second subunit Fc2 capable of associating with each other, wherein the Fc1 comprises the amino acid sequence of SEQ ID NO: 143, and the Fc2 comprises the amino acid sequence of SEQ ID NO: 144.

In some embodiments, provided is the antigen-binding molecule according to any one of the foregoing, wherein the antigen-binding molecule comprises an Fc region comprising a first subunit Fc1 and a second subunit Fc2 capable of associating with each other, wherein the Fc1 has an amino acid C at position 349, an amino acid W at position 366, and an amino acid A at position 237, and the Fc2 has an amino acid C at position 354, an amino acid S at position 366, an amino acid A at position 368, an amino acid V at position 407, and an amino acid A at position 237, with numbering in accordance with the EU index.

In some embodiments, provided is the antigen-binding molecule according to any one of the foregoing, wherein the antigen-binding molecule comprises an Fc region comprising a first subunit Fc1 and a second subunit Fc2 capable of associating with each other, wherein the Fc1 comprises the amino acid sequence of SEQ ID NO: 145, and the Fc2 comprises the amino acid sequence of SEQ ID NO: 146.

It should be understood that the ordinal numbers “first”, “second”, “formula a”, “formula b”, “Fc1”, “linker 3”, etc. in the present disclosure are used only to distinguish between different technical features, elements, components, or steps and are not intended to limit the level, order, or number.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3, wherein:
the antigen-binding moiety binding specifically to DLL3 is a Fab, and the antigen-binding moiety binding specifically to CD3 is a replaced Fab comprising a Titin chain and an Obscurin chain capable of forming a dimer.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3, wherein:
the antigen-binding moiety binding specifically to CD3 is a Fab, and the antigen-binding moiety binding specifically to DLL3 is a replaced Fab comprising a Titin chain and an Obscurin chain capable of forming a dimer.

In some embodiments, provided is the antigen-binding molecule according to any one of the foregoing, wherein the antigen-binding molecule comprises one first chain having a structure represented by formula (a), one second chain having a structure represented by formula (b), one third chain having a structure represented by formula (c), and one fourth chain having a structure represented by formula (d):

$$[ DLL 3 - VH ] - [ linker 1 ] - [ Titin chain ] / [ Obscurin chain ] - [ Fc 2 ] / [ Fc 1 ] , \qquad ( a )$$

$$[ DLL 3 - VL ] - [ linker 2 ] - [ Obscurin chain ] / [ Titin chain ] , \qquad ( b )$$

$$[ CD 3 - VH ] - [ CH 1 ] - [ Fc 1 ] / [ Fc 2 ] , and \qquad ( c )$$

$$[ CD 3 - VL ] - [ CL ] . \qquad ( d )$$

wherein the linker 1 and the linker 2 are identical or different peptide linkers; the structures represented by formulas (a), (b), (c), and (d) are arranged from N-terminus to C-terminus. “/” means “or”; that is, the Fc1 and Fc2 can be interchanged, and the Titin chain and Obscurin chain can be interchanged.

In some embodiments, provided is the antigen-binding molecule according to any one of the foregoing, wherein the antigen-binding molecule comprises one first chain having a structure represented by formula (a), one second chain having a structure represented by formula (b), one third chain having a structure represented by formula (c), and one fourth chain having a structure represented by formula (d):

$$[ DLL 3 - VH ] - [ linker 1 ] - [ Titin chain ] - [ Fc 2 ] , \qquad ( a )$$

$$[ DLL 3 - VL ] - [ linker 2 ] - [ Obscurin chain ] , \qquad ( b )$$

$$[ CD 3 - VH ] - [ CH 1 ] - [ Fc 1 ] , and \qquad ( c )$$

$$[ CD 3 - VL ] - [ CL ] , \qquad ( d )$$

wherein the linker 1 and the linker 2 are identical or different peptide linkers; the structures represented by formulas (a), (b), (c), and (d) are arranged from N-terminus to C-terminus; the Fc1 and Fc2 can be interchanged, and the Titin chain and Obscurin chain can be interchanged.

In some embodiments, provided is the antigen-binding molecule according to any one of the foregoing, wherein the antigen-binding molecule comprises one first chain having a structure represented by formula (a), one second chain having a structure represented by formula (b), one third chain having a structure represented by formula (c), and one fourth chain having a structure represented by formula (d):

$$[ DLL 3 - VH ] - [ linker 1 ] - [ Titin chain ] - [ Fc 2 ] , \qquad ( a )$$

$$[ DLL 3 - VL ] - [ linker 2 ] - [ Obscurin chain ] , \qquad ( b )$$

$$[ CD 3 - VH ] - [ CH 1 ] - [ Fc 1 ] , and \qquad ( c )$$

$$[ CD 3 - VL ] - [ CL ] , \qquad ( d )$$

wherein the linker 1 and the linker 2 are identical or different peptide linkers; the structures represented by formulas (a), (b), (c), and (d) are arranged from N-terminus to C-terminus.

In some embodiments, provided is the antigen-binding molecule according to any one of the foregoing, wherein the antigen-binding molecule comprises one first chain having a structure represented by formula (a′), one second chain having a structure represented by formula (b′), one third chain having a structure represented by formula (c′), and one fourth chain having a structure represented by formula (d′):

$$[ CD 3 - VH ] - [ linker 1 ] - [ Titin chain ] / Obscurin chain ] - [ Fc 2 ] / [ Fc 1 ] , \qquad ( {a}^{′} )$$

$$[ CD 3 - VL ] - [ linker 2 ] - [ Obscurin chain ] / [ Titin chain ] , \qquad ( {b}^{′} )$$

$$[ DLL 3 - VH ] - [ CH 1 ] - [ Fc 1 ] / [ Fc 2 ] , and \qquad ( {c}^{′} )$$

$$[ DLL 3 - VL ] - [ CL ] . \qquad ( {d}^{′} )$$

wherein the linker 1 and the linker 2 are absent; that is, both the linker 1 and the linker 2 are peptide bonds; the structures represented by formulas (a′), (b′), (c′), and (d′) are arranged from N-terminus to C-terminus. “/” means “or”; that is, the Fc1 and Fc2 can be interchanged, and the Titin chain and Obscurin chain can be interchanged.

In some embodiments, provided is the antigen-binding molecule according to any one of the foregoing, wherein the antigen-binding molecule comprises one first chain having a structure represented by formula (a′), one second chain having a structure represented by formula (b′), one third chain having a structure represented by formula (c′), and one fourth chain having a structure represented by formula (d′):

$$[ CD 3 - VH ] - [ linker 1 ] - [ Titin chain ] - [ Fc 2 ] , \qquad ( {a}^{′} )$$

$$[ CD 3 - VL ] - [ linker 2 ] - [ Obscurin chain ] , \qquad ( {b}^{′} )$$

$$[ DLL 3 - VH ] - [ CH 1 ] - [ Fc 1 ] , and \qquad ( {c}^{′} )$$

$$[ DLL 3 - VL ] - [ CL ] . \qquad ( {d}^{′} )$$

wherein the linker 1 and the linker 2 are absent; that is, both the linker 1 and the linker 2 are peptide bonds; the structures represented by formulas (a′), (b′), (c′), and (d′) are arranged from N-terminus to C-terminus; the Fc1 and Fc2 can be interchanged, and the Titin chain and Obscurin chain can be interchanged.

In some embodiments, provided is the antigen-binding molecule according to any one of the foregoing, wherein the antigen-binding molecule comprises one first chain having a structure represented by formula (a), one second chain having a structure represented by formula (b), one third chain having a structure represented by formula (c), and one fourth chain having a structure represented by formula (d):

$$[ CD 3 - VH ] - [ linker 1 ] - [ Titin chain ] - [ Fc 2 ] , \qquad ( {a}^{′} )$$

$$[ CD 3 - VL ] - [ linker 2 ] - [ Obscurin chain ] , \qquad ( {b}^{′} )$$

$$[ DLL 3 - VH ] - [ CH 1 ] - [ Fc 1 ] , and \qquad ( {c}^{′} )$$

$$[ DLL 3 - VL ] - [ CL ] . \qquad ( {d}^{′} )$$

wherein the linker 1 and the linker 2 are absent; that is, both the linker 1 and the linker 2 are peptide bonds; the structures represented by formulas (a), (b), (c), and (d) are arranged from N-terminus to C-terminus.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; and
the CD3-VH comprises the amino acid sequence of SEQ ID NO: 136, and the CD3-VL comprises the amino acid sequence of SEQ ID NO: 137.

In some embodiments, provided is the antigen-binding molecule according to any one of the foregoing, wherein:
the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 61; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 63; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 63; and the CD3-VH comprises the amino acid sequence of SEQ ID NO: 136, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 81, and the DLL3-VL comprises the amino acid sequence of SEQ ID NO: 77; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 91, and the DLL3-VL comprises the amino acid sequence of SEQ ID NO: 87; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 109, and the DLL3-VL comprises the amino acid sequence of SEQ ID NO: 100; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 91, and the DLL3-VL comprises the amino acid sequence of SEQ ID NO: 87; and the CD3-VH comprises the amino acid sequence of SEQ ID NO: 136, and the CD3-VL comprises the amino acid sequence of SEQ ID NO: 137.

In some embodiments, provided is the antigen-binding molecule according to any one of the foregoing, wherein the Titin chain and the Obscurin chain are any Titin chain and any Obscurin chain from Tables 3-1 and 3-2 in the present disclosure that can form a dimer. In some embodiments, provided is the antigen-binding molecule according to any one of the foregoing, wherein the Titin chain comprises the amino acid sequence of SEQ ID NO: 208. In some embodiments, provided is the antigen-binding molecule according to any one of the foregoing, wherein the Obscurin chain comprises the amino acid sequence of SEQ ID NO: 246.

In some embodiments, provided is the antigen-binding molecule according to any one of the foregoing, wherein both the linker 1 and the linker 2 are peptide linkers known in the art, as long as the antigen-binding molecule is capable of exhibiting the desired antigen binding activity. For example, the peptide linkers may be flexible peptides having 1-50 or 3-20 amino acid residues. In some embodiments, the peptide linkers are 3-15 amino acid residues in length. In some embodiments, the peptide linker 1 and linker 2 each independently have a structure of (GGGGS)n, wherein n is 1, 2, or 3. In some embodiments, the sequences of the linker 1 and linker 2 are both GGGGS (SEQ ID NO: 147).

In some embodiments, provided is the antigen-binding molecule according to any one of the foregoing, wherein the CH1 is a CH1 sequence of an IgG. In some embodiments, the CH1 is the CH1 of IgG1. In some embodiments, the CH1 comprises the amino acid sequence of SEQ ID NO: 141.

In some embodiments, provided is the antigen-binding molecule according to any one of the foregoing, wherein the CL is a light chain constant region of an antibody. In some embodiments, provided is the antigen-binding molecule according to any one of the foregoing, wherein the CL is a light chain constant region of a kappa chain or a lambda chain. In some embodiments, the CL comprises the amino acid sequence of SEQ ID NO: 142.

In some embodiments, provided is the antigen-binding molecule according to any one of the foregoing, wherein the antigen-binding molecule comprises:
one first chain comprising the amino acid sequence of SEQ ID NO: 171, one second chain comprising the amino acid sequence of SEQ ID NO: 172, one third chain comprising the amino acid sequence of SEQ ID NO: 154, and one fourth chain comprising the amino acid sequence of SEQ ID NO: 155; or
one first chain comprising the amino acid sequence of SEQ ID NO: 171, one second chain comprising the amino acid sequence of SEQ ID NO: 172, one third chain comprising the amino acid sequence of SEQ ID NO: 156, and one fourth chain comprising the amino acid sequence of SEQ ID NO: 155; or
one first chain comprising the amino acid sequence of SEQ ID NO: 152, one second chain comprising the amino acid sequence of SEQ ID NO: 153, one third chain comprising the amino acid sequence of SEQ ID NO: 154, and one fourth chain comprising the amino acid sequence of SEQ ID NO: 155; or
one first chain comprising the amino acid sequence of SEQ ID NO: 152, one second chain comprising the amino acid sequence of SEQ ID NO: 153, one third chain comprising the amino acid sequence of SEQ ID NO: 156, and one fourth chain comprising the amino acid sequence of SEQ ID NO: 155; or
one first chain comprising the amino acid sequence of SEQ ID NO: 164, one second chain comprising the amino acid sequence of SEQ ID NO: 165, one third chain comprising the amino acid sequence of SEQ ID NO: 154, and one fourth chain comprising the amino acid sequence of SEQ ID NO: 155; or
one first chain comprising the amino acid sequence of SEQ ID NO: 164, one second chain comprising the amino acid sequence of SEQ ID NO: 165, one third chain comprising the amino acid sequence of SEQ ID NO: 156, and one fourth chain comprising the amino acid sequence of SEQ ID NO: 155; or
one first chain comprising the amino acid sequence of SEQ ID NO: 178, one second chain comprising the amino acid sequence of SEQ ID NO: 179, one third chain comprising the amino acid sequence of SEQ ID NO: 154, and one fourth chain comprising the amino acid sequence of SEQ ID NO: 155; or
one first chain comprising the amino acid sequence of SEQ ID NO: 178, one second chain comprising the amino acid sequence of SEQ ID NO: 179, one third chain comprising the amino acid sequence of SEQ ID NO: 156, and one fourth chain comprising the amino acid sequence of SEQ ID NO: 155.

In some embodiments, provided is the antigen-binding molecule according to any one of the foregoing, wherein the antigen-binding molecule comprises: one first chain comprising the amino acid sequence of SEQ ID NO: 171, one second chain comprising the amino acid sequence of SEQ ID NO: 172, one third chain comprising the amino acid sequence of SEQ ID NO: 154, and one fourth chain comprising the amino acid sequence of SEQ ID NO: 155.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3; the antigen-binding moiety binding specifically to DLL3 is a Fab, and the antigen-binding moiety binding specifically to CD3 is an scFv. In some embodiments, provided is the antigen-binding molecule according to any one of the foregoing, wherein the antigen-binding molecule comprises one first chain having a structure represented by formula (e), one second chain having a structure represented by formula (f), and one third chain having a structure represented by formula (g):

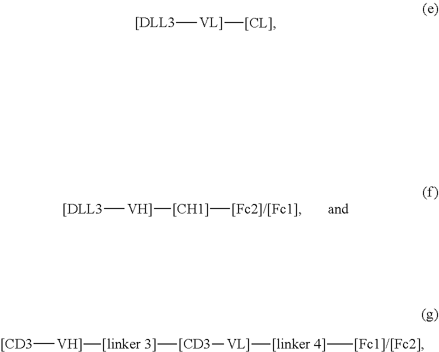

wherein the linker 3 and the linker 4 are identical or different peptide linkers;
the structures represented by formulas (e), (f), and (g) are arranged from N-terminus to C-terminus. “/” means “or”; that is, the Fc1 and Fc2 can be interchanged.

In some embodiments, provided is the antigen-binding molecule according to any one of the foregoing, wherein the antigen-binding molecule comprises one first chain having a structure represented by formula (e), one second chain having a structure represented by formula (f), and one third chain having a structure represented by formula (g):

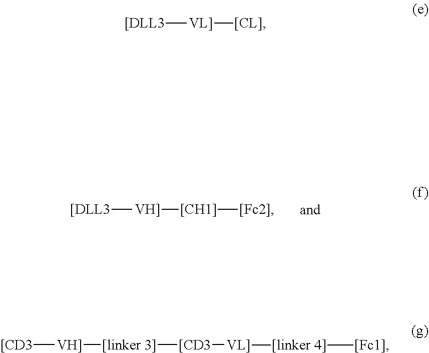

wherein the linker 3 and the linker 4 are identical or different peptide linkers;
the structures represented by formulas (e), (f), and (g) are arranged from N-terminus to C-terminus; the Fc1 and Fc2 can be interchanged.

In some embodiments, provided is the antigen-binding molecule according to any one of the foregoing, wherein the antigen-binding molecule comprises one first chain having a structure represented by formula (e), one second chain having a structure represented by formula (f), and one third chain having a structure represented by formula (g):

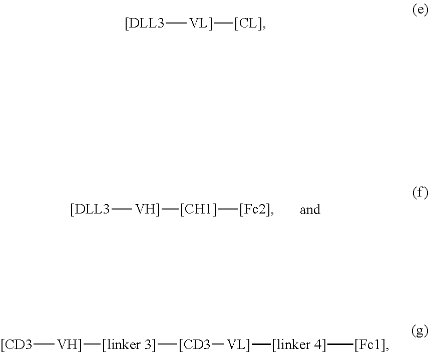

wherein the linker 3 and the linker 4 are identical or different peptide linkers;
the structures represented by formulas (e), (f), and (g) are arranged from N-terminus to C-terminus.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3; the antigen-binding moiety binding specifically to DLL3 is an scFv, and the antigen-binding moiety binding specifically to CD3 is a Fab.

In some embodiments, provided is the antigen-binding molecule according to any one of the foregoing, wherein the antigen-binding molecule comprises one first chain having a structure represented by formula (e′), one second chain having a structure represented by formula (f′), and one third chain having a structure represented by formula (g′):

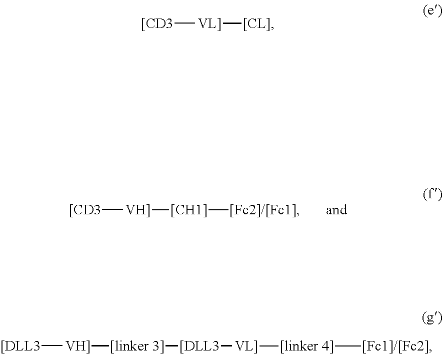

wherein the linker 3 and the linker 4 are identical or different peptide linkers;
the structures represented by formulas (e′), (f′), and (g′) are arranged from N-terminus to C-terminus. “/” means “or”.

In some embodiments, provided is the antigen-binding molecule according to any one of the foregoing, wherein the antigen-binding molecule comprises one first chain having a structure represented by formula (e′), one second chain having a structure represented by formula (f′), and one third chain having a structure represented by formula (g′):

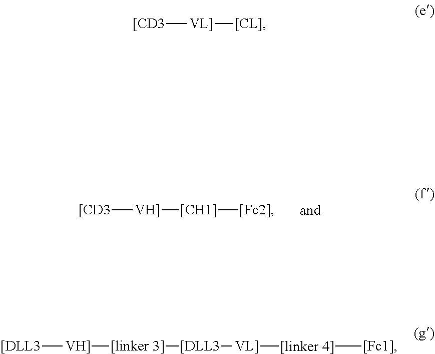

wherein the linker 3 and the linker 4 are identical or different peptide linkers;
the structures represented by formulas (e′), (f′), and (g′) are arranged from N-terminus to C-terminus; the Fc1 and Fc2 can be interchanged.

In some embodiments, provided is the antigen-binding molecule according to any one of the foregoing, wherein the antigen-binding molecule comprises one first chain having a structure represented by formula (e′), one second chain having a structure represented by formula (f), and one third chain having a structure represented by formula (g′):

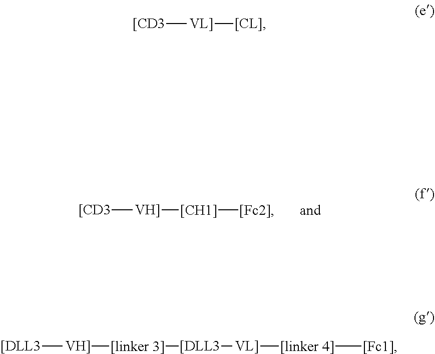

wherein the linker 3 and the linker 4 are identical or different peptide linkers;
the structures represented by formulas (e′), (f′), and (g′) are arranged from N-terminus to C-terminus.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 61; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 63; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 91, and the DLL3-VL comprises the amino acid sequence of SEQ ID NO: 87; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 81, and the DLL3-VL comprises the amino acid sequence of SEQ ID NO: 77; and the CD3-VH comprises the amino acid sequence of SEQ ID NO: 136 or 138, and the CD3-VL comprises the amino acid sequence of SEQ ID NO: 137.
   In some embodiments, provided is the antigen-binding molecule according to any one of the foregoing, wherein:
the DLL3-VH comprises the amino acid sequence of SEQ ID NO: 109, and the DLL3-VL comprises the amino acid sequence of SEQ ID NO: 100; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 91, and the DLL3-VL comprises the amino acid sequence of SEQ ID NO: 87; and the CD3-VH comprises the amino acid sequence of SEQ ID NO: 138, and the CD3-VL comprises the amino acid sequence of SEQ ID NO: 137.

In some embodiments, provided is the antigen-binding molecule according to any one of the foregoing, wherein both the linker 3 and the linker 4 are peptide linkers known in the art, as long as the antigen-binding molecule is capable of exhibiting the desired antigen binding activity. For example, the peptide linkers may be flexible peptides having 1-50 or 3-20 amino acid residues. In some embodiments, the peptide linkers are 3-15 amino acid residues in length. In some embodiments, the peptide linker 3 and linker 4 each independently have a structure of (GGGGS)n, wherein n is 1-5. In some embodiments, n is 1, 2, 3, or 4. In some embodiments, the sequence of the linker 3 is GGGGSGGGGSGGGGS (SEQ ID NO: 148). In some embodiments, the sequence of the linker 4 is GGGGS (SEQ ID NO: 147).

In some embodiments, provided is the antigen-binding molecule according to any one of the foregoing, wherein the CH1 is a CH1 sequence of an IgG. In some embodiments, the CH1 is the CH1 of IgG1. In some embodiments, the CH1 comprises the amino acid sequence of SEQ ID NO: 141.

In some embodiments, provided is the antigen-binding molecule according to any one of the foregoing, wherein the CL is a light chain constant region of an antibody. In some embodiments, provided is the antigen-binding molecule according to any one of the foregoing, wherein the CL is a light chain constant region of a kappa chain or a lambda chain. In some embodiments, the CL comprises the amino acid sequence of SEQ ID NO: 142.

In some embodiments, provided is the antigen-binding molecule according to any one of the foregoing, wherein the antigen-binding molecule comprises:
one first chain comprising the amino acid sequence of SEQ ID NO: 173, one second chain comprising the amino acid sequence of SEQ ID NO: 174, and one third chain comprising the amino acid sequence of SEQ ID NO: 160; or
one first chain comprising the amino acid sequence of SEQ ID NO: 173, one second chain comprising the amino acid sequence of SEQ ID NO: 174, and one third chain comprising the amino acid sequence of SEQ ID NO: 159; or
one first chain comprising the amino acid sequence of SEQ ID NO: 157, one second chain comprising the amino acid sequence of SEQ ID NO: 158, and one third chain comprising the amino acid sequence of SEQ ID NO: 159; or
one first chain comprising the amino acid sequence of SEQ ID NO: 157, one second chain comprising the amino acid sequence of SEQ ID NO: 158, and one third chain comprising the amino acid sequence of SEQ ID NO: 160; or
one first chain comprising the amino acid sequence of SEQ ID NO: 166, one second chain comprising the amino acid sequence of SEQ ID NO: 167, and one third chain comprising the amino acid sequence of SEQ ID NO: 159; or
one first chain comprising the amino acid sequence of SEQ ID NO: 166, one second chain comprising the amino acid sequence of SEQ ID NO: 167, and one third chain comprising the amino acid sequence of SEQ ID NO: 160; or
one first chain comprising the amino acid sequence of SEQ ID NO: 180, one second chain comprising the amino acid sequence of SEQ ID NO: 181, and one third chain comprising the amino acid sequence of SEQ ID NO: 159; or
one first chain comprising the amino acid sequence of SEQ ID NO: 180, one second chain comprising the amino acid sequence of SEQ ID NO: 181, and one third chain comprising the amino acid sequence of SEQ ID NO: 160.

In some embodiments, provided is the antigen-binding molecule according to any one of the foregoing, wherein the antigen-binding molecule comprises:
one first chain comprising the amino acid sequence of SEQ ID NO: 173, one second chain comprising the amino acid sequence of SEQ ID NO: 174, and one third chain comprising the amino acid sequence of SEQ ID NO: 160.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3;
the antigen-binding molecule comprises one first chain having a structure represented by formula (h) and one second chain having a structure represented by formula (i):

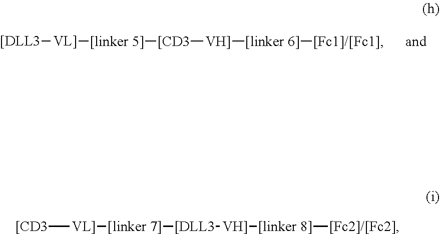

wherein the linker 5, linker 6, linker 7, and linker 8 are identical or different peptide linkers;
the structures represented by formulas (h) and (i) are arranged from N-terminus to C-terminus. “/” means “or”; that is, the Fc1 and Fc2 can be interchanged.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3;
the antigen-binding molecule comprises one first chain having a structure represented by formula (h) and one second chain having a structure represented by formula (i):

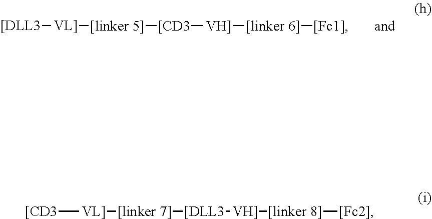

wherein the linker 5, linker 6, linker 7, and linker 8 are identical or different peptide linkers;
the structures represented by formulas (h) and (i) are arranged from N-terminus to C-terminus; the Fc1 and Fc2 can be interchanged.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3;
the antigen-binding molecule comprises one first chain having a structure represented by formula (h) and one second chain having a structure represented by formula (i):

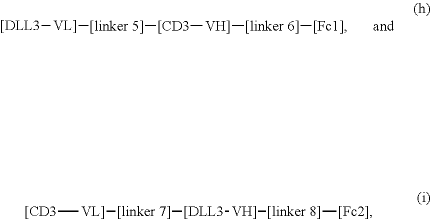

wherein the linker 5, linker 6, linker 7, and linker 8 are identical or different peptide linkers;
the structures represented by formulas (h) and (i) are arranged from N-terminus to C-terminus.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3;
the antigen-binding molecule comprises one first chain having a structure represented by formula (h′) and one second chain having a structure represented by formula (i′):

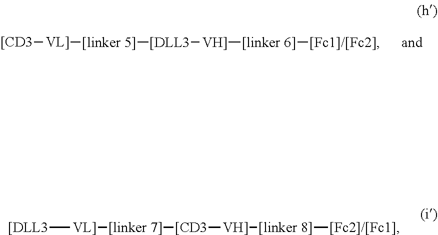

wherein the linker 5, linker 6, linker 7, and linker 8 are identical or different peptide linkers;
the structures represented by formulas (h′) and (i′) are arranged from N-terminus to C-terminus. “/” means “or”.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3;
the antigen-binding molecule comprises one first chain having a structure represented by formula (h′) and one second chain having a structure represented by formula (i′):

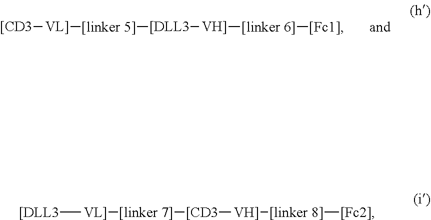

wherein the linker 5, linker 6, linker 7, and linker 8 are identical or different peptide linkers;
the structures represented by formulas (h′) and (i′) are arranged from N-terminus to C-terminus; the Fc1 and Fc2 can be interchanged.

In some embodiments, provided is the antigen-binding molecule according to any one of the foregoing, wherein the antigen-binding molecule comprises one antigen-binding moiety binding specifically to DLL3 and one antigen-binding moiety binding specifically to CD3;
the antigen-binding molecule comprises one first chain having a structure represented by formula (h′) and one second chain having a structure represented by formula (i′):

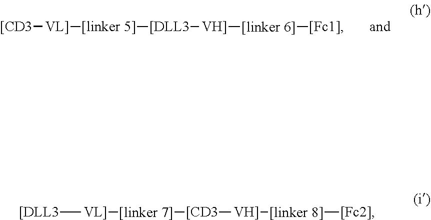

wherein the linker 5, linker 6, linker 7, and linker 8 are identical or different peptide linkers;
   the structures represented by formulas (h′) and (i′) are arranged from N-terminus to C-terminus.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131 or 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; and
the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 61; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 63; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 64; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 56, and the DLL3-VL comprises the amino acid sequence of SEQ ID NO: 65; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 56, and the DLL3-VL comprises the amino acid sequence of SEQ ID NO: 66; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 56, and the DLL3-VL comprises the amino acid sequence of SEQ ID NO: 67; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 57, and the DLL3-VL comprises the amino acid sequence of SEQ ID NO: 65; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 57, and the DLL3-VL comprises the amino acid sequence of SEQ ID NO: 66; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 57, and the DLL3-VL comprises the amino acid sequence of SEQ ID NO: 67; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 91, and the DLL3-VL comprises the amino acid sequence of SEQ ID NO: 87; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 81, and the DLL3-VL comprises the amino acid sequence of SEQ ID NO: 77; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 109, and the DLL3-VL comprises the amino acid sequence of SEQ ID NO: 100; and the CD3-VH comprises the amino acid sequence of SEQ ID NO: 136 or 138, and the CD3-VL comprises the amino acid sequence of SEQ ID NO: 137.

In some embodiments, provided is the antigen-binding molecule according to any one of the foregoing, wherein:
the DLL3-VH comprises the amino acid sequence of SEQ ID NO: 54, and the DLL3-VL comprises the amino acid sequence of SEQ ID NO: 63; and the CD3-VH comprises the amino acid sequence of SEQ ID NO: 136, and the CD3-VL comprises the amino acid sequence of SEQ ID NO: 137;
In some embodiments, provided is the antigen-binding molecule according to any one of the foregoing, wherein the linker 5, linker 6, linker 7, and linker 8 are all peptide linkers known in the art, as long as the antigen-binding molecule is capable of exhibiting the desired antigen binding activity. For example, the peptide linkers may be flexible peptides comprising 1-50 or 3-20 amino acid residues. In some embodiments, the peptide linkers are 1-15 amino acid residues in length. In some embodiments, the linker 5 has a structure of (GGGS)nGm, wherein n is 1-5, preferably 1, 2, or 3; m is 1-10, preferably 4, 5, 6, 7, or 8. In some embodiments, the sequence of the linker 5 is GGGSGGGG (SEQ ID NO: 149). In some embodiments, the linker 6 and linker 8 have a structure of Gm, wherein m is 1-5, preferably 1, 2, or 3. In some embodiments, the sequences of linker 6 and linker 8 are G (SEQ ID NO: 150).

In some embodiments, the linker 7 has a structure of (GGGGS)nGm, wherein n is 1-5, preferably 1, 2, or 3; m is 1-10, preferably 4, 5, 6, 7, or 8. In some embodiments, the sequence of linker 7 is GGGGSGGGG (SEQ ID NO: 151).

In some embodiments, provided is the antigen-binding molecule according to any one of the foregoing, wherein the antigen-binding molecule comprises:
one first chain comprising the amino acid sequence of SEQ ID NO: 185 and one second chain comprising the amino acid sequence of SEQ ID NO: 162; or
one first chain comprising the amino acid sequence of SEQ ID NO: 161 and one second chain comprising the amino acid sequence of SEQ ID NO: 162; or
one first chain comprising the amino acid sequence of SEQ ID NO: 163 and one second chain comprising the amino acid sequence of SEQ ID NO: 162; or
one first chain comprising the amino acid sequence of SEQ ID NO: 259 and one second chain comprising the amino acid sequence of SEQ ID NO: 162; or
one first chain comprising the amino acid sequence of SEQ ID NO: 260 and one second chain comprising the amino acid sequence of SEQ ID NO: 162; or
one first chain comprising the amino acid sequence of SEQ ID NO: 168 and one second chain comprising the amino acid sequence of SEQ ID NO: 169; or
one first chain comprising the amino acid sequence of SEQ ID NO: 170 and one second chain comprising the amino acid sequence of SEQ ID NO: 169; or
one first chain comprising the amino acid sequence of SEQ ID NO: 175 and one second chain comprising the amino acid sequence of SEQ ID NO: 176; or
one first chain comprising the amino acid sequence of SEQ ID NO: 177 and one second chain comprising the amino acid sequence of SEQ ID NO: 176; or
one first chain comprising the amino acid sequence of SEQ ID NO: 182 and one second chain comprising the amino acid sequence of SEQ ID NO: 183; or
one first chain comprising the amino acid sequence of SEQ ID NO: 184 and one second chain comprising the amino acid sequence of SEQ ID NO: 183; or
one first chain comprising the amino acid sequence of SEQ ID NO: 186 and one second chain comprising the amino acid sequence of SEQ ID NO: 162; or
one first chain comprising the amino acid sequence of SEQ ID NO: 187 and one second chain comprising the amino acid sequence of SEQ ID NO: 188; or
one first chain comprising the amino acid sequence of SEQ ID NO: 187 and one second chain comprising the amino acid sequence of SEQ ID NO: 189; or
one first chain comprising the amino acid sequence of SEQ ID NO: 190 and one second chain comprising the amino acid sequence of SEQ ID NO: 188; or
one first chain comprising the amino acid sequence of SEQ ID NO: 190 and one second chain comprising the amino acid sequence of SEQ ID NO: 189; or
one first chain comprising the amino acid sequence of SEQ ID NO: 191 and one second chain comprising the amino acid sequence of SEQ ID NO: 188; or
one first chain comprising the amino acid sequence of SEQ ID NO: 191 and one second chain comprising the amino acid sequence of SEQ ID NO: 189.

In some embodiments, provided is the antigen-binding molecule according to any one of the foregoing, wherein the antigen-binding molecule comprises: one first chain comprising the amino acid sequence of SEQ ID NO: 185 and one second chain comprising the amino acid sequence of SEQ ID NO: 162.

In another aspect, the present disclosure also provides an anti-DLL3 antibody capable of binding specifically to DLL3, the anti-DLL3 antibody comprising a heavy chain variable region DLL3-VH and a light chain variable region DLL3-VL, wherein:
(i) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 12, 56, or 57, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 13, 63, 64, 65, 66, or 67, respectively; or
(ii) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 14, 79, 80, 81, or 82, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 15, respectively; or
(iii) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 16, 91, 92, 93, or 94, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 17, respectively; or
(iv) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 18, 107, 108, 109, 110, 112, 113, 114, or 115, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 19, respectively.

In some embodiments, provided is the anti-DLL3 antibody according to any one of the foregoing, wherein:
(i) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 12, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 13, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 54, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 64, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 54, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 63, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 56, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 65, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 56, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 66, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 56, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 67, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 57, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 65, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 57, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 66, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 57, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 67, respectively; or
(ii) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 14, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 15, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 81, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 77, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 79, 80, or 82, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 77, respectively; or
(iii) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 16, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 17, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 91, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 87, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 92, 93, or 94, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 87, respectively; or
(iv) a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 18, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 19, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 109, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 100, respectively; or
a DLL3-HCDR1, a DLL3-HCDR2, and a DLL3-HCDR3 in the DLL3-VH comprise the amino acid sequences of a DLL3-HCDR1, a DLL3-HCDR2, and a DLL3-HCDR3 in SEQ ID NO: 107, 108, 110, 112, 113, 114, or 115, respectively, and a DLL3-LCDR1, a DLL3-LCDR2, and a DLL3-LCDR3 in the DLL3-VL comprise the amino acid sequences of a DLL3-LCDR1, a DLL3-LCDR2, and a DLL3-LCDR3 in SEQ ID NO: 100, respectively.

In some embodiments, the DLL3-HCDR1, DLL3-HCDR2, DLL3-HCDR3, DLL3-LCDR1, DLL3-LCDR2, and DLL3-LCDR3 are defined according to the Kabat, IMGT, Chothia, AbM, or Contact numbering scheme.

In some embodiments, provided is the anti-DLL3 antibody according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 69 or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25;
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
ii) the DLL3-VH comprises: a DLL3-HCDR1 comprising the amino acid sequence of SEQ ID NO: 26 or 84, a DLL3-HCDR2 comprising the amino acid sequence of SEQ ID NO: 27 or 85, and a DLL3-HCDR3 comprising the amino acid sequence of SEQ ID NO: 28 or 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
iii) the DLL3-VH comprises: a DLL3-HCDR1 comprising the amino acid sequence of SEQ ID NO: 20, a DLL3-HCDR2 comprising the amino acid sequence of SEQ ID NO: 32, 96, 97, 98, or 99, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; or
iv) the DLL3-VH comprises: a DLL3-HCDR1 comprising the amino acid sequence of SEQ ID NO: 20, a DLL3-HCDR2 comprising the amino acid sequence of SEQ ID NO: 37, 117, 118, 119, or 120, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40.

In some embodiments, provided is the anti-DLL3 antibody according to any one of the foregoing, wherein:
i) the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74.

In some embodiments, provided is the anti-DLL3 antibody according to any one of the foregoing, wherein:
i) the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
ii) the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 84, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 84, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 28; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
iii) the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; or
iv) the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40.

In some embodiments, provided is the anti-DLL3 antibody according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25.

In some embodiments, provided is the anti-DLL3 antibody according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25.

In some embodiments, provided is the anti-DLL3 antibody according to any one of the foregoing, wherein:
the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31.

In some embodiments, provided is the anti-DLL3 antibody according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36.

In some embodiments, provided is the anti-DLL3 antibody according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40.

In some embodiments, in the anti-DLL3 antibody according to any one of the foregoing, the DLL3-HCDR1, DLL3-HCDR2, DLL3-HCDR3, DLL3-LCDR1, DLL3-LCDR2, and DLL3-LCDR3 are defined according to the Kabat numbering scheme.

In some embodiments, the anti-DLL3 antibody according to any one of the foregoing binds to human DLL3 at 25° C. with a KD of less than 4×10⁻⁹ M, 3×10⁻⁹ M, 2×10-9 M, 1×10⁻⁹ M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M, 3×10⁻¹⁰ M, 2×10⁻¹⁰ M, 1×10⁻¹⁰ M, or 9×10⁻¹¹ M, as measured by surface plasmon resonance.

In some embodiments, provided is the anti-DLL3 antibody according to the foregoing, wherein:
(i) the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and
the DLL3-VL comprises: a DLL3-LCDR1 comprising the amino acid sequence of SEQ ID NO: 23, a DLL3-LCDR2 comprising the amino acid sequence of SEQ ID NO: 24, 71, 72, or 73, and a DLL3-LCDR3 comprising the amino acid sequence of SEQ ID NO: 25 or 74, and FR regions of the DLL3-VL comprise one or more amino acid substitutions selected from the group consisting of 1N, 36L, 42K, 43S, 44F, 46G, 69A, 71Y, 79Q, and 85D; or
(ii) the DLL3-VH comprises: a DLL3-HCDR1 comprising the amino acid sequence of SEQ ID NO: 26 or 84, a DLL3-HCDR2 comprising the amino acid sequence of SEQ ID NO: 27 or 85, and a DLL3-HCDR3 comprising the amino acid sequence of SEQ ID NO: 28 or 83; and
the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y; or
(iii) the DLL3-VH comprises: a DLL3-HCDR1 comprising the amino acid sequence of SEQ ID NO: 20, a DLL3-HCDR2 comprising the amino acid sequence of SEQ ID NO: 32, 96, 97, 98, or 99, and a DLL3-HCDR3 comprising the amino acid sequence of SEQ ID NO: 33, and FR regions of the DLL3-VH comprise one or more amino acid substitutions selected from the group consisting of 1E, 24T, 44S, 71V, and 73K; and
the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36, and FR regions of the DLL3-VL comprise one or more amino acid substitutions selected from the group consisting of 1N, 9K, 43S, 68A, and 85D; or
(iv) the DLL3-VH comprises: a DLL3-HCDR1 comprising the amino acid sequence of SEQ ID NO: 20, a DLL3-HCDR2 comprising the amino acid sequence of SEQ ID NO: 37, 117, 118, 119, or 120, and a DLL3-HCDR3 comprising the amino acid sequence of SEQ ID NO: 38, and FR regions of the DLL3-VH comprise one or more amino acid substitutions selected from the group consisting of 1E, 3R, 5Q, 7F, 9P, 10V, 12V, 40S, 41H, 44S, 48I, 67A, 69L, 71V, 73K, 75S, and 83T; and
the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40, and FR regions of the DLL3-VL comprise one or more amino acid substitutions selected from the group consisting of 9K, 43S, 60D, 68A, 87H, and 100S.

In some embodiments, the antigen-binding molecule according to any one of the foregoing binds to human DLL3 at 25° C. with a KD of less than 4×10⁻⁹ M, 3×10⁻⁹ M, 2×10⁻⁹M, 1×10⁻⁹M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M, 3×10⁻¹° M, 2×10⁻¹° M, 1×10⁻¹° M, or 9×10⁻¹¹ M, as measured by surface plasmon resonance.

In some embodiments, provided is the anti-DLL3 antibody according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D.

In some embodiments, provided is the anti-DLL3 antibody according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22, and FR regions of the DLL3-VH comprise one or more amino acid substitutions selected from the group consisting of 1E, 5Q, 27Y, 30T, 38K, 43K, 48I, 67A, 68A, 69L, 71V, 73K, 75S, 76N, and 93A; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25, and FR regions of the DLL3-VL comprise one or more amino acid substitutions selected from the group consisting of 1N, 36L, 42K, 43S, 44F, 46G, 69A, 71Y, 79Q, and 85D.

In some embodiments, provided is the anti-DLL3 antibody according to any one of the foregoing, wherein:
the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y; or
the DLL3-VH comprises: a DLL3-HCDR1 comprising the amino acid sequence of SEQ ID NO: 84, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 28; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31, and FR regions of the DLL3-VL comprise one or more amino acid substitutions selected from the group consisting of 42G, 44V, and 71Y.

In some embodiments, provided is the anti-DLL3 antibody according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33, and FR regions of the DLL3-VH comprise one or more amino acid substitutions selected from the group consisting of 1E, 24T, 44S, 71V, and 73K; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36, and FR regions of the DLL3-VL comprise one or more amino acid substitutions selected from the group consisting of 1N, 9K, 43S, 68A, and 85D.

In some embodiments, provided is the anti-DLL3 antibody according to any one of the foregoing, wherein:
the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38, and FR regions of the DLL3-VH comprise one or more amino acid substitutions selected from the group consisting of 1E, 3R, 5Q, 7F, 9P, 10V, 12V, 40S, 41H, 44S, 48I, 67A, 69L, 71V, 73K, 75S, and 83T; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40, and FR regions of the DLL3-VL comprise one or more amino acid substitutions selected from the group consisting of 9K, 43S, 60D, 68A, 87H, and 100S.

In some embodiments, provided is the anti-DLL3 antibody according to any one of the foregoing, wherein:
the DLL3-VH comprises the amino acid sequence of SEQ ID NO: 50 or a variant thereof. In some embodiments, the variant comprises one or more amino acid substitutions selected from the group consisting of Q1E, Q43K, M48I, V67A, I69L, R71V, T73K, and S76N in FR regions of SEQ ID NO: 50.

In some embodiments, provided is the anti-DLL3 antibody according to any one of the foregoing, wherein:
the DLL3-VH comprises the amino acid sequence of SEQ ID NO: 51 or a variant thereof. In some embodiments, the variant comprises one or more amino acid substitutions selected from the group consisting of Q1E, 5Q, R38K, V68A, F69L, L71V, T73K, V75S, and S76N in FR regions of SEQ ID NO: 51.

In some embodiments, provided is the anti-DLL3 antibody according to any one of the foregoing, wherein:
the DLL3-VH comprises the amino acid sequence of SEQ ID NO: 52 or a variant thereof. In some embodiments, the variant comprises one or more amino acid substitutions selected from the group consisting of F27Y, S30T, I69L, R71V, D73K, and T93A in FR regions of SEQ ID NO: 52.

In some embodiments, provided is the anti-DLL3 antibody according to any one of the foregoing, wherein:
the DLL3-VL comprises the amino acid sequence of SEQ ID NO: 62 or a variant thereof. In some embodiments, the variant comprises one or more amino acid substitutions selected from the group consisting of F36L, A43S, P44F, S46G, T69A, F71Y, and T85D in FR regions of SEQ ID NO: 62.

In some embodiments, provided is the anti-DLL3 antibody according to any one of the foregoing, wherein:
the DLL3-VL comprises the amino acid sequence of SEQ ID NO: 68 or a variant thereof. In some embodiments, the variant comprises one or more amino acid substitutions selected from the group consisting of Y36L, Q42K, L46G, T69A, F71Y, E79Q, and V85D in FR regions of SEQ ID NO: 68.

In some embodiments, provided is the anti-DLL3 antibody according to any one of the foregoing, wherein:
the DLL3-VH comprises the amino acid sequence of SEQ ID NO: 78 or a variant thereof. In some embodiments, the variant comprises one or more amino acid substitutions in FR regions of SEQ ID NO: 78.

In some embodiments, provided is the anti-DLL3 antibody according to any one of the foregoing, wherein:
the DLL3-VL comprises the amino acid sequence of SEQ ID NO: 75 or a variant thereof. In some embodiments, the variant comprises one or more amino acid substitutions selected from the group consisting of K42G, P44V, and F71Y in FR regions of SEQ ID NO: 75.

In some embodiments, provided is the anti-DLL3 antibody according to any one of the foregoing, wherein:
the DLL3-VH comprises the amino acid sequence of SEQ ID NO: 95 or a variant thereof. In some embodiments, the variant comprises one or more amino acid substitutions selected from the group consisting of Q1E, A24T, R44S, R71V, and T73K in FR regions of SEQ ID NO: 95.

In some embodiments, provided is the anti-DLL3 antibody according to any one of the foregoing, wherein:
the DLL3-VL comprises the amino acid sequence of SEQ ID NO: 88 or a variant thereof. In some embodiments, the variant comprises one or more amino acid substitutions selected from the group consisting of DIN, D9K, P43S, G68A, and V85D in FR regions of SEQ ID NO: 88.

In some embodiments, provided is the anti-DLL3 antibody according to any one of the foregoing, wherein:
the DLL3-VH comprises the amino acid sequence of SEQ ID NO: 116 or a variant thereof.

In some embodiments, the variant comprises one or more amino acid substitutions selected from the group consisting of Q1E, Q3R, V5Q, S7F, A9P, E10V, K12V, A40S, P41H, R44S, M48I, V67A, I69L, R71V, T73K, A75S, and R83T in FR regions of SEQ ID NO: 116.

In some embodiments, provided is the anti-DLL3 antibody according to any one of the foregoing, wherein:
the DLL3-VL comprises the amino acid sequence of SEQ ID NO: 100 or a variant thereof. In some embodiments, the variant comprises one or more amino acid substitutions selected from the group consisting of D9K, P43S, G68A, and Y87H in FR regions of SEQ ID NO: 100.

In some embodiments, provided is the anti-DLL3 antibody according to any one of the foregoing, wherein:
the DLL3-VL comprises the amino acid sequence of SEQ ID NO: 103 or a variant thereof. In some embodiments, the variant comprises one or more amino acid substitutions selected from the group consisting of A43S, S60D, and Q100S in FR regions of SEQ ID NO: 103.

In some embodiments, provided is the anti-DLL3 antibody according to any one of the foregoing, wherein:
the DLL3-VH comprises the amino acid sequence of SEQ ID NO: 56 or a variant thereof. In some embodiments, the variant comprises one or more amino acid substitutions in FR regions of SEQ ID NO: 56.

In some embodiments, provided is the anti-DLL3 antibody according to any one of the foregoing, wherein:
the DLL3-VH comprises the amino acid sequence of SEQ ID NO: 57 or a variant thereof. In some embodiments, the variant comprises one or more amino acid substitutions in FR regions of SEQ ID NO: 57.

In some embodiments, provided is the anti-DLL3 antibody according to any one of the foregoing, wherein:
the DLL3-VL comprises the amino acid sequence of SEQ ID NO: 63 or a variant thereof. In some embodiments, the variant comprises one or more amino acid substitutions in FR regions of SEQ ID NO: 63; in some embodiments, the amino acid substitutions are one or more amino acid substitutions selected from the group consisting of A43S and P44F.

In some embodiments, provided is the anti-DLL3 antibody according to any one of the foregoing, wherein:
the DLL3-VL comprises the amino acid sequence of SEQ ID NO: 64 or a variant thereof. In some embodiments, the variant comprises one or more amino acid substitutions in FR regions of SEQ ID NO: 64; in some embodiments, the amino acid substitutions are one or more amino acid substitutions selected from the group consisting of A43S and P44F.

In some embodiments, provided is the anti-DLL3 antibody according to any one of the foregoing, wherein:
the DLL3-VL comprises the amino acid sequence of SEQ ID NO: 65 or a variant thereof. In some embodiments, the variant comprises one or more amino acid substitutions in FR regions of SEQ ID NO: 65; in some embodiments, the amino acid substitutions are one or more amino acid substitutions selected from the group consisting of A43S and P44F.

In some embodiments, provided is the anti-DLL3 antibody according to any one of the foregoing, wherein:
the DLL3-VL comprises the amino acid sequence of SEQ ID NO: 66 or a variant thereof. In some embodiments, the variant comprises one or more amino acid substitutions in FR regions of SEQ ID NO: 66; in some embodiments, the amino acid substitutions are one or more amino acid substitutions selected from the group consisting of A43S and P44F.

In some embodiments, provided is the anti-DLL3 antibody according to any one of the foregoing, wherein:
the DLL3-VL comprises the amino acid sequence of SEQ ID NO: 67 or a variant thereof. In some embodiments, the variant comprises one or more amino acid substitutions in FR regions of SEQ ID NO: 67.

In some embodiments, provided is the anti-DLL3 antibody according to any one of the foregoing, wherein:
the DLL3-VH comprises the amino acid sequence of SEQ ID NO: 79, 80, 81, or 82, or a variant of SEQ ID NO: 79, 80, 81, or 82. In some embodiments, the variant comprises one or more amino acid substitutions in FR regions of SEQ ID NO: 79, 80, 81, or 82; and
the DLL3-VL comprises the amino acid sequence of SEQ ID NO: 75 or a variant thereof. In some embodiments, the variant comprises one or more amino acid substitutions selected from the group consisting of K42G, P44V, and F71Y in FR regions of SEQ ID NO: 75.

In some embodiments, provided is the anti-DLL3 antibody according to any one of the foregoing, wherein:
the DLL3-VH comprises the amino acid sequence of SEQ ID NO: 91, 92, 93, or 94, or a variant of SEQ ID NO: 91, 92, 93, or 94. In some embodiments, the variant comprises one or more amino acid substitutions in FR regions of SEQ ID NO: 91, 92, 93, or 94; and
the DLL3-VL comprises the amino acid sequence of SEQ ID NO: 88 or a variant thereof. In some embodiments, the variant comprises one or more amino acid substitutions selected from the group consisting of DIN, D9K, P43S, G68A, and V85D in FR regions of SEQ ID NO: 88.

In some embodiments, provided is the anti-DLL3 antibody according to any one of the foregoing, wherein:
the DLL3-VH comprises the amino acid sequence of SEQ ID NO: 107, 108, 109, or 110, or a variant of SEQ ID NO: 107, 108, 109, or 110. In some embodiments, the variant comprises one or more amino acid substitutions in FR regions of SEQ ID NO: 107, 108, 109, or 110; in some embodiments, the amino acid substitutions are one or more amino acid substitutions selected from the group consisting of V5Q, S7F, A9P, E10V, K12V, A40S, P41H, M48I, V67A, A75S, and R83T.

In some embodiments, provided is the anti-DLL3 antibody according to any one of the foregoing, wherein:
the DLL3-VH comprises the amino acid sequence of SEQ ID NO: 112, or a variant of SEQ ID NO: 112. In some embodiments, the variant comprises one or more amino acid substitutions in FR regions of SEQ ID NO: 112; in some embodiments, the amino acid substitutions are one or more amino acid substitutions selected from the group consisting of V5Q, A9P, K12V, A40S, P41H, 4M8I, V67A, and R83T.

In some embodiments, provided is the anti-DLL3 antibody according to any one of the foregoing, wherein:
the DLL3-VH comprises the amino acid sequence of SEQ ID NO: 113, or a variant of SEQ ID NO: 113. In some embodiments, the variant comprises one or more amino acid substitutions in FR regions of SEQ ID NO: 113; in some embodiments, the amino acid substitutions are one or more amino acid substitutions selected from the group consisting of V5Q, S7F, A9P, E10V, K12V, A40S, P41H, A75S, and R83T.

In some embodiments, provided is the anti-DLL3 antibody according to any one of the foregoing, wherein:
the DLL3-VH comprises the amino acid sequence of SEQ ID NO: 114, or a variant of SEQ ID NO: 114. In some embodiments, the variant comprises one or more amino acid substitutions in FR regions of SEQ ID NO: 114; in some embodiments, the amino acid substitutions are one or more amino acid substitutions selected from the group consisting of V5Q, S7F, A9P, E10V, K12V, M48I, V67A, A75S, and R83T.

In some embodiments, provided is the anti-DLL3 antibody according to any one of the foregoing, wherein:
the DLL3-VH comprises the amino acid sequence of SEQ ID NO: 115, or a variant of SEQ ID NO: 115. In some embodiments, the variant comprises one or more amino acid substitutions in FR regions of SEQ ID NO: 115; in some embodiments, the amino acid substitutions are one or more amino acid substitutions selected from the group consisting of V5Q, A9P, K12V, A40S, P41H, M48I, and V67A.

In some embodiments, provided is the anti-DLL3 antibody according to any one of the foregoing, wherein:
the DLL3-VL comprises the amino acid sequence of SEQ ID NO: 100 or a variant thereof. In some embodiments, the variant comprises one or more amino acid substitutions selected from the group consisting of D9K, P43S, G68A, and Y87H in FR regions of SEQ ID NO: 100.

In some embodiments, provided is the anti-DLL3 antibody according to any one of the foregoing, wherein:
the DLL3-VL comprises the amino acid sequence of SEQ ID NO: 103 or a variant thereof. In some embodiments, the variant comprises one or more amino acid substitutions selected from the group consisting of A43S, S60D, and Q100S in FR regions of SEQ ID NO: 103.

In some embodiments, provided is the anti-DLL3 antibody according to any one of the foregoing, wherein:
i) the DLL3-VH comprises the amino acid sequence of SEQ ID NO: 43, 44, 45, 46, 47, 48, 49, 50, 51, 52, 53, 54, 55, 56, or 57, and the DLL3-VL comprises the amino acid sequence of SEQ ID NO: 63, 64, 65, 66, or 67; or
the DLL3-VH comprises the amino acid sequence of SEQ ID NO: 56 or 57, and the DLL3-VL comprises the amino acid sequence of SEQ ID NO: 58, 59, 60, 61, 62, 63, 64, 65, 66, 67, or 68; or
ii) the DLL3-VH comprises the amino acid sequence of SEQ ID NO: 78, 79, 80, 81, or 82, and the DLL3-VL comprises the amino acid sequence of SEQ ID NO: 75, 76, or 77; or
iii) the DLL3-VH comprises the amino acid sequence of SEQ ID NO: 89, 90, 91, 92, 93, 94, or 95, and the DLL3-VL comprises the amino acid sequence of SEQ ID NO: 86, 87, or 88; or
iv) the DLL3-VH comprises the amino acid sequence of SEQ ID NO: 105, 106, 107, 108, 109, 110, 111, 112, 113, 114, 115, or 116, and the DLL3-VL comprises the amino acid sequence of SEQ ID NO: 100, 101, 102, 103, or 104.

In some embodiments, provided is the anti-DLL3 antibody according to any one of the foregoing, wherein:
the DLL3-VH comprises the amino acid sequence of SEQ ID NO: 43, 44, or 45, and the DLL3-VL comprises the amino acid sequence of SEQ ID NO: 58, 59, or 60; or
the DLL3-VH comprises the amino acid sequence of SEQ ID NO: 47, 48, 49, 53, 54, or 55, and the DLL3-VL comprises the amino acid sequence of SEQ ID NO: 59; or
the DLL3-VH comprises the amino acid sequence of SEQ ID NO: 43, 44, or 45, and the DLL3-VL comprises the amino acid sequence of SEQ ID NO: 60; or
the DLL3-VH comprises the amino acid sequence of SEQ ID NO: 46, 48, 53, 54, or 55, and the DLL3-VL comprises the amino acid sequence of SEQ ID NO: 61; or
the DLL3-VH comprises the amino acid sequence of SEQ ID NO: 54, and the DLL3-VL comprises the amino acid sequence of SEQ ID NO: 63 or 64; or
the DLL3-VH comprises the amino acid sequence of SEQ ID NO: 56 or 57, and the DLL3-VL comprises the amino acid sequence of SEQ ID NO: 65, 66, or 67.

In some embodiments, provided is the anti-DLL3 antibody according to any one of the foregoing, wherein:
the DLL3-VH comprises the amino acid sequence of SEQ ID NO: 78 or 79, and the DLL3-VL comprises the amino acid sequence of SEQ ID NO: 75, 76, or 77; or
the DLL3-VH comprises the amino acid sequence of SEQ ID NO: 78, 79, 80, 81, or 82, and the DLL3-VL comprises the amino acid sequence of SEQ ID NO: 77.

In some embodiments, provided is the anti-DLL3 antibody according to any one of the foregoing, wherein:
the DLL3-VH comprises the amino acid sequence of SEQ ID NO: 89, 90, 91, 92, 93, or 94, and the DLL3-VL comprises the amino acid sequence of SEQ ID NO: 86 or 87; or
the DLL3-VH comprises the amino acid sequence of SEQ ID NO: 95, and the DLL3-VL comprises the amino acid sequence of SEQ ID NO: 87.

In some embodiments, provided is the anti-DLL3 antibody according to any one of the foregoing, wherein:
the DLL3-VH comprises the amino acid sequence of SEQ ID NO: 105, 106, 107, 108, 109, 110, or 111, and the DLL3-VL comprises the amino acid sequence of SEQ ID NO: 100, 101, or 102; or
the DLL3-VH comprises the amino acid sequence of SEQ ID NO: 112, 113, 114, or 115, and the DLL3-VL comprises the amino acid sequence of SEQ ID NO: 100, 103, or 104; or the DLL3-VH comprises the amino acid sequence of SEQ ID NO: 109, and the DLL3-VL comprises the amino acid sequence of SEQ ID NO: 103.

In some embodiments, provided is the anti-DLL3 antibody according to any one of the foregoing, wherein:
i) the DLL3-VH comprises the amino acid sequence of SEQ ID NO: 54, and the DLL3-VL comprises the amino acid sequence of SEQ ID NO: 63; or
the DLL3-VH comprises the amino acid sequence of SEQ ID NO: 54, and the DLL3-VL comprises the amino acid sequence of SEQ ID NO: 61; or
the DLL3-VH comprises the amino acid sequence of SEQ ID NO: 54, and the DLL3-VL comprises the amino acid sequence of SEQ ID NO: 64; or
the DLL3-VH comprises the amino acid sequence of SEQ ID NO: 56, and the DLL3-VL comprises the amino acid sequence of SEQ ID NO: 65; or
the DLL3-VH comprises the amino acid sequence of SEQ ID NO: 56, and the DLL3-VL comprises the amino acid sequence of SEQ ID NO: 66; or
the DLL3-VH comprises the amino acid sequence of SEQ ID NO: 56, and the DLL3-VL comprises the amino acid sequence of SEQ ID NO: 67; or
the DLL3-VH comprises the amino acid sequence of SEQ ID NO: 57, and the DLL3-VL comprises the amino acid sequence of SEQ ID NO: 65; or
the DLL3-VH comprises the amino acid sequence of SEQ ID NO: 57, and the DLL3-VL comprises the amino acid sequence of SEQ ID NO: 66; or
the DLL3-VH comprises the amino acid sequence of SEQ ID NO: 57, and the DLL3-VL comprises the amino acid sequence of SEQ ID NO: 67; or
ii) the DLL3-VH comprises the amino acid sequence of SEQ ID NO: 81, and the DLL3-VL comprises the amino acid sequence of SEQ ID NO: 77; or
iii) the DLL3-VH comprises the amino acid sequence of SEQ ID NO: 91, and the DLL3-VL comprises the amino acid sequence of SEQ ID NO: 87; or
iv) the DLL3-VH comprises the amino acid sequence of SEQ ID NO: 109, and the DLL3-VL comprises the amino acid sequence of SEQ ID NO: 100.

In some embodiments, provided is the anti-DLL3 antibody according to the foregoing, wherein:
i) the DLL3-VH comprises the amino acid sequence of SEQ ID NO: 12, and the DLL3-VL comprises the amino acid sequence of SEQ ID NO: 13; or
ii) the DLL3-VH comprises the amino acid sequence of SEQ ID NO: 14, and the DLL3-VL comprises the amino acid sequence of SEQ ID NO: 15; or
iii) the DLL3-VH comprises the amino acid sequence of SEQ ID NO: 16, and the DLL3-VL comprises the amino acid sequence of SEQ ID NO: 17; or
iv) the DLL3-VH comprises the amino acid sequence of SEQ ID NO: 18, and the DLL3-VL comprises the amino acid sequence of SEQ ID NO: 19.

In some embodiments, provided is the anti-DLL3 antibody according to any one of the foregoing, wherein:
the DLL3-VH comprises the amino acid sequence of SEQ ID NO: 54, and the DLL3-VL comprises the amino acid sequence of SEQ ID NO: 63; or
the DLL3-VH comprises the amino acid sequence of SEQ ID NO: 54, and the DLL3-VL comprises the amino acid sequence of SEQ ID NO: 61.

In some embodiments, provided is the anti-DLL3 antibody according to any one of the foregoing, wherein:
the DLL3-VH comprises the amino acid sequence of SEQ ID NO: 81, and the DLL3-VL comprises the amino acid sequence of SEQ ID NO: 77.

In some embodiments, provided is the anti-DLL3 antibody according to any one of the foregoing, wherein:
the DLL3-VH comprises the amino acid sequence of SEQ ID NO: 91, and the DLL3-VL comprises the amino acid sequence of SEQ ID NO: 87.

In some embodiments, provided is the anti-DLL3 antibody according to any one of the foregoing, wherein:
the DLL3-VH comprises the amino acid sequence of SEQ ID NO: 109, and the DLL3-VL comprises the amino acid sequence of SEQ ID NO: 100.

In some embodiments, provided is the anti-DLL3 antibody according to any one of the foregoing, wherein the anti-DLL3 antibody is an antibody fragment.

In some embodiments, provided is the anti-DLL3 antibody according to any one of the foregoing, wherein the antibody fragment is a Fab, Fab′, F(ab′)2, Fd, Fv, scFv, dsFv, or dAb.

In some embodiments, provided is the anti-DLL3 antibody according to any one of the foregoing, wherein the anti-DLL3 antibody comprises a heavy chain constant region and a light chain constant region.

In some embodiments, provided is the anti-DLL3 antibody according to any one of the foregoing, wherein the heavy chain constant region is a heavy chain constant region of a human IgG.

In some embodiments, provided is the anti-DLL3 antibody according to any one of the foregoing, wherein the light chain constant region is a human κ or λ light chain constant region.

In some embodiments, provided is the anti-DLL3 antibody according to any one of the foregoing, wherein the heavy chain constant region is a heavy chain constant region of human IgG1.

In some embodiments, provided is the anti-DLL3 antibody according to any one of the foregoing, wherein the light chain constant region is a light chain constant region of human K.

In some embodiments, provided is the anti-DLL3 antibody according to any one of the foregoing, wherein the heavy chain constant region comprises the amino acid sequence of SEQ ID NO: 41.

In some embodiments, provided is the anti-DLL3 antibody according to any one of the foregoing, wherein the light chain constant region comprises the amino acid sequence of SEQ ID NO: 42.

In some embodiments, provided is the anti-DLL3 antibody according to any one of the foregoing, wherein the anti-DLL3 antibody comprises a heavy chain and a light chain, wherein:
the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 121, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 122; or
the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 123, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 124; or
the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 125, and the light chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 126; or
the heavy chain comprises an amino acid sequence having at least 85%, 86%, 87%, 88%, 89%, 90%, 91%, 92%, 93%, 94%, 95%, 96%, 97%, 98%, or 99% sequence identity to SEQ ID NO: 127, and the light chain comprises an amino acid sequence having at least 85%, 86%, 87%, 88%, 89%, 90%, 91%, 92%, 93%, 94%, 95%, 96%, 97%, 98%, or 99% identity to SEQ ID NO: 128.

In some embodiments, provided is the anti-DLL3 antibody according to any one of the foregoing, wherein the anti-DLL3 antibody comprises a heavy chain and a light chain, wherein:
the heavy chain comprises the amino acid sequence of SEQ ID NO: 121, and the light chain comprises the amino acid sequence of SEQ ID NO: 122; or
the heavy chain comprises the amino acid sequence of SEQ ID NO: 123, and the light chain comprises the amino acid sequence of SEQ ID NO: 124;
the heavy chain comprises the amino acid sequence of SEQ ID NO: 125, and the light chain comprises the amino acid sequence of SEQ ID NO: 126; or
the heavy chain comprises the amino acid sequence of SEQ ID NO: 127, and the light chain comprises the amino acid sequence of SEQ ID NO: 128.

In some embodiments, provided is the anti-DLL3 antibody according to any one of the foregoing, wherein the anti-DLL3 antibody comprises a heavy chain and a light chain, wherein the heavy chain comprises the amino acid sequence of SEQ ID NO: 121, and the light chain comprises the amino acid sequence of SEQ ID NO: 122.

In some embodiments, provided is the anti-DLL3 antibody according to any one of the foregoing, wherein the anti-DLL3 antibody comprises a heavy chain and a light chain, wherein the heavy chain comprises the amino acid sequence of SEQ ID NO: 125, and the light chain comprises the amino acid sequence of SEQ ID NO: 126.

In some embodiments, provided is the anti-DLL3 antibody according to any one of the foregoing, wherein the anti-DLL3 antibody is a bispecific antibody.

In some embodiments, provided is the anti-DLL3 antibody according to any one of the foregoing, wherein the anti-DLL3 antibody is a bispecific antibody binding specifically to DLL3 and CD3.

In another aspect, the present disclosure provides a pharmaceutical composition comprising: a therapeutically effective amount of the antigen-binding molecule according to any one of the foregoing or the anti-DLL3 antibody according to any one of the foregoing, and one or more pharmaceutically acceptable carriers, diluents, buffers, or excipients. In some embodiments, the pharmaceutical composition also comprises at least one second therapeutic agent. In one example, the second therapeutic agent is any agent that is advantageously combined with a CD3/DLL3 bispecific antigen-binding molecule. In some embodiments, the second therapeutic agent is an antibody capable of binding specifically to CD28. In some embodiments, the second therapeutic agent is a bispecific antibody capable of binding specifically to CD28 and a tumor cell surface antigen (TAA). In some embodiments, the second therapeutic agent is a bispecific antibody capable of binding specifically to CD28 and DLL3.

In another aspect, the present disclosure also provides an isolated nucleic acid encoding the antigen-binding molecule according to any one of the foregoing or the anti-DLL3 antibody according to any one of the foregoing.

In another aspect, the present disclosure also provides a vector comprising the aforementioned isolated nucleic acid.

In another aspect, the present disclosure also provides a host cell comprising the aforementioned isolated nucleic acid or vector.

In another aspect, the present disclosure also provides a method for treating a tumor or cancer, the method comprising administering to a subject a therapeutically effective amount of the antigen-binding molecule according to any one of the foregoing or the anti-DLL3 antibody according to any one of the foregoing or the pharmaceutical composition according to any one of the foregoing.

In another aspect, the present disclosure also provides use of the antigen-binding molecule according to any one of the foregoing or the antibody according to any one of the foregoing or the pharmaceutical composition according to any one of the foregoing in the preparation of a medicament for treating a tumor or cancer.

In another aspect, the present disclosure also provides the antigen-binding molecule according to any one of the foregoing or the anti-DLL3 antibody according to any one of the foregoing or the pharmaceutical composition according to any one of the foregoing for use as a medicament. In some embodiments, the medicament is used for treating a tumor or cancer.

In some embodiments, the tumor or cancer according to any one of the foregoing is selected from the group consisting of: head and neck squamous cell carcinoma, head and neck cancer, brain cancer, neuroglioma, glioblastoma multiforme, neuroblastoma, central nervous system cancer, neuroendocrine tumor, throat cancer, pharyngeal squamous cell carcinoma, oral squamous cell carcinoma, nasopharyngeal cancer, esophageal cancer, thyroid cancer, malignant pleural mesothelioma, lung cancer, breast cancer, liver cancer, hepatobiliary cancer, pancreatic cancer, gastric cancer, gastrointestinal cancer, intestinal cancer, colon cancer, colorectal cancer, renal cancer, clear cell renal cell carcinoma, ovarian cancer, endometrial cancer, cervical cancer, bladder cancer, prostate cancer, testicular cancer, skin cancer, melanoma, large cell lung cancer, triple-negative breast cancer, lymphoma, and small cell lung cancer.

In some embodiments, the tumor or cancer according to the foregoing is a DLL3-expressing cancer.

In some embodiments, the tumor or cancer according to the foregoing is a solid tumor.

In some embodiments, the tumor or cancer according to the foregoing is lung cancer.

In some embodiments, the tumor or cancer according to the foregoing is small cell lung cancer.

In some embodiments, the method for treating a tumor or cancer according to the foregoing further comprises the use of a second therapeutic agent. In some embodiments, the second therapeutic agent comprises an anti-tumor agent, radiotherapy, an antibody-drug conjugate, a bispecific antibody, a bispecific antibody conjugated to an anti-tumor agent, an immune checkpoint inhibitor, or a combination thereof.

In some embodiments, in the method for treating a tumor or cancer according to the foregoing, the second therapeutic agent is any agent that is advantageously combined with a CD3/DLL3 bispecific antigen-binding molecule. In some embodiments, the second therapeutic agent is an antibody capable of binding specifically to CD28. In some embodiments, the second therapeutic agent is a bispecific antibody capable of binding specifically to CD28 and a tumor cell surface antigen (TAA). In some embodiments, the second therapeutic agent is a bispecific antibody capable of binding specifically to CD28 and DLL3.

In some embodiments, in the method for treating a tumor or cancer according to the foregoing, the second therapeutic agent and the antigen-binding molecule according to any one of the present disclosure are administered simultaneously, sequentially, or separately.

The antigen-binding molecule provided by the present disclosure has the following characteristics: good therapeutic activity, safety, pharmacokinetic properties, and druggability (e.g., stability).

### BRIEF DESCRIPTION OF THE DRAWINGS

FIG. 1A shows a schematic diagram of the structure of Format1 of DLL3-CD3 bispecific antibodies, wherein Ob stands for Obscurin.

FIG. 1B shows a schematic diagram of the structure of Format2 of DLL3-CD3 bispecific antibodies.

FIG. 1C shows a schematic diagram of the structure of Format3 of DLL3-CD3 bispecific antibodies.

FIG. 2A shows experimental results for the binding of bispecific antibodies to DLL1. The results show that the DLL3-CD3 bispecific antibodies of the present disclosure do not bind to DLL1.

FIG. 2B shows experimental results for the binding of bispecific antibodies to DLL4. The results show that the DLL3-CD3 bispecific antibodies of the present disclosure do not bind to DLL4.

FIG. 3 shows experimental results for the killing effects of bispecific antibodies on cells. The results show that the bispecific antibodies of the present disclosure have no killing effects on H460 cells not expressing DLL3.

FIG. 4A shows experimental results for the activation of T cells by bispecific antibodies, and the results show that the bispecific antibodies of the present disclosure have significant T cell activating effects in the presence of DLL3-expressing DLL3/H82 cells.

FIG. 4B shows that the bispecific antibodies of the present disclosure do not activate T cells in the presence of H460 negative cells not expressing DLL3.

FIG. 5A shows results for cytokine release experiments for bispecific antibodies, and the results show that PBMCs stimulated by the bispecific antibodies of the present disclosure secreted very low levels of IFNγ in the presence of H1184 cells.

FIG. 5B shows that PBMCs stimulated by the bispecific antibodies of the present disclosure secreted very low levels of IL-6 in the presence of H1184 cells.

### DETAILED DESCRIPTION

### Terminology

The terminology used herein is for the purpose of describing embodiments only and is not intended to be limiting. Unless defined otherwise, all technical and scientific terms used herein have the same meaning as commonly understood by one of ordinary skill in the art to which the present disclosure pertains.

Unless the context clearly requires otherwise, throughout the description and the claims, the words “comprise”, “have”, “include”, and the like are to be understood in an inclusive sense as opposed to an exclusive or exhaustive sense; that is to say, in the sense of “including, but not limited to”. Unless otherwise specified, “comprise” includes “consist of”. For example, for a DLL3-HCDR1 comprising the amino acid sequence of SEQ ID NO: 20, it specifically encompasses a DLL3-HCDR1 the amino acid sequence of which is set forth in SEQ ID NO: 20.

The three-letter and single-letter codes used in the present disclosure for amino acids are as described in J. Biol. Chem, 243, p3558 (1968).

The term “and/or”, e.g., “X and/or Y” should be understood to mean “X and Y” or “X or Y” and should be used to provide explicit support for both meanings or either meaning.

As used herein, “CD3” and “CD3 fragment” refer to the well-known human CD3 protein or a fragment thereof unless specified as being from a non-human species (e.g., “mouse CD3”, “mouse CD3 fragment”, “monkey CD3”, or “monkey CD3 fragment”).

The term “amino acid” refers to naturally occurring and synthetic amino acids, as well as amino acid analogs and amino acid mimics that function in a manner similar to the naturally occurring amino acids. Naturally occurring amino acids are those encoded by genetic codes and those amino acids later modified, e.g., hydroxyproline, y-carboxyglutamic acid, and O-phosphoserine. Amino acid analogs refer to compounds that have a substantially identical chemical structure (i.e., an a carbon that binds to hydrogen, carboxyl, amino, and an R group) to naturally occurring amino acids, e.g., homoserine, norleucine, methionine sulfoxide, and methioninemethyl sulfonium. Such analogs have a modified R group (e.g., norleucine) or a modified peptide skeleton, but retain a substantially identical chemical structure to naturally occurring amino acids. Amino acid mimics refer to chemical compounds that have a different structure from amino acids, but function in a manner similar to naturally occurring amino acids.

The term “amino acid mutation” includes amino acid substitutions, deletions, insertions, and modifications. Any combination of substitutions, deletions, insertions, and modifications can be made to obtain a final construct, as long as the final construct possesses the desired properties, such as reduced binding to the Fc receptor. Amino acid sequence deletions and insertions include deletions and insertions at the amino terminus and/or the carboxyl terminus of a polypeptide chain. Specific amino acid mutations may be amino acid substitutions. In one embodiment, the amino acid mutation is a non-conservative amino acid substitution, i.e., replacement of one amino acid with another amino acid having different structural and/or chemical properties. Amino acid substitutions include replacement with non-naturally occurring amino acids or with derivatives of the 20 native amino acids (e.g., 4-hydroxyproline, 3-methylhistidine, ornithine, homoserine, and 5-hydroxylysine). Amino acid mutations can be generated using genetic or chemical methods well known in the art. Genetic methods may include site-directed mutagenesis, PCR, gene synthesis, and the like. It is contemplated that methods for altering amino acid side chain groups other than genetic engineering, such as chemical modification, may also be used. Various names may be used herein to indicate the same amino acid mutation. Herein, the expression of “position+amino acid residue” may be used to denote an amino acid residue at a specific position. For example, 366W indicates that an amino acid residue at position 366 is W. T366W indicates that an amino acid residue at position 366 is mutated from the original T to W.

The term “antigen-binding molecule” is used in the broadest sense and encompasses a variety of molecules binding specifically to an antigen, including but not limited to antibodies, other polypeptides having antigen-binding activity, and antibody fusion proteins in which the two are fused, as long as they exhibit desired antigen-binding activity. The antigen-binding molecule herein comprises a variable region (VH) and a variable region (VL) which together comprise an antigen-binding domain. Illustratively, the antigen-binding molecules herein are bispecific antigen-binding molecules (e.g., bispecific antibodies).

The term “antibody” is used in the broadest sense and encompasses a variety of antibody structures, including but not limited to monoclonal antibodies, polyclonal antibodies; monospecific antibodies, multispecific antibodies (e.g., bispecific antibodies), and full-length antibodies, and antibody fragments (or antigen-binding fragments, or antibody fragments, or antigen-binding moieties), as long as they exhibit the desired antigen-binding activity. According to the context, those skilled can determine the specific meaning of “antibody”.

“Natural antibody” refers to a naturally-occurring immunoglobulin molecule. For example, a native IgG antibody is a heterotetrameric glycoprotein of about 150,000 Daltons composed of two light chains and two heavy chains linked by a disulfide bond. From N-terminus to C-terminus, each heavy chain has one variable region (VH, also known as variable heavy domain or heavy chain variable region), followed by three constant domains (CH1, CH2, and CH3). Similarly, from N-terminus to C-terminus, each light chain has one variable region (VL, also known as variable light domain or light chain variable domain), followed by one constant light domain (light chain constant region, CL). The term “full-length antibody”, “intact antibody”, and “whole antibody” are used herein interchangeably and refer to an antibody having a substantially similar structure to a native antibody structure or having heavy chains comprising an Fc region as defined herein.

The term “bispecific antibody” refers to an antibody (including an antibody or an antigen-binding fragment thereof, such as a single-chain antibody) capable of binding specifically to two different antigens or at least two different epitopes of the same antigen. Bispecific antibodies of various structures have been disclosed in the prior art; the bispecific antibodies can be classified into IgG-like bispecific antibodies and antibody-fragment-type bispecific antibodies according to the integrity of IgG molecules; the bispecific antibodies can be classified into bivalent, trivalent, tetravalent or higher-valent bispecific antibodies according to the number of the antigen-binding regions; the bispecific antibodies can be classified into symmetric bispecific antibodies and asymmetric bispecific antibodies according to the presence of horizontal symmetry in their structures. Among them, the bispecific antibodies based on antibody fragments, such as Fab fragments lacking Fc fragments, formed by binding 2 or more Fab fragments in one molecule, have low immunogenicity, small molecular weight and high tumor tissue permeability, and typical antibody structures of this type comprise bispecific antibodies, such as F(ab)2, scFv-Fab and (scFv)2-Fab; IgG-like bispecific antibodies (e.g., having Fc fragments) have relatively large molecular weight, and the Fc fragments facilitate later purification of the antibody and increase their solubility and stability, and the Fc fragments may further bind to the receptor FcRn and increase the serum half-life of the antibody, and typical structural models of bispecific antibodies comprise bispecific antibodies such as KiH, CrossMAb, Triomab quadroma, FcΔAdp, ART-Ig, BiMAb, Biclonics, BEAT, DuoBody, Azymetric, XmAb, 2:1 TCBs, 1Fab-IgG TDB, FynomAb, two-in-one/DAF, scFv-Fab-IgG, DART-Fc, LP-DART, CODV-Fab-TL, HLE-BiTE, F(ab)2-CrossMAb, IgG-(scFv)2, Bs4Ab, DVD-Ig, Tetravalent-DART-Fc, (scFv)4-Fc, CODV-Ig, mAb2, and F(ab)4-CrossMAb (see Aran F. Labrijn et al., Nature Reviews Drug Discovery, volume 18, pages 585-608 (2019); Chen S1 et al., J Immunol Res., Feb. 11, 2019; 2019:4516041).

The term “variable region” or “variable domain” refers to a domain in an antigen-binding molecule that binds to an antigen. Herein, a heavy chain variable region in the antigen-binding moiety binding specifically to CD3 is denoted by CD3-VH, and a light chain variable region is denoted by CD3-VL; a heavy chain variable region in the antigen-binding moiety binding specifically to CD3 is denoted by CD3-VH, and a light chain variable region is denoted by CD3-VL. VH and VL each comprise four conserved framework regions (FRs) and three complementarity determining regions (CDRs). The term “complementarity determining region” or “CDR” refers to a region in the variable domain that primarily contributes to antigen binding; “framework” or “FR” refers to variable domain residues other than CDR residues. A VH comprises 3 CDRs: HCDR1, HCDR2, and HCDR3; a VL comprises 3 CDRs: LCDR1, LCDR2, and LCDR3. Herein, the 3 CDRs in CD3-VH are denoted by CD3-HCDR1, CD3-HCDR2, and CD3-HCDR3, respectively; the 3 CDRs in CD3-VL are denoted by CD3-LCDR1, CD3-LCDR2, and CD3-LCDR3, respectively; the 3 CDRs in CD3-VH are denoted by CD3-HCDR1, CD3-HCDR2, and CD3-HCDR3, respectively; the 3 CDRs in CD3-VL are denoted by CD3-LCDR1, CD3-LCDR2, and CD3-LCDR3, respectively. Each VH and VL is, when viewed from N-terminus to C-terminus: FR1, CDR1, FR2, CDR2, FR3, CDR3, FR4. A single VH or VL may be sufficient to provide antigen-binding specificity.

The amino acid sequence boundaries of the CDRs can be determined by a variety of well-known schemes, for example, the “Kabat” numbering scheme (see Kabat et al. (1991), “Sequences of Proteins of Immunological Interest”, 5th ed., Public Health Service, National Institutes of Health, Bethesda, MD), the “Chothia” numbering scheme, the “ABM” numbering scheme, the “Contact” numbering scheme (see Martin, ACR. Protein Sequence and Structure Analysis of Antibody Variable Domains[J]. 2001), and the ImMunoGenTics (IMGT) numbering scheme (Lefranc, M. P. et al., Dev. Comp. Immunol., 27, 55-77 (2003); Front Immunol., Oct. 16, 2018; 9: 2278), and the like. The corresponding relationships between the various numbering schemes are well known to those skilled in the art. The numbering schemes of the present disclosure are shown in Table 1 below.

TABLE 1
Relationships between CDR numbering schemes

| CDR | IMGT | Kabat | AbM | Chothia | Contact |
|---|---|---|---|---|---|
| HCDR1 | 27-38 | 31-35 | 26-35 | 26-32 | 30-35 |
| HCDR2 | 56-65 | 50-65 | 50-58 | 52-56 | 47-58 |
| HCDR3 | 105-117 | 95-102 | 95-102 | 95-102 | 93-101 |
| LCDR1 | 27-38 | 24-34 | 24-34 | 24-34 | 30-36 |
| LCDR2 | 56-65 | 50-56 | 50-56 | 50-56 | 46-55 |
| LCDR3 | 105-117 | 89-97 | 89-97 | 89-97 | 89-96 |

Unless otherwise stated, the “Kabat” numbering scheme is applied to the sequences of the variable regions and CDRs in the embodiments of the present disclosure.

The term “antibody fragment” or “antigen-binding fragment” refers to a molecule different from an intact antibody, which comprises a moiety of an intact antibody that retains the antigen-binding ability of the intact antibody. Examples of antibody fragments include, but are not limited to, Fv, Fab, Fab′, Fab′-SH, F(ab′)₂, single-domain antibodies, single-chain Fab (scFab), diabodies, linear antibodies, single-chain antibody molecules (e.g., scFv), and multispecific antibodies formed from antibody fragments.

The term “Fc region” or “fragment crystallizable region” is used to define the C-terminal region of the heavy chain of an antibody, including native Fc regions and engineered Fc regions. In some embodiments, the Fc region comprises two subunits, which may be identical or different. In some embodiments, the Fc region of the human IgG heavy chain is defined as extending from the amino acid residue at position Cys226 or from Pro230 to its carboxyl terminus. Suitable native sequence Fc regions for the antibodies described herein include human IgG1, IgG2 (IgG2A, IgG2B), IgG3, and IgG4. Unless otherwise indicated, the numbering scheme for the Fc region is the EU index.

The term “Titin chain” refers to a peptide fragment of 78-118 amino acids in length comprising a Titin Ig-like 152 domain in a Titin protein or a functional variant thereof, wherein the Titin chain is capable of binding to an Obscurin Ig-like 1 domain or an Obscurin-like Ig-like 1 domain to form a dimerized complex.

The term “Obscurin chain” refers to, in the Obscurin protein, a peptide fragment that is 87-117 amino acids in length and comprises an Obscurin Ig-like 1 domain, or a functional variant thereof, or to, in the Obscurin-like 1 protein, a peptide fragment that is 78-118 amino acids in length and comprises an Obscurin-like Ig-like 1 domain, or a functional variant thereof.

The Obscurin chain is capable of binding to the Titin chain to form a dimerized complex. The Titin chain and the Obscurin chain of the present disclosure can be used to replace the CH1 and CL in a Fab, respectively, to form a replaced Fab (Fab-S), and the replacement does not affect the binding of the antigen-binding molecule to an antigen.

The term “chimeric” antibody refers to an antibody in which a portion of the heavy and/or light chain is derived from a particular source or species and the remainder of the heavy and/or light chain is derived from a different source or species.

The term “humanized” antibody refers to an antibody that retains the reactivity of a non-human antibody while having low immunogenicity in humans. For example, this can be achieved by retaining the non-human CDRs and replacing the remainder of the antibody with its human counterparts (i.e., the constant regions and the framework region portion of the variable regions).

The term “affinity” refers to the overall strength of the non-covalent interaction between a single binding site of a molecule (e.g., an antibody) and its binding ligand (e.g., an antigen). Unless otherwise indicated, as used herein, “binding affinity” refers to an internal binding affinity that reflects a 1:1 interaction between members of a binding pair (e.g., an antibody and an antigen). The affinity of a molecule X for its ligand Y can be generally expressed by the equilibrium dissociation constant (KD). Affinity can be determined by conventional methods known in the art, including those described herein. The term “kassoc” or “ka” refers to the association rate of a particular antibody-antigen interaction, while the term “kdis” or “kd” as used herein refers to the dissociation rate of a particular antibody-antigen interaction. As used herein, the term “KD” refers to the equilibrium dissociation constant, which is obtained from the ratio of kd to ka (i.e., kd/ka) and expressed as molar concentration (M). The KD value of an antibody can be measured using methods known in the art, such as surface plasmon resonance, ELISA, or solution equilibrium titration (SET).

The term “monoclonal antibody” refers to a population of substantially homogeneous antibodies, that is, the amino acid sequences of the antibody molecules comprised in the population are identical, except for a small number of natural mutations that may exist. In contrast, polyclonal antibody formulations generally comprise a plurality of different antibodies having different amino acid sequences in their variable domains, which are generally specific for different epitopes. In some embodiments, the antibody provided by the present disclosure is a monoclonal antibody.

The term “antigen” refers to a molecule or a portion of a molecule that is capable of being bound by a selective binding agent of an antigen-binding molecule (e.g., an antibody). An antigen may have one or more epitopes capable of interacting with different antigen-binding molecules (e.g., antibodies).

The term “epitope” refers to an area or region on an antigen that is capable of binding specifically to an antibody or an antigen-binding fragment thereof. Epitopes can be formed by contiguous amino acids (linear epitopes) or comprise non-contiguous amino acids (conformational epitopes), e.g., non-contiguous amino acids that are in spatial proximity to each other due to folding of an antigen (i.e., by tertiary folding of an antigen of a protein nature). The difference between the conformational epitope and the linear epitope is that in the presence of denaturing solvents, the binding of the antibody to the conformational epitope is lost. An epitope comprises at least 3, at least 4, at least 5, at least 6, at least 7, or 8-10 amino acids in a unique spatial conformation. Screening for antibodies that bind to particular epitopes (i.e., those that bind to identical epitopes) can be performed using routine methods in the art, such as but not limited to, alanine scanning, peptide blotting (see Meth. Mol. Biol. 248 (2004) 443-463), peptide cleavage analysis, epitope excision, epitope extraction, chemical modification of the antigen (see Prot. Sci. 9 (2000) 487-496), and cross-blocking (see “Antibodies”, Harlow and Lane (Cold Spring Harbor Press, Cold Spring Harb., NY)).

The term “capable of binding specifically”, “binding specifically”, or “binding” means that an antigen-binding molecule is capable of binding to a certain antigen or epitope with higher affinity than to other antigens or epitopes. Generally, an antibody binds to an antigen or epitope with an equilibrium dissociation constant (KD) of about 1×10⁻⁷ M or less (e.g., about 1×10⁻⁸ M or less). In some embodiments, the KD for the binding of an antibody to an antigen is 10% or less (e.g., 1%) of the KD for the binding of the antibody to a non-specific antigen (e.g., BSA or casein). KD may be determined using known methods, for example, by FACS or surface plasmon resonance. However, an antibody binding specifically to an antigen or an epitope thereof may have cross-reactivity to other related antigens, e.g. to corresponding antigens from other species (homologous), such as humans or monkeys, e.g., Macaca fascicularis (cynomolgus, cyno), Pan troglodytes (chimpanzee, chimp), or Callithrix jacchus (commonmarmoset, marmoset).

The term “not bind” or “no binding” means that the antigen-binding molecule is not capable of binding to a certain antigen or an epitope thereof in the manner of the specific binding described above, for example, when the antibody binds to the antigen or the epitope thereof with an equilibrium dissociation constant (KD) of about 1×10⁻⁶ M or greater.

The term “antigen-binding moiety” refers to a polypeptide molecule binding specifically to a target antigen. A specific antigen-binding moiety includes an antigen-binding domain of an antibody, e.g., comprises a heavy chain variable region and a light chain variable region. The term “antigen-binding moiety binding specifically to CD3” refers to a moiety that is capable of binding to CD3 or an epitope thereof with sufficient affinity, such that a molecule containing the moiety can be used as a diagnostic agent and/or therapeutic agent targeting CD3. For example, the antigen-binding moiety binding specifically to CD3 has the following equilibrium dissociation constant (KD): <about 10 nM, as measured by surface plasmon resonance. Antigen-binding moieties include antibody fragments as defined herein, e.g., a Fab, replaced Fab, or scFv.

The term “linker” refers to a linker unit that links two polypeptide fragments. Herein, the linkers present in the same structural formula may be identical or different. The linker may be a peptide linker comprising one or more amino acids, typically about 1-30, 2-24, or 3-15 amino acids. The linkers used herein may be identical or different. When “-” appears in a structural formula, it means that the units on both sides are directly linked by a covalent bond.

“Tm” is the temperature of dissolution and denaturation (endogenous fluorescence). When the protein is denatured (by heating or by a denaturant), the tertiary structure is opened and the aromatic amino acid microenvironment changes, resulting in a change in the emission fluorescence spectrum. In the present disclosure, Tm1 refers to the temperature at which the fluorescence value changes to half of the maximum value.

“Tonset” is the onset temperature of denaturation. It refers to the temperature at which the protein begins to denature, i.e., the temperature at which the fluorescence value begins to change.

“Tagg” is the onset temperature of aggregation. The temperature at which the sample begins to aggregate is monitored by detecting aggregates at two wavelengths of 266 nm and 473 nm by static light scattering. Tagg 266 refers to the onset temperature of aggregation monitored at 266 nm.

The term “nucleic acid” is used interchangeably herein with the term “polynucleotide” and refers to deoxyribonucleotide or ribonucleotide and a polymer thereof in either single-stranded or double-stranded form. The term encompasses nucleic acids comprising known nucleotide analogs or modified backbone residues or linkages, which are synthetic, naturally occurring, and non-naturally occurring, have similar binding properties to the reference nucleic acid, and are metabolized in a manner similar to the reference nucleotide. Examples of such analogs include, but are not limited to, phosphorothioate, phosphoramidate, methylphosphonate, chiral-methylphosphonate, 2-O-methyl ribonucleotide, and peptide-nucleic acid (PNA). “Isolated” nucleic acid refers to a nucleic acid molecule that has been separated from components of its natural environment. The isolated nucleic acid includes a nucleic acid molecule comprised in a cell that generally comprises the nucleic acid molecule, but the nucleic acid molecule is present extrachromosomally or at a chromosomal position different from its natural chromosomal position. An isolated nucleic acid encoding the antigen-binding molecule refers to one or more nucleic acid molecules encoding antibody heavy and light chains (or fragments thereof), including such nucleic acid molecule(s) in a single vector or separate vectors, and such nucleic acid molecule(s) present at one or more locations in a host cell. Unless otherwise stated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be obtained by generating sequences in which the third position of one or more selected (or all) codons is substituted with degenerate bases and/or deoxyinosine residues.

It should be understood that although specific coding nucleotide sequences are not provided in the present disclosure, those skilled can determine the corresponding coding nucleotide sequences based on amino acid sequences according to codon rules and host codon preferences.

The terms “polypeptide” and “protein” are used interchangeably herein and refer to a polymer of amino acid residues. The terms apply to amino acid polymers in which one or more amino acid residues are corresponding artificial chemical mimics of naturally occurring amino acids, as well as to naturally occurring amino acid polymers and non-naturally occurring amino acid polymers. Unless otherwise stated, a particular polypeptide sequence also implicitly encompasses conservatively modified variants thereof.

The term “sequence identity” refers to the degree (percentage) to which the amino acids/nucleic acids of two sequences are identical at equivalent positions when the two sequences are optimally aligned. In the alignment process, gaps may be allowed to be introduced, if necessary, to achieve the maximum percent sequence identity, but any conservative substitution is not considered a part that constitutes sequence identity. To determine percent sequence identity, alignments can be accomplished by techniques known to those skilled in the art, for example, using publicly available computer software, such as BLAST, BLAST-2, ALIGN, ALIGN-2, or Megalign (DNASTAR) software. Those skilled in the art can determine parameters suitable for measuring alignment, including any algorithms required to achieve maximum alignment of the full length of the aligned sequences. The term “fusion” or “linkage” means that components (e.g., antigen-binding moieties and Fc domains) are covalently linked directly or via a linker.

The term “vector” means a polynucleotide molecule capable of transporting another polynucleotide linked thereto. One type of vector is a “plasmid”, which refers to a circular double-stranded DNA loop into which additional DNA segments can be ligated. Another type of vector is a viral vector, such as an adeno-associated viral vector (AAV or AAV2), wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. The term “expression vector” or “expression construct” refers to a vector that is applied to transform a host cell and comprises a nucleic acid sequence that directs and/or controls (along with the host cell) the expression of one or more heterologous coding regions operably linked thereto. Expression constructs may include, but are not limited to, sequences that affect or control transcription and translation and affect RNA splicing of a coding region operably linked thereto in the presence of an intron.

The terms “host cell”, “host cell line”, and “host cell culture” are used interchangeably and refer to cells into which exogenous nucleic acids have been introduced, including progenies of such cells. Host cells include “transformants” and “transformed cells”, which include primary transformed cells and progeny derived therefrom, regardless of the number of passages. Progeny may not be completely identical to parent cells in terms of nucleic acid content and may contain mutations. As used herein, the term includes mutant progenies that have the same function or biological activity as the cells screened or selected from the primary transformed cells. Host cells include prokaryotic and eukaryotic host cells, wherein eukaryotic host cells include, but are not limited to, mammalian cells, insect cell lines, plant cells, and fungal cells. Mammalian host cells include human, mouse, rat, canine, monkey, porcine, goat, bovine, equine, and hamster cells, including but not limited to, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, and HEK-293 cells. Fungal cells include yeast and filamentous fungal cells, including, for example, Pichiapastoris, Pichia finlandica, Pichia trehalophila, Pichia koclamae, Pichia membranaefaciens, Pichia minuta (Ogataea minuta, Pichia lindneri), Pichiaopuntiae, Pichia thermotolerans, Pichia salictaria, Pichia guercuum, Pichia pijperi, Pichia stiptis, Pichia methanolica, Pichia, Saccharomycescerevisiae, Saccharomyces, Hansenula polymorpha, Kluyveromyces, Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens, and Neurospora crassa. Pichia, any Saccharomyces, Hansenula polymorpha, any Kluyveromyces, Candida albicans, any Aspergillus, Trichoderma reesei, Chrysosporium lucknowense, any Fusarium, Yarrowia lipolytica, and Neurospora crassa. The host cell of the present patent does not include objects not authorized by the Patent Laws.

“Optional” or “optionally” means that the event or circumstance subsequently described may, but does not necessarily, occur, and this description includes instances where the event or circumstance occurs or does not occur.

The term “pharmaceutical composition” refers to a mixture comprising one or more of the antigen-binding molecules or antibodies described herein and other chemical components; the other components are, for example, physiological/pharmaceutically acceptable carriers and excipients.

The term “pharmaceutically acceptable carrier, diluent, buffer, or excipient” refers to an ingredient in a pharmaceutical formulation that is different from the active ingredient and is not toxic to the subject. Pharmaceutically acceptable carriers, diluents, buffers, or excipients include, but are not limited to, buffers, excipients, stabilizers, or preservatives.

The term “subject” or “individual” includes humans and non-human animals. Non-human animals include all vertebrates (e.g., mammals and non-mammals) such as non-human primates (e.g., cynomolgus monkeys), sheep, dogs, cows, chickens, amphibians, and reptiles. Unless clearly indicated, the terms “patient” and “subject” are used interchangeably herein. As used herein, the term “cynomolgus monkey (cyno)” or “cynomolgus” refers to Macaca fascicularis. In certain embodiments, the individual or subject is a human.

“Administrating” or “giving”, when applied to animals, humans, experimental subjects, cells, tissue, organs, or biological fluids, refers to contact of an exogenous drug, a therapeutic agent, a diagnostic agent, or a composition with the animals, humans, subjects, cells, tissue, organs, or biological fluids.

The term “sample” refers to a collection of similar fluids, cells or tissues isolated from a subject, as well as fluids, cells or tissues present within a subject. Exemplary samples are biological fluids (such as blood; serum; serosal fluids; plasma; lymph; urine; saliva; cystic fluids; tears; excretions; sputum; mucosal secretions of secretory tissue and organs; vaginal secretions; ascites; fluids in the pleura, pericardium, peritoneum, abdominal cavity, and other body cavities; fluids collected from bronchial lavage; synovial fluids; liquid solutions in contact with a subject or biological source, e.g., cell and organ culture media (including cell or organ conditioned culture media); lavage fluids; and the like), tissue biopsy samples, fine needle punctures, surgically excised tissues, organ cultures, or cell cultures.

“Treatment” or “treat” (and grammatical variations thereof) refers to clinical intervention intended to be applied to the treated individual, which may be performed either for prophylactic purposes or during the course of clinical pathology. Desirable effects of the treatment include, but are not limited to, preventing the occurrence or recurrence of a disease, alleviating symptoms, alleviating/reducing any direct or indirect pathological consequences of the disease, preventing metastasis, decreasing the rate of disease progression, ameliorating or alleviating the disease state, and regressing or improving prognosis. In some embodiments, the antigen-binding molecule of the present disclosure is used to delay the development of or slow the progression of disease.

The terms “recurrence”, “relapse”, or “relapsed” refer to the return of a cancer or disease after clinical assessment of the disappearance of disease. A diagnosis of distant cancer metastasis or local recurrence can be considered a relapse.

“Effective amount” is generally an amount sufficient to reduce the severity and/or frequency of symptoms, eliminate symptoms and/or underlying causes, prevent the appearance of symptoms and/or their underlying causes, and/or ameliorate or improve damage caused by or associated with a disease state. In some examples, the effective amount is a therapeutically effective amount or a prophylactically effective amount.

“Therapeutically effective amount” is an amount sufficient to treat a disease state or symptom, particularly a state or symptom associated with the disease state, or to otherwise prevent, hinder, delay, or reverse the progression of the disease state or any other undesirable symptoms associated with the disease in any way.

“Prophylactically effective amount” is an amount that, when administered to a subject, will have a predetermined prophylactic effect, e.g., preventing or delaying the onset (or recurrence) of the disease state, or reducing the likelihood of the onset (or recurrence) of the disease state or associated symptoms. A complete therapeutic or prophylactic effect does not necessarily occur after administration of one dose and may occur after administration of a series of doses. Thus, a therapeutically or prophylactically effective amount may be administered in one or more doses. “Therapeutically effective amount” and “prophylactically effective amount” may vary depending on a variety of factors such as the disease state, age, sex, and weight of the individual, and the ability of a therapeutic agent or combination of therapeutic agents to elicit a desired response in the individual. Exemplary indicators of an effective therapeutic agent or combination of therapeutic agents include, for example, improved health condition of a subject.

### Antigen-Binding Molecules of the Present Disclosure

The present disclosure provides an antigen-binding molecule having a number of advantageous properties, such as good in vitro killing activity, therapeutic activity, safety, pharmacokinetic properties, and druggability (e.g., yield, purity, and stability).

### Exemplary Antigen-Binding Molecules

The antigen-binding molecule of the present disclosure includes bispecific antigen-binding molecules (e.g., bispecific antibodies) binding specifically to DLL3 and CD3 and anti-DLL3 antibodies. Particularly, the antigen-binding molecule of the present disclosure has any one of the following properties:
a. A high affinity for DLL3. In some embodiments, the anti-DLL3 antibody or antigen-binding molecule binds to human DLL3 with a KD of less than 4×10⁻⁹ M, 3×10⁻⁹ M, 2×10⁻⁹ M, 1×10⁻⁹ M, 9×10⁻¹⁰ M, 8×10⁻¹⁰ M, 7×10⁻¹⁰ M, 6×10⁻¹⁰ M, 5×10⁻¹⁰ M, 4×10⁻¹⁰ M, 3×10⁻¹⁰ M, 2×10⁻¹⁰ M, 1×10⁻¹⁰ M, or 9×10⁻¹¹M, as measured by surface plasmon resonance. In some embodiments, the antigen-binding molecule binds to DLL3 protein with an EC50 of less than 0.5 nM, 0.4 nM, 0.3 nM, 0.2 nM, 0.1 nM, 0.09 nM, 0.08 nM, or 0.07 nM, as determined by ELISA.

b. A high affinity for DLL3 expressed on cell surfaces. In some embodiments, the anti-DLL3 antibody binds to H1184 cells with an EC50 of less than 0.2 nM, 0.1 nM, 0.09 nM, 0.08 nM, 0.07 nM, 0.06 nM, 0.05 nM, or 0.04 nM.

c. A specific killing effect on DLL3-expressing cells. In some embodiments, the antigen-binding molecule may specifically kill DLL3-expressing SHP77 cells and H1184 cells, wherein the killing IC50 against H1184 cells is less than 30 pM, 25 pM, 20 pM, 15 pM, 14 pM, 13 pM, 12 pM, 11 pM, 10 pM, 9 pM, 8 pM, or 7 pM.

d. In vitro activating activity for T cells. In some embodiments, the antigen-binding molecule activates Jurkat Lucia™ NFAT cells with an EC50 of less than 20 pM, 19 pM, 18 pM, 17 pM, 16 pM, 15 pM, 14 pM, 13 pM, 12 pM, 11 pM, 10 pM, 9 pM, 8 pM, or 7 pM.

e. Induction of low-level release of cytokines (IL6 and IFNγ).

e. Stronger in vivo therapeutic activity. In some embodiments, the antigen-binding molecule may significantly inhibit the growth of subcutaneous xenograft tumors of SHP77 and H1184 cells in mice.

The present disclosure provides an antigen-binding molecule comprising at least one antigen-binding moiety binding specifically to DLL3 and at least one antigen-binding moiety binding specifically to CD3; the antigen-binding moiety binding specifically to DLL3 comprises a DLL3-VH and a DLL3-VL, and the antigen-binding moiety binding specifically to CD3 comprises a CD3-VH and a CD3-VL. The present disclosure also provides an anti-DLL3 antibody capable of binding specifically to DLL3, the antibody comprising a DLL3-VH and a DLL3-VL. Specifically, the examples herein disclose antibody series 6, 98, 110, and 30. Taking antibodies 6 and 110 as examples, the antibodies or antigen-binding molecules herein are described below.

Provided is an antigen-binding molecule binding specifically to DLL3 and CD3, wherein:
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, 71, 72, or 73, and a DLL3-LCDR3 comprising the amino acid sequence of SEQ ID NO: 25 or 74.

Provided is an antigen-binding molecule binding specifically to DLL3 and CD3 or an anti-DLL3 antibody, wherein:
the DLL3-VH comprises: a DLL3-HCDR1 comprising the amino acid sequence of SEQ ID NO: 20, a DLL3-HCDR2 comprising the amino acid sequence of SEQ ID NO: 32, 96, 97, 98, or 99, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36.

Provided is an antigen-binding molecule binding specifically to DLL3 and CD3, wherein:
the DLL3-VH comprises a DLL3-HCDR1 set forth in SEQ ID NO: 20, a DLL3-HCDR2 set forth in SEQ ID NO: 21, 69, or 70, and a DLL3-HCDR3 set forth in SEQ ID NO: 22, and the DLL3-VL comprises a DLL3-LCDR1 set forth in SEQ ID NO: 23, a DLL3-LCDR2 set forth in SEQ ID NO: 24, 71, 72, or 73, and a DLL3-LCDR3 set forth in SEQ ID NO: 25 or 74.

Provided is an antigen-binding molecule binding specifically to DLL3 and CD3 or an anti-DLL3 antibody, wherein:
the DLL3-VH comprises a DLL3-HCDR1 set forth in SEQ ID NO: 20, a DLL3-HCDR2 set forth in SEQ ID NO: 32, 96, 97, 98, or 99, and a DLL3-HCDR3 set forth in SEQ ID NO: 33, and the DLL3-VL comprises a DLL3-LCDR1 set forth in SEQ ID NO: 34, a DLL3-LCDR2 set forth in SEQ ID NO: 35, and a DLL3-LCDR3 set forth in SEQ ID NO: 36.

Provided is an antigen-binding molecule binding specifically to DLL3 and CD3, wherein:
the DLL3-VH comprises a DLL3-HCDR1 set forth in SEQ ID NO: 20, a DLL3-HCDR2 set forth in SEQ ID NO: 21, and a DLL3-HCDR3 set forth in SEQ ID NO: 22; and the DLL3-VL comprises a DLL3-LCDR1 set forth in SEQ ID NO: 23, a DLL3-LCDR2 set forth in SEQ ID NO: 71, and a DLL3-LCDR3 set forth in SEQ ID NO: 25.

Provided is an antigen-binding molecule binding specifically to DLL3 and CD3 or an anti-DLL3 antibody, wherein:
the DLL3-VH comprises a DLL3-HCDR1 set forth in SEQ ID NO: 20, a DLL3-HCDR2 set forth in SEQ ID NO: 21, and a DLL3-HCDR3 set forth in SEQ ID NO: 22; and the DLL3-VL comprises a DLL3-LCDR1 set forth in SEQ ID NO: 23, a DLL3-LCDR2 set forth in SEQ ID NO: 24, and a DLL3-LCDR3 set forth in SEQ ID NO: 25; or
the DLL3-VH comprises a DLL3-HCDR1 set forth in SEQ ID NO: 20, a DLL3-HCDR2 set forth in SEQ ID NO: 21, and a DLL3-HCDR3 set forth in SEQ ID NO: 22; and the DLL3-VL comprises a DLL3-LCDR1 set forth in SEQ ID NO: 23, a DLL3-LCDR2 set forth in SEQ ID NO: 72, and a DLL3-LCDR3 set forth in SEQ ID NO: 25; or
the DLL3-VH comprises a DLL3-HCDR1 set forth in SEQ ID NO: 20, a DLL3-HCDR2 set forth in SEQ ID NO: 69, and a DLL3-HCDR3 set forth in SEQ ID NO: 22; and the DLL3-VL comprises a DLL3-LCDR1 set forth in SEQ ID NO: 23, a DLL3-LCDR2 set forth in SEQ ID NO: 71, and a DLL3-LCDR3 set forth in SEQ ID NO: 74; or
the DLL3-VH comprises a DLL3-HCDR1 set forth in SEQ ID NO: 20, a DLL3-HCDR2 set forth in SEQ ID NO: 69, and a DLL3-HCDR3 set forth in SEQ ID NO: 22; and the DLL3-VL comprises a DLL3-LCDR1 set forth in SEQ ID NO: 23, a DLL3-LCDR2 set forth in SEQ ID NO: 24, and a DLL3-LCDR3 set forth in SEQ ID NO: 74; or
the DLL3-VH comprises a DLL3-HCDR1 set forth in SEQ ID NO: 20, a DLL3-HCDR2 set forth in SEQ ID NO: 69, and a DLL3-HCDR3 set forth in SEQ ID NO: 22; and the DLL3-VL comprises a DLL3-LCDR1 set forth in SEQ ID NO: 23, a DLL3-LCDR2 set forth in SEQ ID NO: 71, and a DLL3-LCDR3 set forth in SEQ ID NO: 25; or
the DLL3-VH comprises a DLL3-HCDR1 set forth in SEQ ID NO: 20, a DLL3-HCDR2 set forth in SEQ ID NO: 70, and a DLL3-HCDR3 set forth in SEQ ID NO: 22; and the DLL3-VL comprises a DLL3-LCDR1 set forth in SEQ ID NO: 23, a DLL3-LCDR2 set forth in SEQ ID NO: 71, and a DLL3-LCDR3 set forth in SEQ ID NO: 25; or
the DLL3-VH comprises a DLL3-HCDR1 set forth in SEQ ID NO: 20, a DLL3-HCDR2 set forth in SEQ ID NO: 70, and a DLL3-HCDR3 set forth in SEQ ID NO: 22; and the DLL3-VL comprises a DLL3-LCDR1 set forth in SEQ ID NO: 23, a DLL3-LCDR2 set forth in SEQ ID NO: 24, and a DLL3-LCDR3 set forth in SEQ ID NO: 74.

Provided is an antigen-binding molecule binding specifically to DLL3 and CD3 or an anti-DLL3 antibody, wherein:
the DLL3-VH comprises a DLL3-HCDR1 set forth in SEQ ID NO: 20, a DLL3-HCDR2 set forth in SEQ ID NO: 96, and a DLL3-HCDR3 set forth in SEQ ID NO: 33; and the DLL3-VL comprises a DLL3-LCDR1 set forth in SEQ ID NO: 34, a DLL3-LCDR2 set forth in SEQ ID NO: 35, and a DLL3-LCDR3 set forth in SEQ ID NO: 36.

In some embodiments, in the antigen-binding molecule or the anti-DLL3 antibody according to the foregoing, the DLL3-VH and/or the DLL3-VL are/is murine or humanized. In some embodiments, the DLL3-VH and/or the DLL3-VL are/is humanized.

In some embodiments, the FR1, FR2, FR3, and FR4 of the humanized DLL3-VH have at least 60%, 70%, 80%, or 90% sequence identity to the FR1, FR2, FR3, and FR4 of SEQ ID NO: 50, 51, or 52; the FR1, FR2, FR3, and FR4 of the humanized DLL3-VL have at least 60%, 70%, 80%, or 90% sequence identity to the FR1, FR2, FR3, and FR4 of SEQ ID NO: 62 or 68.

In some embodiments, the humanized DLL3-VH comprises a FR1, a FR2, and a FR3 derived from IGHV1-3*01, IGHV7-4-1*02, or IGHV3-73*01 and a FR4 derived from IGHJ6*01, and is unsubstituted or has one or more amino acid substitutions selected from the group consisting of 1E, 5Q, 27Y, 30T, 38K, 43K, 48I, 67A, 68A, 69L, 71V, 73K, 75S, 76N, and 93A; and/or the humanized DLL3-VL comprises a FR1, a FR2, and a FR3 derived from IGKV1-16*01 or IGKV3-20*02 and a FR4 derived from IGKJ4*01, and is unsubstituted or has one or more amino acid substitutions selected from the group consisting of 1N, 36L, 42K, 43S, 44F, 46G, 69A, 71Y, 79Q, and 85D. In some embodiments, the amino acid positions in the above variable regions are defined according to the Kabat numbering scheme.

In some embodiments, the humanized DLL3-VH comprises the amino acid sequence of SEQ ID NO: 50 or a variant thereof. In some embodiments, the variant comprises one or more amino acid substitutions selected from the group consisting of 1E, 43K, 48I, 67A, 69L, 71V, 73K, and 76N in FR regions of SEQ ID NO: 50.

In some embodiments, the humanized DLL3-VH comprises the amino acid sequence of SEQ ID NO: 51 or a variant thereof. In some embodiments, the variant comprises one or more amino acid substitutions selected from the group consisting of 1E, 5Q, 38K, 68A, 69L, 71V, 73K, 75S, and 76N in FR regions of SEQ ID NO: 51.

In some embodiments, the humanized DLL3-VH comprises the amino acid sequence of SEQ ID NO: 52 or a variant thereof. In some embodiments, the variant comprises one or more amino acid substitutions selected from the group consisting of 27Y, 30T, 69L, 71V, 73K, and 93A in FR regions of SEQ ID NO: 52.

In some embodiments, the humanized DLL3-VL comprises the amino acid sequence of SEQ ID NO: 62 or a variant thereof. In some embodiments, the variant comprises one or more amino acid substitutions selected from the group consisting of 36L, 43S, 44F, 46G, 69A, 71Y, and 85D in FR regions of SEQ ID NO: 62.

In some embodiments, the humanized DLL3-VL comprises the amino acid sequence of SEQ ID NO: 68 or a variant thereof. In some embodiments, the variant comprises one or more amino acid substitutions selected from the group consisting of 36L, 42K, 46G, 69A, 71Y, 79Q, and 85D in FR regions of SEQ ID NO: 68.

In some embodiments, the FR1, FR2, FR3, and FR4 of the humanized DLL3-VH have at least 60%, 70%, 80%, or 90% sequence identity to the FR1, FR2, FR3, and FR4 of SEQ ID NO: 95; the FR1, FR2, FR3, and FR4 of the humanized DLL3-VL have at least 60%, 70%, 80%, or 90% sequence identity to the FR1, FR2, FR3, and FR4 of SEQ ID NO: 88.

In some embodiments, the humanized DLL3-VH comprises a FR1, a FR2, and a FR3 derived from IGHV1-3*01 and a FR4 derived from IGHJ6*01, and is unsubstituted or has one or more amino acid substitutions selected from the group consisting of 1E, 24T, 44S, 71V, and 73K; and/or the humanized DLL3-VL comprises a FR1, a FR2, and a FR3 derived from IGKV4-1*01 and a FR4 derived from IGKJ2*01, and is unsubstituted or has one or more amino acid substitutions selected from the group consisting of 1N, 9K, 43S, 68A, and 85D. In some embodiments, the amino acid positions in the above variable regions are defined according to the Kabat numbering scheme.

In some embodiments, the humanized DLL3-VH comprises the amino acid sequence of SEQ ID NO: 95 or a variant thereof. In some embodiments, the variant comprises one or more amino acid substitutions selected from the group consisting of 1E, 24T, 44S, 71V, and 73K in FR regions of SEQ ID NO: 95. In some embodiments, the humanized DLL3-VL comprises the amino acid sequence of SEQ ID NO: 88 or a variant thereof. In some embodiments, the variant comprises one or more amino acid substitutions selected from the group consisting of 1N, 9K, 43S, 68A, and 85D in FR regions of SEQ ID NO: 88.

In some embodiments, the humanized DLL3-VH comprises the amino acid sequence of SEQ ID NO: 51 or a variant thereof; in some embodiments, the variant comprises one or more amino acid substitutions selected from the group consisting of 1E, 38K, 68A, 69L, 71V, 73K, 75S, and 76N in FR regions of SEQ ID NO: 51; and the humanized DLL3-VL comprises the amino acid sequence of SEQ ID NO: 63 or a variant thereof; in some embodiments, the variant comprises one or more amino acid substitutions in FR regions of SEQ ID NO: 63; in some embodiments, the amino acid substitutions are one or more amino acid substitutions selected from the group consisting of 43S and 44F.

In some embodiments, the humanized DLL3-VH comprises the amino acid sequence of SEQ ID NO: 91 or a variant of SEQ ID NO: 91; in some embodiments, the variant comprises one or more amino acid substitutions in FR regions of SEQ ID NO: 91; and
the humanized DLL3-VL comprises the amino acid sequence of SEQ ID NO: 88 or a variant thereof; in some embodiments, the variant comprises one or more amino acid substitutions selected from the group consisting of 1N, 9K, 43S, 68A, and 85D in FR regions of SEQ ID NO: 88.

In some embodiments, the variable regions and CDRs are defined according to the Kabat numbering scheme.

In some embodiments, provided is the antigen-binding molecule according to any one of the foregoing, wherein the amino acid sequence of the DLL3-VH has at least 90%, 95%, 96%, 97%, 98%, or 99% sequence identity to SEQ ID NO: 12, 43, 44, 45, 46, 47, 48, 49, 50, 51, 52, 53, 54, 55, 56, or 57, and the amino acid sequence of the DLL3-VL has at least 90%, 95%, 96%, 97%, 98%, or 99% sequence identity to SEQ ID NO: 13, 58, 59, 60, 61, 62, 63, 64, 65, 66, 67, or 68. In some embodiments, the amino acid sequence of the DLL3-VH is set forth in SEQ ID NO: 12, 43, 44, 45, 46, 47, 48, 49, 50, 51, 52, 53, 54, 55, 56, or 57, and the amino acid sequence of the DLL3-VL is set forth in SEQ ID NO: 13, 58, 59, 60, 61, 62, 63, 64, 65, 66, 67, or 68.

In some embodiments, provided is the anti-DLL3 antibody according to any one of the foregoing, wherein the amino acid sequence of the DLL3-VH has at least 90%, 95%, 96%, 97%, 98%, or 99% sequence identity to SEQ ID NO: 43, 44, 45, 46, 47, 48, 49, 50, 51, 52, 53, 54, 55, 56, or 57, and the amino acid sequence of the DLL3-VL has at least 90%, 95%, 96%, 97%, 98%, or 99% sequence identity to SEQ ID NO: 63, 64, 65, 66, or 67. In some embodiments, the amino acid sequence of the DLL3-VH is set forth in SEQ ID NO: 43, 44, 45, 46, 47, 48, 49, 50, 51, 52, 53, 54, 55, 56, or 57, and the amino acid sequence of the DLL3-VL is set forth in SEQ ID NO: 63, 64, 65, 66, or 67.

In some embodiments, provided is the anti-DLL3 antibody according to any one of the foregoing, wherein the amino acid sequence of the DLL3-VH has at least 90%, 95%, 96%, 97%, 98%, or 99% sequence identity to SEQ ID NO: 56 or 57, and the amino acid sequence of the DLL3-VL has at least 90%, 95%, 96%, 97%, 98%, or 99% sequence identity to SEQ ID NO: 58, 59, 60, 61, 62, 63, 64, 65, 66, 67, or 68. In some embodiments, the amino acid sequence of the DLL3-VH is set forth in SEQ ID NO: 56 or 57, and the amino acid sequence of the DLL3-VL is set forth in SEQ ID NO: 58, 59, 60, 61, 62, 63, 64, 65, 66, 67, or 68.

In some embodiments, in the antigen-binding molecule or the anti-DLL3 antibody according to any one of the foregoing, the amino acid sequence of the DLL3-VH is set forth in SEQ ID NO: 54, and the amino acid sequence of the DLL3-VL is set forth in SEQ ID NO: 63; or
the amino acid sequence of the DLL3-VH is set forth in SEQ ID NO: 54, and the amino acid sequence of the DLL3-VL is set forth in SEQ ID NO: 61; or
the amino acid sequence of the DLL3-VH is set forth in SEQ ID NO: 54, and the amino acid sequence of the DLL3-VL is set forth in SEQ ID NO: 64; or
the amino acid sequence of the DLL3-VH is set forth in SEQ ID NO: 56, and the amino acid sequence of the DLL3-VL is set forth in SEQ ID NO: 65; or
the amino acid sequence of the DLL3-VH is set forth in SEQ ID NO: 56, and the amino acid sequence of the DLL3-VL is set forth in SEQ ID NO: 67; or
the amino acid sequence of the DLL3-VH is set forth in SEQ ID NO: 56, and the amino acid sequence of the DLL3-VL is set forth in SEQ ID NO: 65; or
the amino acid sequence of the DLL3-VH is set forth in SEQ ID NO: 57, and the amino acid sequence of the DLL3-VL is set forth in SEQ ID NO: 65; or
the amino acid sequence of the DLL3-VH is set forth in SEQ ID NO: 57, and the amino acid sequence of the DLL3-VL is set forth in SEQ ID NO: 67; or
the amino acid sequence of the DLL3-VH is set forth in SEQ ID NO: 57, and the amino acid sequence of the DLL3-VL is set forth in SEQ ID NO: 65.

In some embodiments, provided is the antigen-binding molecule or the anti-DLL3 antibody according to any one of the foregoing, wherein the amino acid sequence of the DLL3-VH has at least 90%, 95%, 96%, 97%, 98%, or 99% sequence identity to SEQ ID NO: 16, 89, 90, 91, 92, 93, 94, or 95, and the amino acid sequence of the DLL3-VL has at least 90%, 95%, 96%, 97%, 98%, or 99% sequence identity to SEQ ID NO: 17, 86, 87, or 88. In some embodiments, the amino acid sequence of the DLL3-VH is set forth in SEQ ID NO: 16, 89, 90, 91, 92, 93, 94, or 95, and the amino acid sequence of the DLL3-VL is set forth in SEQ ID NO: 17, 86, 87, or 88.

In some embodiments, in the antigen-binding molecule or the anti-DLL3 antibody according to any one of the foregoing, the amino acid sequence of the DLL3-VH is set forth in SEQ ID NO: 91, and the amino acid sequence of the DLL3-VL is set forth in SEQ ID NO: 87.

In some embodiments, the anti-DLL3 antibody according to any one of the foregoing comprises a heavy chain and a light chain, wherein:
the amino acid sequence of the heavy chain is set forth in SEQ ID NO: 121, and the amino acid sequence of the light chain is set forth in SEQ ID NO: 122; or
the amino acid sequence of the heavy chain is set forth in SEQ ID NO: 125, and the amino acid sequence of the light chain is set forth in SEQ ID NO: 126.

In some embodiments, provided is the antigen-binding molecule according to the above, wherein:
i) the CD3-VH comprises a CD3-HCDR1 set forth in SEQ ID NO: 129, a CD3-HCDR2 set forth in SEQ ID NO: 130, and a CD3-HCDR3 set forth in SEQ ID NO: 131; and the CD3-VL comprises a CD3-LCDR1 set forth in SEQ ID NO: 132, a CD3-LCDR2 set forth in SEQ ID NO: 133, and a CD3-LCDR3 set forth in SEQ ID NO: 134; or
ii) the CD3-VH comprises a CD3-HCDR1 set forth in SEQ ID NO: 129, a CD3-HCDR2 set forth in SEQ ID NO: 130, and a CD3-HCDR3 set forth in SEQ ID NO: 135; and the CD3-VL comprises a CD3-LCDR1 set forth in SEQ ID NO: 132, a CD3-LCDR2 set forth in SEQ ID NO: 133, and a CD3-LCDR3 set forth in SEQ ID NO: 134.

In some embodiments, the variable regions and CDRs are defined according to the Kabat numbering scheme.

In some embodiments, provided is the antigen-binding molecule according to any one of the foregoing, wherein the CD3-VH and/or the CD3-VL are/is murine or humanized. In some embodiments, the CD3-VH and/or the CD3-VL are/is humanized.

In some embodiments, provided is the antigen-binding molecule according to any one of the foregoing, wherein the amino acid sequence of the CD3-VH is set forth in SEQ ID NO: 136 or 138, and the amino acid sequence of the CD3-VL is set forth in SEQ ID NO: 137.

In some embodiments, the antigen-binding molecule according to any one of the foregoing comprises:
a first chain set forth in SEQ ID NO: 152, a second chain set forth in SEQ ID NO: 153, a third chain set forth in SEQ ID NO: 154, and a fourth chain set forth in SEQ ID NO: 155; or
a first chain set forth in SEQ ID NO: 152, a second chain set forth in SEQ ID NO: 153, a third chain set forth in SEQ ID NO: 156, and a fourth chain set forth in SEQ ID NO: 155.

In some embodiments, the antigen-binding molecule according to any one of the foregoing comprises:
a first chain set forth in SEQ ID NO: 157, a second chain set forth in SEQ ID NO: 158, and a third chain set forth in SEQ ID NO: 159; or
a first chain set forth in SEQ ID NO: 157, a second chain set forth in SEQ ID NO: 158, and a third chain set forth in SEQ ID NO: 160.

In some embodiments, the antigen-binding molecule according to any one of the foregoing comprises:
a first chain set forth in SEQ ID NO: 161 and a second chain set forth in SEQ ID NO: 162; or
a first chain set forth in SEQ ID NO: 163 and a second chain set forth in SEQ ID NO: 162; or
a first chain set forth in SEQ ID NO: 185 and a second chain set forth in SEQ ID NO: 162; or
a first chain set forth in SEQ ID NO: 186 and a second chain set forth in SEQ ID NO: 162; or
a first chain set forth in SEQ ID NO: 259 and a second chain set forth in SEQ ID NO: 162; or
a first chain set forth in SEQ ID NO: 260 and a second chain set forth in SEQ ID NO: 162; or
a first chain set forth in SEQ ID NO: 187 and a second chain set forth in SEQ ID NO: 188; or
a first chain set forth in SEQ ID NO: 187 and a second chain set forth in SEQ ID NO: 189; or
a first chain set forth in SEQ ID NO: 190 and a second chain set forth in SEQ ID NO: 188; or
a first chain set forth in SEQ ID NO: 190 and a second chain set forth in SEQ ID NO: 189; or
a first chain set forth in SEQ ID NO: 191 and a second chain set forth in SEQ ID NO: 188; or
a first chain set forth in SEQ ID NO: 191 and a second chain set forth in SEQ ID NO: 189.

In some embodiments, the antigen-binding molecule according to any one of the foregoing comprises:
a first chain set forth in SEQ ID NO: 171, a second chain set forth in SEQ ID NO: 172, a third chain set forth in SEQ ID NO: 154, and a fourth chain set forth in SEQ ID NO: 155; or
a first chain set forth in SEQ ID NO: 171, a second chain set forth in SEQ ID NO: 172, a third chain set forth in SEQ ID NO: 156, and a fourth chain set forth in SEQ ID NO: 155.

In some embodiments, the antigen-binding molecule according to any one of the foregoing comprises:
a first chain set forth in SEQ ID NO: 173, a second chain set forth in SEQ ID NO: 174, and a third chain set forth in SEQ ID NO: 159; or
a first chain set forth in SEQ ID NO: 173, a second chain set forth in SEQ ID NO: 174, and a third chain set forth in SEQ ID NO: 160.

In some embodiments, the antigen-binding molecule according to any one of the foregoing comprises:
a first chain set forth in SEQ ID NO: 175 and a second chain set forth in SEQ ID NO: 176; or
a first chain set forth in SEQ ID NO: 177 and a second chain set forth in SEQ ID NO: 176.

The antigen-binding molecules comprising antibody series 98 and 30 disclosed according to the examples herein have a technical solution scope equivalent to that of the antibody series 6 and 110 described above.

### Structure of Antigen-Binding Molecule

The bispecific antigen-binding molecule of the present disclosure is not limited to a particular molecular structure, as long as it has the desired antigen-binding function. For example, the bispecific antigen-binding molecule herein may be bivalent (1+1), trivalent (2+1), or tetravalent (2+2). The antigen-binding moieties in the antigen-binding molecule may be any antibody fragments having antigen-binding activity that are fused via a peptide linker. The peptide linkers of the present disclosure (e.g., linkers 1 to 8) can be any suitable peptide chains, as long as the antigen-binding molecules are capable of exhibiting the desired antigen-binding activity. For example, the peptide linkers may be flexible peptides comprising 1-50 or 3-20 amino acid residues. In some embodiments, the peptide linkers each independently have a structure of (GGGGS)n, wherein n is 1, 2, or 3. In some embodiments, the sequences of the linker 1 and linker 2 are both GGGGS (SEQ ID NO: 147). In some embodiments, the sequence of the linker 3 is GGGGSGGGGSGGGGS (SEQ ID NO: 148). In some embodiments, the sequence of the linker 4 is GGGGS (SEQ ID NO: 147). In some embodiments, the sequence of the linker 5 is GGGSGGGG (SEQ ID NO: 149). In some embodiments, the sequences of linker 6 and linker 8 are G (SEQ ID NO: 150). In some embodiments, the sequence of linker 7 is GGGGSGGGG (SEQ ID NO: 151).

Illustratively, the antigen-binding molecule of the present disclosure comprises one first chain having a structure represented by formula (a-1), one second chain having a structure represented by formula (b-1), one third chain having a structure represented by formula (c), and one fourth chain having a structure represented by formula (d):

$$[ DLL 3 - VH ] - [ GGGGS ] - [ Titin chain ] - [ Fc 2 ] , \qquad ( a - 1 )$$

$$[ DLL 3 - VL ] - [ GGGGS ] - [ Obscurin chain ] , \qquad ( b - 1 )$$

$$[ CD 3 - VH ] - [ CH 1 ] - [ Fc 1 ] , and \qquad ( c )$$

$$[ CD 3 - VL ] - [ CL ] , \qquad ( d )$$

the structures represented by formulas (a-1), (b-1), (c), and (d) are arranged from N-terminus to C-terminus.

An exemplary antigen-binding molecule comprises:
one first chain set forth in SEQ ID NO: 171, one second chain set forth in SEQ ID NO: 172, one third chain set forth in SEQ ID NO: 154, and one fourth chain set forth in SEQ ID NO: 155; or
one first chain set forth in SEQ ID NO: 171, one second chain set forth in SEQ ID NO: 172, one third chain set forth in SEQ ID NO: 156, and one fourth chain set forth in SEQ ID NO: 155; or
one first chain set forth in SEQ ID NO: 152, one second chain set forth in SEQ ID NO: 153, one third chain set forth in SEQ ID NO: 154, and one fourth chain set forth in SEQ ID NO: 155; or
one first chain set forth in SEQ ID NO: 152, one second chain set forth in SEQ ID NO: 153, one third chain set forth in SEQ ID NO: 156, and one fourth chain set forth in SEQ ID NO: 155; or
one first chain set forth in SEQ ID NO: 164, one second chain set forth in SEQ ID NO: 165, one third chain set forth in SEQ ID NO: 154, and one fourth chain set forth in SEQ ID NO: 155; or
one first chain set forth in SEQ ID NO: 164, one second chain set forth in SEQ ID NO: 165, one third chain set forth in SEQ ID NO: 156, and one fourth chain set forth in SEQ ID NO: 155; or
one first chain set forth in SEQ ID NO: 178, one second chain set forth in SEQ ID NO: 179, one third chain set forth in SEQ ID NO: 154, and one fourth chain set forth in SEQ ID NO: 155; or
one first chain set forth in SEQ ID NO: 178, one second chain set forth in SEQ ID NO: 179, one third chain set forth in SEQ ID NO: 156, and one fourth chain set forth in SEQ ID NO: 155.

Illustratively, the antigen-binding molecule comprises one first chain having a structure represented by formula (e), one second chain having a structure represented by formula (f), and one third chain having a structure represented by formula (g-1):

$$[ DLL 3 - VL ] - [ CL ] , \qquad ( e )$$

$$[ DLL 3 - VL ] - [ CH 1 ] - [ Fc 2 ] , and \qquad ( f )$$

$$[ CD 3 - VH ] - [ GGGGSGGGGSGGGGS ] - [ CD 3 - VL ] - [ GGGGS ] - [ Fc 1 ] ; \qquad ( g - 1 )$$

the structures represented by formulas (e), (f), and (g-1) are arranged from N-terminus to C-terminus.

An exemplary antigen-binding molecule comprises:
one first chain set forth in SEQ ID NO: 173, one second chain set forth in SEQ ID NO: 174, and one third chain set forth in SEQ ID NO: 160; or
one first chain set forth in SEQ ID NO: 173, one second chain set forth in SEQ ID NO: 174, and one third chain set forth in SEQ ID NO: 159; or
one first chain set forth in SEQ ID NO: 157, one second chain set forth in SEQ ID NO: 158, and one third chain set forth in SEQ ID NO: 159; or
one first chain set forth in SEQ ID NO: 157, one second chain set forth in SEQ ID NO: 158, and one third chain set forth in SEQ ID NO: 160; or
one first chain set forth in SEQ ID NO: 166, one second chain set forth in SEQ ID NO: 167, and one third chain set forth in SEQ ID NO: 159; or
one first chain set forth in SEQ ID NO: 166, one second chain set forth in SEQ ID NO: 167, and one third chain set forth in SEQ ID NO: 160; or
one first chain set forth in SEQ ID NO: 180, one second chain set forth in SEQ ID NO: 181, and one third chain set forth in SEQ ID NO: 159; or
one first chain set forth in SEQ ID NO: 180, one second chain set forth in SEQ ID NO: 181, and one third chain set forth in SEQ ID NO: 160.

Illustratively, the antigen-binding molecule comprises one first chain having a structure represented by formula (i-1) and one second chain having a structure represented by formula (h-1):

$$[ DLL 3 - VL ] - [ GGGSGGGG ] - [ CD 3 - VH ] - [ G ] - [ Fc 1 ] , and \qquad ( h - 1 )$$

$$[ CD 3 - VL ] - [ GGGGSGGGG ] - [ DLL 3 - VH ] - [ G ] - [ Fc 2 ] ; \qquad ( i - 1 )$$

the structures represented by formulas (h-1) and (i-1) are arranged from N-terminus to C-terminus.

An exemplary antigen-binding molecule comprises:
one first chain set forth in SEQ ID NO: 185 and one second chain set forth in SEQ ID NO: 162; or
one first chain set forth in SEQ ID NO: 161 and one second chain set forth in SEQ ID NO: 162; or
one first chain set forth in SEQ ID NO: 163 and one second chain set forth in SEQ ID NO: 162; or
one first chain set forth in SEQ ID NO: 259 and one second chain set forth in SEQ ID NO: 162; or
one first chain set forth in SEQ ID NO: 260 and one second chain set forth in SEQ ID NO: 162; or
one first chain set forth in SEQ ID NO: 168 and one second chain set forth in SEQ ID NO: 169; or
one first chain set forth in SEQ ID NO: 170 and one second chain set forth in SEQ ID NO: 169; or
one first chain set forth in SEQ ID NO: 175 and one second chain set forth in SEQ ID NO: 176; or
one first chain set forth in SEQ ID NO: 177 and one second chain set forth in SEQ ID NO: 176; or
one first chain set forth in SEQ ID NO: 182 and one second chain set forth in SEQ ID NO: 183; or
one first chain set forth in SEQ ID NO: 184 and one second chain set forth in SEQ ID NO: 183; or
one first chain set forth in SEQ ID NO: 186 and one second chain set forth in SEQ ID NO: 162; or
one first chain set forth in SEQ ID NO: 187 and one second chain set forth in SEQ ID NO: 188; or
one first chain set forth in SEQ ID NO: 187 and one second chain set forth in SEQ ID NO: 189; or
one first chain set forth in SEQ ID NO: 190 and one second chain set forth in SEQ ID NO: 188; or
one first chain set forth in SEQ ID NO: 190 and one second chain set forth in SEQ ID NO: 189; or
one first chain set forth in SEQ ID NO: 191 and one second chain set forth in SEQ ID NO: 188; or
one first chain set forth in SEQ ID NO: 191 and one second chain set forth in SEQ ID NO: 189.

### Variants of Antigen-Binding Molecules

In certain embodiments, amino acid sequence variants of the antigen-binding molecules provided herein are encompassed. For example, it may be desirable to improve the binding affinity and/or other biological properties of the antibody. Amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequence of the antigen-binding molecule. Any combination of deletion, insertion, and substitution can be made to obtain the final construct, provided that the final construct possesses the desired characteristics, e.g., antigen binding.

Variants with Substitutions, Insertions, and Deletions

In certain embodiments, antigen-binding molecule variants having one or more amino acid substitutions are provided. Substitutions may be made in the CDRs and FRs. Conservative substitutions are shown in Table 2 under the heading of “Preferred substitution”. More substantial changes are provided in Table 2 under the heading of “exemplary substitution”, and as further described below with reference to amino acid side chain classes. Amino acid substitutions can be introduced into an antibody of interest and the products are screened for a desired activity, e.g., retained/improved antigen binding, reduced immunogenicity, or improved ADCC or CDC.

TABLE 2
Amino acid substitutions

| Original | | |
|---|---|---|
| residue | Exemplary substitution | Preferred substitution |
| Ala(A) | Val; Leu; Ile | Val |
| Arg(R) | Lys; Gln; Asn | Lys |
| Asn(N) | Gln; His; Asp, Lys; Arg | Gln |
| Asp(D) | Glu; Asn | Glu |
| Cys(C) | Ser; Ala | Ser |
| Gln(Q) | Asn; Glu | Asn |
| Glu(E) | Asp; Gln | Asp |
| Gly(G) | Ala | Ala |
| His(H) | Asn; Gln; Lys; Arg | Arg |
| Ile(I) | Leu; Val; Met; Ala; Phe; norleucine | Leu |
| Leu(L) | Norleucine; Ile; Val; Met; Ala; Phe | Ile |
| Lys(K) | Arg; Gln; Asn | Arg |
| Met(M) | Leu; Phe; Ile | Leu |
| Phe(F) | Trp; Leu; Val; Ile; Ala; Tyr | Tyr |
| Pro(P) | Ala | Ala |
| Ser(S) | Thr | Thr |
| Thr(T) | Ser | Ser |
| Trp(W) | Tyr; Phe | Tyr |
| Tyr(Y) | Trp; Phe; Thr; Ser | Phe |
| Val(V) | Ile; Leu; Met; Phe; Ala; norleucine | Leu |

According to common side-chain properties, amino acids can be grouped as follows:
(1) hydrophobic: norleucine, Met, Ala, Val, Leu, Ile;
(2) neutral, hydrophilic: Cys, Ser, Thr, Asn, Gln;
(3) acidic: Asp, Glu;
(4) basic: His, Lys, Arg;
(5) residues affecting chain orientation: Gly, Pro;
(6) aromatic: Trp, Tyr, Phe.

A non-conservative substitution refers to using a member of one class to replace a member of another class.

One type of substitutional variant involves substituting one or more CDR residues of a parent antibody (e.g., a humanized or human antibody). Generally, the resulting variant selected for further study will have changes (e.g., improvements) in certain biological properties (e.g., increased affinity or reduced immunogenicity) relative to the parent antibody, and/or will have substantially retained certain biological properties of the parent antibody. An exemplary substitutional variant is an affinity-matured antibody, which can be conveniently produced, for example, using phage display-based affinity maturation techniques such as those described herein. Briefly, one or more CDR residues are mutated and the variant antibodies are displayed on phages and screened for a particular biological activity (e.g. binding affinity). Changes (e.g., substitutions) can be made in CDRs, for example, to improve antibody affinity. Such changes can be made in CDR “hotspots”, i.e., residues encoded by codons that undergo mutation at high frequency during the somatic maturation process, and/or antigen-contacting residues, and meanwhile, the resulting variant VH or VL is tested for binding affinity. In some embodiments of affinity maturation, diversity is introduced into the variable genes selected for maturation by any one of a variety of methods (e.g., error-prone PCR, chain shuffling, or oligonucleotide-directed mutagenesis). A secondary library is then created. The library is then screened to identify any antibody variants with the desired affinity. Another method for introducing diversity involves CDR-directed methods in which several CDR residues (e.g., 4-6 residues at a time) are randomized. CDR residues involved in antigen binding can be specifically identified, for example, using alanine scanning mutagenesis or modeling.

In certain embodiments, substitutions, insertions or deletions may occur within one or more CDRs, as long as such changes do not substantially reduce the ability of the antibody to bind to the antigen. For example, conservative changes (e.g., conservative substitutions as provided herein) that do not substantially reduce binding affinity may be made in CDRs. In certain embodiments of the variant VH and VL sequences provided above, each CDR is unaltered or contains no more than 1, 2, or 3 amino acid substitutions.

A useful method for identifying residues or regions of an antibody that can be used as mutagenesis targets is called “alanine scanning mutagenesis”. In this method, a residue or group of residues (e.g., charged residues such as Arg, Asp, His, Lys, and Glu) are identified and replaced with a neutral or negatively charged amino acid (e.g., Ala or polyalanine) to determine whether the interaction of the antibody with the antigen is affected. Further substitutions may be introduced at amino acid locations that show functional sensitivity to the initial substitutions. In addition, a crystal structure of an antigen-antibody complex may be studied to identify contact points between the antibody and antigen. These contact residues and neighboring residues may be targeted or eliminated as substitution candidates. Variants may be screened to determine whether they contain the desired properties.

Amino acid sequence insertions include: amino- and/or carboxy-terminal fusions to 1 residue or polypeptides of 100 or more residues in length, and intrasequence insertions of single or multiple amino acid residues. Examples of terminal insertions include an antibody with an N-terminal methionyl residue. Other insertional variants of the antibody molecule include the fusion of the N- or C-terminus of the antibody fused to an enzyme (or a polypeptide that increases the serum half-life of the antibody).

### Engineering of Fab

In one aspect, in the antigen-binding molecule of the present disclosure, one of the antigen-binding moiety binding specifically to CD3 and the antigen-binding moiety binding specifically to DLL3 is a replaced Fab, and the replaced Fab comprises a heavy chain variable region, a light chain variable region, a Titin chain, and an Obscurin chain. In the replaced Fab, the original CH1 and CL of the Fab are replaced with the Titin chain and the Obscurin chain. Illustratively, the sequences of the Titin chain and the Obscurin chain are shown in Tables 3-1 and 3-2. In the present disclosure, the combinations of the Titin chain and the Obscurin chain with the heavy chain variable region and the light chain variable region are interchangeable.

TABLE 3-1
The amino acid sequences of Titin chains

| | SEQ | |
|---|---|---|
| | ID | |
| No. | NO | Amino acid sequence |
| T.0 | 192 | GIPPKIEALPSDISIDEGKVLTVACAFTGEPTPEVTWSCG |
| | | GRKIHSQEQGRFHIENTDDLTTLIIMDVQKQDGGLYTLS |
| | | LGNEFGSDSATVNIHIRSI |
| T.1 | 193 | GIPPKIECLPSDISIDEGKVLTVASAFTGEPTPEVTWSTGG |
| | | RKIHSQEQGRFHIENTDDLTTLIIMDVQKQDGGLYTLSL |
| | | GNEFGSDSATVNIHIRSI |
| T.2 | 194 | GIPPKIEALPSDISIDEGKCLTVASAFTGEPTPEVTWSTGG |
| | | RKIHSQEQGRFHIENTDDLTTLIIMDVQKQDGGLYTLSL |
| | | GNEFGSDSATVNIHIRSI |
| T.3 | 195 | GIPPKIEALPSDISIDEGKVLTVASCFTGEPTPEVTWSTGG |
| | | RKIHSQEQGRFHIENTDDLTTLIIMDVQKQDGGLYTLSL |
| | | GNEFGSDSATVNIHIRSI |
| T.4 | 196 | GIPPKIEALPSDISIDEGKVLCVASAFTGEPTPEVTWSTGG |
| | | RKIHSQEQGRFHIENTDDLTTLIIMDVQKQDGGLYTLSL |
| | | GNEFGSDSATVNIHIRSI |
| T.5 | 197 | KAGIRGIPPKIECLPSDISIDEGKVLTVASAFTGEPTPEVT |
| | | WSTGGRKIHSQEQGRFHIENTDDLTTLIIMDVQKQDGGL |
| | | YTLSLGNEFGSDSATVNIHIRSI |
| T.6 | 198 | KAGIRGIPPKIEALPSDISIDEGKVLTVACAFTGEPTPEVT |
| | | WSCGGRKIHSQEQGRFHIENTDDLTTLIIMDVQKQDGGL |
| | | YTLSLGNEFGSDSATVNIHIRSI |
| T.7 | 199 | GIPPKIECLPSDISIDEGKVLTVASAFTGEPTPEVTWSTGG |
| | | RKIHSQEQGRFHIENTDDLTTLIISDVQKQDGGLYSLSLG |
| | | NEFGSDSATVNIHIRSI |
| T.8 | 200 | GIPPKIECLPSDISIDEGKVLTVASAFTGEPTPEVTWSTGG |
| | | RKIHSQEQGRFHIENTDDLTTLIIKDVQRQDGGLYTLTLR |
| | | NEFGSDSATVNIHIRSI |
| T.9 | 201 | GIWPKIECLPIDLSIDEGKVLMVASAFTGEPTPEVTWSTG |
| | | GRKIHSQEQGRFHIENTDDLTTLIIMDVQKQDGGLYTLS |
| | | LGMEFGSDSATVNIHIRSI |
| T.10 | 202 | GIPPKIECLPIDISIDEGKVLTVASAFTGEPTPEVTWSTGG |
| | | RKIHSQEQGRFHIENTDDLTTLIIKDVQKQDGGLYTLTLR |
| | | NEFGSDSATVNIHIRSI |
| T.11 | 203 | GIPPKIECLPSDISIDEGKVLTVASAFTGEPTPEVTWSTSG |
| | | KKISSEEGGRFHIESTEDLTTLIIMDVQKQDGGVYTLSLG |
| | | NDLGSDSATVNIHIRSI |
| T.12 | 204 | GIPPKIECLPSDISIDEGKVLTVASAFTGEPTPEVTWSTGG |
| | | RKIHSEEGGRFHIESTEDLTTLIIMDVQKQDGGVYTLSLG |
| | | NEFGSDSATVNIHIRSI |
| T.13 | 205 | GIPPKIECLPSDISIDEGKVLTVASAFTGEPTPEVTWSTGG |
| | | RKIHSQEQGRFHIESTEDLTTLIIMDVQKQDGGVYTLSLG |
| | | NEFGSDSATVNIHIRSI |
| T.14 | 206 | GIPPKIECLPSDISIDEGKVLTVASAFTGEPTPEVTWSTGG |
| | | RKIHSQEQGRFHIESTEDLTTLIISDVQKQDGGLYSLSLG |
| | | NEFGSDSATVNIHIRSI |
| T.15 | 207 | KAGIRGIPPKIECLPSDISIDEGKVLTVASAFTGEPTPEVT |
| | | WSTGGRKIHSQEQGRFHIESTEDLTTLIISDVQKQDGGLY |
| | | SLSLGNEFGSDSATVNIHIRSI |
| T.16 | 208 | GIPPKIECLPIDISIDEGKVLTVASAFTGEPTPEVTWSTGG |
| | | RKIHSQEQGRFHIENTDDSTTLTIKDVQKQDGGLYTLTL |
| | | RNEFGSDSATVNIHIRSI |
| T.17 | 209 | GIPPKIECLPIDISIDEGKCLTVASAFTGEPTPEVTWSTGG |
| | | RKIHSQEQGRFHIENTDDSTTLTIKDVQKQDGGLYTLTL |
| | | RNEFGSDSATVNIHIRSI |
| T.18 | 210 | GIPPKIECLPIDISIDEGKVLTVASCFTGEPTPEVTWSTGG |
| | | RKIHSQEQGRFHIENTDDSTTLTIKDVQKQDGGLYTLTL |
| | | RNEFGSDSATVNIHIRSI |

TABLE 3-2
The amino acid sequences of Obscurin chains

| | SEQ | |
|---|---|---|
| | ID | |
| No. | NO | Amino acid sequence |
| 0.0 | 211 | SGAPRFLTRPKAFVVSVGKDATLSCQIVGNPTPQVSWEK |
| | | DQQPVAAGARFRLAQDGDLYRLTILDLALGDSGQYVCR |
| | | ARNAIGEAFAAVGLQVDAEA |
| OL.0 | 212 | QGSPPCFLRFPRPVRVVSGAEAELKCVVLGEPPPVVVWE |
| | | KGGQQLAASERLSFPADGAEHGLLLTAALPTDAGVYVC |
| | | RARNAAGEAYAAAAVTVLEPP |
| O.1 | 213 | SGAPRFLTRPKAFVVSVGKDATLSCQIVGNPTPQVSWEK |
| | | DQQPVAAGARFRLAQDGDLYRLTILDLALGDSGQYVCR |
| | | ARNAIGEAFACVGLQVDAEA |
| O.2 | 214 | SGCPRFLTRPKAFVVSVGKDATLSCQIVGNPTPQVSWEK |
| | | DQQPVAAGARFRLAQDGDLYRLTILDLALGDSGQYVCR |
| | | ARNAIGEAFAAVGLQVDAEA |
| O.3 | 215 | SGAPRFLTCPKAFVVSVGKDATLSCQIVGNPTPQVSWEK |
| | | DQQPVAAGARFRLAQDGDLYRLTILDLALGDSGQYVCR |
| | | ARNAIGEAFAAVGLQVDAEA |
| O.4 | 216 | SGAPRFLTRPKAFVVSVGKDATLSSQIVGNPTPQVSWEK |
| | | DQQPVAAGARFRLAQDGDLYRLTILDLALGDSGQYVSR |
| | | ARNAIGEAFACVGLQVDAEA |
| O.5 | 217 | SGCPRFLTRPKAFVVSVGKDATLSSQIVGNPTPQVSWEK |
| | | DQQPVAAGARFRLAQDGDLYRLTILDLALGDSGQYVSR |
| | | ARNAIGEAFAAVGLQVDAEA |
| O.6 | 218 | SGAPRFLTCPKAFVVSVGKDATLSSQIVGNPTPQVSWEK |
| | | DQQPVAAGARFRLAQDGDLYRLTILDLALGDSGQYVSR |
| | | ARNAIGEAFAAVGLQVDAEA |
| O.7 | 219 | SGAPRFKTRPKAFVVSVGKDATLSSQIVGNPTPQVSWEK |
| | | DQQPVAAGARFRLAQDGDLYRLKILDLALGDSGQYVSR |
| | | ARNAIGEAFACVGLQVDAEA |
| O.8 | 220 | SGAPRFKTRPKAFVVSVGKDATLSSQIVGNPTPQVSWEK |
| | | DQQPVAAGARFRLAQDGDLYRLHILDLALGDSGQYVSR |
| | | ARNAIGEAFACVGLQVDAEA |
| O.9 | 221 | SGAPRFLTRPLAFVVSVGKDATLSSQIVGNPTPQVSWEK |
| | | DQQPVAAGARFRLAQDGDLYRLKILDLALGDSGQYVSR |
| | | ARNAIGEAFACVGLQVDAEA |
| O.10 | 222 | SGAPRFLTRPLAFVVSVGKDATLSSQIVGNPTPQVSWEK |
| | | DQQPVAAGARFRLAQDGDLYRLHILDLALGDSGQYVSR |
| | | ARNAIGEAFACVGLQVDAEA |
| O.11 | 223 | DQPQFSGAPRFLTRPKAFVVSVGKDATLSSQIVGNPTPQ |
| | | VSWEKDQQPVAAGARFRLAQDGDLYRLTILDLALGDS |
| | | GQYVSRARNAIGEAFACVGLQVDAEA |
| O.12 | 224 | DQPQFSGAPRFKTRPKAFVVSVGKDATLSSQIVGNPTPQ |
| | | VSWEKDQQPVAAGARFRLAQDGDLYRLKILDLALGDS |
| | | GQYVSRARNAIGEAFACVGLQVDAEA |
| O.13 | 225 | DQPQFSGAPRFKTRPKAFVVSVGKDATLSSQIVGNPTPQ |
| | | VSWEKDQQPVAAGARFRLAQDGDLYRLHILDLALGDS |
| | | GQYVSRARNAIGEAFACVGLQVDAEA |
| O.14 | 226 | DQPQFSGAPRFRTRPKAFVVSVGKDATLSSQIVGNPTPQ |
| | | VSWEKDQQPVAAGARFRLAQDGDLYRLKILDLALGDS |
| | | GQYVSRARNAIGEAFACVGLQVDAEA |
| O.15 | 227 | DQPQFSGAPRFRTRPKAFVVSVGKDATLSSQIVGNPTPQ |
| | | VSWEKDQQPVAAGARFRLAQDGDLYRLHILDLALGDS |
| | | GQYVSRARNAIGEAFACVGLQVDAEA |
| O.16 | 228 | DQPQFSGAPRFLTRPKAFVVSVGKDATLSCQIVGNPTPQ |
| | | VSWEKDQQPVAAGARFRLAQDGDLYRLTILDLALGDS |
| | | GQYVCRARNAIGEAFAAVGLQVDAEA |
| OL.1 | 229 | QGSPPEFLRFPRPVRVVSGAEAELKCVVLGEPPPVVVWE |
| | | KGGQQLAASERLSFPADGAEHGLLLTAALPTDAGCYVC |
| | | RARNAAGEAYAAAAVTVLEPP |
| OL.2 | 230 | QGSPPEFLRFPRPVRVVSGAEAELKCVVLGEPPPVVVWE |
| | | KGGQQLAASERLSFPADGAEHGLLLTAALPTDAGVYVC |
| | | RARNAACEAYAAAAVTVLEPP |
| OL.3 | 231 | QGSPPEFLRFPRPVRVVSGAEAELKCVVLGEPPPVVVWE |
| | | KGGQQLAASERLSFPADGAEHGLLLTAALPTDAGVYVC |
| | | RARNAAGECYAAAAVTVLEPP |
| OL.4 | 232 | QGSPPEFLRFPRPVRVVSGAEAELKSVVLGEPPPVVVWE |
| | | KGGQQLAASERLSFPADGAEHGLLLTAALPTDAGCYVS |
| | | RARNAAGEAYAAAAVTVLEPP |
| OL.5 | 233 | QGSPPEFLRFPRPVRVVSGAEAELKSVVLGEPPPVVVWE |
| | | KGGQQLAASERLSFPADGAEHGLLLTAALPTDAGVYVS |
| | | RARNAACEAYAAAAVTVLEPP |
| OL.6 | 234 | QGSPPEFLRFPRPVRVVSGAEAELKSVVLGEPPPVVVWE |
| | | KGGQQLAASERLSFPADGAEHGLLLTAALPTDAGVYVS |
| | | RARNAAGECYAAAAVTVLEPP |
| O.17 | 235 | SGAPRFLTRPKSYTVSVGKDASLSSQIVGNPTPQVSWEK |
| | | DQQPVAAGARFRLAQDGDLYRLKILDLALGDSGQYVSR |
| | | ARNAIGEAFACVGLQVDAEA |
| O.18 | 236 | SEAPRFLTRPLAFVVSEGKDATLSSQIVGDPPPEVTWEK |
| | | DQQPITAGARFRLAQDGDVYRLEILDLQLSDSGQYVSR |
| | | ARNAIGEAFACVGLQVDAEG |
| O.19 | 237 | SGAPRFLTRPLAFVVSVGKLAMLSSQIVGNPTPQVSWEK |
| | | DQQPVAAGARFRLLQDGDLYRLKILDLALGDSGQYVSR |
| | | ARNAIGEAFACVGLQVDAEA |
| O.20 | 238 | SGAPRFLTRPLAFVVSVGKDATLSSQIVGNPTPQVSWEK |
| | | DKQPVTAGARFRLAQDGDLYRLKILDLQLSDSGQYVSR |
| | | ARNAIGEAFACLGLQVDAEA |
| O.21 | 239 | SGAPRFLTRPLAFVVSVGKDATLSSQIVGNPTPQVSWEK |
| | | DQLPVTAGARFRLAQDGDLYRLKILDLTLSDSGQYVSR |
| | | ARNAIGEAFACVGLEVGAEA |
| O.22 | 240 | SGAPRFLTRPLSYVVSVGKDASLSSQIVGNPTPQVSWEK |
| | | DQLPVTAGARFRLAQDGDLYRLKILDLQLSDSGQYVSR |
| | | ARNAIGEAFACVGLEVGAEA |
| O.23 | 241 | DQPQFSGAPRFLTRPLSYVVSVGKDASLSSQIVGNPTPQ |
| | | VSWEKDQLPVTAGARFRLAQDGDLYRLKILDLQLSDSG |
| | | QYVSRARNAIGEAFACVGLEVGAEA |
| O.24 | 242 | SGAPRFLTRPKAFVVSVGKDATLSSQIVGNPFPQVSWEK |
| | | DKQPVTAGVRFRLAQDGDLYRLKILDLQLSDSGQYVSR |
| | | ARNAIGEAFACLGLQVDAEA |
| O.25 | 243 | SGAPRFLTRPKASVVSVGKDATLSSQIVGNPFPQVSWEK |
| | | DKQPVTAGVRFRLAQDGDLYRLKILDLQLSDSGQYVSR |
| | | ARNAHGEAFACLGLQVDAEA |
| O.26 | 244 | SGCPRFLTRPKASVVSVGKDATLSSQIVGNPFPQVSWEK |
| | | DKQPVTAGVRFRLAQDGDLYRLKILDLQLSDSGQYVSR |
| | | ARNAHGEAFACLGLQVDAEA |
| O.27 | 245 | SGAPRFLTCPKASVVSVGKDATLSSQIVGNPFPQVSWEK |
| | | DKQPVTAGVRFRLAQDGDLYRLKILDLQLSDSGQYVSR |
| | | ARNAHGEAFACLGLQVDAEA |
| O.28 | 246 | SGAPRFLTRPKASVVSVGKDATLSCQIVGNPFPQVSWEK |
| | | DKQPVTAGVRFRLAQDGDLYRLKILDLQLSDSGQYVCR |
| | | ARNAHGEAFACLGLQVDAEA |
| O.29 | 247 | SGCPRFLTRPKASVVSVGKDATLSCQIVGNPFPQVSWEK |
| | | DKQPVTAGVRFRLAQDGDLYRLKILDLQLSDSGQYVCR |
| | | ARNAHGEAFACLGLQVDAEA |
| O.30 | 248 | SGAPRFLTCPKASVVSVGKDATLSCQIVGNPFPQVSWEK |
| | | DKQPVTAGVRFRLAQDGDLYRLKILDLQLSDSGQYVCR |
| | | ARNAHGEAFACLGLQVDAEA |
| O.31 | 249 | SGAPRFLTRPKASVVSVGKDATLSSQIVGNPFPQVSWEK |
| | | DKQPVTAGVRFRLAQDGDLYRLKILDCQLSDSGQYVSR |
| | | ARNAHGEAFACLGLQCDAEA |
| O.32 | 250 | SGCPRFLTRPKASVVSVGKDATLSSQIVGNPFPQVSWEK |
| | | DKQPVTAGVRFRLAQDGDLYRLKILDCQLSDSGQYVSR |
| | | ARNAHGEAFACLGLQCDAEA |
| O.33 | 251 | SGAPRFLTCPKASVVSVGKDATLSSQIVGNPFPQVSWEK |
| | | DKQPVTAGVRFRLAQDGDLYRLKILDCQLSDSGQYVSR |
| | | ARNAHGEAFACLGLQCDAEA |

### Engineering of Fc Region

In one aspect, the Fc region of the antigen-binding molecule of the present disclosure comprises one or more amino acid substitutions that reduce its binding to an Fc receptor, e.g., its binding to an Fcγ receptor, and reduce or eliminate effector functions. A natural IgG Fc region, specifically an IgG₁ Fc region or an IgG₄ Fc region, may cause the antigen-binding molecule of the present disclosure to target cells expressing an Fc receptor, rather than cells expressing an antigen. The engineered Fc region of the present disclosure exhibits reduced binding affinity for an Fc receptor and/or reduced effector functions. In some embodiments, the engineered Fc region shows no less than 50%, 80%, 90%, or 95% reduction in binding affinity for an Fc receptor as compared to the native Fc region. In some embodiments, the Fc receptor is an Fcγ receptor. In some embodiments, the Fc receptor is a human Fcγ receptor, e.g., FcγRI, FcγRIIa, FcγRIIB, or FcγRIIIa. In some embodiments, the engineered Fc region also has reduced binding affinity for a complement (e.g., C1q) as compared to the native Fc region. In some embodiments, the engineered Fc region has no reduced binding affinity for a neonatal Fc receptor (FcRn) as compared to the native Fc region. In some examples, the engineered Fc region has reduced effector functions, which may include, but are not limited to, one or more of the following: reduced complement-dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen presenting cells, reduced binding to NK cells, reduced binding to macrophages, reduced binding to monocytes, reduced binding to polymorphonuclear cells, reduced direct signaling-induced apoptosis, reduced dendritic cell maturation, and reduced T cell priming. For the IgG1 Fc region, amino acid residue substitutions at positions such as positions 238, 265, 269, 270, 297, 327, and 329 can reduce effector functions. In some embodiments, the Fc region is a human IgG1 Fc region, and the amino acid residues at positions 234 and 235 are A, as numbered according to the EU index. For the IgG4 Fc region, amino acid residue substitutions at positions such as position 228 can reduce effector functions.

The antigen-binding molecule may also comprise disulfide bond engineering, such as 354C in the first subunit and 349C in the second subunit. To increase the serum half-life of the antigen-binding molecule, 252Y, 254T, and 256E mutations may be introduced.

When the antigen-binding molecule comprises different binding moieties fused to the two subunits of the Fc region, it may lead to undesired homodimerization. To improve yield and purity, it is thus advantageous to introduce modifications that promote heterodimerization in the Fc region of the antigen-binding molecule of the present disclosure. In some embodiments, the Fc region of the present disclosure comprises engineering according to the knob-into-hole (KIH) technique; the method relates to introducing a knob structure at the interface of the first subunit and introducing a hole structure at the interface of the second subunit. As such, the knob structure can be positioned in the hole structure, promoting the formation of a heterodimer and inhibiting the production of a homodimer. The knob structure is constructed by substituting a small amino acid side chain at the interface of the first subunit with a larger side chain (e.g., tyrosine or tryptophan). The hole structure is created at the interface of the second subunit by substituting a large amino acid side chain with a smaller amino acid side chain (e.g., alanine or threonine). The knob structure and the hole structure are prepared by altering the nucleic acid encoding the polypeptide. Optional amino acid substitutions are shown in the table below:

TABLE 4
Combinations of KIH mutations

| | | | | | | | | |
|---|---|---|---|---|---|---|---|---|
| First | T366Y | T366W | T394W | F405W | T366W | T366Y | T366W | F405W |
| subunit | | | | | | F405A | F405W | Y407A |
| Second | Y407T | Y407A | F405A | T394S | T366S | T394W | T394W | T366W |
| subunit | | | | | L368A | Y407T | Y407A | T394S |
| | | | | | Y407V | | | |

In addition to the knob-into-hole technique, other techniques for modifying the CH3 domain of the heavy chain to achieve heterodimerization are also known in the art, e.g., WO1996027011A1, WO1998050431, EP1870459, WO2007110205, WO2009089004, WO2010129304, WO201190754, WO2011143545, WO2012058768, WO2013157954, and WO2013096291.

The C-terminus of the Fc region may be an intact C-terminus ending with amino acid residues PGK or a truncated C-terminus, e.g., a truncated C-terminus in which one or two C-terminal amino acid residues are removed. In a preferred aspect, the C-terminus of the heavy chain is a shortened C-terminus ending with PG. Thus, in some embodiments, a composition of intact antibodies may comprise antibody populations with all K447 residues and/or G446+K447 residues removed. In some embodiments, a composition of intact antibodies may comprise antibody populations without a K447 residue and/or G446+K447 residues removed. In some embodiments, a composition of intact antibodies comprises antibody populations having a mixture of antibodies with and without a K447 residue and/or G446+K447 residues.

### Recombination Method

The antigen-binding molecule may be produced using recombination methods. For these methods, one or more isolated nucleic acids encoding the antigen-binding molecule are provided.

In the case of a native antibody, a native antibody fragment, or a bispecific antibody with homodimeric heavy chains, two nucleic acids are required, one for the light chain or a fragment thereof and the other for the heavy chain or a fragment thereof. Such nucleic acids encode an amino acid sequence comprising the VL of the antibody and/or an amino acid sequence comprising the VH of the antibody (e.g., the light and/or heavy chain(s) of the antibody). These nucleic acids can be on the same expression vector or different expression vectors.

In the case of a bispecific antibody with heterodimeric heavy chains, four nucleic acids, for example, are required, one for a first light chain, one for a first heavy chain comprising a first heteromonomeric Fc-region polypeptide, one for a second light chain, and one for a second heavy chain comprising a second heteromonomeric Fc-region polypeptide. The four nucleic acids can be contained in one or more nucleic acid molecules or expression vectors. Generally, these nucleic acids are located on two or three expression vectors; that is, one vector can comprise more than one of these nucleic acids.

In one embodiment, the present disclosure provides an isolated nucleic acid encoding the aforementioned antibody. Such nucleic acids may independently encode any one of the aforementioned polypeptide chains. In another aspect, the present disclosure provides one or more vectors (e.g., expression vectors) comprising such nucleic acids. In another aspect, the present disclosure provides a host cell comprising such nucleic acids. In one embodiment, provided is a method for preparing an antigen-binding molecule, wherein the method comprises culturing a host cell comprising a nucleic acid encoding the antibody, as provided above, under conditions suitable for antibody expression, and optionally collecting the antibody from the host cell (or host cell culture medium).

For recombinant production of the antigen-binding molecule, the nucleic acid encoding the protein is isolated and inserted into one or more vectors for further cloning and/or expression in a host cell. Such nucleic acids can be readily isolated and sequenced using conventional procedures (e.g., by using oligonucleotide probes that are capable of binding specifically to genes encoding the heavy and light chains of an antibody), or produced by recombination methods or obtained by chemical synthesis.

Suitable host cells for cloning or expressing a vector encoding the antibody include prokaryotic or eukaryotic cells described herein. For example, the antibody can be produced in bacteria, particularly when the antibody does not require glycosylation and Fc effector functions. After expression, the antibody can be isolated from the bacterial cell paste in a soluble fraction and can be further purified.

In addition to prokaryotes, eukaryotic microorganisms such as filamentous fungi or yeast are suitable cloning or expression hosts for antibody-encoding vectors, including fungal and yeast strains, whose glycosylation pathways have been “humanized”, such that an antibody with a partially or fully human glycosylation pattern is produced. Suitable host cells for expression of (glycosylated) antibodies may also be derived from multicellular organisms (invertebrates and vertebrates); examples of invertebrate cells include plant and insect cells. A number of baculovirus strains have been identified, which can be used in combination with insect cells, particularly for transfection of Spodoptera frugiperda cells; plant cell cultures may also be used as hosts, see, e.g., U.S. Pat. Nos. 5,959,177, 6,040,498, 6,420,548, 7,125,978 and 6,417,429; vertebrate cells can also be used as hosts, e.g., mammalian cell lines adapted for growth in a suspension. Other examples of suitable mammalian host cell lines include an SV40-transformed monkey kidney CV1 line (COS-7); a human embryonic kidney line (293 or 293T cells); baby hamster kidney cells (BHK); mouse sertoli cells (TM4 cells); monkey kidney cells (CV1); African green monkey kidney cells (VERO-76); human cervical cancer cells (HELA); canine kidney cells (MDCK); buffalo rat liver cells (BRL3A); human lung cells (W138); human liver cells (Hep G2); mouse mammary tumor cells (MMT 060562); TRI cells; MRC 5 cells; and FS4 cells. Other suitable mammalian host cell lines include Chinese hamster ovary (CHO) cells, including DHFR-CHO cells; and myeloma cell lines such as Y0, NS0, and Sp2/0. For reviews of certain mammalian host cell lines suitable for the production of the antibody, see, e.g., Yazaki, P. and Wu, A. M., Methods in Molecular Biology, Vol. 248, Lo, B. K. C. (eds.), Humana Press, Totowa, NJ (2004), pp. 255-268.

### Diagnostic and Therapeutic Compositions

In certain embodiments, the antigen-binding molecule provided in the present disclosure can be used to detect the presence of DLL3 and/or DLL3 in a biological sample. As used herein, the term “detection” encompasses quantitative or qualitative detection. In certain embodiments, the biological sample includes a cell or tissue, such as tumor tissue.

In one embodiment, provided is an antigen-binding molecule for use in a diagnostic or detection method. In yet another aspect, provided is a method for detecting the presence of DLL3 and/or DLL3 in a biological sample. In certain embodiments, the method comprises contacting the biological sample with an antigen-binding molecule under suitable conditions and detecting whether a complex is formed between the detection agent and the antigen. Such methods may be in vitro or in vivo methods. In one embodiment, an antigen-binding molecule is used to select a subject suitable for treatment; for example, DLL3 and/or DLL3 are biomarkers for selecting a patient.

Exemplary disorders that can be diagnosed using the antigen-binding molecule of the present disclosure are, for example, tumors or cancers.

In certain embodiments, provided is a labeled antigen-binding molecule. Labels include, but are not limited to, labels or moieties that are detected directly (such as fluorescent, chromophoric, electron-dense, chemiluminescent, and radioactive labels), and moieties that are detected indirectly (e.g., moieties that are detected indirectly through an enzymatic reaction or molecular interaction, such as enzymes or ligands).

In an additional aspect, provided is a pharmaceutical composition comprising the antigen-binding molecule, for example, for use in any of the following treatment methods. In one aspect, the pharmaceutical composition comprises any of the antigen-binding molecules provided herein and a pharmaceutically acceptable carrier. In another aspect, the pharmaceutical composition comprises any of the antigen-binding molecules provided herein and at least one additional therapeutic agent.

The pharmaceutical composition of the antigen-binding molecule described in the present disclosure is prepared by: mixing such an antigen-binding molecule having desired purity with one or more optional pharmaceutically acceptable carriers. The pharmaceutical composition is in the form of a lyophilized composition or an aqueous solution. Formulations for in vivo administration are generally sterile. Sterility can be readily achieved, for example, by filtration through a sterile filter membrane.

### Treatment Method and Route of Administration

Any of the antigen-binding molecules provided herein can be used in the treatment method.

In yet another aspect, the present disclosure provides use of the antigen-binding molecule in the manufacture or preparation of a medicament. In one embodiment, the medicament is used for treating a proliferative disease or tumor. And the medicament is present in an amount effective for the diseases described above. In some embodiments, the effective amount is a unit daily dose or a unit weekly dose. In one such embodiment, the use further comprises administering to a subject an effective amount of at least one additional therapeutic agent (e.g., one, two, three, four, five, or six additional therapeutic agents). The “subject” according to any of the above embodiments may be a human.

In yet another aspect, provided is a pharmaceutical composition comprising the antigen-binding molecule, e.g., for any of the pharmaceutical uses or treatment methods described above. In another embodiment, the pharmaceutical composition also comprises at least one additional therapeutic agent.

The antigen-binding molecule of the present disclosure may be used alone or in combination with other agents for the treatment. For example, the antigen-binding molecule of the present disclosure may be co-administered with at least one additional therapeutic agent. In some embodiments, the additional therapeutic agent is a bispecific antibody binding specifically to DLL3 and CD28.

The antigen-binding molecule of the present disclosure (and any additional therapeutic agents) may be administered by any suitable means, including parenteral, intrapulmonary, and intranasal administration, and if required for local treatment, intralesional administration. Parenteral infusion includes intramuscular, intravenous, intra-arterial, intraperitoneal, or subcutaneous administration. Administration may be performed by any suitable route, e.g., by injection, such as intravenous or subcutaneous injection, depending in part on whether the administration is short-term or long-term. Various administration schedules are contemplated herein, including but not limited to, single administration or multiple administrations at multiple time points, bolus administration, and pulse infusion.

The antigen-binding molecule of the present disclosure will be formulated, administered, and applied in a manner consistent with Good Medical Practice. Factors considered in this context include the specific disorder being treated, the specific mammal being treated, the clinical state of the individual patient, the cause of the disorder, the site of delivery of the agent, the method of administration, the timing of administration, and other factors known to medical practitioners. The antigen-binding molecule needs not be, but is optionally, formulated along with one or more agents currently used for preventing or treating the disorder. The effective amount of such additional agents depends on the amount of the antigen-binding molecule present in the pharmaceutical composition, the type of the disorder or treatment, and other factors discussed above. These are generally used in the same dosages and with routes of administration as described herein, or in about 1 to 99% of the dosages described herein, or in any dosage and by any route that is empirically/clinically determined to be appropriate.

For the prevention or treatment of a disease, the appropriate dosage of the antigen-binding molecule of the present disclosure (when used alone or in combination with one or more additional therapeutic agents) will depend on the type of disease to be treated, the type of therapeutic molecule, the severity and course of the disease, the purpose of the administration (prophylactic or therapeutic), previous treatment, clinical history and response to the therapeutic molecule of the patient, and the discretion of the attending physician. The therapeutic molecule is suitably administered to a patient in one or a series of treatments. Depending on the type and severity of the disease, about 1 μg/kg to 15 mg/kg of the antigen-binding molecule can be an initial candidate dosage for administration to the patient, whether, for example, by one or more separate administrations, or by continuous infusion. One typical daily dosage may range from about 1 μg/kg to 100 mg/kg or more, depending on the factors mentioned above. Accordingly, taking 50 kg body weight as an example, an exemplary unit daily dosage is 50 pg-5 g.

### Product (Kit)

In another aspect of the present disclosure, provided is a product, which comprises materials useful for the treatment, prevention, and/or diagnosis of the disorders described above. The product comprises a container and a label or package insert on or associated with the container. Suitable containers include, for example, bottles, vials, syringes, IV solution bags, and the like. The container may be formed from a variety of materials such as glass or plastic. The container contains a composition effective in the treatment, prevention, and/or diagnosis of a disease, either alone or in combination with another composition, and may have a sterile access port (e.g., the container may be an intravenous solution bag or vial with a stopper pierceable by a hypodermic injection needle). At least one active agent in the composition is the antigen-binding molecule of the present disclosure. The label or package insert indicates that the composition is used to treat the selected condition. Further, the product may comprise: (a) a first container containing a composition, wherein the composition comprises the antigen-binding molecule of the present disclosure; and (b) a second container containing a composition, wherein the composition comprises other cytotoxic agents or additional therapeutic agents. The product in this embodiment of the present disclosure may further comprise a package insert indicating that the composition may be used to treat a particular condition. Alternatively or additionally, the product may further comprise a second (or third) container containing a pharmaceutically acceptable buffer. From a commercial and user standpoint, it may further contain other materials as required, including other buffers, diluents, filters, needles, and syringes.

### EXAMPLES AND TEST EXAMPLES

The present disclosure is further described below with reference to the following examples and test examples, which, however, do not limit the present disclosure. The experimental methods in the examples and test examples of the present disclosure in which specific conditions are not specified are generally performed under conventional conditions such as Antibodies: A Laboratory Manual and Molecular Cloning: A Laboratory Manual by Cold Spring Harbor Laboratory, or under conditions recommended by the manufacturers of the starting materials or commercial products. Reagents without specific origins indicated are commercially available conventional reagents.

### Example 1. Antigen-Binding Molecules Comprising Titin Chain/Obscurin Chain

The Titin/Obscurin chains of the present disclosure may be derived from any suitable polypeptide, including polypeptides from WO2021139758 (incorporated herein by reference in its entirety) and CN202110527339.7 and the patents which refer to it as a priority document (incorporated herein by reference in their entirety). Bispecific antibodies were constructed, where the CL was the kappa light chain constant region in WO2021139758, the amino acid sequences of the Titin and Obscurin chains are shown in Tables 3-1 and 3-2, and the linker sequences include GGGGS (SEQ ID NO: 147), ASTKG (SEQ ID NO: 255), or RTVAS (SEQ ID NO: 256). The amino acid sequences of the Fc1, Fc2, and CH1 in this example are shown below.

> Example 1-Fc1
(knob, SEQ ID NO: 143)
DKTHTCPPCPAPEAAGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPCR
EEMTKNQVSLWCLVKGFYPSDIAVEWESNGQPENNYKTTPPVLDS
DGSFFLYSKLTVDKSRWQQGNVFSCSVMHEALHNHYTQKSLSLSP
GK
> Example 1-Fc2
(hole, SEQ ID NO: 144)
DKTHTCPPCPAPEAAGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVCTLPPSR
EEMTKNQVSLSCAVKGFYPSDIAVEWESNGQPENNYKTTPPVLDS
DGSFFLVSKLTVDKSRWQQGNVFSCSVMHEALHNHYTQKSLSLSP
GK

### >Example 1-CH1 (SEQ ID NO: 141)

### 1.1. DI Bispecific Antibodies

DI bispecific antibodies against hNGF and hRANKL, DI-2 to DI-20, which comprise a first heavy chain, a second heavy chain, a first light chain, and a second light chain as described below, were constructed with reference to Example 5 in WO2021139758:
the first heavy chain: when viewed from N-terminus to C-terminus, [VH1-I]-[linker 1]-[Obscurin chain]-[Fc2];
the first light chain: when viewed from N-terminus to C-terminus, [VL1-I]-[linker 2]-[Titin chain];
the second heavy chain: when viewed from N-terminus to C-terminus, [VH2-D]-[CH1]-[Fc1]; and
the second light chain: when viewed from N-terminus to C-terminus, [VL2-D]-[CL];
where the VH1-I and VL1-I are the heavy chain variable region and the light chain variable region of I0 in WO2021139758, respectively, and the VH2-D and VL2-D are the heavy chain variable region and the light chain variable region of DO in WO2021139758, respectively. The structures of the Obscurin chain, the Titin chain, the linker 1, and the linker 2 in the DI bispecific antibodies of this example are shown in Table 5 below.

TABLE 5
The Obscurin/Titin chains and linkers in the DI bispecific antibodies

| | First heavy chain | | First light chain | |
|---|---|---|---|---|
| No. | Obscurin chain | Linker 1 | Titin chain | Linker 2 |
| DI-2 | O.20 | GGGGS | T.10 | GGGGS |
| DI-3 | O.25 | GGGGS | T.16 | GGGGS |
| DI-4 | O.26 | GGGGS | T.17 | GGGGS |
| DI-5 | O.27 | GGGGS | T.18 | GGGGS |
| DI-6 | O.28 | GGGGS | T.16 | GGGGS |
| DI-7 | O.29 | GGGGS | T.17 | GGGGS |
| DI-8 | O.30 | GGGGS | T.18 | GGGGS |
| DI-9 | O.31 | GGGGS | T.16 | GGGGS |
| DI-10 | O.32 | GGGGS | T.17 | GGGGS |
| DI-11 | O.33 | GGGGS | T.18 | GGGGS |
| DI-12 | O.25 | ASTKG | T.16 | RTVAS |
| DI-13 | O.26 | ASTKG | T.17 | RTVAS |
| DI-14 | O.27 | ASTKG | T.18 | RTVAS |
| DI-15 | O.28 | ASTKG | T.16 | RTVAS |
| DI-16 | O.29 | ASTKG | T.17 | RTVAS |
| DI-17 | O.30 | ASTKG | T.18 | RTVAS |
| DI-18 | O.31 | ASTKG | T.16 | RTVAS |
| DI-19 | O.32 | ASTKG | T.17 | RTVAS |
| DI-20 | O.33 | ASTKG | T.18 | RTVAS |

Note:
The numbering of the Titin and Obscurin chains in the table is shown in Tables 3-1 and 3-2.

The binding activity of the bispecific antibodies DI-2 to DI-20 to their antigens was measured using the method in Test Example 4 of WO2021139758. The thermal stability of the antibodies was studied. Study method: The antibodies were diluted with PBS to a concentration of 5 mg/mL, and their thermal stability was measured using a high throughput differential scanning fluorimeter (UNCHAINED; model: Unit). The experimental results show that there was no significant change in the antigen-binding activity of the engineered bispecific antibodies; additionally, there were significant increases in the Tm1 (° C.) and Tonset (° C.) of DI-4 to DI-8, DI-10 to DI-16, and DI-20 as compared to DI-2, suggesting that the thermal stability of the bispecific antibodies is better.

TABLE 6
The binding activity of the DI bispecific antibodies

| | |
|---|---|
| RANKL | NGF |

| | |
|---|---|
| No. | EC50 (nM) |

| | | |
|---|---|---|
| DI-2 | 0.3832 | 6.633 |
| DI-3 | 0.3613 | 5.7730 |
| DI-4 | 0.3959 | 6.2930 |
| DI-5 | 0.3290 | 6.1890 |
| DI-6 | 0.2509 | 5.6720 |
| DI-7 | 0.2557 | 6.6430 |
| DI-8 | 0.3643 | 7.6250 |
| DI-9 | 0.2944 | 8.4950 |
| DI-10 | 0.3460 | 7.1660 |
| DI-11 | 0.3721 | 10.9600 |
| DI-12 | 0.4125 | 6.5156 |
| DI-13 | 0.4440 | 5.5420 |
| DI-14 | 0.4182 | 3.2610 |
| DI-15 | 0.2206 | 5.2800 |
| DI-16 | 0.1474 | 5.5140 |
| DI-17 | 0.2329 | 6.7270 |
| DI-18 | 0.2662 | 5.9080 |
| DI-19 | 0.1843 | 5.9280 |
| DI-20 | 0.3184 | 6.4250 |

TABLE 7
The thermal stability of the DI bispecific antibodies

| No. | Tm1 (° C.) | Tonset (° C.) |
|---|---|---|
| DI-2 | 55.6 | 48.3 |
| DI-4 | 60.1 | 52.493 |
| DI-5 | 61 | 51.967 |
| DI-6 | 60.8 | 53.012 |
| DI-7 | 60.34 | 52.003 |
| DI-8 | 60.61 | 50.425 |
| DI-10 | 60.2 | 52.766 |
| DI-11 | 57.35 | — |
| DI-12 | 59.9 | 51.726 |
| DI-13 | 61 | 50.988 |
| DI-14 | 61.2 | 52.191 |
| DI-15 | 60.41 | 50.558 |
| DI-16 | 61.5 | 50.691 |
| DI-20 | 60.7 | 51.859 |

Solutions of the DI bispecific antibodies were prepared using a pH 5.5 buffer containing 10 mM acetic acid and 9% sucrose, and the solutions were incubated in a 40 C incubator for four weeks. Following the incubation, the antibody solutions were concentrated to the concentration at the start of the incubation, and the solutions were observed for precipitate formation. The experimental results show that a precipitate formed in the solution of the DI-2 bispecific antibody group. DI-3 to DI-7 are more stable than DI-2.

TABLE 8
Precipitate formation in the solutions
of the DI bispecific antibodies

| | | Concentration after | Precipitate |
|---|---|---|---|
| | Initial | solution concentration | formation in |
| No. | concentration | at week 4 | solution |
| DI-2 | 20 mg/mL | 20 mg/mL | Precipitate formed |
| DI-3 | 20 mg/mL | 20 mg/mL | No precipitation |
| DI-4 | 60 mg/mL | 60 mg/mL | No precipitation |
| DI-5 | 25 mg/mL | 25 mg/mL | No precipitation |
| DI-6 | 60 mg/mL | 60 mg/mL | No precipitation |
| DI-7 | 16 mg/mL | 16 mg/mL | No precipitation |

### 1.2. PL Bispecific Antibodies

PL bispecific antibodies against hPDL1 and hCTLA4, PL-1 to PL-19, which comprise a first heavy chain, a second heavy chain, a first light chain, and a second light chain as described below, were constructed:
the first heavy chain: when viewed from N-terminus to C-terminus, [VH1-P]-[linker 1]-[Obscurin chain]-[Fc1];
the first light chain: when viewed from N-terminus to C-terminus, [VL1-P]-[linker 2]-[Titin chain];
the second heavy chain: when viewed from N-terminus to C-terminus, [VH2-L]-[CH1]-[Fc2]; and
the second light chain: when viewed from N-terminus to C-terminus, [VL2-L]-[CL];
where the VH1-P and VL1-P were the heavy chain variable region and the light chain variable region of the h1831K antibody in WO2020177733A1, respectively. The amino acid sequences of the VH2-L and VL2-L are shown below.

>VH2-L
(SEQ ID NO: 257)
QVQLVESGGGVVQPGRSLRLSCAASGFTFSSYTMHWVRQAPGKGLEWVTF
ISYDGNNKYYADSVKGRFTISRDNSKNTLYLQMNSLRAEDTAIYYCARTG
WLGPFDYWGQGTLVTVSS
>VL2-L
(SEQ ID NO: 258)
EIVLTQSPGTLSLSPGERATLSCRASQSVGSSYLAWYQQKPGQAPRLLIY
GAFSRATGIPDRFSGSGSGTDFTLTISRLEPEDFAVYYCQQYGSSPWTFG
QGTKVEIK.

The structures of the Obscurin chain, the Titin chain, the linker 1, and the linker 2 in the PL bispecific antibodies of this example are shown in the table below.

TABLE 9
The Obscurin/Titin chains and linkers in the PL bispecific antibodies

| | First heavy chain | | First light chain | |
|---|---|---|---|---|
| No. | Obscurin chain | Linker 1 | Titin chain | Linker 2 |
| PL-1 | O.20 | GGGGS | T.10 | GGGGS |
| PL-2 | O.25 | GGGGS | T.16 | GGGGS |
| PL-3 | O.26 | GGGGS | T.17 | GGGGS |
| PL-4 | O.27 | GGGGS | T.18 | GGGGS |
| PL-5 | O.28 | GGGGS | T.16 | GGGGS |
| PL-6 | O.29 | GGGGS | T.17 | GGGGS |
| PL-7 | O.30 | GGGGS | T.18 | GGGGS |
| PL-8 | O.31 | GGGGS | T.16 | GGGGS |
| PL-9 | O.32 | GGGGS | T.17 | GGGGS |
| PL-10 | O.33 | GGGGS | T.18 | GGGGS |
| PL-11 | O.25 | ASTKG | T.16 | RTVAS |
| PL-12 | O.26 | ASTKG | T.17 | RTVAS |
| PL-13 | O.27 | ASTKG | T.18 | RTVAS |
| PL-14 | O.28 | ASTKG | T.16 | RTVAS |
| PL-15 | O.29 | ASTKG | T.17 | RTVAS |
| PL-16 | O.30 | ASTKG | T.18 | RTVAS |
| PL-17 | O.31 | ASTKG | T.16 | RTVAS |
| PL-18 | O.32 | ASTKG | T.17 | RTVAS |
| PL-19 | O.33 | ASTKG | T.18 | RTVAS |

Note:
The numbering of the Titin and Obscurin chains in the table is shown in Tables 3-1 and 3-2.

The binding activity of the PL bispecific antibodies was measured with reference to the ELISA method in Test Example 4 of WO2021139758, where the hPDL1 and hCTLA4 antigens were purchased from Sino biology. The thermal stability of the antibodies was studied. Method: The antibodies were diluted with PBS to a concentration of 1.4-3 mg/mL, and their thermal stability was measured using a high throughput differential scanning fluorimeter (UNCHAINED; model: Unit). The experimental results show that the PL bispecific antibodies retained good antigen-binding activity; additionally, there were significant increases in the Tm1 (° C.), Tagg 266 (° C.), and Tonset (° C.) of PL-2 to PL-19 as compared to PL-1, suggesting that the thermal stability of the bispecific antibodies is better.

TABLE 10
The binding activity of the PL bispecific antibodies

| | |
|---|---|
| hPDL1 | hCTLA4 |

| | |
|---|---|
| No. | EC50 (nM) |

| | | |
|---|---|---|
| PL-1 | 1.6 | 16.5 |
| PL-2 | 0.5 | 8.0 |
| PL-3 | 0.6 | 7.9 |
| PL-4 | 0.7 | 6.5 |
| PL-5 | 0.7 | 6.7 |
| PL-6 | 0.8 | 5.3 |
| PL-7 | 0.5 | 7.3 |
| PL-8 | 1.4 | 14.5 |
| PL-9 | 0.6 | 5.5 |
| PL-10 | 0.6 | 6.9 |
| PL-11 | 0.6 | 5.6 |
| PL-12 | 0.5 | 8.0 |
| PL-13 | 0.5 | 4.4 |
| PL-14 | 1.3 | 6.0 |
| PL-15 | 1.4 | 3.2 |
| PL-16 | 1.7 | 34.9 |
| PL-17 | 1.4 | 6.2 |
| PL-18 | 1.6 | 6.7 |
| PL-19 | 1.6 | 6.5 |

TABLE 11
The thermal stability of the PL bispecific antibodies

| | Tm1 | Tagg 266 | Tonset |
|---|---|---|---|
| No. | (° C.) | (° C.) | (° C.) |
| PL-1 | 61.64 | 64.82 | 52.566 |
| PL-2 | 66.20 | 66.55 | 58.317 |
| PL-3 | 64.07 | 68.28 | 57.661 |
| PL-4 | 70.71 | 67.29 | 56.246 |
| PL-5 | 74.56 | 68.11 | 58.407 |
| PL-6 | 70.43 | 70.12 | 61.069 |
| PL-7 | 68.46 | 67.41 | 63.031 |
| PL-8 | 65.00 | — | — |
| PL-9 | 69.24 | 67.83 | 58.597 |
| PL-10 | 69.63 | 68.12 | 56.788 |
| PL-11 | 65.88 | — | 57.976 |
| PL-12 | 65.54 | 67.94 | — |
| PL-13 | 71.85 | 68.17 | 58.581 |
| PL-14 | 74.18 | 69.42 | 58.589 |
| PL-15 | 70.96 | 69.91 | 58.622 |
| PL-16 | 63.48 | 68.98 | 58.702 |
| PL-17 | 70.15 | 69.86 | 56.193 |
| PL-18 | — | 69.43 | — |
| PL-19 | — | 69.52 | 57.766 |

### 1.3. HJ Bispecific Antibodies

HJ bispecific antibodies against hIL5 and hTSLP, HJ-3 to HJ11, which comprise a first heavy chain, a second heavy chain, a first light chain, and a second light chain as described below, were constructed:
the first heavy chain: when viewed from N-terminus to C-terminus, [VH1-H]-[linker 1]-[Titin chain]-[Fc1];
the first light chain: when viewed from N-terminus to C-terminus, [VL1-H]-[linker 2]-[Obscurin chain];
the second heavy chain: when viewed from N-terminus to C-terminus, [VH2-J]-[CH1]-[Fc2]; and
the second light chain: when viewed from N-terminus to C-terminus, [VL2-J]-[CL];
where the VH1-H and VL1-I are the heavy chain variable region and the light chain variable region of H0 in WO2021139758, respectively, and the VH2-J and VL2-J are the heavy chain variable region and the light chain variable region of J1 in WO2021139758, respectively. The structures of the Obscurin chain, the Titin chain, the linker 1, and the linker 2 in the HJ bispecific antibodies of this example are shown in the table below.

TABLE 12
The Obscurin/Titin chains and linkers in the HJ bispecific antibodies

| | First heavy chain | | First light chain | |
|---|---|---|---|---|
| No. | Titin chain | Linker 1 | Obscurin chain | Linker 2 |
| HJ-3 | T.10 | GGGGS | O.20 | GGGGS |
| HJ-5 | T.16 | GGGGS | O.25 | GGGGS |
| HJ-6 | T.16 | GGGGS | O.27 | GGGGS |
| HJ-7 | T.16 | GGGGS | O.28 | GGGGS |
| HJ-8 | T.16 | ASTKG | O.25 | RTVAS |
| HJ-9 | T.16 | ASTKG | O.27 | RTVAS |
| HJ-10 | T.16 | ASTKG | O.28 | RTVAS |
| HJ-11 | T.16 | ASTKG | O.29 | RTVAS |

The antigen-binding activity of the HJ bispecific antibodies was measured with reference to the method in Test Example 4 in WO2021139758. The thermal stability of the antibodies was studied. Method: Diluted solutions of the HJ bispecific antibodies were prepared with a pH 5.5 buffer containing 10 mM acetic acid and 9% sucrose and then concentrated by ultrafiltration to obtain HJ bispecific antibody solutions with different concentrations (the concentrations of the HJ bispecific antibodies are shown in Table 13-2). Subsequently, the concentrated solutions were incubated in a 40° C. incubator. At day 0 (i.e., before the 40° C. incubation, DO), day 7 (the 7th day of the 40° C. incubation, D7), day 14 (the 14th day of the 40° C. incubation, D14), day 21 (the 21st day of the 40° C. incubation, D21), and day 28 (the 28th day of the 40° C. incubation, D28), samples were taken and their SEC purity was tested. Immediately after 28 days of incubation at 40° C., samples were taken and their CE-SDS purity was tested. The experimental results show that there was no significant change in the antigen-binding activity of the HJ bispecific antibodies constructed in the present disclosure; additionally, the thermal stability of the HJ-5 to HJ-11 bispecific antibodies is better than that of HJ-3.

TABLE 13-1
The binding activity of the HJ bispecific antibodies

| | |
|---|---|
| hIL5 | hTSLP |

| | |
|---|---|
| No. | EC50 (nM) |

| | | |
|---|---|---|
| HJ-3 | 17.38 | 3.234 |
| HJ-5 | 15.96 | 2.639 |
| HJ-6 | 37.05 | 2.884 |
| HJ-7 | 17.69 | 2.182 |
| HJ-8 | 18.74 | 3.332 |
| HJ-9 | 10.86 | 3.184 |
| HJ-10 | 20.81 | 3.173 |
| HJ-11 | 9.464 | 3.005 |

TABLE 13-2
The results for the accelerated stability
testing of the HJ bispecific antibodies

| | | | | | D 28 non- |
|---|---|---|---|---|---|
| | D 0 | D 0 | D 28 | D 28Δ | reduced |
| | concentration | SEC | SEC | SEC | CE-SDS |
| No. | (mg/mL) | purity (%) | purity (%) | purity (%) | purity (%) |
| HJ-3 | 50 | 98 | 84 | 14 | 67 |
| HJ-5 | 60.8 | 98.09 | 93.86 | 4.23 | 82.93 |
| HJ-6 | 54 | 98.14 | 89.68 | 8.46 | 80.76 |
| HJ-7 | 56.8 | 97.7 | 92.72 | 4.98 | 85.47 |
| HJ-8 | 62.6 | 93.39 | 84.28 | 9.11 | 73.03 |
| HJ-9 | 53.5 | 90.01 | 85.93 | 4.08 | 80.32 |
| HJ-10 | 69.8 | 90.9 | 88.31 | 2.59 | 80.4 |
| HJ-11 | 50.4 | 92.55 | 90.89 | 1.66 | 82.96 |

### Example 2. Preparation of DLL3 Antigens, Proteins for Detection, and Stably Transfected Cell Strains

### 2.1. Construction of Cell Strain Highly Expressing DLL3

A pCDH lentiviral expression vector plasmid comprising SEQ ID NO: 1-2 (synthesized by GENEWIZ), along with a pVSVG or pCMV lentiviral packaging vector, was transfected into 293T cells (Cell Bank of the Chinese Academy of Sciences, GNHu17) using Lipofectamine 3000 (Invitrogen, L3000015). The virus-containing culture medium supernatants were collected, filtered, and subjected to ultracentrifugation. After the supernatants were discarded, resuspension was performed in 0.2 mL of sterile PBS. The concentrated viruses were then used to infect Chinese hamster ovary cells CHO-s (Invitrogen, R80007), DMS53 (ATCC, CRL-2062), and H82 (ATCC, HTB-175). After two to three weeks of screening with puromycin, FACS single-cell sorting was performed. The selected monoclonal cell strains were expanded and cryopreserved for subsequent experiments.

A pCDH-CMV-MCS-EF1-puro lentiviral expression vector plasmid comprising the LUC and GFP genes (synthesized by GENEWIZ), along with a pVSVG or pCMV lentiviral packaging vector, was transfected into 293T cells (Cell Bank of the Chinese Academy of Sciences, GNHu17) using Lipofectamine 3000 (Invitrogen, L3000015). The virus-containing culture medium supernatants were collected, filtered, and subjected to ultracentrifugation. After the supernatants were discarded, resuspension was performed in 0.2 mL of sterile PBS. SHP77 (ATCC, CRL-2195), H1184 (ATCC, CRL-5858), and H460 (Cell Bank of Chinese Academy of Sciences, TCHu205) were separately infected with the concentrated virus, screened using puromycin for two to three weeks, and subjected to FACS single-cell sorting. The selected monoclonal cell strains were expanded and cryopreserved for subsequent experiments.

The sequences of the related proteins used are as follows:

1. Human DLL3 full-length protein (SEQ ID NO: 1):
MVSPRMSGLLSQTVILALIFLPQTRPAGVFELQIHSFGPGPGPGAPRSPCSARLPCRLFF
RVCLKPGLSEEAAESPCALGAALSARGPVYTEQPGAPAPDLPLPDGLLQVPFRDAWP
GTFSFIIETWREELGDQIGGPAWSLLARVAGRRRLAAGGPWARDIQRAGAWELRFSY
RARCEPPAVGTACTRLCRPRSAPSRCGPGLRPCAPLEDECEAPLVCRAGCSPEHGFCE
QPGECRCLEGWTGPLCTVPVSTSSCLSPRGPSSATTGCLVPGPGPCDGNPCANGGSCS
ETPRSFECTCPRGFYGLRCEVSGVTCADGPCFNGGLCVGGADPDSAYICHCPPGFQG
SNCEKRVDRCSLQPCRNGGLCLDLGHALRCRCRAGFAGPRCEHDLDDCAGRACAN
GGTCVEGGGAHRCSCALGFGGRDCRERADPCAARPCAHGGRCYAHFSGLVCACAP
GYMGARCEFPVHPDGASALPAAPPGLRPGDPQRYLLPPALGLLVAAGVAGAALLLV
HVRRRGHSQDAGSRLLAGTPEPSVHALPDALNNLRTQEGSGDGPSSSVDWNRPEDV
DPQGIYVISAPSIYAREVATPLFPPLHTGRAGQRQHLLFPYPSSILSVK
2. Cynomolgus monkey DLL3 full-length protein (SEQ ID NO: 2):
MVSPRMSRLLSQTVILALIFIPQARPAGVFELQIHSFGPGPGPGAPRSPCSARGPCRLFF
RVCLKPGLSEEAAESPCALGAALSARGPVYTEQPEAPAPDLPLPNGLLQVPFRDAWP
GTFSLIIETWREELGDQIGGPAWSLLARVTRRRRLAAGGPWARDIQRAGAWELRFSY
RARCELPAVGTACTRLCRPRSAPSRCGPGLRPCAPLEDECEAPPVCRAGCSLEHGFCE
QPGECRCLEGWTGPLCMVPVSTSSCLGLRGPSSTTTGCLVPGPGPCDGNPCANGGSC
SETPGSFECTCPRGFYGLRCEVSGVTCADGPCFNGGLCVGGADPDSAYICHCPPGFQ
GSNCEKRVDRCSLQPCRNGGLCLDLGHALRCRCRAGFAGPRCEHDLDDCAGRACA
NGGTCVEGGGAHRCSCALGFGGRNCRERADPCAARPCAHGGRCYAHFSGLVCACA
PGYMGARCEFPVHPDGVSALPAAPPGLRPGDPQRYLLPPALGLLVAAGVAGAALLL
VHVRRRGHAQDAGSRLLAGTPEPSVHALPDALNNLRTQEGPGDVPSSSVDWNRPED
VDSRGIYVISAPSIYAREVAMPLFPPLHTGRAGQRQNLLFPFPSSILSVK
3. Rat DLL3 full-length protein (SEQ ID NO: 3):
MVSLQVSSLPQTLILAFLLPQALPAGVFELQIHSFGPGPGPGTPRSPCNARGPCRLFFR
VCLKPGVSQEAAESLCALGAALSTSGPVYTEQPGVPAAALSLPDGLVRVPFLDAWP
GTFSLIIETWREQLGERAAGPAWNLLARVAGRRRLAAGAPWARDVQRTGAWELHF
SYRARCEPPAVGAACARLCRSRSAPSRCGPGLRPCTPFPDECEAPRESLTVCRAGCSP
EHGYCEEPDECHCLEGWTGPLCTVPVSTSSCLNSRVSGPAGTGCLLPGPGPCDGNPC
ANGGSCSETPGSFECACPRGFYGPRCEVSGVTCADGPCFNGGLCVGGEDPDSAYVC
HCPPAFQGSNCERRVDRCSLQPCQNGGLCLDLGHALRCRCRAGFAGPRCEHDLDDC
AGRACANGGTCVEGGGARRCSCALGFGGRDCRERADPCASRPCAHGGRCYAHFSG
LVCACAPGYMGVRCEFAVRPDGADAVPAAPRGLRQADSQRFLLPPALGLLAAAAL
AGAALLLIHVRRRGPGRDTGTRLLSGTREPSVHTLPDALNNLRLQDGAGDGPTSSAD
WNHPEDGDSRSIYVIPAPSIYAREA
4. Mouse DLL3 full-length protein (SEQ ID NO: 4):
MVSLQVSPLSQTLILAFLLPQALPAGVFELQIHSFGPGPGLGTPRSPCNARGPCRLFFR
VCLKPGVSQEATESLCALGAALSTSVPVYTEHPGESAAALPLPDGLVRVPFRDAWPG
TFSLVIETWREQLGEHAGGPAWNLLARVVGRRRLAAGGPWARDVQRTGTWELHFS
YRARCEPPAVGAACARLCRSRSAPSRCGPGLRPCTPFPDECEAPSVCRPGCSPEHGYC
EEPDECRCLEGWTGPLCTVPVSTSSCLNSRVPGPASTGCLLPGPGPCDGNPCANGGS
CSETSGSFECACPRGFYGLRCEVSGVTCADGPCFNGGLCVGGEDPDSAYVCHCPPGF
QGSNCEKRVDRCSLQPCQNGGLCLDLGHALRCRCRAGFAGPRCEHDLDDCAGRAC
ANGGTCVEGGGSRRCSCALGFGGRDCRERADPCASRPCAHGGRCYAHFSGLVCAC
APGYMGVRCEFAVRPDGADAVPAAPRGLRQADPQRFLLPPALGLLVAAGLAGAAL
LVIHVRRRGPGQDTGTRLLSGTREPSVHTLPDALNNLRLQDGAGDGPSSSADWNHPE
DGDSRSIYVIPAPSIYAREDWLIQVLF
5. Human DLL1 full-length protein (SEQ ID NO: 5):
MGSRCALALAVLSALLCQVWSSGVFELKLQEFVNKKGLLGNRNCCRGGAGPPPCA
CRTFFRVCLKHYQASVSPEPPCTYGSAVTPVLGVDSFSLPDGGGADSAFSNPIRFPFG
FTWPGTFSLIIEALHTDSPDDLATENPERLISRLATQRHLTVGEEWSQDLHSSGRTDL
KYSYRFVCDEHYYGEGCSVFCRPRDDAFGHFTCGERGEKVCNPGWKGPYCTEPICL
PGCDEQHGFCDKPGECKCRVGWQGRYCDECIRYPGCLHGTCQQPWQCNCQEGWG
GLFCNQDLNYCTHHKPCKNGATCTNTGQGSYTCSCRPGYTGATCELGIDECDPSPCK
NGGSCTDLENSYSCTCPPGFYGKICELSAMTCADGPCFNGGRCSDSPDGGYSCRCPV
GYSGFNCEKKIDYCSSSPCSNGAKCVDLGDAYLCRCQAGFSGRHCDDNVDDCASSP
CANGGTCRDGVNDFSCTCPPGYTGRNCSAPVSRCEHAPCHNGATCHERGHRYVCEC
ARGYGGPNCQFLLPELPPGPAVVDLTEKLEGQGGPFPWVAVCAGVILVLMLLLGCA
AVVVCVRLRLQKHRPPADPCRGETETMNNLANCQREKDISVSIIGATQIKNTNKKAD
FHGDHSADKNGFKARYPAVDYNLVQDLKGDDTAVRDAHSKRDTKCQPQGSSGEEK
GTPTTLRGGEASERKRPDSGCSTSKDTKYQSVYVISEEKDECVIATEV
6. Human DLL4 full-length protein (SEQ ID NO: 6):
MAAASRSASGWALLLLVALWQQRAAGSGVFQLQLQEFINERGVLASGRPCEPGCRT
FFRVCLKHFQAVVSPGPCTFGTVSTPVLGTNSFAVRDDSSGGGRNPLQLPFNFTWPG
TFSLIIEAWHAPGDDLRPEALPPDALISKIAIQGSLAVGQNWLLDEQTSTLTRLRYSYR
VICSDNYYGDNCSRLCKKRNDHFGHYVCQPDGNLSCLPGWTGEYCQQPICLSGCHE
QNGYCSKPAECLCRPGWQGRLCNECIPHNGCRHGTCSTPWQCTCDEGWGGLFCDQ
DLNYCTHHSPCKNGATCSNSGQRSYTCTCRPGYTGVDCELELSECDSNPCRNGGSC
KDQEDGYHCLCPPGYYGLHCEHSTLSCADSPCFNGGSCRERNQGANYACECPPNFT
GSNCEKKVDRCTSNPCANGGQCLNRGPSRMCRCRPGFTGTYCELHVSDCARNPCAH
GGTCHDLENGLMCTCPAGFSGRRCEVRTSIDACASSPCFNRATCYTDLSTDTFVCNC
PYGFVGSRCEFPVGLPPSFPWVAVSLGVGLAVLLVLLGMVAVAVRQLRLRRPDDGS
REAMNNLSDFQKDNLIPAAQLKNTNQKKELEVDCGLDKSNCGKQQNHTLDYNLAP
GPLGRGTMPGKFPHSDKSLGEKAPLRLHSEKPECRISAICSPRDSMYQSVCLISEERNE
CVIATEV.

### 2.2. Preparation of Antigens

Human DLL3 (Uniprot, Q9NYJ7), cynomolgus monkey DLL3 (Uniprot, A0A2K5WSR4), and mouse DLL3 (Uniprot, 088516) sequences were used as templates to design human DLL3 ECD fusion proteins containing different tags, and the fusion proteins were cloned onto pTT5 vectors. After expression by 293E cells, antigens were obtained. The amino acid sequences of the related proteins are as follows:

1. His-hDLL3 (ECD) (SEQ ID NO: 7):

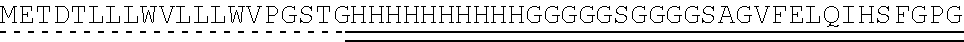

PGPGAPRSPCSARLPCRLFFRVCLKPGLSEEAAESPCALGAALSARGPVYTEQPGAPA
PDLPLPDGLLQVPFRDAWPGTFSFIIETWREELGDQIGGPAWSLLARVAGRRRLAAG
GPWARDIQRAGAWELRFSYRARCEPPAVGTACTRLCRPRSAPSRCGPGLRPCAPLED
ECEAPLVCRAGCSPEHGFCEQPGECRCLEGWTGPLCTVPVSTSSCLSPRGPSSATTGC
LVPGPGPCDGNPCANGGSCSETPRSFECTCPRGFYGLRCEVSGVTCADGPCFNGGLC
VGGADPDSAYICHCPPGFQGSNCEKRVDRCSLQPCRNGGLCLDLGHALRCRCRAGE
AGPRCEHDLDDCAGRACANGGTCVEGGGAHRCSCALGFGGRDCRERADPCAARPC
AHGGRCYAHFSGLVCACAPGYMGARCEFPVHPDGASALPAAPPGLRPGDPQRYL
Note: The signal peptide sequence is highlighted with a dotted line; the his
tag and the linker are single underlined; and the extracellular region of DLL3 is double
underlined.
2. Fc-hDLL3 (ECD) (SEQ ID NO: 8):

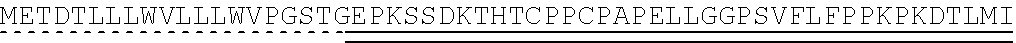

SRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGN
VFSCSVMHEALHNHYTQKSLSLSPGGGGGSGGGGSAGVFELQIHSEGPGPGPGAPRS
PCSARLPCRLFFRVCLKPGLSEEAAESPCALGAALSARGPVYTEQPGAPAPDLPLPDG
LLQVPERDAWPGTFSFIIETWREELGDQIGGPAWSLLARVAGRRRLAAGGPWARDIQ
RAGAWELRFSYRARCEPPAVGTACTRLCRPRSAPSRCGPGLRPCAPLEDECEAPLVC
RAGCSPEHGFCEQPGECRCLEGWTGPLCTVPVSTSSCLSPRGPSSATTGCLVPGPGPC
DGNPCANGGSCSETPRSFECTCPRGFYGLRCEVSGVTCADGPCENGGLCVGGADPDS
AYICHCPPGFQGSNCEKRVDRCSLQPCRNGGLCLDLGHALRCRCRAGFAGPRCEHD
LDDCAGRACANGGTCVEGGGAHRCSCALGFGGRDCRERADPCAARPCAHGGRCY
AHESGLVCACAPGYMGARCEFPVHPDGASALPAAPPGLRPGDPQRYL
Note: The signal peptide sequence is highlighted with a dotted line; the Fc
tag and the linker are single underlined; and the extracellular region of DLL3 is double
underlined.
3. hDLL3 (ECD)-strep twin (SEQ ID NO: 9):

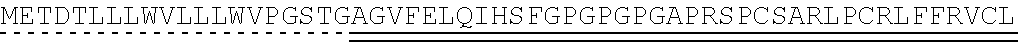

KPGLSEEAAESPCALGAALSARGPVYTEQPGAPAPDLPLPDGLLQVPFRDAWPGTES
FIIETWREELGDQIGGPAWSLLARVAGRRRLAAGGPWARDIQRAGAWELRFSYRAR
CEPPAVGTACTRLCRPRSAPSRCGPGLRPCAPLEDECEAPLVCRAGCSPEHGFCEQPG
ECRCLEGWTGPLCTVPVSTSSCLSPRGPSSATTGCLVPGPGPCDGNPCANGGSCSETP
RSFECTCPRGFYGLRCEVSGVTCADGPCFNGGLCVGGADPDSAYICHCPPGFQGSNC
EKRVDRCSLQPCRNGGLCLDLGHALRCRCRAGFAGPRCEHDLDDCAGRACANGGT
CVEGGGAHRCSCALGFGGRDCRERADPCAARPCAHGGRCYAHFSGLVCACAPGYM
GARCEFPVHPDGASALPAAPPGLRPGDPQRYLWSHPQFEKGGGSGGGSGGSAWSHP
QFEK
Note: The signal peptide sequence is highlighted with a dotted line; the
extracellular region of DLL3 is double-underlined; and the strep twin tag is single-underlined.
4. cynoDLL3 (ECD)-strep twin (SEQ ID NO: 10):

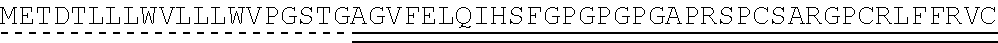

LKPGLSEEAAESPCALGAALSARGPVYTEQPEAPAPDLPLPNGLLQVPERDAWPGTE
SLIIETWREELGDQIGGPAWSLLARVTRRRRLAAGGPWARDIQRAGAWELRFSYRAR
CELPAVGTACTRLCRPRSAPSRCGPGLRPCAPLEDECEAPPVCRAGCSLEHGECEQPG
ECRCLEGWTGPLCMVPVSTSSCLGLRGPSSTTTGCLVPGPGPCDGNPCANGGSCSET
PGSFECTCPRGFYGLRCEVSGVTCADGPCFNGGLCVGGADPDSAYICHCPPGFQGSN
CEKRVDRCSLQPCRNGGLCLDLGHALRCRCRAGEAGPRCEHDLDDCAGRACANGG
TCVEGGGAHRCSCALGFGGRNCRERADPCAARPCAHGGRCYAHESGLVCACAPGY
MGARCEFPVHPDGVSALPAAPPGLRPGDPQRYLWSHPQFEKGGGSGGGSGGSAWS
HPQFEK
Note: The signal peptide sequence is highlighted with a dotted line; the
extracellular region of DLL3 is double-underlined; and the strep twin tag is single-underlined.
5. mouDLL3 (ECD)-strep twin (SEQ ID NO: 11):

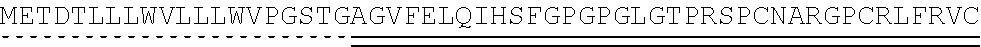

LKPGVSQEATESLCALGAALSTSVPVYTEHPGESAAALPLPDGLVRVPERDAWPGTF
SLVIETWREQLGEHAGGPAWNLLARVVGRRRLAAGGPWARDVQRTGTWELHFSYR
ARCEPPAVGAACARLCRSRSAPSRCGPGLRPCTPFPDECEAPSVCRPGCSPEHGYCEE
PDECRCLEGWTGPLCTVPVSTSSCLNSRVPGPASTGCLLPGPGPCDGNPCANGGSCSE
TSGSFECACPRGFYGLRCEVSGVTCADGPCENGGLCVGGEDPDSAYVCHCPPGFQGS
NCEKRVDRCSLQPCQNGGLCLDLGHALRCRCRAGFAGPRCEHDLDDCAGRACANG
GTCVEGGGSRRCSCALGFGGRDCRERADPCASRPCAHGGRCYAHESGLVCACAPGY
MGVRCEFAVRPDGADAVPAAPRGLRQADPQRELWSHPQFEKGGGSGGGSGGSAWS
HPQFEK
Note: The signal peptide sequence is highlighted with a dotted line; the
extracellular region of DLL3 is double-underlined; and the strep twin tag is single-underlined.

### Example 3. Preparation of Anti-Human DLL3 Antibodies

### 3.1. Screening and Identification of Anti-Human DLL3 Hybridoma Antibodies

Monoclonal antibodies against human DLL3 were prepared using the hybridoma technique. The immunization method was as follows:

For the first group, cross-immunization was performed using the adjuvants TiterMaxR Gold Adjuvant (Sigma Cat No. T2684) and Thermo Imject® Alum (Thermo Cat No. 77161), with His-hDLL3 (ECD) (SEQ ID NO: 7) as the immunogen. The ratio of the antigen to the adjuvant (TiterMax® Gold Adjuvant) was 1:1, and the ratio of the antigen to the adjuvant (Thermo Imject® Alum) was 3:1. The dose for the first immunization was 50 μg/mouse/immunization, and the dose for boost immunization was 25 μg/mouse/immunization. The antibody titer in the mouse serum was determined by ELISA.

The immunizing antigens for the second group of mice were DLL3 CHO-s cells and Fc-hDLL3 (ECD) (SEQ ID NO: 8), and immunizations were alternately performed with cell and protein antigens. Cell immunization was performed by injecting intraperitoneally into each mouse 0.1 mL of a cell suspension diluted with normal saline to a concentration of 10⁸/mL. For the Fc-hDLL3 (ECD) antigen, cross-immunizations were performed using the adjuvants TiterMax® Gold Adjuvant (Sigma Cat No. T2684) and Thermo Imject® Alum (Thermo Cat No. 77161). The ratio of the antigen to the adjuvant (TiterMax® Gold Adjuvant) was 1:1, and the ratio of the antigen to the adjuvant (Thermo Imject® Alum) was 3:1. The dose for the first immunization was 50 μg/mouse/immunization, and the dose for boost immunization was 25 μg/mouse/immunization. The antibody titer in the mouse serum was determined by ELISA.

Spleen lymphocytes and the myeloma cells, Sp2/0 cells (ATCC® CRL-8287™), were fused by following an optimized electrofusion procedure to obtain hybridoma cells. Based on the growth density of hybridoma cells, the hybridoma culture supernatant was assayed using an ELISA method with binding to DLL3 protein and a FACS method with binding to DLL3 CHO-s cells. Clones that bind to human DLL3 protein, monkey DLL3 protein, and DLL3 CHO-s cells but do not bind to wild-type CHO-s cells were selected, cryopreserved, expanded, conserved, and subcloned one to two times in time until single cell clones were obtained. Through the screening experiment above, hybridoma clones mAb6, mAb98, and mAb110 were obtained.

The hybridoma clones were expanded, and RNA was extracted. Reverse transcription amplification (RT-PCR) was performed using a degenerate primer of mouse-Ig, and the variable region sequences of the antibodies were finally obtained.

The heavy chain variable region of mAb6 (SEQ ID NO: 12):
EVQLQQSGPVLVKPGASVKMSCKASGYTFTDYYMNWVKQSHGKSLEWIGIINPYSG
VTIYNHKFKGKATLTVDKSSNTAYMELNSLTSEDSAVYYCARQDSLLDYAMDYWG
QGTSVTVSS
The light chain variable region of mAb6 (SEQ ID NO: 13):
DILMTQSPSSMSVSLGDTVSITCHASQGISGNMGWLQQKPGKSFKGLIYHGANLEDG
VPSRFSGSGSGADYSLTISSLESEDFADYYCVQYAQFPYTFGGGTKLEIK
The heavy chain variable region of mAb98 (SEQ ID NO: 14):
EVQLVESGGGLVKPGGSLKLSCAASGFTFSDYGIHWVRQAPEKGLEWVAYISSGGY
TIYY ADTVKGRFTISRDNAKNTLFLQMTSLRSEDTAMYYCARGDYDDIFYTMDYWG
QGTSVTVSS
The light chain variable region of mAb98 (SEQ ID NO: 15):
DIQMTQTTSSLSASLGDRVTISCRASRDITKFLNWYQQKPDGTVKLLIYYTSRLHSGV
PSRFSGSGSGTDYSLTISNLDQEDIATYFCQQGNTLPWTFGGGTKLDIK
The heavy chain variable region of mAb110 (SEQ ID NO: 16):
EVQLQQSGPVLVKPGASVKMSCKTSGYTFTDYYMNWVKQSHGKSLEWIGIINPYNG
GTSYTQKFKGKATLTVDKSSSTAYMELNSLTSEDSTVYFCARHFYDSSYLTYSTMDY
WGQGTSVTVSS
The light chain variable region of mAb110 (SEQ ID NO: 17):
NIVMTQSPKSMSMSVGERVTLSCKASENVGIFVSWYQQKPKQSPKLLIYGASNRYTG
VPDRFTGSGSATDFTLTISSVQAEDLADYYCGQSHSYPLTFGTGTKLELK.

### 3.2. Protocol for Screening Immune Repertoire for Antibodies, and Antibody Sequences

Total RNA was extracted from spleen cells of immunized mice using the Trizol reagent and reverse-transcribed into cDNA. Degenerate primers were used to amplify the heavy and light chain variable region sequences of the antibodies, with the cDNA as a template, and the amplified fragments were constructed into an optimized phage display vector. The constructed vector was then introduced into an E. coli TG1 strain through electroporation to obtain a DLL3 immune library. The obtained DLL3 immune library was packaged into phages, and two runs of library screening were conducted using biotinylated human DLL3 protein. The single clones obtained from the screening were inoculated into 96-well plates for phage packaging. Phages that could bind to human DLL3 protein and monkey DLL3 protein were identified by ELISA, and the corresponding single clones were sequenced to finally obtain the variable region sequences of the anti-DLL3 antibodies.

The heavy chain variable region of mAb30
(SEQ ID NO: 18):
EVRLQQFGPVLVKPGASVKMSCKASGYTFTDYYMNWVKQSHGKSLEWIGI
INPYNGDTSYNQKFRGKATLTVDKSSSTAYMELNSLTSEDSAVYYCARYH
YGPHLEGYFDVWGTGTTLTVSS
The light chain variable region of mAb30
(SEQ ID NO: 19):
DIVMTQSPKSMSMSVGERVTLSCKASENVGYVSWYQQKPEQSPKLLIYGA
SNRYTGVPDRFTGSGSATDFTLTISSVQAEDLADYHCGQSYSYPLTFGSG
TKLEIK.

TABLE 14
The CDRs of antibodies

| Antibody | Name | Sequence | Sequence No. |
|---|---|---|---|
| mAb6 | HCDR1 | DYYMN | SEQ ID NO: 20 |
| | HCDR2 | IINPYSGVTIYNHKFKG | SEQ ID NO: 21 |
| | HCDR3 | QDSLLDYAMDY | SEQ ID NO: 22 |
| | LCDR1 | HASQGISGNMG | SEQ ID NO: 23 |
| | LCDR2 | HGANLED | SEQ ID NO: 24 |
| | LCDR3 | VQYAQFPYT | SEQ ID NO: 25 |
| mAb98 | HCDR1 | DYGIH | SEQ ID NO: 26 |
| | HCDR2 | YISSGGYTIYY ADTVKG | SEQ ID NO: 27 |
| | HCDR3 | GDYDDIFYTMDY | SEQ ID NO: 28 |
| | LCDR1 | RASRDITKFLN | SEQ ID NO: 29 |
| | LCDR2 | YTSRLHS | SEQ ID NO: 30 |
| | LCDR3 | QQGNTLPWT | SEQ ID NO: 31 |
| mAb110 | HCDR1 | DYYMN | SEQ ID NO: 20 |
| | HCDR2 | IINPYNGGTSYTQKFKG | SEQ ID NO: 32 |
| | HCDR3 | HFYDSSYLTYSTMDY | SEQ ID NO: 33 |
| | LCDR1 | KASENVGIFVS | SEQ ID NO: 34 |
| | LCDR2 | GASNRYT | SEQ ID NO: 35 |
| | LCDR3 | GQSHSYPLT | SEQ ID NO: 36 |
| mAb30 | HCDR1 | DYYMN | SEQ ID NO: 20 |
| | HCDR2 | IINPYNGDTSYNQKFRG | SEQ ID NO: 37 |
| | HCDR3 | YHYGPHLEGYFDV | SEQ ID NO: 38 |
| | LCDR1 | KASENVGYVS | SEQ ID NO: 39 |
| | LCDR2 | GASNRYT | SEQ ID NO: 35 |
| | LCDR3 | GQSYSYPLT | SEQ ID NO: 40 |

Note:
The amino acid residues of the CDRs of the VH/VL were determined and annotated using the Kabat numbering scheme.

The heavy chain variable regions and the light chain variable regions of the murine antibodies were cloned into pTT5 vector plasmids containing a human IgG1 heavy chain constant region set forth in SEQ ID NO: 41 and a κ light chain constant region set forth in SEQ ID NO: 42, and HEK293 cells were transfected with the resulting plasmids to obtain anti-DLL3 chimeric antibodies M6CH1, M98CH1, M110CH1, and P30CH1.

The heavy chain constant region of human
IgGI (SEQ ID NO: 41):
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
The light chain constant region of human
κ (hκ) (SEQ ID NO: 42):
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

### Example 4. Humanization of Anti-DLL3 Monoclonal Antibodies

Through comparison with a Kabat human antibody heavy and light chain variable region germline gene database, heavy and light chain variable region germline genes with high homology were selected as templates, and the CDRs of the murine antibodies were grafted onto the corresponding human templates to form variable region sequences structured as FR1-CDR1-FR2-CDR2-FR3-CDR3-FR4. Certain amino acids in the variable regions were subjected to amino acid substitution, and the variable regions were recombined with constant regions (for example, the human IgG1 heavy chain constant region set forth in SEQ ID NO: 41 and the human κ light chain constant region set forth in SEQ ID NO: 42) to obtain full-length humanized antibodies.

4-1. Humanization of Antibody mAb6

For the antibody mAb6, the human germline light chain templates were IGKV1G16*01 or IGKV3-20*02 and IGKJ4*01, and the human germline heavy chain templates were IGHV1-3*01, IGHV7-4-1*02, or IGHV3G73*01 and IGHJ6*01. Optionally, the amino acid residue(s) at position(s) 1, 36, 42, 43, 44, 46, 52, 56, 69, 71, 79, 85, and/or 94 in the light chain variable region of the humanized antibody were/was substituted; and/or the amino acid residue(s) at position(s) 1, 27, 30, 38, 43, 48, 67, 68, 69, 71, 73, 75, 76, 93, 56, 58, and/or 5 in the heavy chain variable region of the humanized antibody were/was substituted.

TABLE 15
Humanization templates and corresponding point
mutations for the mAb6 murine antibody

| hu VL | | hu VH | |
|---|---|---|---|
| VL1 | Graft (IGKV1-16*01) + | VH1 | Graft(IGHV1-3*01) + |
| | F36L, S46G | | Q1E, R71V, T73K |
| VL2 | Graft(IGKV1-16*01) + | VH2 | Graft(IGHV1-3*01) + |
| | F36L, S46G, T69A, | | Q1E, I69L, R71V, T73K, |
| | F71Y | | S76N |
| VL3 | Graft(IGKV1-16*01) + | VH3 | Graft(IGHV1-3*01) + |
| | F36L, A43S, P44F, | | Q1E, M48I, V67A, I69L, |
| | S46G, T69A, F71Y | | R71V, T73K, S76N |
| VL4 | Graft(IGKV1-16*01) + | VH4 | Graft(IGHV1-3*01) + |
| | F36L, S46G, T69A, | | Q1E, Q43K, I69L, R71V, |
| | F71Y, T85D | | T73K, S76N |
| VL5 | Graft (IGKV1-16*01) | VH5 | Graft(IGHV7-4-1*02) + |
| | | | Q1E, L71V, T73K, F69L |
| VL6 | Graft(IGKV1-16*01) + | VH6 | Graft(IGHV7-4-1*02) + |
| | F36L, S46G, T69A, | | Q1E, F69L, L71V, T73K, |
| | F71Y, T85D + D56E | | V75S, S76N |
| VL7 | Graft(IGKV1-16*01) + | VH7 | Graft(IGHV3-73*01) + |
| | F36L, S46G, T69A, | | F27Y, S30T, I69L, R71V, |
| | F71Y, T85D + D56S | | D73K, T93A |
| VL8 | Graft(IGKV1-16*01) + | VH8 | Graft(IGHV1-3*01) |
| | F36L, S46G, T69A, | | |
| | F71Y, T85D + F94Y, | | |
| | D56E | | |
| VL9 | Graft(IGKV1-16*01) + | VH9 | Graft(IGHV7-4-1*02) |
| | F36L, S46G, T69A, | | |
| | F71Y, T85D + F94Y, | | |
| | D1N, A52R, D56E | | |
| VL10 | Graft(IGKV3-20*02) + | VH10 | Graft(IGHV3-73*01) |
| | Y36L, Q42K, L46G, | | |
| | T69A, F71Y, E79Q, | | |
| | V85D + D56E | | |
| VL11 | Graft(IGKV3-20*02) + | VH11 | Graft(IGHV7-4-1*02) + |
| | D56E | | Q1E, R38K, F69L, L71V, |
| | | | T73K, V75S, S76N |
| | | VH12 | Graft(IGHV7-4-1*02) + |
| | | | Q1E, V68A, F69L, L71V, |
| | | | T73K, V75S, S76N, |
| | | VH13 | Graft(IGHV7-4-1*02) + |
| | | | Q1E, F69L, L71V, T73K, |
| | | | V75S, S76N, R38K, V68A |
| | | VH14 | Graft(IGHV7-4-1*02) + |
| | | | Q1E, F69L, L71V, T73K, |
| | | | V75S, S76N, −V68A + |
| | | | I58T |
| | | VH15 | Graft(IGHV7-4-1*02) + |
| | | | Q1E, F69L, L71V, T73K, |
| | | | V75S, S76N, −V68A + |
| | | | V56S, V5Q |
| FR4 | IGKJ4*01 | FR4 | IGHJ6*01 |

Note:
The amino acid positions in the table are numbered according to the Kabat numbering scheme. F36L means that the F at position 36, according to the Kabat numbering scheme, is mutated to L. The same applies hereinafter.

The sequences of the variable regions of the hu6 humanized antibodies obtained are as follows:

hu6 VH1
(SEQ ID NO: 43) [symbol]
VQLVQSGAEVKKPGASVKVSCKASGYTFTDYYMNWVRQAPGQRLEWMGIINPYS
GVTIYNHKFKGRVTIT [symbol] D [symbol] SASTAYMELSSLRSEDTAVYYCARQDSLLDYAMDYW
GQGTTVTVSS
hu6 VH2
(SEQ ID NO: 44) [symbol]
VQLVQSGAEVKKPGASVKVSCKASGYTFTDYYMNWVRQAPGQRLEWMGIINPYS
GVTIYNHKFKGRVT [symbol] T [symbol] D [symbol] SA [symbol] TAYMELSSLRSEDTAVYYCARQDSLLDYAMDYW
GQGTTVTVSS
hu6 VH3
(SEQ ID NO: 45) [symbol]
LVQSGAEVKKPGASVKVSCKASGYTFTDYYMNWVRQAPGQRLEWIGIINPYSG
VTIYNHKFKGR [symbol] T [symbol] T [symbol] D [symbol] SA [symbol] TAYMELSSLRSEDTAVYYCARQDSLLDYAMDYW
GQGTTVTVSS
hu6 VH4
(SEQ ID NO: 46) [symbol]
LVQSGAEVKKPGASVKVSCKASGYTFTDYYMNWVRQAPG [symbol] RLEWMGIINPYS
GVTIYNHKFKGRVT [symbol] T [symbol] D [symbol] SA [symbol] TAYMELSSLRSEDTAVYYCARQDSLLDYAMDYW
GQGTTVTVSS
hu6 VH5
(SEQ ID NO: 47) [symbol]
LVQSGSELKKPGASVKVSCKASGYTFTDYYMNWVRQAPGQGLEWMGIINPYS
GVTIYNHKFKGRFV [symbol] S [symbol] D [symbol] SVSTAYLQISSLKAEDTAVYYCARQDSLLDYAMDYW
GQGTTVTVSS
hu6 VH6
(SEQ ID NO: 48) [symbol]
LVQSGSELKKPGASVKVSCKASGYTFTDYYMNWVRQAPGQGLEWMGIINPYS
GVTIYNHKFKGRFV [symbol] S [symbol] D [symbol] S [symbol] TAYLQISSLKAEDTAVYYCARQDSLLDYAMDYW
GQGTTVTVSS
hu6 VH7
(SEQ ID NO: 49) [symbol]
LVESGGGLVQPGGSLKLSCAASG [symbol] TF [symbol] DYYMNWVRQASGKGLEWVGIINPYS
GVTIYNHKFKGRFT [symbol] S [symbol] D [symbol] SKNTAYLQMNSLKTEDTAVYYC [symbol] RQDSLLDYAMDY
WGQGTTVTVSS
hu6VH8
(Graft IGHV1-3*01)
(SEQ ID NO: 50)
QVQLVQSGAEVKKPGASVKVSCKASGYTFTDYYMNWVRQAPGQRLEWMGIINPYS
GVTIYNHKFKGRVTITRDTSASTAYMELSSLRSEDTAVYYCARQDSLLDYAMDYWG
QGTTVTVSS
hu6VH9
(Graft IGHV7-4-1*02)
(SEQ ID NO: 51)
QVQLVQSGSELKKPGASVKVSCKASGYTFTDYYMNWVRQAPGQGLEWMGIINPYS
GVTIYNHKFKGRFVFSLDTSVSTAYLQISSLKAEDTAVYYCARQDSLLDYAMDYWG
QGTTVTVSS
hu6VH10
(Graft IGHV3-73*01)
(SEQ ID NO: 52)
EVQLVESGGGLVQPGGSLKLSCAASGFTFSDYYMNWVRQASGKGLEWVGIINPYSG
VTIYNHKFKGRFTISRDDSKNTAYLQMNSLKTEDTAVYYCTRQDSLLDYAMDYWG
QGTTVTVSS
hu6VH11
(SEQ ID NO: 53) [symbol]
LVQSGSELKKPGASVKVSCKASGYTFTDYYMNWV [symbol] QAPGQGLEWMGIINPYS
GVTIYNHKFKGRFV [symbol] S [symbol] D [symbol] S [symbol] TAYLQISSLKAEDTAVYYCARQDSLLDYAMDYW
GQGTTVTVSS
hu6VH12
(SEQ ID NO: 54) [symbol]
VQSGSELKKPGASVKVSCKASGYTFTDYYMNWVRQAPGQGLEWMGIINPYS
GVTIYNHKFKGRF [symbol] S [symbol] D [symbol] S [symbol] TAYLQISSLKAEDTAVYYCARQDSLLDYAMDYW
GQGTTVTVSS
hu6VH13
(SEQ ID NO: 55) [symbol]
LVQSGSELKKPGASVKVSCKASGYTFTDYYMNWV [symbol] QAPGQGLEWMGIINPYS
GVTIYNHKFKGRF [symbol] S [symbol] D [symbol] S [symbol] TAYLQISSLKAEDTAVYYCARQDSLLDYAMDYW
GQGTTVTVSS
hu6VH14
(SEQ ID NO: 56) [symbol]
LVQSGSELKKPGASVKVSCKASGYTFTDYYMNWVRQAPGQGLEWMGIINPYS
GVTTYNHKFKGRF [symbol] S [symbol] D [symbol] S [symbol] TAYLQISSLKAEDTAVYYCARQDSLLDYAMDYW
GQGTTVTVSS
hu6VH15
(SEQ ID NO: 57) [symbol]
VQL [symbol] QSGSELKKPGASVKVSCKASGYTFTDYYMNWVRQAPGQGLEWMGIINPYS
G [symbol] TIYNHKFKGRF [symbol] S [symbol] D [symbol] S [symbol] TAYLQISSLKAEDTAVYYCARQDSLLDYAMDYWG
QGTTVTVSS
hu6 VL1
(SEQ ID NO: 58)
DIQMTQSPSSLSASVGDRVTITCHASQGISGNMGW [symbol] QQKPGKAPK [symbol] LIYHGANLED
GVPSRFSGSGSGTDFTLTISSLQPEDFATYYCVQYAQFPYTFGGGTKVEIK
hu6 VL2
(SEQ ID NO: 59)
DIQMTQSPSSLSASVGDRVTITCHASQGISGNMGW [symbol] QQKPGKAPK [symbol] LIYHGANLED
GVPSRFSGSGSG [symbol] D [symbol] TLTISSLQPEDFATYYCVQYAQFPYTFGGGTKVEIK
hu6 VL3
(SEQ ID NO: 60)
DIQMTQSPSSLSASVGDRVTITCHASQGISGNMGW [symbol] QQKPGK [symbol] K [symbol] LIYHGANLEDG
VPSRFSGSGSG [symbol] D [symbol] TLTISSLOPEDFATYYCVQYAQFPYTFGGGTKVEIK
hu6 VL4
(SEQ ID NO: 61)
DIQMTQSPSSLSASVGDRVTITCHASQGISGNMGW [symbol] QQKPGKAPK [symbol] LIYHGANLED
GVPSRFSGSGSG [symbol] D [symbol] TLTISSLQPEDFA [symbol] YYCVQYAQFPYTFGGGTKVEIK
hu6 VL5
(Graft IGKV1-16*01)
(SEQ ID NO: 62)
DIQMTQSPSSLSASVGDRVTITCHASQGISGNMGWFQQKPGKAPKSLIYHGANLEDG
VPSRFSGSGSGTDFTLTISSLQPEDFATYYCVQYAQFPYTFGGGTKVEIK
hu6VL6
(SEQ ID NO: 63)
DIQMTQSPSSLSASVGDRVTITCHASQGISGNMGW [symbol] QQKPGKAPK [symbol] LIYHGANLE [symbol] G
VPSRFSGSGSG [symbol] D [symbol] TLTISSLQPEDFA [symbol] YYCVQYAQFPYTFGGGTKVEIK
hu6VL7
(SEQ ID NO: 64)
DIQMTQSPSSLSASVGDRVTITCHASQGISGNMGW [symbol] QQKPGKAPK [symbol] LIYHGANLE [symbol] G
VPSRFSGSGSG [symbol] D [symbol] TLTISSLQPEDFA [symbol] YYCVQYAQFPYTFGGGTKVEIK
hu6VL8
(SEQ ID NO: 65)
DIQMTQSPSSLSASVGDRVTITCHASQGISGNMGW [symbol] QQKPGKAPK [symbol] LIYHGANLE [symbol] G
VPSRFSGSGSG [symbol] D [symbol] TLTISSLOPEDFA [symbol] YYCVQYAQ [symbol] PYTFGGGTKVEIK
hu6VL9
(SEQ ID NO: 66) [symbol]
IQMTQSPSSLSASVGDRVTITCHASQGISGNMGW [symbol] QQKPGKAPK [symbol] LIYHG [symbol] NLE [symbol] G
VPSRFSGSGSG [symbol] D [symbol] TLTISSLQPEDFA [symbol] YYCVQYAQ [symbol] PYTFGGGTKVEIK
hu6VL10
Graft (IGKV3-20*02) + Y36L, Q42K, L46G, T69A, F71Y, E79Q,
V85D + D56E
(SEQ ID NO: 67)
EIVLTQSPATLSLSPGERATLSCHASQGISGNMGW [symbol] QQKPG [symbol] APR [symbol] LIYHGANLE [symbol] GI
PARFSGSGSG [symbol] D [symbol] TLTISRL [symbol] PEDFA [symbol] YYCVQYAQFPYTFGGGTKVEIK
hu6VL11
Graft (IGKV3-20*02) + D56E
(SEQ ID NO: 68)
EIVLTQSPATLSLSPGERATLSCHASQGISGNMGWYQQKPGQAPRLLIYHGANLEEGI
PARFSGSGSGTDFTLTISRLEPEDFAVYYCVQYAQFPYTFGGGTKVEIK

Note: CDRs are single underlined, amino acid substitution sites are double underlined, and the rest are FR regions. The same applies hereinafter.

The CDRs of the humanized antibodies of mAb6 are as follows:

TABLE 16
The CDRs of the humanized antibodies of mAb6

| Variable region | CDR | Sequence | SEQ ID NO |
|---|---|---|---|
| hu6VH14 | HCDR2 | INPYSGVTTYNHKFKG | 69 |
| hu6VH15 | HCDR2 | INPYSGSTIYNHKFKG | 70 |
| hu6VL6/VL8/ | LCDR2 | HGANLEE | 71 |
| VL10/VL11 | | | |
| hu6VL7 | LCDR2 | HGANLES | 72 |
| hu6VL9 | LCDR2 | HGRNLEE | 73 |
| hu6VL8/VL9 | LCDR3 | VQYAQYPYT | 74 |

3-2. Humanization of mAb98

For the antibody mAb98, the human germline light chain templates were IGKV1-39*01 and IGKJ4*01, and the human germline heavy chain templates were IGHV3-7*01 and IGHJ6*01. Optionally, the amino acid residue(s) at position(s) 42, 44, and/or 71 in the light chain variable region of the humanized antibody were/was substituted; and/or the amino acid residue(s) at position(s) 34, 57, and/or 98 in the heavy chain variable region of the humanized antibody were/was substituted.

TABLE 17
Humanization templates and corresponding point
mutations for the mAb98 murine antibody

| hu98VL | | hu98VH | |
|---|---|---|---|
| VL1 | Graft(IGKV1-39*01) | VH1 | Graft(IGHV3-7*01) |
| VL2 | Graft(IGKV1-39*01) + | VH2 | Graft(IGHV3-7*01) + |
| | P44V, F71Y | | D98E |
| VL3 | Graft(IGKV1-39*01) + | VH3 | Graft(IGHV3-7*01) + |
| | K42G, P44V, F71Y | | I34M, D98E |
| | | VH4 | Graft(IGHV3-7*01) + |
| | | | I57S, D98E |
| | | VH5 | Graft(IGHV3-7*01) + |
| | | | I34M, I57S, D98E |
| FR4 | IGKJ4*01 | FR4 | IGHJ6*01 |

The sequences of the variable regions of the hu98 humanized antibodies obtained are as follows:

hu98VL1 Graft (IGKV1-39*01)
(SEQ ID NO: 75)
DIQMTQSPSSLSASVGDRVTITCRASRDITKFLNWYQQKPGKAPKLLIYYTSRLHSGV
PSRFSGSGSGTDFTLTISSLQPEDFATYYCQQGNTLPWTFGGGTKVEIK
hu98VL2 Graft (IGKV1-39*01) + P44V, F71Y
(SEQ ID NO: 76)
DIQMTQSPSSLSASVGDRVTITCRASRDITKFLNWYQQKPGKAVKLLIYYTSRLHSGV
PSRFSGSGSGTDYTLTISSLQPEDFATYYCQQGNTLPWTFGGGTKVEIK
hu98VL3 Graft (IGKV1-39*01) + K42G, P44V, F71Y
(SEQ ID NO: 77)
DIQMTQSPSSLSASVGDRVTITCRASRDITKFLNWYQQKPGGAVKLLIYYTSRLHSGV
PSRFSGSGSGTDYTLTISSLQPEDFATYYCQQGNTLPWTFGGGTKVEIK
hu98VH1 Graft (IGHV3-7*01)
(SEQ ID NO: 78)
EVQLVESGGGLVQPGGSLRLSCAASGFTFSDYGIHWVRQAPGKGLEWVAYISSGGY
TIYYADTVKGRFTISRDNAKNSLYLQMNSLRAEDTAVYYCARGDYDDIFYTMDYW
GQGTTVTVSS
hu98VH2 Graft (IGHV3-7*01) + D98E
(SEQ ID NO: 79)
EVQLVESGGGLVQPGGSLRLSCAASGFTFSDYGIHWVRQAPGKGLEWVAYISSGGY
TIYYADTVKGRFTISRDNAKNSLYLQMNSLRAEDTAVYYCARGDYEDIFYTMDYW
GQGTTVTVSS
hu98VH3
(SEQ ID NO: 80)
EVQLVESGGGLVQPGGSLRLSCAASGFTFSDYGMHWVRQAPGKGLEWVAYISSGG
YTIYYADTVKGRFTISRDNAKNSLYLQMNSLRAEDTAVYYCARGDYEDIFYTMDY
WGQGTTVTVSS
hu98VH4
(SEQ ID NO: 81)
EVQLVESGGGLVQPGGSLRLSCAASGFTFSDYGIHWVRQAPGKGLEWVAYISSGGY
TSYYADTVKGRFTISRDNAKNSLYLQMNSLRAEDTAVYYCARGDYEDIFYTMDYW
GQGTTVTVSS
hu98VH5
(SEQ ID NO: 82)
EVQLVESGGGLVQPGGSLRLSCAASGFTFSDYGMHWVRQAPGKGLEWVAYISSGG
YTSYYADTVKGRFTISRDNAKNSLYLQMNSLRAEDTAVYYCARGDYEDIFYTMDY
WGQGTTVTVSS.

TABLE 18
The CDRs of hu98

| Variable region | CDR | Sequence | SEQ ID NO |
|---|---|---|---|
| hu98VH2/VH3/ | HCDR3 | GDYEDIFYTMDY | 83 |
| VH4/VH5 | | | |
| hu98VH3/VH5 | HCDR1 | DYGMH | 84 |
| hu98VH4/VH5 | HCDR2 | YISSGGYTSYYADTVKG | 85 |

3-3. Humanization of mAb110

For the antibody mAb110, the human germline light chain templates were IGKV4-1*01 and IGKJ2*01, and the human germline heavy chain templates were IGHV1-3*01 and IGHJ6*01. Optionally, the amino acid residue(s) at position(s) 1, 9, 43, 68, and/or 85 in the light chain variable region of the humanized antibody were/was substituted; and/or the amino acid residue(s) at position(s) 1, 24, 44, 71, 73, 54, and/or 55 in the heavy chain variable region of the humanized antibody were/was substituted.

TABLE 19
Humanization templates and corresponding point
mutations for the mAb110 murine antibody

| hu110VL | | hu110VH | |
|---|---|---|---|
| VL1 | Graft(IGKV4-1*01) + | VH1 | Graft(IGHV1-3*01) + R71V, |
| | P43S, G68A | | T73K + Q1E |
| VL2 | Graft(IGKV4-1*01) + | VH2 | Graft(IGHV1-3*01) + A24T, |
| | D1N, D9K, P43S, G68A, | | R44S, R71V, T73K + Q1E |
| | V85D | | |
| VL3 | Graft(IGKV4-1*01) | VH3 | Graft(IGHV1-3*01) + A24T, |
| | | | R44S, R71V, T73K + Q1E, |
| | | | N54Q |
| | | VH4 | Graft(IGHV1-3*01) + A24T, |
| | | | R44S, R71V, T73K + Q1E, |
| | | | N54S |
| | | VH5 | Graft(IGHV1-3*01) + A24T, |
| | | | R44S, R71V, T73K + Q1E, |
| | | | N54T |
| | | VH6 | Graft(IGHV1-3*01) + A24T, |
| | | | R44S, R71V, T73K + Q1E, |
| | | | G55V |
| | | VH7 | Graft(IGHV1-3*01) |
| FR4 | IGKJ2*01 | FR4 | IGHJ6*01 |

The sequences of the variable regions of the hu110 humanized antibodies obtained are as follows:

hu110VL1 Graft (IGKV4-1*01) + P43S, G68A
(SEQ ID NO: 86)
DIVMTQSPDSLAVSLGERATINCKASENVGIFVSWYQQKPGQSPKLLIYGASNRYTG
VPDRFSGSGSATDFTLTISSLQAEDVAVYYCGQSHSYPLTFGQGTKLEIK
hu110VL2 Graft (IGKV4-1*01) + DIN, D9K, P43S, G68A, V85D
(SEQ ID NO: 87)
NIVMTQSPKSLAVSLGERATINCKASENVGIFVSWYQQKPGQSPKLLIYGASNRYTG
VPDRFSGSGSATDFTLTISSLQAEDVADYYCGQSHSYPLTFGQGTKLEIK
hu110VL3 Graft (IGKV4-1*01):
(SEQ ID NO: 88)
DIVMTQSPDSLAVSLGERATINCKASENVGIFVSWYQQKPGQPPKLLIYGASNRYTG
VPDRFSGSGSGTDFTLTISSLQAEDVAVYYCGQSHSYPLTFGQGTKLEIK
hu110VH1 Graft (IGHV1-3*01) + R71V, T73K + Q1E
(SEQ ID NO: 89)
ELVQSGAEVKKPGASVKVSCKASGYTFTDYYMNWVRQAPGQRLEWMGIINPYN
GGTSYTQKFKGRVTITVDKSASTAYMELSSLRSEDTAVYYCARHFYDSSYLTYSTM
DYWGQGTTVTVSS
hu110VH2 Graft (IGHV1-3*01) + A24T, R44S, R71V, T73K + Q1E
(SEQ ID NO: 90)
ELVQSGAEVKKPGASVKVSCKTSGYTFTDYYMNWVRQAPGQSLEWMGIINPYN
GGTSYTQKFKGRVTITVDKSASTAYMELSSLRSEDTAVYYCARHFYDSSYLTYSTM
DYWGQGTTVTVSS
hu110VH3 Graft (IGHV1-3*01) + A24T, R44S, R71V, T73K + Q1E,
N54Q
(SEQ ID NO: 91)
ELVQSGAEVKKPGASVKVSCKTSGYTFTDYYMNWVRQAPGQSLEWMGIINPYQ
GGTSYTQKFKGRVTITVDKSASTAYMELSSLRSEDTAVYYCARHFYDSSYLTYSTM
DYWGQGTTVTVSS
hu110VH4 Graft (IGHV1-3*01) + A24T, R44S, R71V, T73K + Q1E,
N54S
(SEQ ID NO: 92)
ELVQSGAEVKKPGASVKVSCKTSGYTFTDYYMNWVRQAPGQSLEWMGIINPYS
GGTSYTQKFKGRVTITVDKSASTAYMELSSLRSEDTAVYYCARHFYDSSYLTYSTM
DYWGQGTTVTVSS
hu110VH5 Graft (IGHV1-3*01) + A24T, R44S, R71V, T73K + Q1E,
N54T
(SEQ ID NO: 93)
ELVQSGAEVKKPGASVKVSCKTSGYTFTDYYMNWVRQAPGQSLEWMGIINPYT
GGTSYTQKFKGRVTITVDKSASTAYMELSSLRSEDTAVYYCARHFYDSSYLTYSTM
DYWGQGTTVTVSS
hu110VH6 Graft (IGHV1-3*01) + A24T, R44S, R71V, T73K + Q1E,
G55V
(SEQ ID NO: 94)
ELVQSGAEVKKPGASVKVSCKTSGYTFTDYYMNWVRQAPGQSLEWMGIINPYN
VGTSYTQKFKGRVTITVDKSASTAYMELSSLRSEDTAVYYCARHFYDSSYLTYSTM
DYWGQGTTVTVSS
hu110VH7 Graft (IGHV1-3*01)
(SEQ ID NO: 95)
QVQLVQSGAEVKKPGASVKVSCKASGYTFTDYYMNWVRQAPGQRLEWMGIINPYN
GGTSYTQKFKGRVTITRDTSASTAYMELSSLRSEDTAVYYCARHFYDSSYLTYSTMD
YWGQGTTVTVSS.

TABLE 20
The CDRs of hul 10

| Variable | | | Sequence |
|---|---|---|---|
| region | CDR | Sequence | No. |
| hu110VH3 | HCDR2 | IINPYQGGTSYTQKFKG | 96 |
| hu110VH4 | HCDR2 | IINPYSGGTSYTQKFKG | 97 |
| hu110VH5 | HCDR2 | IINPYTGGTSYTQKFKG | 98 |
| hu110VH6 | HCDR2 | IINPYNVGTSYTQKFKG | 99 |

3-4. Humanization of mAb3

For the antibody mAb30, the human germline light chain templates were IGKV4-1*1 or IGKV1-39*1 and IGKJ2*01, and the human germline heavy chain templates were IGHV1G3*01 and IGHJ6*01. Optionally, the amino acid residue(s) at position(s) 9, 43, 60, 68, 87, and/or 100 in the light chain variable region of the humanized antibody were/was substituted; and/or the amino acid residue(s) at position(s) 1, 3, 5, 7, 9, 10, 12, 40, 44, 41, 48, 67, 69, 71, 73, 75, 83, 54, and/or 55 in the heavy chain variable region of the humanized antibody were/was substituted.

TABLE 21
Humanization templates and corresponding point
mutations for the mAb30 murine antibody

| hu30VL | | hu30VH | |
|---|---|---|---|
| VL1 | Graft(IGKV4- | VH1 | Graft(IGHV1-3*01) + R44S, |
| | 1*01) | | R71V, T73K + Q1E |
| VL2 | Graft(IGKV4- | VH2 | Graft(IGHV1-3*01) + Q3R, |
| | 1*01) + G68A, | | R44S, I69L, R71V, T73K+ Q1E |
| | Y87H | | |
| VL3 | Graft(IGKV4- | VH3 | Graft(IGHV1-3*01) + Q3R, |
| | 1*01) + D9K, P43S, | | R44S, I69L, R71V, T73K + Q1E, |
| | G68A, Y87H | | N54Q |
| VL4 | Graft(IGKV1- | VH4 | Graft(IGHV1-3*01) + Q3R, |
| | 39*01) | | R44S, I69L, R71V, T73K + Q1E, |
| | | | N54S |
| VL5 | Graft(IGKV1- | VH5 | Graft(IGHV1-3*01) + Q3R, |
| | 39*01) + A43S, | | R44S, I69L, R71V, T73K + Q1E, |
| | S60D, Q100S | | N54T |
| | | VH6 | Graft(IGHV1-3*01) + Q3R, |
| | | | R44S, I69L, R71V, T73K + Q1E, |
| | | | G55V |
| | | VH7 | Graft(IGHV1-3*01) + Q3R, |
| | | | V5Q, A9P, E10V, K12V, R44S, |
| | | | I69L, R71V, T73K + Q1E |
| | | VH8 | Graft(IGHV1-3*01) + Q3R, S7F, |
| | | | E10V, R44S, 169L, R71V, T73K, |
| | | | A75S + Q1E, N54T |
| | | VH9 | Graft(IGHV1-3*01) + Q3R, |
| | | | R44S, M48I, V67A, 169L, R71V, |
| | | | T73K + Q1E, N54T |
| | | VH10 | Graft(IGHV1-3*01) + Q3R, |
| | | | A40S, P41H, R44S, I69L, R71V, |
| | | | T73K + Q1E, N54T |
| | | VH11 | Graft(IGHV1-3*01) + Q3R, S7F, |
| | | | E10V, R44S, 169L, R71V, T73K, |
| | | | A75S, R83T + Q1E, N54T |
| | | VH12 | Graft(IGHV1-3*01) |
| FR4 | IGKJ2*01 | FR4 | IGHJ6*01 |

The sequences of the variable regions of the hu30 humanized antibodies obtained are as follows:

hu30VL1 Graft (IGKV4-1*01)
(SEQ ID NO: 100)
DIVMTQSPDSLAVSLGERATINCKASENVGYVSWYQQKPGQPPKLLIYGA
SNRYTGVPDRFSGSGSGTDFTLTISSLQAEDVAVYYCGQSYSYPLTFGQG
TKLEIK
hu30VL2 Graft (IGKV4-1*01) + G68A, Y87H
(SEQ ID NO: 101)
DIVMTQSPDSLAVSLGERATINCKASENVGYVSWYQQKPGQPPKLLIYGA
SNRYTGVPDRFSGSGS [symbol] TDFTLTISSLQAEDVAVY [symbol] CGQSYSYPLTFGQG
TKLEIK
hu30VL3 Graft (IGKV4-1*01) + D9K, P43S,
G68A, Y87H
(SEQ ID NO: 102)
DIVMTQSP [symbol] SLAVSLGERATINCKASENVGYVSWYQQKPGQSPKLLIYGA
SNRYTGVPDRFSGSGS [symbol] TDFTLTISSLQAEDVAVY [symbol] CGQSYSYPLTFGQG
TKLEIK
hu30VL4 Graft (IGKV1-39*01)
(SEQ ID NO: 103)
DIQMTQSPSSLSASVGDRVTITCKASENVGYVSWYQQKPGKAPKLLIYGA
SNRYTGVPSRFSGSGSGTDFTLTISSLQPEDFATYYCGQSYSYPLTFGQG
TKLEIK
hu30VL5 Graft (IGKV1-39*01) + A43S, S60D,
Q100S
(SEQ ID NO: 104)
DIQMTQSPSSLSASVGDRVTITCKASENVGYVSWYQQKPGK [symbol] PKLLIYGA
SNRYTGVP [symbol] RFSGSGSGTDFTLTISSLQPEDFATYYCGQSYSYPLTFGSG
TKLEIK
hu30VH1 Graft (IGHV1-3*01) + R44S, R71V,
T73K + Q1E
(SEQ ID NO: 105) [symbol]
VQLVQSGAEVKKPGASVKVSCKASGYTFTDYYMNWVRQAPGQ [symbol] LEWMGI
INPYNGDTSYNQKFRGRVTIT [symbol] D [symbol] SASTAYMELSSLRSEDTAVYYCARYH
YGPHLEGYFDVWGQGTTVTVSS
hu30VH2 Graft (IGHV1-3*01) + Q3R, R44S,
169L, R71V, T73K + Q1E
(SEQ ID NO: 106) [symbol]
V [symbol] LVQSGAEVKKPGASVKVSCKASGYTFTDYYMNWVRQAPGQ [symbol] LEWMGI
INPYNGDTSYNQKFRGRVT [symbol] T [symbol] D [symbol] SASTAYMELSSLRSEDTAVYYCARYH
YGPHLEGYFDVWGQGTTVTVSS
hu30VH3 Graft (IGHV1-3*01) + Q3R, R44S,
169L, R71V, T73K + Q1E, N54Q
(SEQ ID NO: 107) [symbol]
V [symbol] LVQSGAEVKKPGASVKVSCKASGYTFTDYYMNWVRQAPGQ [symbol] LEWMGI
INPYGDTSYNQKFRGRVT [symbol] T [symbol] D [symbol] SASTAYMELSSLRSEDTAVYYCARYH
YGPHLEGYFDVWGQGTTVTVSS
hu30VH4 Graft (IGHV1-3*01) + Q3R, R44S,
169L, R71V, T73K + Q1E, N54S
(SEQ ID NO: 108) [symbol]
V [symbol] LVQSGAEVKKPGASVKVSCKASGYTFTDYYMNWVRQAPGQ [symbol] LEWMGI
INPY [symbol] GDTSYNQKFRGRVT [symbol] T [symbol] D [symbol] SASTAYMELSSLRSEDTAVYYCARYH
YGPHLEGYFDVWGQGTTVTVSS
hu30VH5 Graft (IGHV1-3*01) + Q3R, R44S,
169L, R71V, T73K + Q1E, N54T
(SEQ ID NO: 109) [symbol]
V [symbol] LVQSGAEVKKPGASVKVSCKASGYTFTDYYMNWVRQAPGQ [symbol] LEWMGI
INPY [symbol] GDTSYNQKFRGRVT [symbol] T [symbol] D [symbol] SASTAYMELSSLRSEDTAVYYCARYH
YGPHLEGYFDVWGQGTTVTVSS
hu30VH6 Graft (IGHV1-3*01) + Q3R, R44S,
169L, R71V, T73K + Q1E, G55V
(SEQ ID NO: 110) [symbol]
V [symbol] LVQSGAEVKKPGASVKVSCKASGYTFTDYYMNWVRQAPGQ [symbol] LEWMGI
INPYN [symbol] DTSYNQKFRGRVT [symbol] T [symbol] D [symbol] SASTAYMELSSLRSEDTAVYYCARYH
YGPHLEGYFDVWGQGTTVTVSS
hu30VH7 Graft (IGHV1-3*01) + Q3R, V5Q,
A9P, E10V, K12V, R44S, I69L, R71V,
T73K + Q1E
(SEQ ID NO: 111) [symbol]
V [symbol] L [symbol] QSG [symbol] V [symbol] KPGASVKVSCKASGYTFTDYYMNWVRQAPGQ [symbol] LEWMGI
INPYNGDTSYNQKFRGRVT [symbol] T [symbol] D [symbol] SASTAYMELSSLRSEDTAVYYCARYH
YGPHLEGYFDVWGQGTTVTVSS
hu30VH8 Graft (IGHV1-3*01) + Q3R, S7F,
E10V, R44S, I69L, R71V, T73K, A75S
Q1E, N54T
(SEQ ID NO: 112) [symbol]
V [symbol] LVQ [symbol] GA [symbol] VKKPGASVKVSCKASGYTFTDYYMNWVRQAPGQ [symbol] LEWMGI
INPY [symbol] GDTSYNQKFRGRVT [symbol] T [symbol] D [symbol] SSSTAYMELSSLRSEDTAVYYCARYH
YGPHLEGYFDVWGQGTTVTVSS
hu30VH9 Graft (IGHV1-3*01) + Q3R, R44S,
M48I, V67A, 169L, R71V, T73K + Q1E,
N54T
(SEQ ID NO: 113) [symbol]
V [symbol] LVQSGAEVKKPGASVKVSCKASGYTFTDYYMNWVRQAPGQ [symbol] LEWIGI
INPY [symbol] GDTSYNQKFRGRAT [symbol] T [symbol] D [symbol] SASTAYMELSSLRSEDTAVYYCARYH
YGPHLEGYFDVWGQGTTVTVSS
hu30VH10 Graft (IGHV1-3*01) + Q3R, A40S,
P41H, R44S, I69L, R71V, T73K + Q1E,
N54T
(SEQ ID NO: 114) [symbol]
V [symbol] LVQSGAEVKKPGASVKVSCKASGYTFTDYYMNWVRQ [symbol] GQ [symbol] LEWMGI
INPY [symbol] GDTSYNQKFRGRVT [symbol] T [symbol] D [symbol] SASTAYMELSSLRSEDTAVYYCARYH
YGPHLEGYFDVWGQGTTVTVSS
hu30VH11 Graft (IGHV1-3*01) + Q3R, S7F,
E10V, R44S, I69L, R71V, T73K, A75S,
R83T + Q1E, N54T
(SEQ ID NO: 115) [symbol]
V [symbol] LVQ [symbol] GA [symbol] VKKPGASVKVSCKASGYTFTDYYMNWVRQAPGQ [symbol] LEWMGI
INPY [symbol] GDTSYNQKFRGRVT [symbol] T [symbol] D [symbol] SSSTAYMELSSL [symbol] SEDTAVYYCARYH
YGPHLEGYFDVWGQGTTVTVSS
hu30VH12 Graft (IGHV1-3*01)
(SEQ ID NO: 116)
QVQLVQSGAEVKKPGASVKVSCKASGYTFTDYYMNWVRQAPGQRLEWMGI
INPYNGDTSYNQKFRGRVTITRDTSASTAYMELSSLRSEDTAVYYCARYH
YGPHLEGYFDVWGQGTTVTVSS.

TABLE 22
The CDRs of hu30

| Variable | | | |
|---|---|---|---|
| region | CDR | Sequence | Sequence No. |
| hu30VH3 | HCDR2 | IINPY [symbol] GDTSYNQKFRG | SEQ ID NO: 117 |
| hu30VH4 | HCDR2 | IINPY [symbol] GDTSYNQKFRG | SEQ ID NO: 118 |
| hu30VH5/ | HCDR2 | IINPY [symbol] GDTSYNQKFRG | SEQ ID NO: 119 |
| VH8/VH9/ | | | |
| VH10/VH11 | | | |
| hu30VH6 | HCDR2 | IINPYN [symbol] DTSYNQKFRG | SEQ ID NO: 120 |

The heavy chain variable region and the light chain variable region of the humanized antibodies were recombined with a heavy chain constant region and a light chain constant region, respectively. The sequence of an exemplary heavy chain constant region is SEQ ID NO: 41, and the sequence of an exemplary light chain constant region is SEQ ID NO: 42. Full-length humanized antibodies were obtained. Variable region combinations for specific humanized antibodies are shown in the table below.

TABLE 23-1
Humanized antibodies of mAb6

| Variable | | | | | |
|---|---|---|---|---|---|
| region | VL1 | VL2 | VL3 | VL4 | VL5 |
| VH1 | hu6L1H1 | hu6L2H1 | hu6L3H1 | hu6L4H1 | hu6L5H1 |
| VH2 | hu6L1H2 | hu6L2H2 | hu6L3H2 | hu6L4H2 | hu6L5H2 |
| VH3 | hu6L1H3 | hu6L2H3 | hu6L3H3 | hu6L4H3 | hu6L5H3 |
| VH4 | hu6L1H4 | hu6L2H4 | hu6L3H4 | hu6L4H4 | hu6L5H4 |
| VH5 | hu6L1H5 | hu6L2H5 | hu6L3H5 | hu6L4H5 | hu6L5H5 |
| VH6 | hu6L1H6 | hu6L2H6 | hu6L3H6 | hu6L4H6 | hu6L5H6 |
| VH7 | hu6L1H7 | hu6L2H7 | hu6L3H7 | hu6L4H7 | hu6L5H7 |
| VH8 | hu6L1H8 | hu6L2H8 | hu6L3H8 | hu6L4H8 | hu6L5H8 |
| VH9 | hu6L1H9 | hu6L2H9 | hu6L3H9 | hu6L4H9 | hu6L5H9 |
| VH10 | hu6L1H10 | hu6L2H10 | hu6L3H10 | hu6L4H10 | hu6L5H10 |
| VH11 | hu6L1H11 | hu6L2H11 | hu6L3H11 | hu6L4H11 | hu6L5H11 |
| VH12 | hu6L1H12 | hu6L2H12 | hu6L3H12 | hu6L4H12 | hu6L5H12 |
| VH13 | hu6L1H13 | hu6L2H13 | hu6L3H13 | hu6L4VH13 | hu6L5H13 |
| VH14 | hu6L1H14 | hu6L2H14 | hu6L3H14 | hu6L4VH14 | hu6L5H14 |
| VH15 | hu6L1H15 | hu6L2H15 | hu6L3H15 | hu6L4VH15 | hu6L5H15 |

Note:
hu6L1H1 indicates that the antibody comprises the heavy chain variable region hu6VH1 and the light chain variable region hu6VLI and that the sequence of the heavy chain constant region is SEQ ID NO: 41 and the sequence of the light chain constant region is SEQ ID NO: 42. The same applies to the others.

TABLE 23-2
Humanized antibodies of mAb6

| Variable | | | | | | |
|---|---|---|---|---|---|---|
| region | VL6 | VL7 | VL8 | VL9 | VL10 | VL11 |
| VH1 | hu6L6H1 | hu6L7H1 | hu6L8H1 | hu6L9H1 | hu6L10H1 | hu6L11H1 |
| VH2 | hu6L6H2 | hu6L7H2 | hu6L8H2 | hu6L9H2 | hu6L10H2 | hu6L11H2 |
| VH3 | hu6L6H3 | hu6L7H3 | hu6L8H3 | hu6L9H3 | hu6L10H3 | hu6L11H3 |
| VH4 | hu6L6H4 | hu6L7H4 | hu6L8H4 | hu6L9H4 | hu6L10H4 | hu6L11H4 |
| VH5 | hu6L6H5 | hu6L7H5 | hu6L8H5 | hu6L9H5 | hu6L10H5 | hu6L11H5 |
| VH6 | hu6L6H6 | hu6L7H6 | hu6L8H6 | hu6L9H6 | hu6L10H6 | hu6L11H6 |
| VH7 | hu6L6H7 | hu6L7H7 | hu6L8H7 | hu6L9H7 | hu6L10H7 | hu6L11H7 |
| VH8 | hu6L6H8 | hu6L7H8 | hu6L8H8 | hu6L9H8 | hu6L10H8 | hu6L11H8 |
| VH9 | hu6L6H9 | hu6L7H9 | hu6L8H9 | hu6L9H9 | hu6L10H9 | hu6L11H9 |
| VH10 | hu6L6H10 | hu6L7H10 | hu6L8H10 | hu6L9H10 | hu6L10H10 | hu6L11H10 |
| VH11 | hu6L6H11 | hu6L7H11 | hu6L8H11 | hu6L9H11 | hu6L10H11 | hu6L11H11 |
| VH12 | hu6L6H12 | hu6L7H12 | hu6L8H12 | hu6L9H12 | hu6L10H12 | hu6L11H12 |
| VH13 | hu6L6H13 | hu6L7H13 | hu6L8H13 | hu6L9VH13 | hu6L10H13 | hu6L11H13 |
| VH14 | hu6L6H14 | hu6L7H14 | hu6L8H14 | hu6L9VH14 | hu6L10H14 | hu6L11H14 |
| VH15 | hu6L6H15 | hu6LH15 | hu6L8H15 | hu6L9VH15 | hu6L10H15 | hu6L11H15 |

TABLE 23-3
Humanized antibodies of mAb98

| Variable | | | |
|---|---|---|---|
| region | VL1 | VL2 | VL3 |
| VH1 | hu98L1H1 | hu98L2H1 | hu98L3H1 |
| VH2 | hu98L1H2 | hu98L2H2 | hu98L3H2 |
| VH3 | hu98L1H3 | hu98L2H3 | hu98L3H3 |
| VH4 | hu98L1H4 | hu98L2H4 | hu98L3H4 |
| VH5 | hu98L1H5 | hu98L2H5 | hu98L3H5 |

Note:
hu98L1H1 indicates that the antibody comprises the heavy chain variable region hu98VH1 and the light chain variable region hu98VL1 and that the sequence of the heavy chain constant region is SEQ ID NO: 41 and the sequence of the light chain constant region is SEQ ID NO: 42. The same applies to the others.

TABLE 23-4
Humanized antibodies of mAb110

| Variable | | | |
|---|---|---|---|
| region | VL1 | VL2 | VL3 |
| VH1 | hu110L1H1 | hu110L2H1 | hu110L3H1 |
| VH2 | hu110L1H2 | hu110L2H2 | hu110L3H2 |
| VH3 | hu110L1H3 | hu110L2H3 | hu110L3H3 |
| VH4 | hu110L1H4 | hu110L2H4 | hu110L3H4 |
| VH5 | hu110L1H5 | hu110L2H5 | hu110L3H5 |
| VH6 | hu110L1H6 | hu110L2H6 | hu110L3H6 |
| VH7 | hu110L1H7 | hu110L2H7 | hu110L3H7 |

Note:
hu110L1H1 indicates that the antibody comprises the heavy chain variable region hu110VH1 and the light chain variable region hu110VL1 and that the sequence of the heavy chain constant region is SEQ ID NO: 41 and the sequence of the light chain constant region is SEQ ID NO: 42. The same applies to the others.

TABLE 23-5
Humanized antibodies of mAb30

| Variable | | | | | |
|---|---|---|---|---|---|
| region | VL1 | VL2 | VL3 | VL4 | VL5 |
| VH1 | hu30L1H1 | hu30L2H1 | hu30L3H1 | hu30L4H1 | hu30L5H1 |
| VH2 | hu30L1H2 | hu30L2H2 | hu30L3H2 | hu30L4H2 | hu30L5H2 |
| VH3 | hu30L1H3 | hu30L2H3 | hu30L3H3 | hu30L4H3 | hu30L5H3 |
| VH4 | hu30L1H4 | hu30L2H4 | hu30L3H4 | hu30L4H4 | hu30L5H4 |
| VH5 | hu30L1H5 | hu30L2H5 | hu30L3H5 | hu30L4H5 | hu30L5H5 |
| VH6 | hu30L1H6 | hu30L2H6 | hu30L3H6 | hu30L4H6 | hu30L5H6 |
| VH7 | hu30L1H7 | hu30L2H7 | hu30L3H7 | hu30L4H7 | hu30L5H7 |
| VH8 | hu30L1H8 | hu30L2H8 | hu30L3H8 | hu30L4H8 | hu30L5H8 |
| VH9 | hu30L1H9 | hu30L2H9 | hu30L3H9 | hu30L4H9 | hu30L5H9 |
| VH10 | hu30L1H10 | hu30L2H10 | hu30L3H10 | hu30L4H10 | hu30L5H10 |
| VH11 | hu30L1H11 | hu30L2H11 | hu30L3H11 | hu30L4H11 | hu30L5H11 |
| VH12 | hu30L1H12 | hu30L2H12 | hu30L3H12 | hu30L4H12 | hu30L5H12 |

Note:
hu30L1H1 indicates that the antibody comprises the heavy chain variable region hu30VH1 and the light chain variable region hu30VL1 and that the sequence of the heavy chain constant region is SEQ ID NO: 41 and the sequence of the light chain constant region is SEQ ID NO: 42. The same applies to the others.

Note: hu30L1H1 indicates that the antibody comprises the heavy chain variable region hu30VH1 and the light chain variable region hu30VL1 and that the sequence of the heavy chain constant region is SEQ ID NO: 41 and the sequence of the light chain constant region is SEQ ID NO: 42. The same applies to the others.

The above antibodies were cloned, expressed, and purified, and through a protein binding assay (Test Example 3), a cell binding assay (Test Example 2), and Biacore (Test Example 1), humanized antibodies with good activity were finally selected. The heavy and light chain amino acid sequences of exemplary humanized antibodies are as follows:

The heavy chain of Hu6 (also referred to
as hu6L4H12)
(SEQ ID NO: 121):
EVQLVQSGSELKKPGASVKVSCKASGYTFTDYYMNWVRQAPGQGLEWMGI
INPYSGVTIYNHKFKGRFALSVDKSSNTAYLQISSLKAEDTAVYYCARQD
SLLDYAMDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNOVSLTCLVKGFYPSDIAVEWESNGQPENNYKTTPPV
LDSDGSFFLYSKLTVDKSRWQQGNVFSCSVMHEALHNHYTQKSLSLSPGK
The light chain of Hu6 (also referred to
as hu6L4H12)
(SEQ ID NO: 122):
DIQMTQSPSSLSASVGDRVTITCHASQGISGNMGWLQQKPGKAPKGLIYH
GANLEDGVPSRFSGSGSGADYTLTISSLQPEDFADYYCVQYAQFPYTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
The heavy chain of Hu98 (also referred to
as hu98L3H4)
(SEQ ID NO: 123):
EVQLVESGGGLVQPGGSLRLSCAASGFTFSDYGIHWVRQAPGKGLEWVAY
ISSGGYTSYYADTVKGRFTISRDNAKNSLYLOMNSLRAEDTAVYYCARGD
YEDIFYTMDYWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K
The light chain of Hu98 (also referred to
as hu98L3H4)
(SEQ ID NO: 124):
DIQMTQSPSSLSASVGDRVTITCRASRDITKFLNWYQQKPGGAVKLLIYY
TSRLHSGVPSRFSGSGSGTDYTLTISSLQPEDFATYYCQQGNTLPWTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
The heavy chain of Hul10 (also referred to
as hul10L2H3)
(SEQ ID NO: 125):
EVQLVQSGAEVKKPGASVKVSCKTSGYTFTDYYMNWVRQAPGQSLEWMGI
INPYQGGTSYTQKFKGRVTITVDKSASTAYMELSSLRSEDTAVYYCARHF
YDSSYLTYSTMDYWGQGTTVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRDELTKNOVSLTCLVKGFYPSDIAVEWESNGQPENNYKT
TPPVLDSDGSFFLYSKLTVDKSRWQQGNVFSCSVMHEALHNHYTQKSLSL
SPGK
The light chain of Hul 10 (also referred to
as hul 10L2H3)
(SEQ ID NO: 126):
NIVMTQSPKSLAVSLGERATINCKASENVGIFVSWYQQKPGQSPKLLIYG
ASNRYTGVPDRFSGSGSATDFTLTISSLQAEDVADYYCGQSHSYPLTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
The heavy chain of Hu30 (also referred to as
hu30L1H5)
(SEQ ID NO: 127):
EVRLVQSGAEVKKPGASVKVSCKASGYTFTDYYMNWVRQAPGQSLEWMGI
INPYTGDTSYNQKFRGRVTLTVDKSASTAYMELSSLRSEDTAVYYCARYH
YGPHLEGYFDVWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GK
The light chain of Hu30 (also referred to as
hu30L1H5)
(SEQ ID NO: 128):
DIVMTQSPDSLAVSLGERATINCKASENVGYVSWYQQKPGQPPKLLIYGA
SNRYTGVPDRFSGSGSGTDFTLTISSLQAEDVAVYYCGQSYSYPLTFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Note: the underlined parts in the sequences
indicate the variable regions, and the
italicized parts indicate the constant
regions.

### Example 5. Preparation of Anti-DLL3-CD3 Bispecific Antibodies

### 5.1. CD3 Antibodies Used in Present Disclosure

The CD3-binding moiety of the present disclosure may be derived from any suitable antibody. Specifically, S107E and I2C were used in the examples of the present disclosure, and their variable regions and CDR sequences are as follows:

TABLE 24
The CDRs of S107E

| Antibody | Name | Sequence | No. |
|---|---|---|---|
| S107E | HCDR1 | KYAMN | SEQ ID NO: 129 |
| | HCDR2 | RIRSKYNNYATYYA | SEQ ID NO: 130 |
| | | DSVKD | |
| | HCDR3 | HGNFGNEYISYWAY | SEQ ID NO: 131 |
| | LCDR1 | GSSTGAVTSGNYPN | SEQ ID NO: 132 |
| | LCDR2 | GTKFLAP | SEQ ID NO: 133 |
| | LCDR3 | VLWYSNRWV | SEQ ID NO: 134 |
| I2C | HCDR1 | KYAMN | SEQ ID NO: 129 |
| | HCDR2 | RIRSKYNNYATYYA | SEQ ID NO: 130 |
| | | DSVKD | |
| | HCDR3 | HGNFGNSYISYWAY | SEQ ID NO: 135 |
| | LCDR1 | GSSTGAVTSGNYPN | SEQ ID NO: 132 |
| | LCDR2 | GTKFLAP | SEQ ID NO: 133 |
| | LCDR3 | VLWYSNRWV | SEQ ID NO: 134 |

The variable region sequences of S107E are as follows:

>S107E-VH
(SEQ ID NO: 136)
EVQLVESGGGLVQPGGSLKLSCAASGFTFNKYAMNWVRQAPGKGLEWVAR
IRSKYNNYATYYADSVKDRFTISRDDSKNTAYLQMNNLKTEDTAVYYCVR
HGNFGNEYISYWAYWGQGTLVTVSS
>S107E-VL
(SEQ ID NO: 137)
QTVVTQEPSLTVSPGGTVTLTCGSSTGAVTSGNYPNWVQQKPGQAPRGLI
GGTKFLAPGTPARFSGSLLGGKAALTLSGVQPEDEAEYYCVLWYSNRWVF
GGGTKLTVL
The variable region sequences of
I2C are as follows:
>I2C-VH
(SEQ ID NO: 138)
EVQLVESGGGLVQPGGSLKLSCAASGFTFNKYAMNWVRQAPGKGLEWVAR
IRSKYNNYATYYADSVKDRFTISRDDSKNTAYLQMNNLKTEDTAVYYCVR
HGNFGNSYISYWAYWGQGTLVTVSS
>I2C-VL
(SEQ ID NO: 137)
QTVVTQEPSLTVSPGGTVTLTCGSSTGAVTSGNYPNWVQQKPGQAPRGLI
GGTKFLAPGTPARFSGSLLGGKAALTLSGVQPEDEAEYYCVLWYSNRWVF
GGGTKLTVL
Note: CDRs are underlined.
>S107E-scFv
(SEQ ID NO: 139):
EVQLVESGGGLVQPGGSLKLSCAASGFTFNKYAMNWVRQAPGKGLEWVAR
IRSKYNNYATYYADSVKDRFTISRDDSKNTAYLQMNNLKTEDTAVYYCVR
HGNFGNEYISYWAYWGQGTLVTVSS [symbol] QTVVTQEPSL
TVSPGGTVTLTCGSSTGAVTSGNYPNWVQQKPGQAPRGLIGGTKFLAPGT
PARFSGSLLGGKAALTLSGVQPEDEAEYYCVLWYSNRWVFGGGTKLTVL
>I2C-scFv
(SEQ ID NO: 140):
EVQLVESGGGLVQPGGSLKLSCAASGFTFNKYAMNWVRQAPGKGLEWVAR
IRSKYNNYATYYADSVKDRFTISRDDSKNTAYLQMNNLKTEDTAVYYCVR
HGNFGNSYISYWAYWGQGTLVTVSS [symbol] QTVVTQEPSL
TVSPGGTVTLTCGSSTGAVTSGNYPNWVQQKPGQAPRGLIGGTKFLAPGT
PARFSGSLLGGKAALTLSGVQPEDEAEYYCVLWYSNRWVFGGGTKLTVL
Note: Linkers are double underlined.

### 5.2. Structures of DLL3-CD3 Bispecific Antibodies of Present Disclosure

The structures of the DLL3-CD3 bispecific antibody molecules of the present disclosure are Format1, Format2, and Format3, and the specific structures are as follows:

1. Format1 is an asymmetric structure Hot-Ig. An intact molecule has four chains in total, and the four chains are all different and are specifically as follows:
chain 1: VL (anti-DLL3)-linker 1-Obscurin;
chain 2: VH (anti-DLL3)-linker 1-Titin-IgG1Fc (Hole, L234A, L235A, Y349C, T366S, L368A, Y407V);
chain 3: VH (anti-CD3)-IgG1 (CHT)-IgG1Fc (Knob, L234A/L235A/S354C/T366W); and
chain 4: VL (anti-CD3)-hλ.

A schematic diagram of Format1 is shown in FIG. TA, where Ob stands for Obscurin.

2. Format2 is an asymmetric structure molecule Y body. An intact molecule contains 3 chains, and the 3 chains are specifically as follows:
chain 1: VL (anti-DLL3)-hκ;
chain 2: VH (anti-DLL3-IgG1 (CH1)-IgG1Fc (Hole, L234A/L235A/Y349C/T366S/L368A/Y407V); and
chain 3: VH (anti-CD3)-linker 2-VL (anti-CD3)-linker 1-IgG1Fc (Knob, L234A/L235A/S354C/T366W).

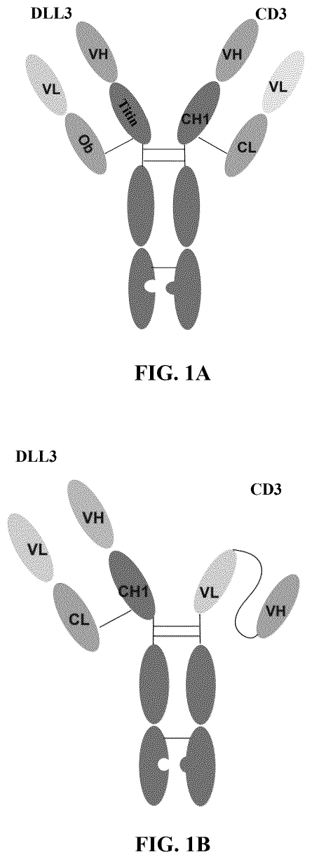

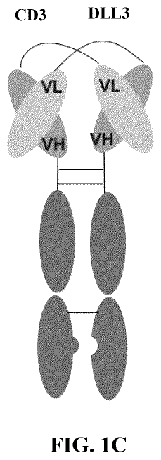

A schematic diagram of Format2 is shown in FIG. 1B.

3. Format3 is a diabody. An intact molecule contains two chains, and the two chains are specifically as follows:
chain 1: VL (anti-DLL3)-linker 3-VH (anti-CD3)-linker 4-Fc (Knob, L234A, L235A, G237A, Y349C, T366W); and
chain 2: VL (anti-CD3)-linker 5-VH (anti-DLL3)-linker 4-Fc (hole, L234A, L235A, G237A, S354C, T366S, L368A, Y407A).

A schematic diagram of Format3 is shown in FIG. 1C.

The related sequences are as follows:

(SEQ ID NO: 141)
> IgG₁ (CH1)
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSC
(SEQ ID NO: 142)
> hλ
GQPKANPTVTLFPPSSEELQANKATLVCLISDFYPGAVTVAWKADGSPVKAGVETTK
PSKQSNNKYAASSYLSLTPEQWKSHRSYSCQVTHEGSTVEKTVAPTEC
(SEQ ID NO: 143)
> IgG1Fc (Knob, L234A/L235A/S354C/T366W)

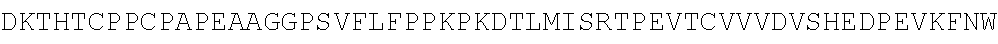

YVDGVEVHNAKTKPREEQYNSTYRVVSVLTVLHQDWLNGKEYKCKVSNKALPAPI

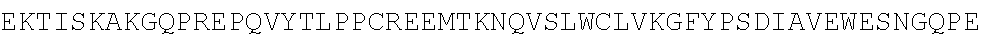

NNYKTTPPVLDSDGSFFLYSKLTVDKSRWQQGNVFSCSVMHEALHNHYTQKSLSLS
PGK
(SEQ ID NO: 144)
> IgGIFc (Hole, L234A, L235A, Y349C, T366S, L368A, Y407V)

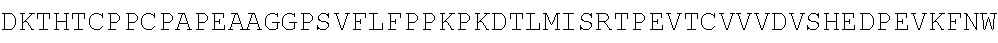

YVDGVEVHNAKTKPREEQYNSTYRVVSVLTVLHQDWLNGKEYKCKVSNKALPAPI

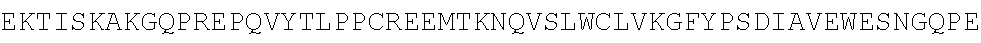

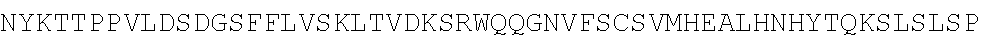

GK
(SEQ ID NO: 246)
> Obscurin
SGAPRFLTRPKASVVSVGKDATLSCQIVGNPFPQVSWEKDKQPVTAGVRFRLAQDG
DLYRLKILDLQLSDSGQYVCRARNAHGEAFACLGLQVDAEA
(SEQ ID NO: 208)
> Titin
GIPPKIECLPIDISIDEGKVLTVASAFTGEPTPEVTWSTGGRKIHSQEQGRFHIENTDDS
TTLTIKDVQKQDGGLYTLTLRNEFGSDSATVNIHIRSI
(SEQ ID NO: 145)
> Fc (Knob, L234A, L235A, G237A, Y349C, T366W)
CPPCPAPEAAGAPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVCTLPPSREEMTKNQVSLWCLVKGFYPSDIAVEWESNGQPENNYKTT
PPVLDSDGSFFLYSKLTVDKSRWQQGNVFSCSVMHEALHNHYTQKSLSLSPGK
(SEQ ID NO: 146)
> Fc (hole, L234A, L235A, G237A, S354C, T366S, L368A, Y407V)
CPPCPAPEAAGAPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVYTLPPCREEMTKNQVSLSCAVKGFYPSDIAVEWESNGQPENNYKTT
PPVLDSDGSFFLVSKLTVDKSRWQQGNVFSCSVMHEALHNHYTQKSLSLSPGK
(SEQ ID NO: 147)
> Linker 1
GGGGS
(SEQ ID NO: 148)
> Linker 2
GGGGSGGGGSGGGGS
(SEQ ID NO: 149)
> Linker 3
GGGSGGGG
(SEQ ID NO: 150)
> Linker 4
G
(SEQ ID NO: 151)
> Linker 5
GGGGSGGGG.

Based on the amino acid sequences of humanized antibodies of mAb6, mAb98, mAb110, and mAb30, the following bispecific antibodies were constructed.

TABLE 25
The bispecific antibodies of the present disclosure

| No. | Chain 1 | Chain 2 | Chain 3 | Chain 4 |
|---|---|---|---|---|
| hu6-S107E-hot Ig | hu6VL-G4S- | hu6VH-G4S- | S107EVH- | CD3VL- |
| Format 1 | Obscurin | Titin-hole | CH1-knob | hλ |
| | SEQ ID NO: 152 | SEQ ID NO: 153 | SEQ ID NO: | SEQ ID |
| | | | 154 | NO: 155 |
| hu6-I2C-hot Ig | hu6VL-G4S- | hu6VH-G4S- | I2CVH-CH1- | CD3VL- |
| Format 1 | Obscurin | Titin-hole | knob | hλ |
| | SEQ ID NO: 152 | SEQ ID NO: 153 | SEQ ID NO: | SEQ ID |
| | | | 156 | NO: 155 |
| hu6-S107E-Y body | hu6VL-hκ | hu6VH-CH1-hole | S107E_scFv- | |
| Format 2 | | | knob | |
| | SEQ ID NO: 157 | SEQ ID NO: 158 | SEQ ID NO: | |
| | | | 159 | |
| hu6-I2C-Y body | hu6VL-hκ | hu6VH-CH1-hole | I2C_scFv-knob | |
| Format 2 | SEQ ID NO: 157 | SEQ ID NO: 158 | SEQ ID NO: | |
| | | | 160 | |
| hu6-S107E-diabody | hu6VL-S107EVH- | CD3VL-hu6VH- | | |
| Format 3 | knob | hole | | |
| | SEQ ID NO: 161 | SEQ ID NO: 162 | | |
| hu6-I2C-diabody | hu6VL-I2CVH- | CD3VL-hu6VH- | | |
| Format 3 | knob | hole | | |
| | SEQ ID NO: 163 | SEQ ID NO: 162 | | |
| hu98-S107E-hot Ig | hu98VL-G4S- | hu98VH-G4S- | S107EVH- | CD3VL- |
| Format 1 | Obscurin | Titin-hole | CH1-knob | hλ |
| | SEQ ID NO: 164 | SEQ ID NO: 165 | SEQ ID NO: | SEQ ID |
| | | | 154 | NO: 155 |
| hu98-I2C-hot Ig | hu98VL-G4S- | hu98VH-G4S- | I2CVH-CH1- | CD3VL |
| Format 1 | Obscurin | Titin-hole | knob | |
| | SEQ ID NO: 164 | SEQ ID NO: 165 | SEQ ID NO: | SEQ ID |
| | | | 156 | NO: 155 |
| hu98-S107E-Y body | hu98VL-hκ | hu98VH-CH1- | S107E scFv- | |
| Format 2 | | hole | knob | |
| | SEQ ID NO: 166 | SEQ ID NO: 167 | SEQ ID NO: | |
| | | | 159 | |
| hu98-I2C-Y body | hu98VL-hk | hu98VH-CH1- | I2C_scFv-knob | |
| Format 2 | | hole | | |
| | SEQ ID NO: 166 | SEQ ID NO: 167 | SEQ ID NO: | |
| | | | 160 | |
| hu98-S107E-diabody | hu98VL- | CD3VL-hu98VH- | | |
| Format 3 | S107EVH-knob | hole | | |
| | SEQ ID NO: 168 | SEQ ID NO: 169 | | |
| hu98-I2C-diabody | hu98VL-I2CVH- | CD3VL-hu98VH- | | |
| Format 3 | knob | hole | | |
| | SEQ ID NO: 170 | SEQ ID NO: 169 | | |
| hu110-S107E-hot Ig | hu110VL-G4S- | hu110VH-G4S- | S107EVH- | CD3VL- |
| Format 1 | Obscurin | Titin-hole | CH1-knob | hλ |
| | SEQ ID NO: 171 | SEQ ID NO: 172 | SEQ ID NO: | SEQ ID |
| | | | 154 | NO: 155 |
| hu110-I2C-hot Ig | hu110VL-G4S- | hu110VH-G4S- | I2CVH-CH1- | CD3VL- |
| Format 1 | Obscurin | Titin-hole | knob | hλ |
| | SEQ ID NO: 171 | SEQ ID NO: 172 | SEQ ID NO: | SEQ ID |
| | | | 156 | NO: 155 |
| hul10-S107E-Y | hul 10VL-hκ | hu110VH-CH1- | S107E_scFv- | |
| body | | hole | knob | |
| Format 2 | SEQ ID NO: 173 | SEQ ID NO: 174 | SEQ ID NO: | |
| | | | 159 | |
| hu110-I2C-Y body | hul 10VL-hκ | hu110VH-CH1- | I2C_scFv-knob | |
| Format 2 | | hole | | |
| | SEQ ID NO: 173 | SEQ ID NO: 174 | SEQ ID NO: | |
| | | | 160 | |
| hu110-S107E- | hu110VL- | CD3VL- | | |
| diabody | S107EVH-knob | hu110VH-hole | | |
| Format 3 | SEQ ID NO: 175 | SEQ ID NO: 176 | | |
| hu110-I2C-diabody | hu110VL-I2CVH- | CD3VL- | | |
| Format 3 | knob | hu110VH-hole | | |
| | SEQ ID NO: 177 | SEQ ID NO: 176 | | |
| hu30-S107E-hot Ig | hu30VL-G4S- | hu30VH-G4S- | S107EVH- | CD3VL- |
| Format 1 | Obscurin | Titin-hole | CH1-knob | hλ |
| | SEQ ID NO: 178 | SEQ ID NO: 179 | SEQ ID NO: | SEQ ID |
| | | | 154 | NO: 155 |
| hu30-I2C-hot Ig | hu30VL-G4S- | hu30VH-G4S- | I2CVH-CH1- | CD3VL- |
| Format 1 | Obscurin | Titin-hole | knob | hλ |
| | SEQ ID NO: 178 | SEQ ID NO: 179 | SEQ ID NO: | SEQ ID |
| | | | 156 | NO: 155 |
| hu30-S107E-Y body | hu30VL-hκ | hu30VH-CH1- | S107E_scFv- | |
| Format 2 | | hole | knob | |
| | SEQ ID NO: 180 | SEQ ID NO: 181 | SEQ ID NO: | |
| | | | 159 | |
| hu30-I2C-Y body | hu30VL-hκ | hu30VH-CH1- | I2C_scFv-knob | |
| Format 2 | | hole | | |
| | SEQ ID NO: 180 | SEQ ID NO: 181 | SEQ ID NO: | |
| | | | 160 | |
| hu30-S107E-diabody | hu30VL- | CD3VL-hu30VH- | | |
| Format 3 | S107EVH-knob | hole | | |
| | SEQ ID NO: 182 | SEQ ID NO: 183 | | |
| hu30-I2C-diabody | hu30VL-I2CVH- | CD3VL-hu30VH- | | |
| Format 3 | knob | hole | | |
| | SEQ ID NO: 184 | SEQ ID NO: 183 | | |
| hu6(D56E)-S107E- | hu6VL (D56E) - | CD3VL-hu6VH- | | |
| diabody | S107EVH-knob | hole | | |
| Format 3 | SEQ ID NO: 185 | SEQ ID NO: 162 | | |
| hu6(D56S)-S107E- | hu6VL (D56S) - | CD3VL-hu6VH- | | |
| diabody | S107EVH-knob | hole | | |
| Format 3 | SEQ ID NO: 186 | SEQ ID NO: 162 | | |
| hu6(L8H14)-S107E- | hu6VL8 - | CD3VL- | | |
| diabody | S107EVH-knob | hu6VH14-hole | | |
| Format 3 | SEQ ID NO: 187 | SEQ ID NO: 188 | | |
| hu6(L8H15)-S107E- | hu6VL8 - | CD3VL- | | |
| diabody | S107EVH-knob | hu6VH15-hole | | |
| Format 3 | SEQ ID NO: 187 | SEQ ID NO: 189 | | |
| hu6(L9H14)-S107E- | hu6VL9- | CD3VL- | | |
| diabody | S107EVH-knob | hu6VH14-hole | | |
| Format 3 | SEQ ID NO: 190 | SEQ ID NO: 188 | | |
| hu6(L9H15)-S107E- | hu6VL9- | CD3VL- | | |
| diabody | S107EVH-knob | hu6VH15-hole | | |
| Format 3 | SEQ ID NO: 190 | SEQ ID NO: 189 | | |
| hu6(L10H14)- | hu6VL10- | CD3VL- | | |
| S107E-diabody | S107EVH-knob | hu6VH14-hole | | |
| Format 3 | SEQ ID NO: 191 | SEQ ID NO: 188 | | |
| hu6(L10H15)- | hu6VL10- | CD3VL- | | |
| S107E-diabody | S107EVH-knob | hu6VH15-hole | | |
| Format 3 | SEQ ID NO: 191 | SEQ ID NO: 189 | | |
| hu6(D56E)-I2C- | hu6VL (D56E) - | CD3VL-hu6VH- | | |
| diabody | I2CVH-knob | hole | | |
| Format 3 | SEQ ID NO: 259 | SEQ ID NO: 162 | | |
| hu6(D56S)-I2C- | hu6VL (D56S) - | CD3VL-hu6VH- | | |
| diabody | I2CVH-knob | hole | | |
| Format 3 | SEQ ID NO: 260 | SEQ ID NO: 162 | | |

Note:
hu6-S107E-hot Ig indicates that the molecule uses variable regions of mAb6 and its humanized antibodies as the DLL3-binding domain, uses a variable region of S107E as the CD3-binding domain, and uses hot Ig as the molecular structure. The same applies to the others.

The amino acid sequences of the DLL3-CD3 bispecific antibodies of the present disclosure are as follows:

(SEQ ID NO: 152)
>hu6VL-G4S-Obscurin
DIQMTQSPSSLSASVGDRVTITCHASQGISGNMGWLQQKPGKAPKGLIYHGANLEDG
VPSRFSGSGSGADYTLTISSLQPEDFADYYCVQYAQFPYTFGGGTKVEIKGGGGSSGA
PRFLTRPKASVVSVGKDATLSCQIVGNPFPQVSWEKDKQPVTAGVRFRLAQDGDLY
RLKILDLQLSDSGQYVCRARNAHGEAFACLGLQVDAEA
(SEQ ID NO: 153)
>hu6VH-G4S-Titin-hole
EVQLVQSGSELKKPGASVKVSCKASGYTFTDYYMNWVRQAPGQGLEWMGIINPYS
GVTIYNHKFKGRFALSVDKSSNTAYLQISSLKAEDTAVYYCARQDSLLDYAMDYWG
QGTTVTVSSGGGGSGIPPKIECLPIDISIDEGKVLTVASAFTGEPTPEVTWSTGGRKIHS
QEQGRFHIENTDDSTTLTIKDVQKQDGGLYTLTLRNEFGSDSATVNIHIRSIDKTHTCP
PCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAK
GQPREPQVCTLPPSREEMTKNQVSLSCAVKGFYPSDIAVEWESNGQPENNYKTTPPV
LDSDGSFFLVSKLTVDKSRWQQGNVFSCSVMHEALHNHYTQKSLSLSPGK
(SEQ ID NO: 154)
>S107EVH-CH1-knob
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNEYIS

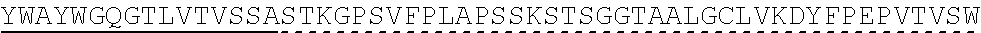

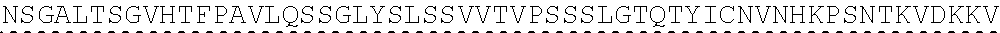

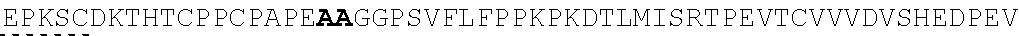

KFNWYVDGVEVHNAKTKPREEQYNSTYRVVSVLTVLHQDWLNGKEYKCKVSNKA
LPAPIEKTISKAKGQPREPQVYTLPPCREEMTKNQVSLWCLVKGFYPSDIAVEWESN
GQPENNYKTTPPVLDSDGSFFLYSKLTVDKSRWQQGNVFSCSVMHEALHNHYTQKS
LSLSPGK
(SEQ ID NO: 155)
>CD3VL-hλ
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGQPK
ANPTVTLFPPSSEELQANKATLVCLISDFYPGAVTVAWKADGSPVKAGVETTKPSKQ
SNNKYAASSYLSLTPEQWKSHRSYSCQVTHEGSTVEKTVAPTEC
(SEQ ID NO: 156)
>I2CVH-CH1-knob
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYISY
WAYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALP
APIEKTISKAKGQPREPQVYTLPPCREEMTKNQVSLWCLVKGFYPSDIAVEWESNGQ
PENNYKTTPPVLDSDGSFFLYSKLTVDKSRWQQGNVFSCSVMHEALHNHYTQKSLS
LSPGK
(SEQ ID NO: 157)
>hu6VL-hκ
DIQMTQSPSSLSASVGDRVTITCHASQGISGNMGWLQQKPGKAPKGLIYHGANLEDG
VPSRFSGSGSGADYTLTISSLQPEDFADYYCVQYAQFPYTFGGGTKVEIKRTVAAPSV
FIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
(SEQ ID NO: 158)
>hu6VH-CH1-hole
EVQLVQSGSELKKPGASVKVSCKASGYTFTDYYMNWVRQAPGQGLEWMGIINPYS
GVTIYNHKFKGRFALSVDKSSNTAYLQISSLKAEDTAVYYCARQDSLLDYAMDYWG
QGTTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDK
THTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVCTLPPSREEMTKNQVSLSCAVKGFYPSDIAVEWESNGQPENNY
KTTPPVLDSDGSFFLVSKLTVDKSRWQQGNVFSCSVMHEALHNHYTQKSLSLSPGK
(SEQ ID NO: 159)
>S107E_scFv-knob
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNEYIS
YWAYWGQGTLVTVSSGGGGSGGGGSGGGGSQTVVTQEPSLTVSPGGTVTLTCGSST
GAVTSGNYPNWVQQKPGQAPRGLIGGTKFLAPGTPARFSGSLLGGKAALTLSGVQP
EDEAEYYCVLWYSNRWVFGGGTKLTVLGGGGSDKTHTCPPCPAPEAAGGPSVFLFP
PKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPCREE
MTKNQVSLWCLVKGFYPSDIAVEWESNGQPENNYKTTPPVLDSDGSFFLYSKLTVD
KSRWQQGNVFSCSVMHEALHNHYTQKSLSLSPGK
(SEQ ID NO: 160)
>I2C_scFv-knob
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYISY
WAYWGQGTLVTVSSGGGGSGGGGSGGGGSQTVVTQEPSLTVSPGGTVTLTCGSSTG
AVTSGNYPNWVQQKPGQAPRGLIGGTKFLAPGTPARFSGSLLGGKAALTLSGVQPE
DEAEYYCVLWYSNRWVFGGGTKLTVLGGGGSDKTHTCPPCPAPEAAGGPSVFLFPP
KPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPCREE
MTKNQVSLWCLVKGFYPSDIAVEWESNGQPENNYKTTPPVLDSDGSFFLYSKLTVD
KSRWQQGNVFSCSVMHEALHNHYTQKSLSLSPGK
(SEQ ID NO: 161)
>hu6VL-S107EVH-knob
DIQMTQSPSSLSASVGDRVTITCHASQGISGNMGWLQQKPGKAPKGLIYHGANLEDG
VPSRFSGSGSGADYTLTISSLQPEDFADYYCVQYAQFPYTFGGGTKVEIKGGGSGGG
GEVQLVESGGGLVQPGGSLKLSCAASGFTFNKYAMNWVRQAPGKGLEWVARIRSK
YNNYATYYADSVKDRFTISRDDSKNTAYLQMNNLKTEDTAVYYCVRHGNFGNEYI
SYWAYWGQGTLVTVSSGCPPCPAPEAAGAPSVFLFPPKPKDTLMISRTPEVTCVVVD
VSHEDPEVKFNWYVDGVEVHNAKTKPREEQYNSTYRVVSVLTVLHQDWLNGKEY
KCKVSNKALPAPIEKTISKAKGQPREPQVCTLPPSREEMTKNQVSLWCLVKGFYPSDI
AVEWESNGQPENNYKTTPPVLDSDGSFFLYSKLTVDKSRWQQGNVFSCSVMHEALH
NHYTQKSLSLSPGK
(SEQ ID NO: 162)
>CD3VL-hu6VH-hole
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGGG
GSGGGGEVQLVQSGSELKKPGASVKVSCKASGYTFTDYYMNWVRQAPGQGLEWM
GIINPYSGVTIYNHKFKGRFALSVDKSSNTAYLQISSLKAEDTAVYYCARQDSLLDYA
MDYWGQGTTVTVSSGCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGKEYKC
KVSNKALPAPIEKTISKAKGQPREPQVYTLPPCREEMTKNQVSLSCAVKGFYPSDIAV
EWESNGQPENNYKTTPPVLDSDGSFFLVSKLTVDKSRWQQGNVFSCSVMHEALHNH
YTQKSLSLSPGK
(SEQ ID NO: 163)
>hu6VL-I2CVH-knob
DIQMTQSPSSLSASVGDRVTITCHASQGISGNMGWLQQKPGKAPKGLIYHGANLEDG
VPSRFSGSGSGADYTLTISSLQPEDFADYYCVQYAQFPYTFGGGTKVEIKGGGSGGG
GEVQLVESGGGLVQPGGSLKLSCAASGFTFNKYAMNWVRQAPGKGLEWVARIRSK
YNNYATYYADSVKDRFTISRDDSKNTAYLQMNNLKTEDTAVYYCVRHGNFGNSYIS
YWAYWGQGTLVTVSSGCPPCPAPEAAGAPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLWCLVKGFYPSDIA
VEWESNGQPENNYKTTPPVLDSDGSFFLYSKLTVDKSRWQQGNVFSCSVMHEALHN
HYTQKSLSLSPGK
(SEQ ID NO: 164)
>hu98VL-G4S-Obscurin
DIQMTQSPSSLSASVGDRVTITCRASRDITKFLNWYQQKPGGAVKLLIYYTSRLHSGV
PSRFSGSGSGTDYTLTISSLQPEDFATYYCQQGNTLPWTFGGGTKVEIKGGGGSSGAP
RFLTRPKASVVSVGKDATLSCQIVGNPFPQVSWEKDKQPVTAGVRFRLAQDGDLYR
LKILDLQLSDSGQYVCRARNAHGEAFACLGLQVDAEA
(SEQ ID NO: 165)
>hu98VH-G4S-Titin-hole
EVQLVESGGGLVQPGGSLRLSCAASGFTFSDYGIHWVRQAPGKGLEWVAYISSGGY
TSYYADTVKGRFTISRDNAKNSLYLQMNSLRAEDTAVYYCARGDYEDIFYTMDYW
GQGTTVTVSSGGGGSGIPPKIECLPIDISIDEGKVLTVASAFTGEPTPEVTWSTGGRKIH
SQEQGRFHIENTDDSTTLTIKDVQKQDGGLYTLTLRNEFGSDSATVNIHIRSIDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVCTLPPSREEMTKNQVSLSCAVKGFYPSDIAVEWESNGQPENNYKTTPP
VLDSDGSFFLVSKLTVDKSRWQQGNVFSCSVMHEALHNHYTQKSLSLSPGK
(SEQ ID NO: 166)
>hu98VL-hκ
DIQMTQSPSSLSASVGDRVTITCRASRDITKFLNWYQQKPGGAVKLLIYYTSRLHSGV
PSRFSGSGSGTDYTLTISSLQPEDFATYYCQQGNTLPWTFGGGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
(SEQ ID NO: 167)
>hu98VH-CH1-hole
EVQLVESGGGLVQPGGSLRLSCAASGFTFSDYGIHWVRQAPGKGLEWVAYISSGGY
TSYYADTVKGRFTISRDNAKNSLYLQMNSLRAEDTAVYYCARGDYEDIFYTMDYW
GQGTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHTCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIE
KTISKAKGQPREPQVCTLPPSREEMTKNQVSLSCAVKGFYPSDIAVEWESNGQPENN
YKTTPPVLDSDGSFFLVSKLTVDKSRWQQGNVFSCSVMHEALHNHYTQKSLSLSPG
K
(SEQ ID NO: 168)
>hu98VL-S107EVH-knob
DIQMTQSPSSLSASVGDRVTITCRASRDITKFLNWYQQKPGGAVKLLIYYTSRLHSGV
PSRFSGSGSGTDYTLTISSLQPEDFATYYCQQGNTLPWTFGGGTKVEIKGGGSGGGGE
VQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGNFGNEYISY
WAYWGQGTLVTVSSGCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGKEYKC
KVSNKALPAPIEKTISKAKGQPREPQVCTLPPSREEMTKNQVSLWCLVKGFYPSDIAV
EWESNGQPENNYKTTPPVLDSDGSFFLYSKLTVDKSRWQQGNVFSCSVMHEALHNH
YTQKSLSLSPGK
(SEQ ID NO: 169)
>CD3VL-hu98VH-hole
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGGG
GSGGGGEVQLVESGGGLVQPGGSLRLSCAASGFTFSDYGIHWVRQAPGKGLEWVA
YISSGGYTSYYADTVKGRFTISRDNAKNSLYLQMNSLRAEDTAVYYCARGDYEDIFY
TMDYWGQGTTVTVSSGCPPCPAPEAAGAPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVYTLPPCREEMTKNQVSLSCAVKGFYPSDIA
VEWESNGQPENNYKTTPPVLDSDGSFFLVSKLTVDKSRWQQGNVFSCSVMHEALHN
HYTQKSLSLSPGK
(SEQ ID NO: 170)
>hu98VL-I2CVH-knob
DIQMTQSPSSLSASVGDRVTITCRASRDITKFLNWYQQKPGGAVKLLIYYTSRLHSGV
PSRFSGSGSGTDYTLTISSLQPEDFATYYCQQGNTLPWTFGGGTKVEIKGGGSGGGGE
VQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGNFGNSYISY
WAYWGQGTLVTVSSGCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGKEYKC
KVSNKALPAPIEKTISKAKGQPREPQVCTLPPSREEMTKNQVSLWCLVKGFYPSDIAV
EWESNGQPENNYKTTPPVLDSDGSFFLYSKLTVDKSRWQQGNVFSCSVMHEALHNH
YTQKSLSLSPGK
(SEQ ID NO: 171)
>hu110VL-G4S-Obscurin
NIVMTQSPKSLAVSLGERATINCKASENVGIFVSWYQQKPGQSPKLLIYGASNRYTG
VPDRESGSGSATDFTLTISSLQAEDVADYYCGQSHSYPLTFGQGTKLEIKGGGGSSGA
PRFLTRPKASVVSVGKDATLSCQIVGNPFPQVSWEKDKQPVTAGVRFRLAQDGDLY
RLKILDLQLSDSGQYVCRARNAHGEAFACLGLQVDAEA
(SEQ ID NO: 172)
>hul10VH-G4S-Titin-hole
EVQLVQSGAEVKKPGASVKVSCKTSGYTFTDYYMNWVRQAPGQSLEWMGIINPYQ
GGTSYTQKFKGRVTITVDKSASTAYMELSSLRSEDTAVYYCARHFYDSSYLTYSTM
DYWGQGTTVTVSSGGGGSGIPPKIECLPIDISIDEGKVLTVASAFTGEPTPEVTWSTGG
RKIHSQEQGRFHIENTDDSTTLTIKDVQKQDGGLYTLTLRNEFGSDSATVNIHIRSIDK
THTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVCTLPPSREEMTKNQVSLSCAVKGFYPSDIAVEWESNGQPENNY
KTTPPVLDSDGSFFLVSKLTVDKSRWQQGNVFSCSVMHEALHNHYTQKSLSLSPGK
(SEQ ID NO: 173)
>hul10VL-hκ
NIVMTQSPKSLAVSLGERATINCKASENVGIFVSWYQQKPGQSPKLLIYGASNRYTG
VPDRFSGSGSATDFTLTISSLQAEDVADYYCGQSHSYPLTFGQGTKLEIKRTVAAPSV
FIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
(SEQ ID NO: 174)
>hu110VH-CH1-hole
EVQLVQSGAEVKKPGASVKVSCKTSGYTFTDYYMNWVRQAPGQSLEWMGIINPYQ
GGTSYTQKFKGRVTITVDKSASTAYMELSSLRSEDTAVYYCARHFYDSSYLTYSTMD
YWGQGTTVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEPKSC
DKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVCTLPPSREEMTKNQVSLSCAVKGFYPSDIAVEWESNGQPENN
YKTTPPVLDSDGSFFLVSKLTVDKSRWQQGNVFSCSVMHEALHNHYTQKSLSLSPGK
(SEQ ID NO: 175)
>hu110VL-S107EVH-knob
NIVMTQSPKSLAVSLGERATINCKASENVGIFVSWYQQKPGQSPKLLIYGASNRYTG
VPDRESGSGSATDFTLTISSLQAEDVADYYCGQSHSYPLTFGQGTKLEIKGGGSGGGG
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNEYIS
YWAYWGQGTLVTVSSGCPPCPAPEAAGAPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLWCLVKGFYPSDIA
VEWESNGQPENNYKTTPPVLDSDGSFFLYSKLTVDKSRWQQGNVFSCSVMHEALHN
HYTQKSLSLSPGK
(SEQ ID NO: 176)
>CD3VL-hul10VH-hole
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGGG
GSGGGGEVQLVQSGAEVKKPGASVKVSCKTSGYTFTDYYMNWVRQAPGQSLEWM
GIINPYQGGTSYTQKFKGRVTITVDKSASTAYMELSSLRSEDTAVYYCARHFYDSSY
LTYSTMDYWGQGTTVTVSSGCPPCPAPEAAGAPSVFLFPPKPKDTLMISRTPEVTCV
VVDVSHEDPEVKFNWYVDGVEVHNAKTKPREEQYNSTYRVVSVLTVLHQDWLNG
KEYKCKVSNKALPAPIEKTISKAKGQPREPQVYTLPPCREEMTKNQVSLSCAVKGFY
PSDIAVEWESNGQPENNYKTTPPVLDSDGSFFLVSKLTVDKSRWQQGNVFSCSVMH
EALHNHYTQKSLSLSPGK
(SEQ ID NO: 177)
>hu110VL-I2CVH-knob
NIVMTQSPKSLAVSLGERATINCKASENVGIFVSWYQQKPGQSPKLLIYGASNRYTG
VPDRESGSGSATDFTLTISSLQAEDVADYYCGQSHSYPLTFGQGTKLEIKGGGSGGGG
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNSYISY
WAYWGQGTLVTVSSGCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGKEYKC
KVSNKALPAPIEKTISKAKGQPREPQVCTLPPSREEMTKNQVSLWCLVKGFYPSDIAV
EWESNGQPENNYKTTPPVLDSDGSFFLYSKLTVDKSRWQQGNVFSCSVMHEALHNH
YTQKSLSLSPGK
(SEQ ID NO: 178)
>hu30VL-G4S-Obscurin
DIVMTQSPDSLAVSLGERATINCKASENVGYVSWYQQKPGQPPKLLIYGASNRYTGV
PDRFSGSGSGTDFTLTISSLQAEDVAVYYCGQSYSYPLTFGQGTKLEIKGGGGSSGAP
RFLTRPKASVVSVGKDATLSCQIVGNPFPQVSWEKDKQPVTAGVRFRLAQDGDLYR
LKILDLQLSDSGQYVCRARNAHGEAFACLGLQVDAEA
(SEQ ID NO: 179)
>hu30VH-G4S-Titin-hole
EVRLVQSGAEVKKPGASVKVSCKASGYTFTDYYMNWVRQAPGQSLEWMGIINPYT
GDTSYNQKFRGRVTLTVDKSASTAYMELSSLRSEDTAVYYCARYHYGPHLEGYFDV
WGQGTTVTVSSGGGGSGIPPKIECLPIDISIDEGKVLTVASAFTGEPTPEVTWSTGGRK
IHSQEQGRFHIENTDDSTTLTIKDVQKQDGGLYTLTLRNEFGSDSATVNIHIRSIDKTH
TCPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTIS
KAKGQPREPQVCTLPPSREEMTKNQVSLSCAVKGFYPSDIAVEWESNGQPENNYKT
TPPVLDSDGSFFLVSKLTVDKSRWQQGNVFSCSVMHEALHNHYTQKSLSLSPGK
(SEQ ID NO: 180)
>hu30VL-hκ
DIVMTQSPDSLAVSLGERATINCKASENVGYVSWYQQKPGQPPKLLIYGASNRYTGV
PDRFSGSGSGTDFTLTISSLQAEDVAVYYCGQSYSYPLTFGQGTKLEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
(SEQ ID NO: 181)
>hu30VH-CH1-hole
EVRLVQSGAEVKKPGASVKVSCKASGYTFTDYYMNWVRQAPGQSLEWMGIINPYT
GDTSYNQKFRGRVTLTVDKSASTAYMELSSLRSEDTAVYYCARYHYGPHLEGYFDV
WGQGTTVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSCD
KTHTCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIE
KTISKAKGQPREPQVCTLPPSREEMTKNQVSLSCAVKGFYPSDIAVEWESNGQPENN
YKTTPPVLDSDGSFFLVSKLTVDKSRWQQGNVFSCSVMHEALHNHYTQKSLSLSPG
K
(SEQ ID NO: 182)
>hu30VL-S107EVH-knob
DIVMTQSPDSLAVSLGERATINCKASENVGYVSWYQQKPGQPPKLLIYGASNRYTGV
PDRFSGSGSGTDFTLTISSLQAEDVAVYYCGQSYSYPLTFGQGTKLEIKGGGSGGGGE
VQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGNFGNEYISY
WAYWGQGTLVTVSSGCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGKEYKC
KVSNKALPAPIEKTISKAKGQPREPQVCTLPPSREEMTKNQVSLWCLVKGFYPSDIAV
EWESNGQPENNYKTTPPVLDSDGSFFLYSKLTVDKSRWQQGNVFSCSVMHEALHNH
YTQKSLSLSPGK
(SEQ ID NO: 183)
>CD3VL-hu30VH-hole
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGGG
GSGGGGEVRLVQSGAEVKKPGASVKVSCKASGYTFTDYYMNWVRQAPGQSLEWM
GIINPYTGDTSYNQKFRGRVTLTVDKSASTAYMELSSLRSEDTAVYYCARYHYGPHL
EGYFDVWGQGTTVTVSSGCPPCPAPEAAGAPSVFLFPPKPKDTLMISRTPEVTCVVV
DVSHEDPEVKFNWYVDGVEVHNAKTKPREEQYNSTYRVVSVLTVLHQDWLNGKE
YKCKVSNKALPAPIEKTISKAKGQPREPQVYTLPPCREEMTKNQVSLSCAVKGFYPS
DIAVEWESNGQPENNYKTTPPVLDSDGSFFLVSKLTVDKSRWQQGNVFSCSVMHEA
LHNHYTQKSLSLSPGK
(SEQ ID NO: 184)
>hu30VL-I2CVH-knob
DIVMTQSPDSLAVSLGERATINCKASENVGYVSWYQQKPGQPPKLLIYGASNRYTGV
PDRFSGSGSGTDFTLTISSLQAEDVAVYYCGQSYSYPLTFGQGTKLEIKGGGSGGGGE
VQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGNFGNSYISY
WAYWGQGTLVTVSSGCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGKEYKC
KVSNKALPAPIEKTISKAKGQPREPQVCTLPPSREEMTKNQVSLWCLVKGFYPSDIAV
EWESNGQPENNYKTTPPVLDSDGSFFLYSKLTVDKSRWQQGNVFSCSVMHEALHNH
YTQKSLSLSPGK
(SEQ ID NO: 185)
>hu6VL (D56E)-S107EVH-knob (hu6 (D56E)-S107E-diabody-C1
DIQMTQSPSSLSASVGDRVTITCHASQGISGNMGWLQQKPGKAPKGLIYHGANLEEG
VPSRFSGSGSGADYTLTISSLQPEDFADYYCVQYAQFPYTFGGGTKVEIKGGGSGGG
GEVQLVESGGGLVQPGGSLKLSCAASGFTFNKYAMNWVRQAPGKGLEWVARIRSK
YNNYATYYADSVKDRFTISRDDSKNTAYLQMNNLKTEDTAVYYCVRHGNFGNEYI
SYWAYWGQGTLVTVSSGCPPCPAPEAAGAPSVFLFPPKPKDTLMISRTPEVTCVVVD
VSHEDPEVKFNWYVDGVEVHNAKTKPREEQYNSTYRVVSVLTVLHQDWLNGKEY
KCKVSNKALPAPIEKTISKAKGQPREPQVCTLPPSREEMTKNQVSLWCLVKGFYPSDI
AVEWESNGQPENNYKTTPPVLDSDGSFFLYSKLTVDKSRWQQGNVFSCSVMHEALH
NHYTQKSLSLSPGK
(SEQ ID NO: 259)
>hu6VL (D56E)-I2CVH-knob
DIQMTQSPSSLSASVGDRVTITCHASQGISGNMGWLQQKPGKAPKGLIYHGANLEEG
VPSRFSGSGSGADYTLTISSLQPEDFADYYCVQYAQFPYTFGGGTKVEIKGGGSGGG
GEVQLVESGGGLVQPGGSLKLSCAASGFTFNKYAMNWVRQAPGKGLEWVARIRSK
YNNYATYYADSVKDRFTISRDDSKNTAYLQMNNLKTEDTAVYYCVRHGNFGNSYIS
YWAYWGQGTLVTVSSGCPPCPAPEAAGAPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLWCLVKGFYPSDIA
VEWESNGQPENNYKTTPPVLDSDGSFFLYSKLTVDKSRWQQGNVFSCSVMHEALHN
HYTQKSLSLSPGK
(SEQ ID NO: 260)
>hu6VL (D56S)-I2CVH-knob
DIQMTQSPSSLSASVGDRVTITCHASQGISGNMGWLQQKPGKAPKGLIYHGANLESG
VPSRFSGSGSGADYTLTISSLQPEDFADYYCVQYAQFPYTFGGGTKVEIKGGGSGGG
GEVQLVESGGGLVQPGGSLKLSCAASGFTFNKYAMNWVRQAPGKGLEWVARIRSK
YNNYATYYADSVKDRFTISRDDSKNTAYLQMNNLKTEDTAVYYCVRHGNFGNSYIS
YWAYWGQGTLVTVSSGCPPCPAPEAAGAPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLWCLVKGFYPSDIA
VEWESNGQPENNYKTTPPVLDSDGSFFLYSKLTVDKSRWQQGNVFSCSVMHEALHN
HYTQKSLSLSPGK
(SEQ ID NO: 186)
>hu6VL (D56S)-S107EVH-knob
DIQMTQSPSSLSASVGDRVTITCHASQGISGNMGWLQQKPGKAPKGLIYHGANLESG
VPSRFSGSGSGADYTLTISSLQPEDFADYYCVQYAQFPYTFGGGTKVEIKGGGSGGG
GEVQLVESGGGLVQPGGSLKLSCAASGFTFNKYAMNWVRQAPGKGLEWVARIRSK
YNNYATYYADSVKDRFTISRDDSKNTAYLQMNNLKTEDTAVYYCVRHGNFGNEYI
SYWAYWGQGTLVTVSSGCPPCPAPEAAGAPSVFLFPPKPKDTLMISRTPEVTCVVVD
VSHEDPEVKFNWYVDGVEVHNAKTKPREEQYNSTYRVVSVLTVLHQDWLNGKEY
KCKVSNKALPAPIEKTISKAKGQPREPQVCTLPPSREEMTKNQVSLWCLVKGFYPSDI
AVEWESNGQPENNYKTTPPVLDSDGSFFLYSKLTVDKSRWQQGNVFSCSVMHEALH
NHYTQKSLSLSPGK
(SEQ ID NO: 187)
>hu6VL8-S107EVH-knob
DIQMTQSPSSLSASVGDRVTITCHASQGISGNMGWLQQKPGKAPKGLIYHGANLEEG
VPSRFSGSGSGADYTLTISSLQPEDFADYYCVQYAQYPYTFGGGTKVEIKGGGSGGG
GEVQLVESGGGLVQPGGSLKLSCAASGFTFNKYAMNWVRQAPGKGLEWVARIRSK
YNNYATYYADSVKDRFTISRDDSKNTAYLQMNNLKTEDTAVYYCVRHGNFGNEYI
SYWAYWGQGTLVTVSSGCPPCPAPEAAGAPSVFLFPPKPKDTLMISRTPEVTCVVVD
VSHEDPEVKFNWYVDGVEVHNAKTKPREEQYNSTYRVVSVLTVLHQDWLNGKEY
KCKVSNKALPAPIEKTISKAKGQPREPQVCTLPPSREEMTKNQVSLWCLVKGFYPSDI
AVEWESNGQPENNYKTTPPVLDSDGSFFLYSKLTVDKSRWQQGNVFSCSVMHEALH
NHYTQKSLSLSPGK
(SEQ ID NO: 188)
>CD3VL-hu6VH14-hole
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGGG
GSGGGGEVQLVQSGSELKKPGASVKVSCKASGYTFTDYYMNWVRQAPGQGLEWM
GIINPYSGVTTYNHKFKGRFALSVDKSSNTAYLQISSLKAEDTAVYYCARQDSLLDY
AMDYWGQGTTVTVSSGCPPCPAPEAAGAPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVYTLPPCREEMTKNQVSLSCAVKGFYPSDIA
VEWESNGQPENNYKTTPPVLDSDGSFFLVSKLTVDKSRWQQGNVFSCSVMHEALHN
HYTQKSLSLSPGK
(SEQ ID NO: 189)
>CD3VL-hu6VH15-hole
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGGG
GSGGGGEVQLQQSGSELKKPGASVKVSCKASGYTFTDYYMNWVRQAPGQGLEWM
GIINPYSGSTIYNHKFKGRFALSVDKSSNTAYLQISSLKAEDTAVYYCARQDSLLDYA
MDYWGQGTTVTVSSGCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGKEYKC
KVSNKALPAPIEKTISKAKGQPREPQVYTLPPCREEMTKNQVSLSCAVKGFYPSDIAV
EWESNGQPENNYKTTPPVLDSDGSFFLVSKLTVDKSRWQQGNVFSCSVMHEALHNH
YTQKSLSLSPGK
(SEQ ID NO: 190)
>hu6VL9-S107EVH-knob
NIQMTQSPSSLSASVGDRVTITCHASQGISGNMGWLQQKPGKAPKGLIYHGRNLEEG
VPSRFSGSGSGADYTLTISSLQPEDFADYYCVQYAQYPYTFGGGTKVEIKGGGSGGG
GEVQLVESGGGLVQPGGSLKLSCAASGFTFNKYAMNWVRQAPGKGLEWVARIRSK
YNNYATYYADSVKDRFTISRDDSKNTAYLQMNNLKTEDTAVYYCVRHGNFGNEYI
SYWAYWGQGTLVTVSSGCPPCPAPEAAGAPSVFLFPPKPKDTLMISRTPEVTCVVVD
VSHEDPEVKFNWYVDGVEVHNAKTKPREEQYNSTYRVVSVLTVLHQDWLNGKEY
KCKVSNKALPAPIEKTISKAKGQPREPQVCTLPPSREEMTKNQVSLWCLVKGFYPSDI
AVEWESNGQPENNYKTTPPVLDSDGSFFLYSKLTVDKSRWQQGNVFSCSVMHEALH
NHYTQKSLSLSPGK
(SEQ ID NO: 191)
>hu6VL10-S107EVH-knob
EIVLTQSPATLSLSPGERATLSCHASQGISGNMGWLQQKPGKAPRGLIYHGANLEEGI
PARFSGSGSGADYTLTISRLQPEDFADYYCVQYAQFPYTFGGGTKVEIKGGGSGGGG
EVQLVESGGGLVQPGGSLKLSCAASGFTFNKYAMNWVRQAPGKGLEWVARIRSKY
NNYATYYADSVKDRFTISRDDSKNTAYLQMNNLKTEDTAVYYCVRHGNFGNEYIS
YWAYWGQGTLVTVSSGCPPCPAPEAAGAPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVCTLPPSREEMTKNQVSLWCLVKGFYPSDIA
VEWESNGQPENNYKTTPPVLDSDGSFFLYSKLTVDKSRWQQGNVFSCSVMHEALHN
HYTQKSLSLSPGK.
The control molecules used in the present disclosure were AMG-75, which
was prepared with reference to WO2017021349, and BI-764532, which was
prepared with reference to WO2019234220. The sequences of the control
molecules are as follows:
(SEQ ID NO: 252):
AMG-757
QVQLQESGPGLVKPSETLSLTCTVSGGSISSYYWSWIRQPPGKCLEWIGYVYYSGTT
NYNPSLKSRVTISVDTSKNQFSLKLSSVTAADTAVYYCASIAVTGFYFDYWGQGTLV
TVSSGGGGSGGGGSGGGGSEIVLTQSPGTLSLSPGERVTLSCRASQRVNNNYLAWYQ
QRPGQAPRLLIYGASSRATGIPDRFSGSGSGTDFTLTISRLEPEDFAVYYCQQYDRSPL
TFGCGTKLEIKSGGGGSEVQLVESGGGLVQPGGSLKLSCAASGFTFNKYAMNWVRQ
APGKGLEWVARIRSKYNNYATYYADSVKDRFTISRDDSKNTAYLQMNNLKTEDTA
VYYCVRHGNFGNSYISYWAYWGQGTLVTVSSGGGGSGGGGSGGGGSQTVVTQEPS
LTVSPGGTVTLTCGSSTGAVTSGNYPNWVQQKPGQAPRGLIGGTKFLAPGTPARFSG
SLLGGKAALTLSGVQPEDEAEYYCVLWYSNRWVFGGGTKLTVLGGGGDKTHTCPP
CPAPELLGGPSVFLFPPKPKDTLMISRTPEVTCVVVDVSHEDPEVKFNWYVDGVEVH
NAKTKPCEEQYGSTYRCVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGGGGSGGGG
SGGGGSGGGGSGGGGSGGGGSDKTHTCPPCPAPELLGGPSVFLFPPKPKDTLMISRTP
EVTCVVVDVSHEDPEVKFNWYVDGVEVHNAKTKPCEEQYGSTYRCVSVLTVLHQD
WLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCS
VMHEALHNHYTQKSLSLSPGK
BI-764532:
(SEQ ID NO: 253):
>BI-764532 chain 1
DIQMTQSPSAMSASVGDRVTITCRASQGISNYLVWFQQKPGKAPKRLIYAVSSLYSG
VPSRFSGSGSGTEFTLTISSLQPEDFATYYCLQHDSYPYTFGQGTKLEIKRTVAAPSVF
IFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGECGGGGSEGKSSGSGSES
KSTEGKSSGSGSESKSTGGGGSQVQLVQSGAEVKKPGASVKVSCKASGYTFTSYYV
HWVRQAPGQGLEWMVIINPGGGTTSYAQKFLGRVTMTRDTSTNTVYMELKSLRSE
DTAVYYCARGEAVTGNYFYYGMDVWGQGTTVTVSSASTKGPSVFPLAPSSKSTSGG
TAALGCLVKDYFPEPVTVSWNSGALTSGVHTFPAVLQSSGLYSLSSVVTVPSSSLGT
QTYICNVNHKPSNTKVDKRVEPKSCDKTHTCPPCPAPEAAGGPSVFLFPPKPKDTLMI
SRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSL
WCLVKGFYPSDIAVEWESNGQPENNYKTTPPVLDSDGSFFLYSKLTVDKSRWQQGN
VFSCSVMHEALHNHYTQKSLSLSPG
(SEQ ID NO: 254):
>BI-764532 chain 2
EAVVTQEPSLTVSPGGTVTLTCRSSTGAVTTSNYANWVQEKPGQLPRGLIGGTNKRA
PWVPARFSGSLLGGKAALTLSGAQPEDEAEYFCALWYSNLWVFGGGTKLTVLGQP
KAAPSVTLFPPSSEELQANKATLVCLISDFYPGAVKVAWKADGSPVNTGVETTTPSK
QSNNKYAASSYLSLTPEQWKSHRSYSCQVTHEGSTVEKTVAPAECSGGGGSEGKSS
GSGSESKSTEGKSSGSGSESKSTGGGGSEVQLVESGGGLVQPGGSLKLSCAASGFTFN
TYAMNWVRQAPGKGLEWVARIRSKYNNYATYYADSVKDRFTISRDDSKNTAYLQ
MNNLKTEDTAVYYCVRHGNFGNSYVSWFAYWGQGTLVTVSAASTKGPSVFPLAPS
SKSTSGGTAALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPEAAGGPSVFLFPPK
PKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMT
KNQVSLSCAVKGFYPSDIAVEWESNGQPENNYKTTPPVLDSDGSFFLVSKLTVDKSR
WQQGNVFSCSVMHEALHNRFTQKSLSLSPG.

### TEST EXAMPLES

### Test Example 1: Biacore Antibody Affinity Assay

Test antibodies were affinity-captured with a Protein A biosensor chip (Cat. #29127556, Cytiva) for 18 seconds, and then the antigens human DLL3 (ACRO, DLL3-H52H4), cynomolgus monkey DLL3 (KACTUS, DLL-RM103), mouse DLL3 (KACTUS, DLL-MM103), human CD3D&3E (ACRO, CDD-H52W1), and monkey CD3D&3E (ACRO, CDD-C52W4) were allowed to flow through the surface of the chip for 180 seconds, followed by 600 seconds of dissociation. Reaction signals were detected in real time using a Biacore 8K (Cytiva) instrument to obtain association and dissociation curves. After the dissociation in each experimental cycle, the chip was washed and regenerated with a 10 mM glycine-hydrochloric acid solution (pH 1.5, Cat. #BR-1003-54, Cytiva). Data fitting was performed using a 1:1 Model. The results are shown in Table 26-1 to Table 26-9.

TABLE 26-1
The affinities of antibodies for human DLL3 (based on clone 6)

| Antibody | ka (1/Ms) | kd (1/s) | KD (M) |
|---|---|---|---|
| M6CHI | 9.65E+05 | 6.57E−04 | 6.81E−10 |
| hu6L1H1 | 7.98E+05 | 2.25E−03 | 2.81E−09 |
| hu6L1H2 | 7.87E+05 | 1.73E−03 | 2.19E−09 |
| hu6L1H3 | 8.12E+05 | 2.24E−03 | 2.76E−09 |
| hu6L2H1 | 7.09E+05 | 1.55E−03 | 2.19E−09 |
| hu6L2H2 | 7.04E+05 | 1.16E−03 | 1.64E−09 |
| hu6L2H3 | 6.95E+05 | 1.39E−03 | 2.01E−09 |
| hu6L3H1 | 7.15E+05 | 1.44E−03 | 2.02E−09 |
| hu6L3H2 | 7.16E+05 | 1.14E−03 | 1.59E−09 |
| hu6L3H3 | 7.18E+05 | 1.35E−03 | 1.88E−09 |
| hu6L4H4 | 7.43E+05 | 1.08E−03 | 1.45E−09 |
| hu6L2H5 | 8.10E+05 | 7.84E−04 | 9.69E−10 |
| hu6L2H6 | 8.63E+05 | 7.56E−04 | 8.77E−10 |
| hu6L2H7 | 6.70E+05 | 2.05E−03 | 3.06E−09 |
| hu6L2H11 | 2.06E+06 | 7.49E−04 | 3.63E−10 |
| hu6L2H12 | 1.98E+06 | 9.13E−04 | 4.61E−10 |
| hu6L2H13 | 2.10E+06 | 8.03E−04 | 3.82E−10 |
| hu6L4H6 | 1.91E+06 | 8.11E−04 | 4.24E−10 |
| hu6L4H11 | 2.14E+06 | 6.72E−04 | 3.14E−10 |
| hu6L4H12 | 2.06E+06 | 8.05E−04 | 3.91E−10 |
| hu6L4H13 | 2.16E+06 | 6.95E−04 | 3.22E−10 |

TABLE 26-2
The affinities of antibodies for human DLL3 (based on clone 98)

| Antibody | ka (1/Ms) | kd (1/s) | KD (M) |
|---|---|---|---|
| M98CHI | 1.59E+06 | 6.82E−04 | 4.29E−10 |
| hu98L1H1 | 1.55E+06 | 1.71E−03 | 1.10E−09 |
| hu98L1H2 | 1.56E+06 | 3.07E−03 | 1.97E−09 |
| hu98L2H1 | 1.45E+06 | 3.89E−04 | 2.69E−10 |
| hu98L2H2 | 1.22E+06 | 6.68E−04 | 5.50E−10 |
| hu98L3H1 | 1.60E+06 | 3.72E−04 | 2.32E−10 |
| hu98L3H2 | 1.58E+06 | 7.32E−04 | 4.64E−10 |
| hu98L3H4 | 1.36E+06 | 1.51E−03 | 1.11E−09 |
| hu98L3H5 | 1.42E+06 | 1.73E−03 | 1.22E−09 |

TABLE 26-3
The affinities of antibodies for human DLL3 (based on clone 110)

| Antibody | ka (1/Ms) | kd (1/s) | KD (M) |
|---|---|---|---|
| hu110L1H1 | 6.14E+05 | 4.17E−05 | 6.80E−11 |
| hu110L1H2 | 5.20E+05 | 4.26E−05 | 8.19E−11 |
| hu110L1H3 | 5.64E+05 | 4.75E−05 | 8.43E−11 |
| hu110L1H4 | 5.27E+05 | 4.80E−05 | 9.11E−11 |
| hu110L1H5 | 4.54E+05 | 6.41E−05 | 1.41E−10 |
| hu110L1H6 | 5.20E+05 | 5.32E−05 | 1.02E−10 |
| hu110L2H1 | 5.09E+05 | 5.84E−05 | 1.15E−10 |
| hu110L2H2 | 5.21E+05 | 6.25E−05 | 1.20E−10 |
| hu110L2H3 | 5.50E+05 | 6.45E−05 | 1.17E−10 |
| hu110L2H4 | 4.96E+05 | 5.11E−05 | 1.03E−10 |
| hu110L2H5 | 4.34E+05 | 8.96E−05 | 2.06E−10 |
| hu110L2H6 | 4.11E+05 | 6.86E−05 | 1.67E−10 |

TABLE 26-4
The affinities of antibodies for human DLL3 (based on clone 30)

| Antibody | ka (1/Ms) | kd (1/s) | KD (M) |
|---|---|---|---|
| hu30L1H1 | 8.57E+05 | 7.87E−04 | 9.19E−10 |
| hu30L1H2 | 9.89E+05 | 1.31E−03 | 1.32E−09 |
| hu30L1H3 | 9.29E+05 | 1.85E−03 | 1.99E−09 |
| hu30L1H4 | 9.05E+05 | 1.16E−03 | 1.28E−09 |
| hu30L1H5 | 1.23E+06 | 1.17E−03 | 9.52E−10 |
| hu30L1H6 | 1.04E+06 | 1.43E−03 | 1.38E−09 |
| hu30L1H7 | 9.85E+05 | 1.15E−03 | 1.16E−09 |
| hu30L1H8 | 4.25E+05 | 1.09E−03 | 2.57E−09 |
| hu30L1H9 | 4.19E+05 | 9.81E−04 | 2.34E−09 |
| hu30L1H10 | 4.40E+05 | 9.64E−04 | 2.19E−09 |
| hu30L1H11 | 4.14E+05 | 1.17E−03 | 2.83E−09 |
| hu30L2H1 | 8.67E+05 | 5.71E−04 | 6.58E−10 |
| hu30L2H2 | 9.14E+05 | 9.21E−04 | 1.01E−09 |
| hu30L2H3 | 9.81E+05 | 1.38E−03 | 1.41E−09 |
| hu30L2H4 | 9.66E+05 | 1.04E−03 | 1.07E−09 |
| hu30L2H5 | 9.35E+05 | 9.07E−04 | 9.69E−10 |
| hu30L2H6 | 9.15E+05 | 9.88E−04 | 1.08E−09 |
| hu30L2H7 | 9.00E+05 | 9.83E−04 | 1.09E−09 |
| hu30L3H1 | 1.63E+06 | 5.90E−04 | 3.62E−10 |
| hu30L3H2 | 8.55E+05 | 7.62E−04 | 8.91E−10 |
| hu30L3H3 | 1.05E+06 | 1.31E−03 | 1.25E−09 |
| hu30L3H4 | 1.08E+06 | 9.56E−04 | 8.82E−10 |
| hu30L3H5 | 1.12E+06 | 8.20E−04 | 7.35E−10 |
| hu30L3H6 | 9.52E+05 | 8.94E−04 | 9.39E−10 |
| hu30L3H7 | 1.05E+06 | 8.53E−04 | 8.15E−10 |
| hu30L4H5 | 4.40E+05 | 9.20E−04 | 2.09E−09 |
| hu30L4H8 | 3.43E+05 | 9.42E−04 | 2.74E−09 |
| hu30L4H9 | 3.92E+05 | 1.01E−03 | 2.57E−09 |
| hu30L4H10 | 4.27E+05 | 9.19E−04 | 2.16E−09 |
| hu30L4H11 | 3.98E+05 | 1.13E−03 | 2.84E−09 |
| hu30L5H5 | 4.46E+05 | 8.82E−04 | 1.98E−09 |
| hu30L5H8 | 4.00E+05 | 1.02E−03 | 2.54E−09 |
| hu30L5H9 | 3.99E+05 | 9.89E−04 | 2.48E−09 |
| hu30L5H10 | 4.64E+05 | 9.49E−04 | 2.05E−09 |
| hu30L5H11 | 3.91E+05 | 1.20E−03 | 3.06E−09 |

TABLE 26-5
The affinities of bispecific antibodies for human DLL3

| Antibody | ka (1/Ms) | kd (1/s) | KD (M) |
|---|---|---|---|
| hu6(D56E)-S107E-diabody | 4.22E+05 | 4.62E−04 | 1.09E−09 |
| hu6(D56E)-I2C-diabody | 4.22E+05 | 4.83E−04 | 1.14E−09 |
| hu110-S107E-hot Ig | 2.49E+05 | 2.82E−05 | 1.13E−10 |
| hu110-I2C-hot Ig | 2.28E+05 | 2.72E−05 | 1.19E−10 |
| hu110-I2C-Y body | 2.13E+05 | 2.66E−05 | 1.25E−10 |

TABLE 26-6
The affinities of bispecific antibodies
for cynomolgus monkey DLL3

| Antibody | ka (1/Ms) | kd (1/s) | KD (M) |
|---|---|---|---|
| hu6(D56E)-S107E-diabody | 2.64E+05 | 4.88E−04 | 1.85E−09 |
| hu6(D56E)-I2C-diabody | 2.51E+05 | 4.93E−04 | 1.96E−09 |
| hu110-S107E-hot Ig | 1.23E+05 | 6.43E−05 | 5.23E−10 |
| hu110-I2C-hot Ig | 1.02E+05 | 6.26E−05 | 6.16E−10 |
| hu110-I2C-Y body | 8.81E+04 | 6.71E−05 | 7.62E−10 |

TABLE 26-7
The affinities of bispecific antibodies for mouse DLL3

| Antibody | ka (1/Ms) | kd (1/s) | KD (M) |
|---|---|---|---|
| hu6(D56E)-S107E-diabody | 3.78E+05 | 4.51E−03 | 1.19E−08 |
| hu6(D56E)-I2C-diabody | 3.47E+05 | 4.85E−03 | 1.40E−08 |

| | |
|---|---|
| hu110-S107E-hot Ig | No binding |
| hu110-I2C-hot Ig | No binding |
| hu110-I2C-Y body | No binding |

TABLE 26-8
The affinities of bispecific antibodies for human CD3

| Antibody | ka (1/Ms) | kd (1/s) | KD (M) |
|---|---|---|---|
| hu6(D56E)-S107E-diabody | 1.93E+05 | 1.12E−02 | 5.82E−08 |
| hu6(D56E)-I2C-diabody | 1.12E+06 | 2.13E−03 | 1.90E−09 |
| hu110-S107E-hot Ig | 1.60E+05 | 7.49E−03 | 4.67E−08 |
| hu110-I2C-hot Ig | 8.81E+05 | 1.68E−03 | 1.91E−09 |
| hu110-I2C-Y body | 9.44E+05 | 2.87E−03 | 3.04E−09 |

TABLE 26-9
The affinities of bispecific antibodies for cynomolgus monkey CD3

| Antibody | ka (1/Ms) | kd (1/s) | KD (M) |
|---|---|---|---|
| hu6(D56E)-S107E-diabody | 2.66E+05 | 1.23E−02 | 4.60E−08 |
| hu6(D56E)-I2C-diabody | 1.32E+06 | 2.04E−03 | 1.54E−09 |
| hu110-S107E-hot Ig | 1.77E+05 | 7.00E−03 | 3.96E−08 |
| hu110-I2C-hot Ig | 9.99E+05 | 1.49E−03 | 1.50E−09 |
| hu110-I2C-Y body | 1.12E+06 | 2.61E−03 | 2.32E−09 |

The results show that all the humanized antibodies and chimeric antibodies binding specifically to DLL3 of the present disclosure have good affinities for human DLL3; the bispecific antibodies have good affinities for both human and cynomolgus monkey DLL3 and CD3.

### Test Example 2: DLL3 Cell-Level FACS Binding Assay

Cells of the DLL3-expressing small cell lung cancer cell lines H1184 (ATCC, Cat. No. CRL-5858), DLL3/H82, DLL3/SHP77, hDLL3/CHO-s, and cynoDLL3/CHO-s and CD3-expressing Jurkat cells (ATCC, TIB-152) were suspended in a FACS buffer (1% BSA+pH 7.4 PBS) to form 1×10⁶/mL cell suspensions, and the suspensions were added to 96-well round-bottom plates at 100 μL/well. The plates were centrifuged at 300 g for 5 min, and the supernatant was removed. Different concentrations of test antibodies were added at 100 μL/well. The plates were incubated in the dark in a refrigerator at 4° C. for 1 h. After 3 centrifugal washes at 300 g, working concentrations of APC anti-human IgG Fc (BioLegend, 410712) or PE F(ab′)2-goat anti-human IgG (invitrogen, H10104) were added. The plates were incubated in the dark in a refrigerator at 4° C. for 40 min. After 3 centrifugal washes at 300 g, the geometric mean fluorescence intensity was measured on an Invitrogen flow cytometer, and the EC50 values for the binding of the antibodies to DLL3-expressing cells were calculated. The results are shown in Table 27-1 to Table 27-6.

TABLE 27-1
The binding activity of antibodies to
H1184 cells expressing human DLL3

| Antibody | Emax | EC50 (nM) |
|---|---|---|
| hu6L2H2 | 2193 | 0.06 |
| hu6L4H4 | 2312 | 0.06 |
| hu6L2H5 | 2486 | 0.06 |
| hu6L2H6 | 2422 | 0.05 |
| hu6L2H7 | 2196 | 0.07 |
| hu6L2H11 | 1959 | 0.04 |
| hu6L2H12 | 2145 | 0.10 |
| hu6L2H13 | 2105 | 0.10 |
| hu6L4H11 | 2188 | 0.07 |
| hu6L4H12 | 2131 | 0.05 |
| hu6L4H13 | 2118 | 0.09 |

TABLE 27-2
The binding activity of antibodies to
H1184 cells expressing human DLL3

| Antibody | Emax | EC50 |
|---|---|---|
| hu98L1H1 | 2818 | 0.09 |
| hu98L2H1 | 2466 | 0.06 |
| hu98L2H2 | 2307 | 0.04 |
| hu98L3H1 | 2439 | 0.05 |
| hu98L3H2 | 2346 | 0.04 |
| hu98L3H4 | 1599 | 0.05 |
| hu98L3H5 | 1543 | 0.05 |

TABLE 27-3
The binding activity of antibodies to
H82 cells overexpressing human DLL3

| Antibody | Emax | EC50 (nM) |
|---|---|---|
| hu110L1H1 | 4467.96 | 1.15 |
| hu110L1H2 | 4697.79 | 1.31 |
| hu110L1H3 | 4562.33 | 1.15 |
| hu110L1H4 | 4581.31 | 1.51 |
| hu110L1H5 | 4610.74 | 1.61 |
| hu110L1H6 | 4616.13 | 1.66 |
| hu110L2H1 | 4719.05 | 1.25 |
| hu110L2H2 | 4754.73 | 1.25 |
| hu110L2H3 | 4574.56 | 1.32 |
| hu110L2H4 | 4597.66 | 1.41 |
| hu110L2H5 | 4673.18 | 1.71 |
| hu110L2H6 | 5193.43 | 1.40 |

TABLE 27-4
The binding activity of antibodies to
H82 cells overexpressing human DLL3

| Antibody | Emax | EC50 (nM) |
|---|---|---|
| hu30L1H1 | 5866 | 0.74 |
| hu30L1H2 | 6049 | 0.86 |
| hu30L1H3 | 5558 | 0.75 |
| hu30L1H4 | 5358 | 0.57 |
| hu30L1H5 | 6799 | 0.88 |
| hu30L1H6 | 5751 | 0.56 |
| hu30L2H1 | 5741 | 0.89 |
| hu30L2H2 | 4914 | 0.61 |
| hu30L2H3 | 4724 | 0.59 |
| hu30L2H4 | 5441 | 0.54 |
| hu30L2H5 | 5573 | 0.65 |
| hu30L2H6 | 5422 | 0.69 |
| hu30L2H7 | 5583 | 0.83 |
| hu30L3H1 | 5530 | 0.51 |
| hu30L3H2 | 5386 | 0.44 |
| hu30L3H3 | 5303 | 0.43 |
| hu30L3H4 | 5338 | 0.43 |
| hu30L3H5 | 5251 | 0.43 |
| hu30L3H6 | 5340 | 0.46 |
| hu30L3H7 | 5448 | 0.43 |

TABLE 27-5
The binding activity of antibodies to
SHP77 cells expressing human DLL3

| Antibody | Emax | EC50 (nM) |
|---|---|---|
| hu30L1H5 | 1543 | 6.45 |
| hu30L1H8 | 1375 | 5.67 |
| hu30L1H10 | 1630 | 7.99 |
| hu30L4H5 | 1460 | 5.44 |
| hu30L4H8 | 1422 | 5.3 |
| hu30L4H9 | 1370 | 9.94 |
| hu30L4H11 | 1519 | 7.82 |
| hu30L5H5 | 1610 | 7.48 |
| hu30L5H9 | 1561 | 9.6 |

TABLE 27-6
The binding activity of bispecific antibodies to cells

| | hDLL3/ | CynoDLL3/ | |
|---|---|---|---|
| | CHO-S | CHO-S | Jurkat |
| Antibody | EC50 (nM) | EC50 (nM) | EC50 (nM) |
| hu6(D56E)-S107E-diabody | 1.00 | 1.17 | 13.62 |
| hu6(D56E)-I2C-diabody | 3.48 | 2.97 | 4.61 |
| hu110-S107E-hot Ig | 5.07 | 5.01 | 7.74 |
| hu110-I2C-Y body | 4.52 | 4.92 | 3.83 |
| hu110-I2C-hot Ig | 4.91 | 4.92 | 3.27 |

The results show that the anti-DLL3 humanized antibodies of the present disclosure have good capacities to bind to cells expressing human DLL3; the bispecific antibodies have good capacities to bind to cells expressing human and cynomolgus monkey DLL3 and CD3.

### Test Example 3: DLL3 Protein-Level ELISA Binding Assay

Streptavidin (abcam, ab136200, 1 μg/mL) was plated at 100 μL/well, and the plates were incubated overnight at 4° C. The plates were washed 3 times with 250 μL of PBST solution (PBS containing 0.1% Tween 20) per well. The plates were blocked with 250 μL of 5% milk per well at 37° C. for 2 h. The plates were washed 3 times with 250 μL of PBST solution per well. Biotinylated DLL3 antigen (1 μg/mL) (SEQ ID NO: 9) was added, and the plates were incubated at 37° C. for 1 h. The plates were washed 3 times with 250 μL of PBST solution per well. Antibodies (maximum concentration 400 nM, serially diluted 4-fold) were formulated, and incubation was performed at 37° C. for 1 h. The plates were washed 6 times with 250 μL of PBST solution per well. Working concentrations of human IgG (H+L)-HRP (Jackson, 109-035-003, 1:4000 dilution) were added at 100 μL/well, and the plates were incubated at 37° C. for 1 h. The plates were washed 6 times with 250 μL of PBST solution per well. TMB (KPL, 5120-0077) substrate solution was added at 100 μL/well, and color development was performed at room temperature for 5-10 min. 1 M H₂SO₄ was added at 100 μL/well to stop the color development, and microplate reader (Molecular Devices, VERSA max) readings at 450 nm were taken.

TABLE 28
The binding activity of bispecific
antibodies to human DLL3 protein

| Antibody | EC50 (nM) | Emax |
|---|---|---|
| hu6(D56E)-S107E-diabody | 0.49 | 0.33 |
| hu6(D56E)-I2C-diabody | 0.49 | 0.36 |
| hu110-S107E-hot Ig | 0.11 | 0.45 |
| hu110-I2C-Y body | 0.07 | 0.48 |
| hu110-I2C-hot Ig | 0.08 | 0.48 |

The results show that the bispecific antibodies of the present disclosure have good affinities for human DLL3 protein.

### Test Example 4: ELISA Binding Assay with DLL1 and DLL4 Proteins

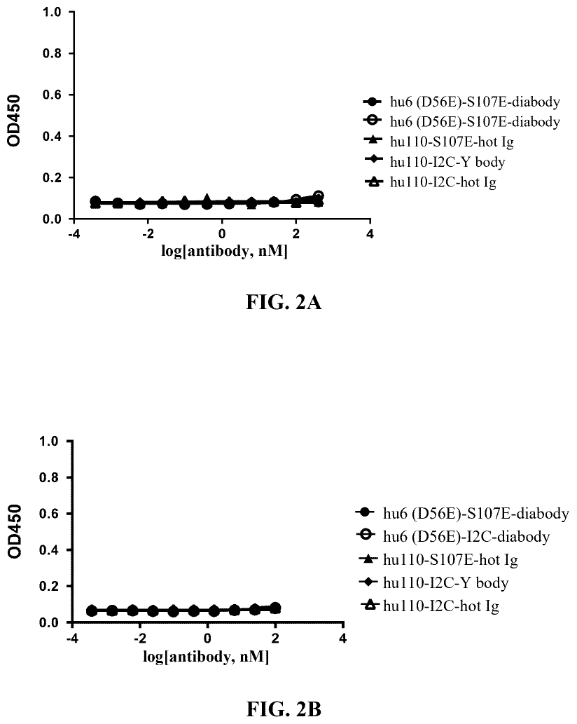

DLL1 (ACRO, DL1-H52H8, 1 μg/mL) and DLL4 (ACRO, DL4-H5227, 1 μg/mL) were plated at 100 μL/well, and the plates were incubated overnight at 4° C. The plates were washed 3 times with 250 μL of PBST solution (PBS containing 0.1% Tween 20) per well. The plates were blocked with 250 μL of 5% milk per well at 37° C. for 2 h. The plates were washed 3 times with 250 μL of PBST solution per well. Test antibodies (maximum concentration 100 nM, serially diluted 4-fold) were added, and the plates were incubated at 37° C. for 1 h. The plates were washed 6 times with 250 μL of PBST solution per well. Working concentrations of human IgG (H+L)-HRP (Jackson, 109-035-003, 1:4000 dilution) were added at 100 μL/well, and the plates were incubated at 37° C. for 1 h. The plates were washed 6 times with 250 μL of PBST solution per well. TMB (KPL, 5120-0077) substrate solution was added at 100 μL/well, and color development was performed at room temperature for 5-10 min. 1 M H₂SO₄ was added at 100 μL/well to stop the color development, and microplate reader (Molecular Devices, VERSA max) readings at 450 nm were taken. The results are shown in FIGS. 2A to 2B.

The results show that the binding of the bispecific antibodies of the present disclosure to human DLL1 and DLL4 proteins was undetectable.

### Test Example 5. Killing Activity of Bispecific Antibodies of Present Disclosure Against Cells

In this test example, the killing activity of bispecific antibodies as T cell engager molecules against tumor cells was studied. The target-specific cytotoxic activity of the bispecific antibodies of the present disclosure was evaluated, with LUC-GFP-expressing SHP77, H1184, and H460 stably transfected cell lines as target cells.

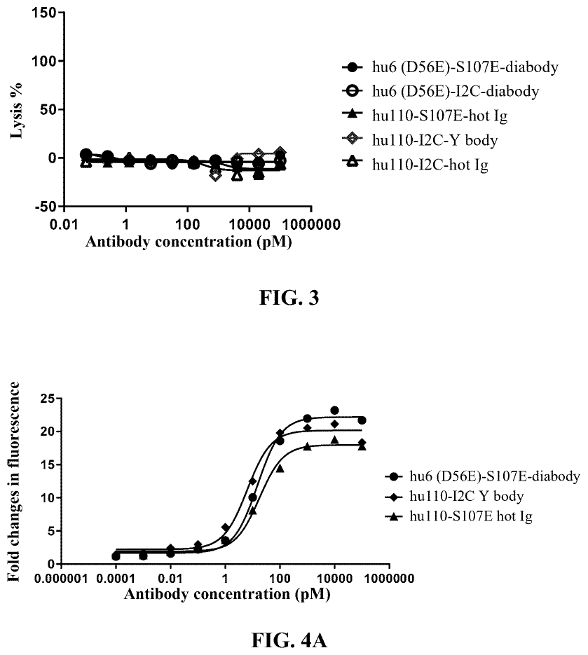

SHP77/lucG cells were cultured in 1640+10% FBS+10 μg/mL puromycin complete culture medium and passaged 2-3 times a week. H1184/lucG cells were cultured in 1640+5% FBS+10 μg/mL puromycin complete culture medium and passaged once a week. H460/lucG cells were cultured in 1640+10% FBS complete culture medium and passaged 2-3 times a week. Cryopreserved PBMCs (Milestone) were thawed and resuspended in 1640+10% FBS complete culture medium. The suspension was placed in a T75 culture flask and incubated for 4 h (at a density of 2E6 cells/mL). PBMCs were harvested, centrifuged, resuspended in 1640+10% FBS, and counted, and the cell count was adjusted to 1.5E6 cells/mL. The above PBMC suspension was mixed with a target cell suspension in equal volumes, and the mixture was added at 100 μL/well to ensure that the E:T Ratio was 10:1. In addition, a PBMC ONLY group was set up, and the above PBMC suspension was mixed with 1640+10% FBS complete culture medium in equal volumes. Antibodies were 5-fold diluted with 1640+10% FBS culture medium with an initial concentration of 600 nM (6× the final concentration), and 9 dose points were obtained and added at 20 μL/well. The treated cells were cultured in a 37° C., 5% CO₂ incubator for 48 h. The plates were taken out and centrifuged at 1000 rpm for 3 min, and 50 μL of the supernatant was pipetted into a new 3788 plate for cytokine detection and stored at −20° C. 50 μL of ONE-GloTMLuciferase (Promega, E6120) was added to the culture plates. After 5 min of incubation at room temperature, luminescence was measured using Vendor, and the killing effects of the antibodies at the different concentrations were calculated. Dose-response curve plots were created based on the logarithmic concentrations and signal values of the antibodies using Graphpad Prism8.0 software, and the IC50 for antibody-mediated killing was obtained. PBMC wells containing the target cells and containing no added antibodies were set to 0% inhibition, and the maximum inhibition rate Imax was calculated for the antibodies. The results are shown in Tables 29-1 to 29-4, Table 30, and FIG. 3.

TABLE 29-1
The cytotoxic activity of antibodies against SHP77

| Experiment 1 | Imax (%) | IC50 (pM) |
|---|---|---|
| hu6-S107E-diabody | 99 | 69.85 |
| hu6-I2C-diabody | 100 | 7.50 |
| hu98-I2C-diabody | 100 | 13.47 |

TABLE 29-2
The cytotoxic activity of antibodies against SHP77

| Experiment 2 | Imax (%) | IC50 (pM) |
|---|---|---|
| AMG-757 | 74 | 74.66 |
| hu6-I2C-diabody | 75 | 33.14 |
| hu6(D56E)-I2C-diabody | 78 | 20.25 |
| hu6(D56S)-I2C-diabody | 78 | 24.49 |

TABLE 29-3
The cytotoxic activity of antibodies against SHP77

| Experiment 3 | Imax (%) | IC50 (pM) |
|---|---|---|
| hu6(D56E)-S107E-diabody | 96 | 83.04 |
| hu6(L8H14)-S107E-diabody | 93 | 3596.22 |
| hu6(L8H15)-S107E-diabody | 97 | 294.78 |
| hu6(L9H14)-S107E-diabody | 98 | 1576.15 |
| hu6(L9H15)-S107E-diabody | 96 | 168.09 |
| hu6(L10H14)-S107E-diabody | 95 | 307.53 |
| hu6(L10H15)-S107E-diabody | 95 | 85.57 |

TABLE 29-4
The cytotoxic activity of antibodies against SHP77

| Experiment 4 | Imax (%) | IC50 (pM) |
|---|---|---|
| hu6(D56E)-I2C-diabody | 99 | 4.17 |
| hu110-I2C-Y body | 100 | 33.82 |
| hu110-I2C-hot Ig | 96 | 22.18 |
| BI-764532 | 97 | 44.63 |

TABLE 30
The killing of H1184 cells by bispecific antibodies

| Antibody | Imax (%) | IC50 (pM) |
|---|---|---|
| hu6(D56E)-S107E-diabody | 97 | 19.68 |
| hu110-I2C-Y body | 98 | 6.42 |
| hu110-S107E-hot Ig | 92 | 29.54 |

The results show that the bispecific antibodies of the present disclosure have relatively strong killing effects on the DLL3-expressing cells SHP77 and H1184 and have no killing effects on H460 cells not expressing DLL3 (FIG. 3).

### Test Example 6. In Vitro T cell Activation Activity of Bispecific Antibodies of Present Disclosure

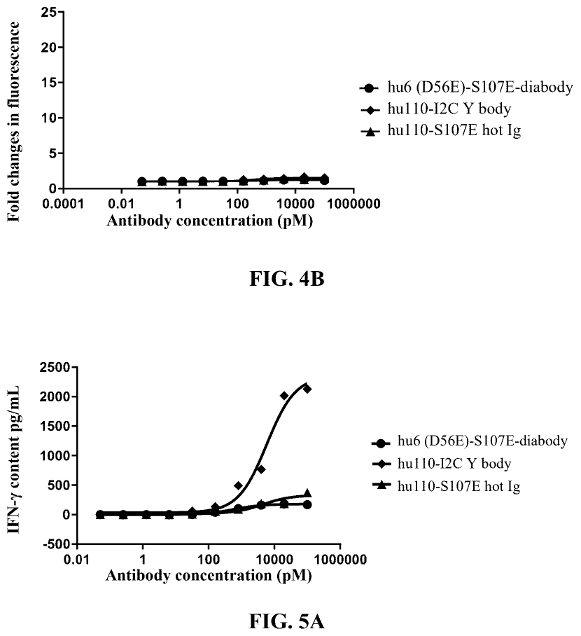

H82/DLL3 cells were cultured in 1640+10% FBS complete culture medium and passaged 2-3 times a week with a passage ratio of about 1:3. H460 cells were cultured in 1640+10% FBS complete culture medium and passaged 2-3 times a week with a passage ratio of about 1:10. Jurkat Lucia™ NFAT cells (InvivoGen) were cultured in 1640+10% FBS+100 μg/mL zeocin (InvivoGen, ant-zn-1) complete culture medium and passaged 2-3 times a week with a passage ratio of about 1:10.Jurkat Lucia™ NFAT cells were harvested, centrifuged at 1000 rpm for 3 min, resuspended, and counted, and the cell count was adjusted to 1E6 cells/mL. Target cells H82/DLL3 cells were harvested, centrifuged at 1000 rpm for 3 min, resuspended, and counted, and the cell count was adjusted to 2E5 cells/mL. H82/DLL3 and H460 cells were harvested, centrifuged at 1000 rpm for 3 min, resuspended, and counted, and the cell count was adjusted to 4E5 cells/mL. The Jurkat Lucia™ NFAT and target cells described above were mixed in equal volumes, and the mixture was added at 100 μL/well to ensure that the E:T Ratio for H82/DLL3 cells was 5:1 and the E:T Ratio for H82/DLL3 and H460 cells was 2.5:1. Antibodies were 5-fold diluted with 1640+10% FBS culture medium with an initial concentration of 600 nM (6× the final concentration), and 9 dose points were obtained and added at 20 μL/well. The treated cells were cultured in a 37° C., 5% CO₂ incubator for 5 h. QUANTI-Luc™ Gold reagent (Invivogen, rep-qlcg5) was dissolved in 25 mL of ddH₂O. 50 μL of QUANTI-Luc™ Gold was added. After 5 min of incubation at room temperature, luminescence was measured using Vendor, and the activating effects of the antibodies at the different concentrations on Jurkat Lucia™ NFAT cells were calculated. Plots were created using Graphpad Prism8.0 software, and the results are shown in FIGS. 4A to 4B and Table 31.

$$Formula for fold changes in activation N = fluorescence reading of test antibody / fluorescence reading of negative well$$

TABLE 31
Results for the activation of T cells by bispecific antibodies

| | Fold changes in | |
|---|---|---|
| Antibody | activation | EC50 (pM) |
| hu6(D56E)-S107E-diabody | 22.20 | 15.21 |
| hu110-I2C Y body | 20.18 | 6.36 |
| hu110-S107E hot Ig | 17.98 | 17.69 |

The results show that the bispecific antibodies of the present disclosure activated T cells only in the presence of DLL3-expressing DLL3/H82 cells and did not activate T cells in the presence of H460 negative cells not expressing DLL3.

### Test Example 7. Cytokine Release Levels for Bispecific Antibodies of Present Disclosure

### 1. Release Levels of Cytokine IFNγ for Bispecific Antibodies of Present Disclosure

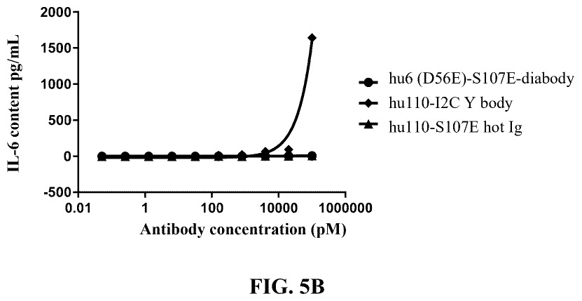

The secretion of cytokine IFNγ in the stimulation of PBMCs with DLL3/CD3 bispecific antibodies in the presence of H1184 cells was evaluated by HTRF. The supernatants collected in the cell killing experiments (Test Example 5), cryopreserved in a freezer at −20° C., were taken out, thawed at room temperature, and shaken for good uniformity. Powdered IFNγ standard was dissolved in sterile water, and the solution of the standard was diluted with 1640+10% FBS culture medium to the desired concentration. A kit for detection of IFNγ (CISBIO, 62HIFNGPEG) was taken out and equilibrated to room temperature, and two detection antibodies in the kit were 20-fold diluted with a detection buffer. 16 μL of a cell supernatant (10-fold diluted for IFNγ detection) and the IFNγ standard were added to a 96-well plate, and 4 μL of the dilution of a corresponding detection antibody was added. The plate was shaken for good uniformity, centrifuged at 1000 rpm for 1 min, and incubated overnight at room temperature. The samples incubated overnight were taken out and centrifuged at 1000 rpm for 1 min, and the absorbance at 665 nm and 620 nm was measured using a PHERAstar multi-mode microplate reader. Data were analyzed using Graphpad Prism 8, and the results are shown in FIG. 5A.

The results show that the bispecific antibodies of the present disclosure caused very low levels of IFNγ release.

### 2. Release Levels of Cytokine IL-6 for Bispecific Antibodies of Present Disclosure

The secretion of cytokine IL-6 in the stimulation of PBMCs with DLL3/CD3 bispecific antibodies in the presence of H1184 cells was evaluated by ELISA. The supernatants collected in the cell killing experiments (Test Example 5), cryopreserved in a freezer at −20° C., were taken out, thawed at room temperature, and shaken for good uniformity. The samples were 5-fold diluted with a sample diluent for later use. Before cytokine detection, an IL-6 ELISA kit (NeoBioscience Technology Co., Ltd., EHC007.96) was taken out and equilibrated to normal temperature. IL-6 standard was diluted and detection was performed according to the instructions. OD450 values were measured, and data were analyzed using Graphpad Prism 8. The results are shown in FIG. 5B.

The results show that the bispecific antibodies of the present disclosure caused very low levels of IL6 release, indicating that the bispecific antibodies of the present disclosure have better safety.

### Evaluation of In Vivo Activity

### Test Example 8. Efficacy of Bispecific Antibodies of Present Disclosure in SHP77 Subcutaneous Xenograft Tumor Model

In the present disclosure, severe combined immunodeficiency NOG mice were inoculated with SHP-77 cells, then intraperitoneally injected with human PBMCs, and, after tumorigenesis, dosed in groups.

Female NOG mice weighing about 15-17 g were purchased from Vital River. SHP-77 cells were from ATCC. Human PBMCs were purchased from Milestone (Shanghai) Biotechnologies Co. Ltd., Cat. No: ID #A10S115034.

100 μL of SHP-77 cells (4×10⁶ cells/mouse) were inoculated subcutaneously into the right flank of NOG mice. On the day of inoculation, cryopreserved hPBMCs were thawed. On day 2 post-inoculation, each mouse was intraperitoneally injected with 100 μL of a cell suspension of hPBMCs (5×10⁶ cells). Eight days after the inoculation, when the tumor volume was ˜120 mm³, mice that were overweight or had tumors that were either too big or too small were excluded. Mice were randomized into 5 groups of 8 according to tumor volume, and administration of an equimolar amount of antibody was started on the day of grouping. The doses are shown in Table 32. The corresponding equimolar amount of antibody was administered by intraperitoneal injection 3 times in total, once every 5 days, until day 15. The tumor volume and body weight were measured twice a week, and data were recorded.

Tumor volume (V) was calculated using the formula: V=½×L_{long}×Lₛₕₒᵣₜ²

Relative tumor proliferation rate T/C (%)=(T−T₀)/(C−C₀)×100%, where T and C represent the tumor volumes of the treatment group and the control group at the end of the experiment; T₀ and C₀ represent the tumor volumes at the beginning of the experiment.

$$Tumor growth inhibition rate ( T G I ) ( \% ) = 100 - T / C ( \% ) .$$

TABLE 32
The efficacy of antibodies in the SHP77 subcutaneous
xenograft tumor model (day 15 post-dose)

| | | Tumor growth |
|---|---|---|
| Group | Dose (mg/kg) | inhibition rate TGI (%) |
| Vehicle | / | / |
| AMG-757 | 1 | 87 |
| hu6(D56E)-S107E-diabody | 0.96 | 107 |
| hu110-S107E-hot Ig | 1.38 | 94 |
| hu110-I2C-Y body | 1.19 | 106 |

The results show that the bispecific antibodies of the present disclosure can significantly inhibit the growth of SHP-77 xenograft tumors.

### Test Example 9. Efficacy of Bispecific Antibodies of Present Disclosure in H1184 Subcutaneous Xenograft Tumor Model

In the present disclosure, severe combined immunodeficiency NOG mice were inoculated with H1184 cells, then intraperitoneally injected with human PBMCs, and, after tumorigenesis, dosed in groups.

Female NOG mice weighing about 15-17 g were purchased from Vital River. H1184 cells were from ATCC. Human PBMCs were purchased from Milestone (Shanghai) Biotechnologies Co. Ltd., Cat. No: ID #A10S115034.

200 μL of H1184 cells (3.5×10⁶ cells, containing 50% MatriGel) were inoculated subcutaneously into the right flank of each NOG mouse. On day 11 post-inoculation, 8 vials of cryopreserved hPBMCs (ID #A10S115034) were thawed. On day 12 post-inoculation, each mouse was intraperitoneally injected with 100 μL of a cell suspension of human PBMCs (5×10⁶ cells). Eighteen days after the inoculation, when the tumor volume was ˜140 mm³, mice that were overweight or had tumors that were either too big or too small were excluded. Mice were randomized into groups of 8 according to tumor volume, and administration of an equimolar amount of antibody was started on the day of grouping. The doses are shown in Table 33. The antibody was administered by intraperitoneal injection 4 times in total, twice a week. The tumor volume and body weight were measured twice a week, and data were recorded.

Data were recorded using Excel statistical software: the average values were calculated as avg; the SD values were calculated as STDEV; the SEM values were calculated as STDEV/SQRT (number of animals per group); GraphPad Prism software was used for plotting, and statistical analysis of the data was performed using Two-way ANOVA, One-way ANOVA, or t-test.

Tumor volume (V) was calculated using the formula: V=½×L_{long}×Lₛₕₒᵣₜ²

Relative tumor proliferation rate T/C (%)=(T −T₀)/(C −C₀)×100%, where T and C represent the tumor volumes of the treatment group and the control group at the end of the experiment; T₀ and C₀ represent the tumor volumes at the beginning of the experiment.

$$Tumor growth inhibition rate ( T G I ) ( \% ) = 100 - T / C ( \% ) .$$

TABLE 33
The efficacy of antibodies in the H1184 subcutaneous
xenograft tumor model (day 15 post-dose)

| | | Tumor growth |
|---|---|---|
| Group | Dose (mg/kg) | inhibition rate TGI (%) |
| Vehicle | / | / |
| AMG-757 | 1 | 13 |
| hu6(D56E)-S107E-diabody | 0.96 | 55 |
| hu110-I2C-Y body | 1.19 | 52 |

The results show that the bispecific antibodies of the present disclosure can significantly inhibit the growth of H1184 xenograft tumors.

Although the foregoing invention has been described in detail by way of drawings and examples for purposes of clarity of understanding, the description and examples should not be construed as limiting the scope of the present disclosure. The disclosures of all the patents and scientific literature cited herein are clearly incorporated by reference in their entirety.

1. An antigen-binding molecule binding specifically to DLL3 and CD3, comprising at least one antigen-binding moiety binding specifically to DLL3 and at least one antigen-binding moiety binding specifically to CD3, wherein:
the antigen-binding moiety binding specifically to DLL3 comprises a heavy chain variable region DLL3-VH and a light chain variable region DLL3-VL, and
the antigen-binding moiety binding specifically to CD3 comprises a heavy chain variable region CD3-VH and a light chain variable region CD3-VL, wherein:
i) the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, 24, 72, or 73, and a DLL3-LCDR3 comprising the amino acid sequence of SEQ ID NO: 25 or 74; or
ii) the DLL3-VH comprises: a DLL3-HCDR1 comprising the amino acid sequence of SEQ ID NO: 26 or 84, a DLL3-HCDR2 comprising the amino acid sequence of SEQ ID NO: 85 or 27, and a DLL3-HCDR3 comprising the amino acid sequence of SEQ ID NO: 83 or 28; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
iii) the DLL3-VH comprises: a DLL3-HCDR1 comprising the amino acid sequence of SEQ ID NO: 20, a DLL3-HCDR2 comprising the amino acid sequence of SEQ ID NO: 96, 32, 97, 98, or 99, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; or
iv) the DLL3-VH comprises: a DLL3-HCDR1 comprising the amino acid sequence of SEQ ID NO: 20, a DLL3-HCDR2 comprising the amino acid sequence of SEQ ID NO: 119, 37, 117, 118, or 120, and a DLL3-HCDR3 comprising the amino acid sequence of SEQ ID NO: 38: and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40; preferably,
i) the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
ii) the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 84, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 84, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 28; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
iii) the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; or
iv) the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40;
more preferably,
the antigen-binding molecule binds to human DLL3 at 25° C. with a KD of less than 4×10⁻⁹ M, as measured by surface plasmon resonance.

2. The antigen-binding molecule binding specifically to DLL3 and CD3 according to claim 1, wherein:
i) the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 131; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134; or
ii) the CD3-VH comprises: a CD3-HCDR1 comprising the amino acid sequence of SEQ ID NO: 129, a CD3-HCDR2 comprising the amino acid sequence of SEQ ID NO: 130, and a CD3-HCDR3 comprising the amino acid sequence of SEQ ID NO: 135; and the CD3-VL comprises: a CD3-LCDR1 comprising the amino acid sequence of SEQ ID NO: 132, a CD3-LCDR2 comprising the amino acid sequence of SEQ ID NO: 133, and a CD3-LCDR3 comprising the amino acid sequence of SEQ ID NO: 134.

3. The antigen-binding molecule binding specifically to DLL3 and CD3 according to claim 1, wherein:
i) the DLL3-VH comprises the amino acid sequence of SEQ ID NO: 54, 12, 43, 44, 45, 46, 47, 48, 49, 50, 51, 52, 53, 55, 56, or 57, and the DLL3-VL comprises the amino acid sequence of SEQ ID NO: 63, 13, 58, 59, 60, 61, 62, 64, 65, 66, 67, or 68; or
ii) the DLL3-VH comprises the amino acid sequence of SEQ ID NO: 81, 14, 78, 79, 80, or 82, and the DLL3-VL comprises the amino acid sequence of SEQ ID NO: 77, 15, 75, or 76; or
iii) the DLL3-VH comprises the amino acid sequence of SEQ ID NO: 91, 16, 89, 90, 92, 93, 94, or 95, and the DLL3-VL comprises the amino acid sequence of SEQ ID NO: 87, 17, 86, or 88; or
iv) the DLL3-VH comprises the amino acid sequence of SEQ ID NO: 109, 18, 105, 106, 107, 108, 110, 111, 112, 113, 114, 115, or 116, and the DLL3-VL comprises the amino acid sequence of SEQ ID NO: 100, 19, 101, 102, 103, or 104;
preferably,
i) the DLL3-VH comprises the amino acid sequence of SEQ ID NO: 54, and the DLL3-VL comprises the amino acid sequence of SEQ ID NO: 63; or
the DLL3-VH comprises the amino acid sequence of SEQ ID NO: 54, and the DLL3-VL comprises the amino acid sequence of SEQ ID NO: 61; or
the DLL3-VH comprises the amino acid sequence of SEQ ID NO: 54, and the DLL3-VL comprises the amino acid sequence of SEQ ID NO: 64; or
the DLL3-VH comprises the amino acid sequence of SEQ ID NO: 56, and the DLL3-VL comprises the amino acid sequence of SEQ ID NO: 65; or
the DLL3-VH comprises the amino acid sequence of SEQ ID NO: 56, and the DLL3-VL comprises the amino acid sequence of SEQ ID NO: 66; or
the DLL3-VH comprises the amino acid sequence of SEQ ID NO: 56, and the DLL3-VL comprises the amino acid sequence of SEQ ID NO: 67; or
the DLL3-VH comprises the amino acid sequence of SEQ ID NO: 57, and the DLL3-VL comprises the amino acid sequence of SEQ ID NO: 65; or
the DLL3-VH comprises the amino acid sequence of SEQ ID NO: 57, and the DLL3-VL comprises the amino acid sequence of SEQ ID NO: 66; or
the DLL3-VH comprises the amino acid sequence of SEQ ID NO: 57, and the DLL3-VL comprises the amino acid sequence of SEQ ID NO: 67; or
ii) the DLL3-VH comprises the amino acid sequence of SEQ ID NO: 81, and the DLL3-VL comprises the amino acid sequence of SEQ ID NO: 77; or
iii) the DLL3-VH comprises the amino acid sequence of SEQ ID NO: 91, and the DLL3-VL comprises the amino acid sequence of SEQ ID NO: 87; or
iv) the DLL3-VH comprises the amino acid sequence of SEQ ID NO: 109, and the DLL3-VL comprises the amino acid sequence of SEQ ID NO: 100.

4. The antigen-binding molecule binding specifically to DLL3 and CD3 according to claim 1, wherein:
i) the CD3-VH comprises the amino acid sequence of SEQ ID NO: 136, and the CD3-VL comprises the amino acid sequence of SEQ ID NO: 137; or
ii) the CD3-VH comprises the amino acid sequence of SEQ ID NO: 138, and the CD3-VL comprises the amino acid sequence of SEQ ID NO: 137.

5. The antigen-binding molecule binding specifically to DLL3 and CD3 according to claim 1, wherein:
the antigen-binding moiety binding specifically to DLL3 comprises a Titin chain and an Obscurin chain capable of forming a dimer; or
the antigen-binding moiety binding specifically to CD3 comprises a Titin chain and an Obscurin chain capable of forming a dimer;
preferably,
the Titin chain comprises an amino acid sequence selected from the group consisting of SEQ ID NO: 192 to SEQ ID NO: 210, and
the Obscurin chain comprises an amino acid sequence selected from the group consisting of SEQ ID NO: 211 to SEQ ID NO: 251;
more preferably,
the Titin chain comprises the amino acid sequence of SEQ ID NO: 208, and the Obscurin chain comprises the amino acid sequence of SEQ ID NO: 246.

6. The antigen-binding molecule binding specifically to DLL3 and CD3 according to claim 1, comprising an Fc region, wherein preferably, the Fc region is a human IgG Fc region; further preferably, the Fc region is a human IgG1 Fc region;
more preferably, the Fc region comprises one or more amino acid substitutions capable of reducing binding of the Fc region to an Fcγ receptor;
most preferably, the Fc region is a human IgG1 Fc region and has amino acids A at positions 234 and 235, with numbering in accordance with the EU index.

7. The antigen-binding molecule binding specifically to DLL3 and CD3 according to claim 1, comprising an Fc region comprising a first subunit Fc1 and a second subunit Fc2 capable of associating with each other, wherein the Fc1 and Fc2 each independently have one or more amino acid substitutions reducing homodimerization of the Fc region;
preferably, the Fc1 has a knob structure according to the knob-into-hole technique, and the Fc2 has a hole structure according to the knob-into-hole technique;
more preferably, the Fc1 has an amino acid W at position 366, and the Fc2 has an amino acid S at position 366, an amino acid A at position 368, and an amino acid V at position 407, with numbering in accordance with the EU index;
most preferably, the Fc1 comprises the amino acid sequence of SEQ ID NO: 143, and the Fc2 comprises the amino acid sequence of SEQ ID NO: 144; or the Fc1 comprises the amino acid sequence of SEQ ID NO: 145, and the Fc2 comprises the amino acid sequence of SEQ ID NO: 146.

8. The antigen-binding molecule binding specifically to DLL3 and CD3 according to claim 7, wherein the antigen-binding molecule comprises one antigen-binding moiety binding specifically to DLL3 and one antigen-binding moiety binding specifically to CD3; the antigen-binding moiety binding specifically to DLL3 is a replaced Fab comprising a Titin chain and an Obscurin chain capable of forming a dimer, and the antigen-binding moiety binding specifically to CD3 is a Fab; or
the antigen-binding moiety binding specifically to DLL3 is a Fab, and the antigen-binding moiety binding specifically to CD3 is a replaced Fab comprising a Titin chain and an Obscurin chain capable of forming a dimer;
preferably,
the antigen-binding molecule binding specifically to DLL3 and CD3 comprises one first chain having a structure represented by formula (a), one second chain having a structure represented by formula (b), one third chain having a structure represented by formula (c), and one fourth chain having a structure represented by formula (d): 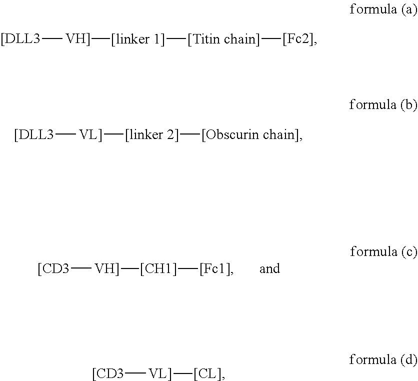
wherein the linker 1 and linker 2 are identical or different peptide linkers; or the linker 1 and/or linker 2 are/is absent;
the structures represented by formulas (a), (b), (c), and (d) are arranged from N-terminus to C-terminus;
the Titin chain and the Obscurin chain are interchangeable;
the Fc1 and the Fc2 are interchangeable;
more preferably, the antigen-binding molecule binding specifically to DLL3 and CD3 comprises:
one first chain comprising the amino acid sequence of SEQ ID NO: 171, one second chain comprising the amino acid sequence of SEQ ID NO: 172, one third chain comprising the amino acid sequence of SEQ ID NO: 154, and one fourth chain comprising the amino acid sequence of SEQ ID NO: 155; or
one first chain comprising the amino acid sequence of SEQ ID NO: 171, one second chain comprising the amino acid sequence of SEQ ID NO: 172, one third chain comprising the amino acid sequence of SEQ ID NO: 156, and one fourth chain comprising the amino acid sequence of SEQ ID NO: 155; or
one first chain comprising the amino acid sequence of SEQ ID NO: 152, one second chain comprising the amino acid sequence of SEQ ID NO: 153, one third chain comprising the amino acid sequence of SEQ ID NO: 154, and one fourth chain comprising the amino acid sequence of SEQ ID NO: 155; or
one first chain comprising the amino acid sequence of SEQ ID NO: 152, one second chain comprising the amino acid sequence of SEQ ID NO: 153, one third chain comprising the amino acid sequence of SEQ ID NO: 156, and one fourth chain comprising the amino acid sequence of SEQ ID NO: 155; or
one first chain comprising the amino acid sequence of SEQ ID NO: 164, one second chain comprising the amino acid sequence of SEQ ID NO: 165, one third chain comprising the amino acid sequence of SEQ ID NO: 154, and one fourth chain comprising the amino acid sequence of SEQ ID NO: 155; or
one first chain comprising the amino acid sequence of SEQ ID NO: 164, one second chain comprising the amino acid sequence of SEQ ID NO: 165, one third chain comprising the amino acid sequence of SEQ ID NO: 156, and one fourth chain comprising the amino acid sequence of SEQ ID NO: 155; or
one first chain comprising the amino acid sequence of SEQ ID NO: 178, one second chain comprising the amino acid sequence of SEQ ID NO: 179, one third chain comprising the amino acid sequence of SEQ ID NO: 154, and one fourth chain comprising the amino acid sequence of SEQ ID NO: 155; or
one first chain comprising the amino acid sequence of SEQ ID NO: 178, one second chain comprising the amino acid sequence of SEQ ID NO: 179, one third chain comprising the amino acid sequence of SEQ ID NO: 156, and one fourth chain comprising the amino acid sequence of SEQ ID NO: 155.

9. The antigen-binding molecule binding specifically to DLL3 and CD3 according to claim 7, wherein the antigen-binding molecule comprises one antigen-binding moiety binding specifically to DLL3 and one antigen-binding moiety binding specifically to CD3; the antigen-binding moiety binding specifically to DLL3 is a Fab, and the antigen-binding moiety binding specifically to CD3 is an scFv;
preferably,
the antigen-binding molecule binding specifically to DLL3 and CD3 comprises one first chain having a structure represented by formula (e), one second chain having a structure represented by formula (f), and one third chain having a structure represented by formula (g): 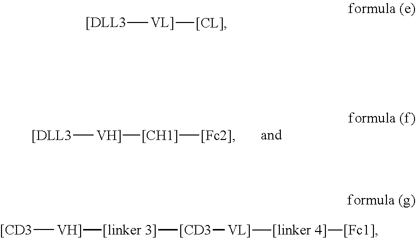
wherein the linker 3 and linker 4 are identical or different peptide linkers;
the structures represented by formulas (e), (f), and (g) are arranged from N-terminus to C-terminus;
the Fc1 and the Fc2 are interchangeable;
more preferably, the antigen-binding molecule binding specifically to DLL3 and CD3 comprises:
one first chain comprising the amino acid sequence of SEQ ID NO: 173, one second chain comprising the amino acid sequence of SEQ ID NO: 174, and one third chain comprising the amino acid sequence of SEQ ID NO: 160; or
one first chain comprising the amino acid sequence of SEQ ID NO: 173, one second chain comprising the amino acid sequence of SEQ ID NO: 174, and one third chain comprising the amino acid sequence of SEQ ID NO: 159; or
one first chain comprising the amino acid sequence of SEQ ID NO: 157, one second chain comprising the amino acid sequence of SEQ ID NO: 158, and one third chain comprising the amino acid sequence of SEQ ID NO: 159; or
one first chain comprising the amino acid sequence of SEQ ID NO: 157, one second chain comprising the amino acid sequence of SEQ ID NO: 158, and one third chain comprising the amino acid sequence of SEQ ID NO: 160; or
one first chain comprising the amino acid sequence of SEQ ID NO: 166, one second chain comprising the amino acid sequence of SEQ ID NO: 167, and one third chain comprising the amino acid sequence of SEQ ID NO: 159; or
one first chain comprising the amino acid sequence of SEQ ID NO: 166, one second chain comprising the amino acid sequence of SEQ ID NO: 167, and one third chain comprising the amino acid sequence of SEQ ID NO: 160; or
one first chain comprising the amino acid sequence of SEQ ID NO: 180, one second chain comprising the amino acid sequence of SEQ ID NO: 181, and one third chain comprising the amino acid sequence of SEQ ID NO: 159; or
one first chain comprising the amino acid sequence of SEQ ID NO: 180, one second chain comprising the amino acid sequence of SEQ ID NO: 181, and one third chain comprising the amino acid sequence of SEQ ID NO: 160.

10. The antigen-binding molecule binding specifically to DLL3 and CD3 according to claim 7, wherein the antigen-binding molecule comprises one antigen-binding moiety binding specifically to DLL3 and one antigen-binding moiety binding specifically to CD3;
the antigen-binding molecule binding specifically to DLL3 and CD3 comprises one first chain having a structure represented by formula (h) and one second chain having a structure represented by formula (i): 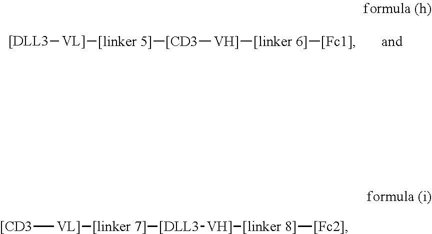
wherein the linker 5, linker 6, linker 7, and linker 8 are identical or different peptide linkers;
the structures represented by formulas (h) and (i) are arranged from N-terminus to C-terminus;
the Fc1 and the Fc2 are interchangeable;
preferably, wherein:
i) the DLL3-VH comprises the amino acid sequence of SEQ ID NO: 54, and the DLL3-VL comprises the amino acid sequence of SEQ ID NO: 63; and the CD3-VH comprises the amino acid sequence of SEQ ID NO: 136 or 138, and the CD3-VL comprises the amino acid sequence of SEQ ID NO: 137; or
the DLL3-VH comprises the amino acid sequence of SEQ ID NO: 54, and the DLL3-VL comprises the amino acid sequence of SEQ ID NO: 61; and the CD3-VH comprises the amino acid sequence of SEQ ID NO: 136 or 138, and the CD3-VL comprises the amino acid sequence of SEQ ID NO: 137; or
the DLL3-VH comprises the amino acid sequence of SEQ ID NO: 54, and the DLL3-VL comprises the amino acid sequence of SEQ ID NO: 64; and the CD3-VH comprises the amino acid sequence of SEQ ID NO: 136 or 138, and the CD3-VL comprises the amino acid sequence of SEQ ID NO: 137; or
the DLL3-VH comprises the amino acid sequence of SEQ ID NO: 56, and the DLL3-VL comprises the amino acid sequence of SEQ ID NO: 65; and the CD3-VH comprises the amino acid sequence of SEQ ID NO: 136 or 138, and the CD3-VL comprises the amino acid sequence of SEQ ID NO: 137; or
the DLL3-VH comprises the amino acid sequence of SEQ ID NO: 56, and the DLL3-VL comprises the amino acid sequence of SEQ ID NO: 66; and the CD3-VH comprises the amino acid sequence of SEQ ID NO: 136 or 138, and the CD3-VL comprises the amino acid sequence of SEQ ID NO: 137; or
the DLL3-VH comprises the amino acid sequence of SEQ ID NO: 56, and the DLL3-VL comprises the amino acid sequence of SEQ ID NO: 67; and the CD3-VH comprises the amino acid sequence of SEQ ID NO: 136 or 138, and the CD3-VL comprises the amino acid sequence of SEQ ID NO: 137; or
the DLL3-VH comprises the amino acid sequence of SEQ ID NO: 57, and the DLL3-VL comprises the amino acid sequence of SEQ ID NO: 65; and the CD3-VH comprises the amino acid sequence of SEQ ID NO: 136 or 138, and the CD3-VL comprises the amino acid sequence of SEQ ID NO: 137; or
the DLL3-VH comprises the amino acid sequence of SEQ ID NO: 57, and the DLL3-VL comprises the amino acid sequence of SEQ ID NO: 66; and the CD3-VH comprises the amino acid sequence of SEQ ID NO: 136 or 138, and the CD3-VL comprises the amino acid sequence of SEQ ID NO: 137; or
the DLL3-VH comprises the amino acid sequence of SEQ ID NO: 57, and the DLL3-VL comprises the amino acid sequence of SEQ ID NO: 67; and the CD3-VH comprises the amino acid sequence of SEQ ID NO: 136 or 138, and the CD3-VL comprises the amino acid sequence of SEQ ID NO: 137; or
ii) the DLL3-VH comprises the amino acid sequence of SEQ ID NO: 91, and the DLL3-VL comprises the amino acid sequence of SEQ ID NO: 87; and the CD3-VH comprises the amino acid sequence of SEQ ID NO: 136 or 138, and the CD3-VL comprises the amino acid sequence of SEQ ID NO: 137; or
iii) the DLL3-VH comprises the amino acid sequence of SEQ ID NO: 81, and the DLL3-VL comprises the amino acid sequence of SEQ ID NO: 77; and the CD3-VH comprises the amino acid sequence of SEQ ID NO: 136 or 138, and the CD3-VL comprises the amino acid sequence of SEQ ID NO: 137; or
iv) the DLL3-VH comprises the amino acid sequence of SEQ ID NO: 109, and the DLL3-VL comprises the amino acid sequence of SEQ ID NO: 100; and the CD3-VH comprises the amino acid sequence of SEQ ID NO: 136 or 138, and the CD3-VL comprises the amino acid sequence of SEQ ID NO: 137;
more preferably, the antigen-binding molecule binding specifically to DLL3 and CD3 comprises:
one first chain comprising the amino acid sequence of SEQ ID NO: 185 and one second chain comprising the amino acid sequence of SEQ ID NO: 162; or
one first chain comprising the amino acid sequence of SEQ ID NO: 161 and one second chain comprising the amino acid sequence of SEQ ID NO: 162; or
one first chain comprising the amino acid sequence of SEQ ID NO: 163 and one second chain comprising the amino acid sequence of SEQ ID NO: 162; or
one first chain comprising the amino acid sequence of SEQ ID NO: 259 and one second chain comprising the amino acid sequence of SEQ ID NO: 162; or
one first chain comprising the amino acid sequence of SEQ ID NO: 260 and one second chain comprising the amino acid sequence of SEQ ID NO: 162; or
one first chain comprising the amino acid sequence of SEQ ID NO: 168 and one second chain comprising the amino acid sequence of SEQ ID NO: 169; or
one first chain comprising the amino acid sequence of SEQ ID NO: 170 and one second chain comprising the amino acid sequence of SEQ ID NO: 169; or
one first chain comprising the amino acid sequence of SEQ ID NO: 175 and one second chain comprising the amino acid sequence of SEQ ID NO: 176; or
one first chain comprising the amino acid sequence of SEQ ID NO: 177 and one second chain comprising the amino acid sequence of SEQ ID NO: 176; or
one first chain comprising the amino acid sequence of SEQ ID NO: 182 and one second chain comprising the amino acid sequence of SEQ ID NO: 183; or
one first chain comprising the amino acid sequence of SEQ ID NO: 184 and one second chain comprising the amino acid sequence of SEQ ID NO: 183; or
one first chain comprising the amino acid sequence of SEQ ID NO: 186 and one second chain comprising the amino acid sequence of SEQ ID NO: 162; or
one first chain comprising the amino acid sequence of SEQ ID NO: 187 and one second chain comprising the amino acid sequence of SEQ ID NO: 188; or
one first chain comprising the amino acid sequence of SEQ ID NO: 187 and one second chain comprising the amino acid sequence of SEQ ID NO: 189; or
one first chain comprising the amino acid sequence of SEQ ID NO: 190 and one second chain comprising the amino acid sequence of SEQ ID NO: 188; or
one first chain comprising the amino acid sequence of SEQ ID NO: 190 and one second chain comprising the amino acid sequence of SEQ ID NO: 189; or
one first chain comprising the amino acid sequence of SEQ ID NO: 191 and one second chain comprising the amino acid sequence of SEQ ID NO: 188; or
one first chain comprising the amino acid sequence of SEQ ID NO: 191 and one second chain comprising the amino acid sequence of SEQ ID NO: 189.

11. An anti-DLL3 antibody capable of binding specifically to DLL3, comprising a heavy chain variable region DLL3-VH and a light chain variable region DLL3-VL, wherein:
i) the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 69 or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 24, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 21, 69, or 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
ii) the DLL3-VH comprises: a DLL3-HCDR1 comprising the amino acid sequence of SEQ ID NO: 26 or 84, a DLL3-HCDR2 comprising the amino acid sequence of SEQ ID NO: 85 or 27, and a DLL3-HCDR3 comprising the amino acid sequence of SEQ ID NO: 83 or 28; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
iii) the DLL3-VH comprises: a DLL3-HCDR1 comprising the amino acid sequence of SEQ ID NO: 20, a DLL3-HCDR2 comprising the amino acid sequence of SEQ ID NO: 96, 32, 97, 98, or 99, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; or
iv) the DLL3-VH comprises: a DLL3-HCDR1 comprising the amino acid sequence of SEQ ID NO: 20, a DLL3-HCDR2 comprising the amino acid sequence of SEQ ID NO: 119, 37, 117, 118, or 120, and a DLL3-HCDR3 comprising the amino acid sequence of SEQ ID NO: 38; and
the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40;
preferably,
i) the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 21, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 72, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 73, and a DLL3-LCDR3 comprising the amino acid sequence of SEQ ID NO: 74; or
the DLL3-VH comprises: a DLL3-HCDR1 comprising the amino acid sequence of SEQ ID NO: 20, a DLL3-HCDR2 comprising the amino acid sequence of SEQ ID NO: 69, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
the DLL3-VH comprises: a DLL3-HCDR1 comprising the amino acid sequence of SEQ ID NO: 20, a DLL3-HCDR2 comprising the amino acid sequence of SEQ ID NO: 70, and a DLL3-HCDR3 comprising the amino acid sequence of SEQ ID NO: 22; and the DLL3-VL comprises: a DLL3-LCDR1 comprising the amino acid sequence of SEQ ID NO: 23, a DLL3-LCDR2 comprising the amino acid sequence of SEQ ID NO: 71, and a DLL3-LCDR3 comprising the amino acid sequence of SEQ ID NO: 25; or
ii) the DLL3-VH comprises: a DLL3-HCDR1 comprising the amino acid sequence of SEQ ID NO: 26, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 84, a DLL3-HCDR2 comprising the amino acid sequence of SEQ ID NO: 27, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 84, a DLL3-HCDR2 comprising the amino acid sequence of SEQ ID NO: 85, and a DLL3-HCDR3 comprising the amino acid sequence of SEQ ID NO: 83; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
the DLL3-VH comprises: a DLL3-HCDR1 comprising the amino acid sequence of SEQ ID NO: 26, a DLL3-HCDR2 comprising the amino acid sequence of SEQ ID NO: 27, and a DLL3-HCDR3 comprising the amino acid sequence of SEQ ID NO: 28; and the DLL3-VL comprises: a DLL3-LCDR1 comprising the amino acid sequence of SEQ ID NO: 29, a DLL3-LCDR2 comprising the amino acid sequence of SEQ ID NO: 30, and a DLL3-LCDR3 comprising the amino acid sequence of SEQ ID NO: 31; or
iii) the DLL3-VH comprises: a DLL3-HCDR1 comprising the amino acid sequence of SEQ ID NO: 20, a DLL3-HCDR2 comprising the amino acid sequence of SEQ ID NO: 96, and a DLL3-HCDR3 comprising the amino acid sequence of SEQ ID NO: 33; and the DLL3-VL comprises: a DLL3-LCDR1 comprising the amino acid sequence of SEQ ID NO: 34, a DLL3-LCDR2 comprising the amino acid sequence of SEQ ID NO: 35, and a DLL3-LCDR3 comprising the amino acid sequence of SEQ ID NO: 36; or
iv) the DLL3-VH comprises: a DLL3-HCDR1 comprising the amino acid sequence of SEQ ID NO: 20, a DLL3-HCDR2 comprising the amino acid sequence of SEQ ID NO: 119, and a DLL3-HCDR3 comprising the amino acid sequence of SEQ ID NO: 38; and the DLL3-VL comprises: a DLL3-LCDR1 comprising the amino acid sequence of SEQ ID NO: 39, a DLL3-LCDR2 comprising the amino acid sequence of SEQ ID NO: 35, and a DLL3-LCDR3 comprising the amino acid sequence of SEQ ID NO: 40;
more preferably,
the anti-DLL3 antibody binds to human DLL3 at 25° C. with a KD of less than 4×10⁻⁹ M, as measured by surface plasmon resonance.

12. The anti-DLL3 antibody according to claim 11, comprising a heavy chain variable region DLL3-VH and a light chain variable region DLL3-VL, wherein:
i) the DLL3-VH comprises the amino acid sequence of SEQ ID NO: 54, 43, 44, 45, 46, 47, 48, 49, 50, 51, 52, 53, 55, 56, or 57, and the DLL3-VL comprises the amino acid sequence of SEQ ID NO: 63, 64, 65, 66, or 67; or
the DLL3-VH comprises the amino acid sequence of SEQ ID NO: 56 or 57, and the DLL3-VL comprises the amino acid sequence of SEQ ID NO: 58, 59, 60, 61, 62, 63, 64, 65, 66, 67, or 68; or
ii) the DLL3-VH comprises the amino acid sequence of SEQ ID NO: 81, 78, 79, 80, or 82, and the DLL3-VL comprises the amino acid sequence of SEQ ID NO: 77, 75, or 76; or
iii) the DLL3-VH comprises the amino acid sequence of SEQ ID NO: 91, 89, 90, 92, 93, 94, or 95, and the DLL3-VL comprises the amino acid sequence of SEQ ID NO: 87, 86, or 88;
or
iv) the DLL3-VH comprises the amino acid sequence of SEQ ID NO: 109, 105, 106, 107, 108, 110, 111, 112, 113, 114, 115, or 116, and the DLL3-VL comprises the amino acid sequence of SEQ ID NO: 100, 101, 102, 103, or 104;
preferably,
i) the DLL3-VH comprises the amino acid sequence of SEQ ID NO: 54, and the DLL3-VL comprises the amino acid sequence of SEQ ID NO: 63; or
the DLL3-VH comprises the amino acid sequence of SEQ ID NO: 54, and the DLL3-VL comprises the amino acid sequence of SEQ ID NO: 61; or
the DLL3-VH comprises the amino acid sequence of SEQ ID NO: 54, and the DLL3-VL comprises the amino acid sequence of SEQ ID NO: 64; or
the DLL3-VH comprises the amino acid sequence of SEQ ID NO: 56, and the DLL3-VL comprises the amino acid sequence of SEQ ID NO: 65; or
the DLL3-VH comprises the amino acid sequence of SEQ ID NO: 56, and the DLL3-VL comprises the amino acid sequence of SEQ ID NO: 66; or
the DLL3-VH comprises the amino acid sequence of SEQ ID NO: 56, and the DLL3-VL comprises the amino acid sequence of SEQ ID NO: 67; or
the DLL3-VH comprises the amino acid sequence of SEQ ID NO: 57, and the DLL3-VL comprises the amino acid sequence of SEQ ID NO: 65; or
the DLL3-VH comprises the amino acid sequence of SEQ ID NO: 57, and the DLL3-VL comprises the amino acid sequence of SEQ ID NO: 66; or
the DLL3-VH comprises the amino acid sequence of SEQ ID NO: 57, and the DLL3-VL comprises the amino acid sequence of SEQ ID NO: 67; or
ii) the DLL3-VH comprises the amino acid sequence of SEQ ID NO: 81, and the DLL3-VL comprises the amino acid sequence of SEQ ID NO: 77; or
iii) the DLL3-VH comprises the amino acid sequence of SEQ ID NO: 91, and the DLL3-VL comprises the amino acid sequence of SEQ ID NO: 87; or
iv) the DLL3-VH comprises the amino acid sequence of SEQ ID NO: 109, and the DLL3-VL comprises the amino acid sequence of SEQ ID NO: 100.

13. The anti-DLL3 antibody according to claim 11-er-12, wherein the anti-DLL3 antibody is an antibody fragment; preferably, the antibody fragment is a Fab, Fab′, F(ab′)2, Fd, Fv, scFv, dsFv, or dAb.

14. The anti-DLL3 antibody according to claim 11, comprising a heavy chain constant region and a light chain constant region, wherein preferably, the heavy chain constant region comprises the amino acid sequence of SEQ ID NO: 41, and/or the light chain constant region comprises the amino acid sequence of SEQ ID NO: 42.

15. The anti-DLL3 antibody according to claim 14, comprising a heavy chain and a light chain, wherein:
the heavy chain comprises an amino acid sequence having at least 85% sequence identity to SEQ ID NO: 121, and the light chain comprises an amino acid sequence having at least 85% sequence identity to SEQ ID NO: 122; or
the heavy chain comprises an amino acid sequence having at least 85% sequence identity to SEQ ID NO: 123, and the light chain comprises an amino acid sequence having at least 85% sequence identity to SEQ ID NO: 124; or
the heavy chain comprises an amino acid sequence having at least 85% sequence identity to SEQ ID NO: 125, and the light chain comprises an amino acid sequence having at least 85% sequence identity to SEQ ID NO: 126; or
the heavy chain comprises an amino acid sequence having at least 85% sequence identity to SEQ ID NO: 127, and the light chain comprises an amino acid sequence having at least 85% sequence identity to SEQ ID NO: 128;
preferably,
the heavy chain comprises the amino acid sequence of SEQ ID NO: 121, and the light chain comprises the amino acid sequence of SEQ ID NO: 122; or
the heavy chain comprises the amino acid sequence of SEQ ID NO: 123, and the light chain comprises the amino acid sequence of SEQ ID NO: 124; or
the heavy chain comprises the amino acid sequence of SEQ ID NO: 125, and the light chain comprises the amino acid sequence of SEQ ID NO: 126; or
the heavy chain comprises the amino acid sequence of SEQ ID NO: 127, and the light chain comprises the amino acid sequence of SEQ ID NO: 128.

16. A pharmaceutical composition, comprising:
a therapeutically effective amount of the antigen-binding molecule binding specifically to DLL3 and CD3 according to claim 1, and one or more pharmaceutically acceptable carriers, diluents, buffers, or excipients.

17. An isolated nucleic acid, wherein the isolated nucleic acid encodes the antigen-binding molecule binding specifically to DLL3 and CD3 according to claim 1.

18. A host cell, comprising the isolated nucleic acid according to claim 17.

19. A method for treating a tumor or cancer, comprising the step of administering to a subject in need thereof a therapeutically effective amount of the antigen-binding molecule binding specifically to DLL3 and CD3 according to claim 1, wherein preferably, the tumor or cancer is selected from the group consisting of:
head and neck squamous cell carcinoma, head and neck cancer, brain cancer, neuroglioma, glioblastoma multiforme, neuroblastoma, central nervous system cancer, neuroendocrine tumor, throat cancer, pharyngeal squamous cell carcinoma, oral squamous cell carcinoma, nasopharyngeal cancer, esophageal cancer, thyroid cancer, malignant pleural mesothelioma, lung cancer, breast cancer, liver cancer, hepatobiliary cancer, pancreatic cancer, gastric cancer, gastrointestinal cancer, intestinal cancer, colon cancer, colorectal cancer, renal cancer, clear cell renal cell carcinoma, ovarian cancer, endometrial cancer, cervical cancer, bladder cancer, prostate cancer, testicular cancer, skin cancer, melanoma, large cell lung cancer, triple-negative breast cancer, lymphoma, and small cell lung cancer;
more preferably, the tumor or cancer is a DLL3-expressing tumor or cancer.

20. A pharmaceutical composition, comprising:
a therapeutically effective amount of the anti-DLL3 antibody according to claim 11, and one or more pharmaceutically acceptable carriers, diluents, buffers, or excipients.

21. An isolated nucleic acid, wherein the isolated nucleic acid encodes the anti-DLL3 antibody according to claim 11.

22. A host cell, comprising the isolated nucleic acid according to claim 21.

23. A method for treating a tumor or cancer, comprising the step of administering to a subject in need thereof a therapeutically effective amount of the anti-DLL3 antibody according to claim 11, wherein preferably, the tumor or cancer is selected from the group consisting of:
head and neck squamous cell carcinoma, head and neck cancer, brain cancer, neuroglioma, glioblastoma multiforme, neuroblastoma, central nervous system cancer, neuroendocrine tumor, throat cancer, pharyngeal squamous cell carcinoma, oral squamous cell carcinoma, nasopharyngeal cancer, esophageal cancer, thyroid cancer, malignant pleural mesothelioma, lung cancer, breast cancer, liver cancer, hepatobiliary cancer, pancreatic cancer, gastric cancer, gastrointestinal cancer, intestinal cancer, colon cancer, colorectal cancer, renal cancer, clear cell renal cell carcinoma, ovarian cancer, endometrial cancer, cervical cancer, bladder cancer, prostate cancer, testicular cancer, skin cancer, melanoma, large cell lung cancer, triple-negative breast cancer, lymphoma, and small cell lung cancer;
more preferably, the tumor or cancer is a DLL3-expressing tumor or cancer.


SEQUENCE LISTING (260 sequences)

SEQ ID NO: 1 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 2 - protein, 618 aa, Macaca fascicularis
MVSPRMSRLLSQTVILALIFIPQARPAGVFELQIHSFGPGPGPGAPRSPCSARGPCRLFFRVCLKPGLSEEAAESPCALGAALSARGPVYTEQPEAPAPDLPLPNGLLQVPFRDAWPGTFSLIIETWREELGDQIGGPAWSLLARVTRRRRLAAGGPWARDIQRAGAWELRFSYRARCELPAVGTACTRLCRPRSAPSRCGPGLRPCAPLEDECEAPPVCRAGCSLEHGFCEQPGECRCLEGWTGPLCMVPVSTSSCLGLRGPSSTTTGCLVPGPGPCDGNPCANGGSCSETPGSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRNCRERADPCAARPCAHGGRCYAHFSGLVCACAPGYMGARCEFPVHPDGVSALPAAPPGLRPGDPQRYLLPPALGLLVAAGVAGAALLLVHVRRRGHAQDAGSRLLAGTPEPSVHALPDALNNLRTQEGPGDVPSSSVDWNRPEDVDSRGIYVISAPSIYAREVAMPLFPPLHTGRAGQRQNLLFPFPSSILSVK

SEQ ID NO: 3 - protein, 589 aa, Rattus norvegicus
MVSLQVSSLPQTLILAFLLPQALPAGVFELQIHSFGPGPGPGTPRSPCNARGPCRLFFRVCLKPGVSQEAAESLCALGAALSTSGPVYTEQPGVPAAALSLPDGLVRVPFLDAWPGTFSLIIETWREQLGERAAGPAWNLLARVAGRRRLAAGAPWARDVQRTGAWELHFSYRARCEPPAVGAACARLCRSRSAPSRCGPGLRPCTPFPDECEAPRESLTVCRAGCSPEHGYCEEPDECHCLEGWTGPLCTVPVSTSSCLNSRVSGPAGTGCLLPGPGPCDGNPCANGGSCSETPGSFECACPRGFYGPRCEVSGVTCADGPCFNGGLCVGGEDPDSAYVCHCPPAFQGSNCERRVDRCSLQPCQNGGLCLDLGHALRCRCRAGFAGPRCEHDLDDCAGRACANGGTCVEGGGARRCSCALGFGGRDCRERADPCASRPCAHGGRCYAHFSGLVCACAPGYMGVRCEFAVRPDGADAVPAAPRGLRQADSQRFLLPPALGLLAAAALAGAALLLIHVRRRGPGRDTGTRLLSGTREPSVHTLPDALNNLRLQDGAGDGPTSSADWNHPEDGDSRSIYVIPAPSIYAREA

SEQ ID NO: 4 - protein, 592 aa, Mus musculus
MVSLQVSPLSQTLILAFLLPQALPAGVFELQIHSFGPGPGLGTPRSPCNARGPCRLFFRVCLKPGVSQEATESLCALGAALSTSVPVYTEHPGESAAALPLPDGLVRVPFRDAWPGTFSLVIETWREQLGEHAGGPAWNLLARVVGRRRLAAGGPWARDVQRTGTWELHFSYRARCEPPAVGAACARLCRSRSAPSRCGPGLRPCTPFPDECEAPSVCRPGCSPEHGYCEEPDECRCLEGWTGPLCTVPVSTSSCLNSRVPGPASTGCLLPGPGPCDGNPCANGGSCSETSGSFECACPRGFYGLRCEVSGVTCADGPCFNGGLCVGGEDPDSAYVCHCPPGFQGSNCEKRVDRCSLQPCQNGGLCLDLGHALRCRCRAGFAGPRCEHDLDDCAGRACANGGTCVEGGGSRRCSCALGFGGRDCRERADPCASRPCAHGGRCYAHFSGLVCACAPGYMGVRCEFAVRPDGADAVPAAPRGLRQADPQRFLLPPALGLLVAAGLAGAALLVIHVRRRGPGQDTGTRLLSGTREPSVHTLPDALNNLRLQDGAGDGPSSSADWNHPEDGDSRSIYVIPAPSIYAREDWLIQVLF

SEQ ID NO: 5 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 6 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 7 - protein, 507 aa, synthetic construct
  REGION 1..507: note = His-hDLL3(ECD)
METDTLLLWVLLLWVPGSTGHHHHHHHHHHGGGGGSGGGGSAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYL

SEQ ID NO: 8 - protein, 727 aa, synthetic construct
  REGION 1..727: note = Fc-hDLL3(ECD)
METDTLLLWVLLLWVPGSTGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYL

SEQ ID NO: 9 - protein, 514 aa, synthetic construct
  REGION 1..514: note = hDLL3(ECD)-strep twin
METDTLLLWVLLLWVPGSTGAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLWSHPQFEKGGGSGGGSGGSAWSHPQFEK

SEQ ID NO: 10 - protein, 514 aa, synthetic construct
  REGION 1..514: note = cynoDLL3(ECD)-strep twin
METDTLLLWVLLLWVPGSTGAGVFELQIHSFGPGPGPGAPRSPCSARGPCRLFFRVCLKPGLSEEAAESPCALGAALSARGPVYTEQPEAPAPDLPLPNGLLQVPFRDAWPGTFSLIIETWREELGDQIGGPAWSLLARVTRRRRLAAGGPWARDIQRAGAWELRFSYRARCELPAVGTACTRLCRPRSAPSRCGPGLRPCAPLEDECEAPPVCRAGCSLEHGFCEQPGECRCLEGWTGPLCMVPVSTSSCLGLRGPSSTTTGCLVPGPGPCDGNPCANGGSCSETPGSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRNCRERADPCAARPCAHGGRCYAHFSGLVCACAPGYMGARCEFPVHPDGVSALPAAPPGLRPGDPQRYLWSHPQFEKGGGSGGGSGGSAWSHPQFEK

SEQ ID NO: 11 - protein, 514 aa, synthetic construct
  REGION 1..514: note = mouDLL3(ECD)-strep twin
METDTLLLWVLLLWVPGSTGAGVFELQIHSFGPGPGLGTPRSPCNARGPCRLFFRVCLKPGVSQEATESLCALGAALSTSVPVYTEHPGESAAALPLPDGLVRVPFRDAWPGTFSLVIETWREQLGEHAGGPAWNLLARVVGRRRLAAGGPWARDVQRTGTWELHFSYRARCEPPAVGAACARLCRSRSAPSRCGPGLRPCTPFPDECEAPSVCRPGCSPEHGYCEEPDECRCLEGWTGPLCTVPVSTSSCLNSRVPGPASTGCLLPGPGPCDGNPCANGGSCSETSGSFECACPRGFYGLRCEVSGVTCADGPCFNGGLCVGGEDPDSAYVCHCPPGFQGSNCEKRVDRCSLQPCQNGGLCLDLGHALRCRCRAGFAGPRCEHDLDDCAGRACANGGTCVEGGGSRRCSCALGFGGRDCRERADPCASRPCAHGGRCYAHFSGLVCACAPGYMGVRCEFAVRPDGADAVPAAPRGLRQADPQRFLWSHPQFEKGGGSGGGSGGSAWSHPQFEK

SEQ ID NO: 12 - protein, 120 aa, synthetic construct
  REGION 1..120: note = mAb6
EVQLQQSGPVLVKPGASVKMSCKASGYTFTDYYMNWVKQSHGKSLEWIGIINPYSGVTIYNHKFKGKATLTVDKSSNTAYMELNSLTSEDSAVYYCARQDSLLDYAMDYWGQGTSVTVSS

SEQ ID NO: 13 - protein, 107 aa, synthetic construct
  REGION 1..107: note = mAb6
DILMTQSPSSMSVSLGDTVSITCHASQGISGNMGWLQQKPGKSFKGLIYHGANLEDGVPSRFSGSGSGADYSLTISSLESEDFADYYCVQYAQFPYTFGGGTKLEIK

SEQ ID NO: 14 - protein, 121 aa, synthetic construct
  REGION 1..121: note = mAb98
EVQLVESGGGLVKPGGSLKLSCAASGFTFSDYGIHWVRQAPEKGLEWVAYISSGGYTIYYADTVKGRFTISRDNAKNTLFLQMTSLRSEDTAMYYCARGDYDDIFYTMDYWGQGTSVTVSS

SEQ ID NO: 15 - protein, 107 aa, synthetic construct
  REGION 1..107: note = mAb98
DIQMTQTTSSLSASLGDRVTISCRASRDITKFLNWYQQKPDGTVKLLIYYTSRLHSGVPSRFSGSGSGTDYSLTISNLDQEDIATYFCQQGNTLPWTFGGGTKLDIK

SEQ ID NO: 16 - protein, 124 aa, synthetic construct
  REGION 1..124: note = mAb110
EVQLQQSGPVLVKPGASVKMSCKTSGYTFTDYYMNWVKQSHGKSLEWIGIINPYNGGTSYTQKFKGKATLTVDKSSSTAYMELNSLTSEDSTVYFCARHFYDSSYLTYSTMDYWGQGTSVTVSS

SEQ ID NO: 17 - protein, 107 aa, synthetic construct
  REGION 1..107: note = mAb110
NIVMTQSPKSMSMSVGERVTLSCKASENVGIFVSWYQQKPKQSPKLLIYGASNRYTGVPDRFTGSGSATDFTLTISSVQAEDLADYYCGQSHSYPLTFGTGTKLELK

SEQ ID NO: 18 - protein, 122 aa, synthetic construct
  REGION 1..122: note = mAb30
EVRLQQFGPVLVKPGASVKMSCKASGYTFTDYYMNWVKQSHGKSLEWIGIINPYNGDTSYNQKFRGKATLTVDKSSSTAYMELNSLTSEDSAVYYCARYHYGPHLEGYFDVWGTGTTLTVSS

SEQ ID NO: 19 - protein, 106 aa, synthetic construct
  REGION 1..106: note = mAb30
DIVMTQSPKSMSMSVGERVTLSCKASENVGYVSWYQQKPEQSPKLLIYGASNRYTGVPDRFTGSGSATDFTLTISSVQAEDLADYHCGQSYSYPLTFGSGTKLEIK

SEQ ID NO: 20 - protein, 5 aa, Mus musculus
  REGION 1..5: note = mAb6 mAb110 mAb30 HCDR1
DYYMN

SEQ ID NO: 21 - protein, 17 aa, Mus musculus
  REGION 1..17: note = mAb6 HCDR2
IINPYSGVTIYNHKFKG

SEQ ID NO: 22 - protein, 11 aa, Mus musculus
  REGION 1..11: note = mAb6 HCDR3
QDSLLDYAMDY

SEQ ID NO: 23 - protein, 11 aa, Mus musculus
  REGION 1..11: note = mAb6 LCDR1
HASQGISGNMG

SEQ ID NO: 24 - protein, 7 aa, Mus musculus
  REGION 1..7: note = mAb6 LCDR2
HGANLED

SEQ ID NO: 25 - protein, 9 aa, Mus musculus
  REGION 1..9: note = mAb6 LCDR3
VQYAQFPYT

SEQ ID NO: 26 - protein, 5 aa, Mus musculus
  REGION 1..5: note = mAb98 HCDR1
DYGIH

SEQ ID NO: 27 - protein, 17 aa, Mus musculus
  REGION 1..17: note = mAb98 HCDR2
YISSGGYTIYYADTVKG

SEQ ID NO: 28 - protein, 12 aa, Mus musculus
  REGION 1..12: note = mAb98 HCDR3
GDYDDIFYTMDY

SEQ ID NO: 29 - protein, 11 aa, Mus musculus
  REGION 1..11: note = mAb98 LCDR1
RASRDITKFLN

SEQ ID NO: 30 - protein, 7 aa, Mus musculus
  REGION 1..7: note = mAb98 LCDR2
YTSRLHS

SEQ ID NO: 31 - protein, 9 aa, Mus musculus
  REGION 1..9: note = mAb98 LCDR3
QQGNTLPWT

SEQ ID NO: 32 - protein, 17 aa, Mus musculus
  REGION 1..17: note = mAb110 HCDR2
IINPYNGGTSYTQKFKG

SEQ ID NO: 33 - protein, 15 aa, Mus musculus
  REGION 1..15: note = mAb110 HCDR3
HFYDSSYLTYSTMDY

SEQ ID NO: 34 - protein, 11 aa, Mus musculus
  REGION 1..11: note = mAb110 LCDR1
KASENVGIFVS

SEQ ID NO: 35 - protein, 7 aa, Mus musculus
  REGION 1..7: note = mAb110 mAb30 LCDR2
GASNRYT

SEQ ID NO: 36 - protein, 9 aa, Mus musculus
  REGION 1..9: note = mAb110 LCDR3
GQSHSYPLT

SEQ ID NO: 37 - protein, 17 aa, Mus musculus
  REGION 1..17: note = mAb30 HCDR2
IINPYNGDTSYNQKFRG

SEQ ID NO: 38 - protein, 13 aa, Mus musculus
  REGION 1..13: note = mAb30 HCDR3
YHYGPHLEGYFDV

SEQ ID NO: 39 - protein, 10 aa, Mus musculus
  REGION 1..10: note = mAb30 LCDR1
KASENVGYVS

SEQ ID NO: 40 - protein, 9 aa, Mus musculus
  REGION 1..9: note = mAb30 LCDR3
GQSYSYPLT

SEQ ID NO: 41 - protein, 330 aa, Homo sapiens [sequence omitted]

SEQ ID NO: 42 - protein, 107 aa, Homo sapiens [sequence omitted]

SEQ ID NO: 43 - protein, 120 aa, synthetic construct
  REGION 1..120: note = hu6 VH1
EVQLVQSGAEVKKPGASVKVSCKASGYTFTDYYMNWVRQAPGQRLEWMGIINPYSGVTIYNHKFKGRVTITVDKSASTAYMELSSLRSEDTAVYYCARQDSLLDYAMDYWGQGTTVTVSS

SEQ ID NO: 44 - protein, 120 aa, synthetic construct
  REGION 1..120: note = hu6 VH2
EVQLVQSGAEVKKPGASVKVSCKASGYTFTDYYMNWVRQAPGQRLEWMGIINPYSGVTIYNHKFKGRVTLTVDKSANTAYMELSSLRSEDTAVYYCARQDSLLDYAMDYWGQGTTVTVSS

SEQ ID NO: 45 - protein, 120 aa, synthetic construct
  REGION 1..120: note = hu6 VH3
EVQLVQSGAEVKKPGASVKVSCKASGYTFTDYYMNWVRQAPGQRLEWIGIINPYSGVTIYNHKFKGRATLTVDKSANTAYMELSSLRSEDTAVYYCARQDSLLDYAMDYWGQGTTVTVSS

SEQ ID NO: 46 - protein, 120 aa, synthetic construct
  REGION 1..120: note = hu6 VH4
EVQLVQSGAEVKKPGASVKVSCKASGYTFTDYYMNWVRQAPGKRLEWMGIINPYSGVTIYNHKFKGRVTLTVDKSANTAYMELSSLRSEDTAVYYCARQDSLLDYAMDYWGQGTTVTVSS

SEQ ID NO: 47 - protein, 120 aa, synthetic construct
  REGION 1..120: note = hu6 VH5
EVQLVQSGSELKKPGASVKVSCKASGYTFTDYYMNWVRQAPGQGLEWMGIINPYSGVTIYNHKFKGRFVLSVDKSVSTAYLQISSLKAEDTAVYYCARQDSLLDYAMDYWGQGTTVTVSS

SEQ ID NO: 48 - protein, 120 aa, synthetic construct
  REGION 1..120: note = hu6 VH6
EVQLVQSGSELKKPGASVKVSCKASGYTFTDYYMNWVRQAPGQGLEWMGIINPYSGVTIYNHKFKGRFVLSVDKSSNTAYLQISSLKAEDTAVYYCARQDSLLDYAMDYWGQGTTVTVSS

SEQ ID NO: 49 - protein, 120 aa, synthetic construct
  REGION 1..120: note = hu6 VH7
EVQLVESGGGLVQPGGSLKLSCAASGYTFTDYYMNWVRQASGKGLEWVGIINPYSGVTIYNHKFKGRFTLSVDKSKNTAYLQMNSLKTEDTAVYYCARQDSLLDYAMDYWGQGTTVTVSS

SEQ ID NO: 50 - protein, 120 aa, synthetic construct
  REGION 1..120: note = hu6VH8
QVQLVQSGAEVKKPGASVKVSCKASGYTFTDYYMNWVRQAPGQRLEWMGIINPYSGVTIYNHKFKGRVTITRDTSASTAYMELSSLRSEDTAVYYCARQDSLLDYAMDYWGQGTTVTVSS

SEQ ID NO: 51 - protein, 120 aa, synthetic construct
  REGION 1..120: note = hu6VH9
QVQLVQSGSELKKPGASVKVSCKASGYTFTDYYMNWVRQAPGQGLEWMGIINPYSGVTIYNHKFKGRFVFSLDTSVSTAYLQISSLKAEDTAVYYCARQDSLLDYAMDYWGQGTTVTVSS

SEQ ID NO: 52 - protein, 120 aa, synthetic construct
  REGION 1..120: note = hu6VH10
EVQLVESGGGLVQPGGSLKLSCAASGFTFSDYYMNWVRQASGKGLEWVGIINPYSGVTIYNHKFKGRFTISRDDSKNTAYLQMNSLKTEDTAVYYCTRQDSLLDYAMDYWGQGTTVTVSS

SEQ ID NO: 53 - protein, 120 aa, synthetic construct
  REGION 1..120: note = hu6VH11
EVQLVQSGSELKKPGASVKVSCKASGYTFTDYYMNWVKQAPGQGLEWMGIINPYSGVTIYNHKFKGRFVLSVDKSSNTAYLQISSLKAEDTAVYYCARQDSLLDYAMDYWGQGTTVTVSS

SEQ ID NO: 54 - protein, 120 aa, synthetic construct
  REGION 1..120: note = hu6VH12
EVQLVQSGSELKKPGASVKVSCKASGYTFTDYYMNWVRQAPGQGLEWMGIINPYSGVTIYNHKFKGRFALSVDKSSNTAYLQISSLKAEDTAVYYCARQDSLLDYAMDYWGQGTTVTVSS

SEQ ID NO: 55 - protein, 120 aa, synthetic construct
  REGION 1..120: note = hu6VH13
EVQLVQSGSELKKPGASVKVSCKASGYTFTDYYMNWVKQAPGQGLEWMGIINPYSGVTIYNHKFKGRFALSVDKSSNTAYLQISSLKAEDTAVYYCARQDSLLDYAMDYWGQGTTVTVSS

SEQ ID NO: 56 - protein, 120 aa, synthetic construct
  REGION 1..120: note = hu6VH14
EVQLVQSGSELKKPGASVKVSCKASGYTFTDYYMNWVRQAPGQGLEWMGIINPYSGVTTYNHKFKGRFALSVDKSSNTAYLQISSLKAEDTAVYYCARQDSLLDYAMDYWGQGTTVTVSS

SEQ ID NO: 57 - protein, 120 aa, synthetic construct
  REGION 1..120: note = hu6VH15
EVQLQQSGSELKKPGASVKVSCKASGYTFTDYYMNWVRQAPGQGLEWMGIINPYSGSTIYNHKFKGRFALSVDKSSNTAYLQISSLKAEDTAVYYCARQDSLLDYAMDYWGQGTTVTVSS

SEQ ID NO: 58 - protein, 107 aa, synthetic construct
  REGION 1..107: note = hu6 VL1
DIQMTQSPSSLSASVGDRVTITCHASQGISGNMGWLQQKPGKAPKGLIYHGANLEDGVPSRFSGSGSGTDFTLTISSLQPEDFATYYCVQYAQFPYTFGGGTKVEIK

SEQ ID NO: 59 - protein, 107 aa, synthetic construct
  REGION 1..107: note = hu6 VL2
DIQMTQSPSSLSASVGDRVTITCHASQGISGNMGWLQQKPGKAPKGLIYHGANLEDGVPSRFSGSGSGADYTLTISSLQPEDFATYYCVQYAQFPYTFGGGTKVEIK

SEQ ID NO: 60 - protein, 107 aa, synthetic construct
  REGION 1..107: note = hu6 VL3
DIQMTQSPSSLSASVGDRVTITCHASQGISGNMGWLQQKPGKSFKGLIYHGANLEDGVPSRFSGSGSGADYTLTISSLQPEDFATYYCVQYAQFPYTFGGGTKVEIK

SEQ ID NO: 61 - protein, 107 aa, synthetic construct
  REGION 1..107: note = hu6 VL4
DIQMTQSPSSLSASVGDRVTITCHASQGISGNMGWLQQKPGKAPKGLIYHGANLEDGVPSRFSGSGSGADYTLTISSLQPEDFADYYCVQYAQFPYTFGGGTKVEIK

SEQ ID NO: 62 - protein, 107 aa, synthetic construct
  REGION 1..107: note = hu6VL5
DIQMTQSPSSLSASVGDRVTITCHASQGISGNMGWFQQKPGKAPKSLIYHGANLEDGVPSRFSGSGSGTDFTLTISSLQPEDFATYYCVQYAQFPYTFGGGTKVEIK

SEQ ID NO: 63 - protein, 107 aa, synthetic construct
  REGION 1..107: note = hu6VL6
DIQMTQSPSSLSASVGDRVTITCHASQGISGNMGWLQQKPGKAPKGLIYHGANLEEGVPSRFSGSGSGADYTLTISSLQPEDFADYYCVQYAQFPYTFGGGTKVEIK

SEQ ID NO: 64 - protein, 107 aa, synthetic construct
  REGION 1..107: note = hu6VL7
DIQMTQSPSSLSASVGDRVTITCHASQGISGNMGWLQQKPGKAPKGLIYHGANLESGVPSRFSGSGSGADYTLTISSLQPEDFADYYCVQYAQFPYTFGGGTKVEIK

SEQ ID NO: 65 - protein, 107 aa, synthetic construct
  REGION 1..107: note = hu6VL8
DIQMTQSPSSLSASVGDRVTITCHASQGISGNMGWLQQKPGKAPKGLIYHGANLEEGVPSRFSGSGSGADYTLTISSLQPEDFADYYCVQYAQYPYTFGGGTKVEIK

SEQ ID NO: 66 - protein, 107 aa, synthetic construct
  REGION 1..107: note = hu6VL9
NIQMTQSPSSLSASVGDRVTITCHASQGISGNMGWLQQKPGKAPKGLIYHGRNLEEGVPSRFSGSGSGADYTLTISSLQPEDFADYYCVQYAQYPYTFGGGTKVEIK

SEQ ID NO: 67 - protein, 107 aa, synthetic construct
  REGION 1..107: note = hu6VL10
EIVLTQSPATLSLSPGERATLSCHASQGISGNMGWLQQKPGKAPRGLIYHGANLEEGIPARFSGSGSGADYTLTISRLQPEDFADYYCVQYAQFPYTFGGGTKVEIK

SEQ ID NO: 68 - protein, 107 aa, synthetic construct
  REGION 1..107: note = hu6VL11
EIVLTQSPATLSLSPGERATLSCHASQGISGNMGWYQQKPGQAPRLLIYHGANLEEGIPARFSGSGSGTDFTLTISRLEPEDFAVYYCVQYAQFPYTFGGGTKVEIK

SEQ ID NO: 69 - protein, 16 aa, synthetic construct
  REGION 1..16: note = hu6VH14 HCDR2
INPYSGVTTYNHKFKG

SEQ ID NO: 70 - protein, 16 aa, synthetic construct
  REGION 1..16: note = hu6VH15 HCDR2
INPYSGSTIYNHKFKG

SEQ ID NO: 71 - protein, 7 aa, synthetic construct
  REGION 1..7: note = hu6VL6/VL8/VL10 LCDR2
HGANLEE

SEQ ID NO: 72 - protein, 7 aa, synthetic construct
  REGION 1..7: note = hu6VL7 LCDR2
HGANLES

SEQ ID NO: 73 - protein, 7 aa, synthetic construct
  REGION 1..7: note = hu6VL9 LCDR2
HGRNLEE

SEQ ID NO: 74 - protein, 9 aa, synthetic construct
  REGION 1..9: note = hu6VL8/VL9 LCDR3
VQYAQYPYT

SEQ ID NO: 75 - protein, 107 aa, synthetic construct
  REGION 1..107: note = hu98VL1
DIQMTQSPSSLSASVGDRVTITCRASRDITKFLNWYQQKPGKAPKLLIYYTSRLHSGVPSRFSGSGSGTDFTLTISSLQPEDFATYYCQQGNTLPWTFGGGTKVEIK

SEQ ID NO: 76 - protein, 107 aa, synthetic construct
  REGION 1..104: note = hu98VL2
DIQMTQSPSSLSASVGDRVTITCRASRDITKFLNWYQQKPGKAVKLLIYYTSRLHSGVPSRFSGSGSGTDYTLTISSLQPEDFATYYCQQGNTLPWTFGGGTKVEIK

SEQ ID NO: 77 - protein, 107 aa, synthetic construct
  REGION 1..107: note = hu98VL3
DIQMTQSPSSLSASVGDRVTITCRASRDITKFLNWYQQKPGGAVKLLIYYTSRLHSGVPSRFSGSGSGTDYTLTISSLQPEDFATYYCQQGNTLPWTFGGGTKVEIK

SEQ ID NO: 78 - protein, 121 aa, synthetic construct
  REGION 1..121: note = hu98VH1
EVQLVESGGGLVQPGGSLRLSCAASGFTFSDYGIHWVRQAPGKGLEWVAYISSGGYTIYYADTVKGRFTISRDNAKNSLYLQMNSLRAEDTAVYYCARGDYDDIFYTMDYWGQGTTVTVSS

SEQ ID NO: 79 - protein, 121 aa, synthetic construct
  REGION 1..121: note = hu98VH2
EVQLVESGGGLVQPGGSLRLSCAASGFTFSDYGIHWVRQAPGKGLEWVAYISSGGYTIYYADTVKGRFTISRDNAKNSLYLQMNSLRAEDTAVYYCARGDYEDIFYTMDYWGQGTTVTVSS

SEQ ID NO: 80 - protein, 121 aa, synthetic construct
  REGION 1..121: note = hu98VH3
EVQLVESGGGLVQPGGSLRLSCAASGFTFSDYGMHWVRQAPGKGLEWVAYISSGGYTIYYADTVKGRFTISRDNAKNSLYLQMNSLRAEDTAVYYCARGDYEDIFYTMDYWGQGTTVTVSS

SEQ ID NO: 81 - protein, 121 aa, synthetic construct
  REGION 1..121: note = hu98VH4
EVQLVESGGGLVQPGGSLRLSCAASGFTFSDYGIHWVRQAPGKGLEWVAYISSGGYTSYYADTVKGRFTISRDNAKNSLYLQMNSLRAEDTAVYYCARGDYEDIFYTMDYWGQGTTVTVSS

SEQ ID NO: 82 - protein, 121 aa, synthetic construct
  REGION 1..121: note = hu98VH5
EVQLVESGGGLVQPGGSLRLSCAASGFTFSDYGMHWVRQAPGKGLEWVAYISSGGYTSYYADTVKGRFTISRDNAKNSLYLQMNSLRAEDTAVYYCARGDYEDIFYTMDYWGQGTTVTVSS

SEQ ID NO: 83 - protein, 12 aa, synthetic construct
  REGION 1..12: note = hu98VH2/VH3/VH4/VH5 HCDR3
GDYEDIFYTMDY

SEQ ID NO: 84 - protein, 5 aa, synthetic construct
  REGION 1..5: note = hu98VH3/VH5 HCDR1
DYGMH

SEQ ID NO: 85 - protein, 17 aa, synthetic construct
  REGION 1..17: note = hu98VH4/VH5 HCDR2
YISSGGYTSYYADTVKG

SEQ ID NO: 86 - protein, 107 aa, synthetic construct
  REGION 1..107: note = hu110VL1
DIVMTQSPDSLAVSLGERATINCKASENVGIFVSWYQQKPGQSPKLLIYGASNRYTGVPDRFSGSGSATDFTLTISSLQAEDVAVYYCGQSHSYPLTFGQGTKLEIK

SEQ ID NO: 87 - protein, 107 aa, synthetic construct
  REGION 1..107: note = hu110VL2
NIVMTQSPKSLAVSLGERATINCKASENVGIFVSWYQQKPGQSPKLLIYGASNRYTGVPDRFSGSGSATDFTLTISSLQAEDVADYYCGQSHSYPLTFGQGTKLEIK

SEQ ID NO: 88 - protein, 107 aa, synthetic construct
  REGION 1..107: note = hu110VL3
DIVMTQSPDSLAVSLGERATINCKASENVGIFVSWYQQKPGQPPKLLIYGASNRYTGVPDRFSGSGSGTDFTLTISSLQAEDVAVYYCGQSHSYPLTFGQGTKLEIK

SEQ ID NO: 89 - protein, 124 aa, synthetic construct
  REGION 1..124: note = hu110VH1
EVQLVQSGAEVKKPGASVKVSCKASGYTFTDYYMNWVRQAPGQRLEWMGIINPYNGGTSYTQKFKGRVTITVDKSASTAYMELSSLRSEDTAVYYCARHFYDSSYLTYSTMDYWGQGTTVTVSS

SEQ ID NO: 90 - protein, 124 aa, synthetic construct
  REGION 1..124: note = hu110VH2
EVQLVQSGAEVKKPGASVKVSCKTSGYTFTDYYMNWVRQAPGQSLEWMGIINPYNGGTSYTQKFKGRVTITVDKSASTAYMELSSLRSEDTAVYYCARHFYDSSYLTYSTMDYWGQGTTVTVSS

SEQ ID NO: 91 - protein, 124 aa, synthetic construct
  REGION 1..124: note = hu110VH3
EVQLVQSGAEVKKPGASVKVSCKTSGYTFTDYYMNWVRQAPGQSLEWMGIINPYQGGTSYTQKFKGRVTITVDKSASTAYMELSSLRSEDTAVYYCARHFYDSSYLTYSTMDYWGQGTTVTVSS

SEQ ID NO: 92 - protein, 124 aa, synthetic construct
  REGION 1..124: note = hu110VH4
EVQLVQSGAEVKKPGASVKVSCKTSGYTFTDYYMNWVRQAPGQSLEWMGIINPYSGGTSYTQKFKGRVTITVDKSASTAYMELSSLRSEDTAVYYCARHFYDSSYLTYSTMDYWGQGTTVTVSS

SEQ ID NO: 93 - protein, 124 aa, synthetic construct
  REGION 1..124: note = hu110VH5
EVQLVQSGAEVKKPGASVKVSCKTSGYTFTDYYMNWVRQAPGQSLEWMGIINPYTGGTSYTQKFKGRVTITVDKSASTAYMELSSLRSEDTAVYYCARHFYDSSYLTYSTMDYWGQGTTVTVSS

SEQ ID NO: 94 - protein, 124 aa, synthetic construct
  REGION 1..124: note = hu110VH6
EVQLVQSGAEVKKPGASVKVSCKTSGYTFTDYYMNWVRQAPGQSLEWMGIINPYNVGTSYTQKFKGRVTITVDKSASTAYMELSSLRSEDTAVYYCARHFYDSSYLTYSTMDYWGQGTTVTVSS

SEQ ID NO: 95 - protein, 124 aa, synthetic construct
  REGION 1..124: note = hu110VH7
QVQLVQSGAEVKKPGASVKVSCKASGYTFTDYYMNWVRQAPGQRLEWMGIINPYNGGTSYTQKFKGRVTITRDTSASTAYMELSSLRSEDTAVYYCARHFYDSSYLTYSTMDYWGQGTTVTVSS

SEQ ID NO: 96 - protein, 17 aa, synthetic construct
  REGION 1..17: note = hu110VH3 HCDR2
IINPYQGGTSYTQKFKG

SEQ ID NO: 97 - protein, 17 aa, synthetic construct
  REGION 1..17: note = hu110VH4 HCDR2
IINPYSGGTSYTQKFKG

SEQ ID NO: 98 - protein, 17 aa, synthetic construct
  REGION 1..17: note = hu110VH5 HCDR2
IINPYTGGTSYTQKFKG

SEQ ID NO: 99 - protein, 17 aa, synthetic construct
  REGION 1..17: note = hu110VH6 HCDR2
IINPYNVGTSYTQKFKG

SEQ ID NO: 100 - protein, 106 aa, synthetic construct
  REGION 1..106: note = hu30VL1
DIVMTQSPDSLAVSLGERATINCKASENVGYVSWYQQKPGQPPKLLIYGASNRYTGVPDRFSGSGSGTDFTLTISSLQAEDVAVYYCGQSYSYPLTFGQGTKLEIK

SEQ ID NO: 101 - protein, 106 aa, synthetic construct
  REGION 1..106: note = hu30VL2
DIVMTQSPDSLAVSLGERATINCKASENVGYVSWYQQKPGQPPKLLIYGASNRYTGVPDRFSGSGSATDFTLTISSLQAEDVAVYHCGQSYSYPLTFGQGTKLEIK

SEQ ID NO: 102 - protein, 106 aa, synthetic construct
  REGION 1..106: note = hu30VL3
DIVMTQSPKSLAVSLGERATINCKASENVGYVSWYQQKPGQSPKLLIYGASNRYTGVPDRFSGSGSATDFTLTISSLQAEDVAVYHCGQSYSYPLTFGQGTKLEIK

SEQ ID NO: 103 - protein, 106 aa, synthetic construct
  REGION 1..106: note = hu30VL4
DIQMTQSPSSLSASVGDRVTITCKASENVGYVSWYQQKPGKAPKLLIYGASNRYTGVPSRFSGSGSGTDFTLTISSLQPEDFATYYCGQSYSYPLTFGQGTKLEIK

SEQ ID NO: 104 - protein, 106 aa, synthetic construct
  REGION 1..106: note = hu30VL5
DIQMTQSPSSLSASVGDRVTITCKASENVGYVSWYQQKPGKSPKLLIYGASNRYTGVPDRFSGSGSGTDFTLTISSLQPEDFATYYCGQSYSYPLTFGSGTKLEIK

SEQ ID NO: 105 - protein, 122 aa, synthetic construct
  REGION 1..122: note = hu30VH1
EVQLVQSGAEVKKPGASVKVSCKASGYTFTDYYMNWVRQAPGQSLEWMGIINPYNGDTSYNQKFRGRVTITVDKSASTAYMELSSLRSEDTAVYYCARYHYGPHLEGYFDVWGQGTTVTVSS

SEQ ID NO: 106 - protein, 122 aa, synthetic construct
  REGION 1..122: note = hu30VH2
EVRLVQSGAEVKKPGASVKVSCKASGYTFTDYYMNWVRQAPGQSLEWMGIINPYNGDTSYNQKFRGRVTLTVDKSASTAYMELSSLRSEDTAVYYCARYHYGPHLEGYFDVWGQGTTVTVSS

SEQ ID NO: 107 - protein, 122 aa, synthetic construct
  REGION 1..122: note = hu30VH3
EVRLVQSGAEVKKPGASVKVSCKASGYTFTDYYMNWVRQAPGQSLEWMGIINPYQGDTSYNQKFRGRVTLTVDKSASTAYMELSSLRSEDTAVYYCARYHYGPHLEGYFDVWGQGTTVTVSS

SEQ ID NO: 108 - protein, 122 aa, synthetic construct
  REGION 1..122: note = hu30VH4
EVRLVQSGAEVKKPGASVKVSCKASGYTFTDYYMNWVRQAPGQSLEWMGIINPYSGDTSYNQKFRGRVTLTVDKSASTAYMELSSLRSEDTAVYYCARYHYGPHLEGYFDVWGQGTTVTVSS

SEQ ID NO: 109 - protein, 122 aa, synthetic construct
  REGION 1..122: note = hu30VH5
EVRLVQSGAEVKKPGASVKVSCKASGYTFTDYYMNWVRQAPGQSLEWMGIINPYTGDTSYNQKFRGRVTLTVDKSASTAYMELSSLRSEDTAVYYCARYHYGPHLEGYFDVWGQGTTVTVSS

SEQ ID NO: 110 - protein, 122 aa, synthetic construct
  REGION 1..122: note = hu30VH6
EVRLVQSGAEVKKPGASVKVSCKASGYTFTDYYMNWVRQAPGQSLEWMGIINPYNVDTSYNQKFRGRVTLTVDKSASTAYMELSSLRSEDTAVYYCARYHYGPHLEGYFDVWGQGTTVTVSS

SEQ ID NO: 111 - protein, 122 aa, synthetic construct
  REGION 1..122: note = hu30VH7
EVRLQQSGPVVVKPGASVKVSCKASGYTFTDYYMNWVRQAPGQSLEWMGIINPYNGDTSYNQKFRGRVTLTVDKSASTAYMELSSLRSEDTAVYYCARYHYGPHLEGYFDVWGQGTTVTVSS

SEQ ID NO: 112 - protein, 122 aa, synthetic construct
  REGION 1..122: note = hu30VH8
EVRLVQFGAVVKKPGASVKVSCKASGYTFTDYYMNWVRQAPGQSLEWMGIINPYTGDTSYNQKFRGRVTLTVDKSSSTAYMELSSLRSEDTAVYYCARYHYGPHLEGYFDVWGQGTTVTVSS

SEQ ID NO: 113 - protein, 122 aa, synthetic construct
  REGION 1..122: note = hu30VH9
EVRLVQSGAEVKKPGASVKVSCKASGYTFTDYYMNWVRQAPGQSLEWIGIINPYTGDTSYNQKFRGRATLTVDKSASTAYMELSSLRSEDTAVYYCARYHYGPHLEGYFDVWGQGTTVTVSS

SEQ ID NO: 114 - protein, 122 aa, synthetic construct
  REGION 1..122: note = hu30VH10
EVRLVQSGAEVKKPGASVKVSCKASGYTFTDYYMNWVRQSHGQSLEWMGIINPYTGDTSYNQKFRGRVTLTVDKSASTAYMELSSLRSEDTAVYYCARYHYGPHLEGYFDVWGQGTTVTVSS

SEQ ID NO: 115 - protein, 122 aa, synthetic construct
  REGION 1..122: note = hu30VH11
EVRLVQFGAVVKKPGASVKVSCKASGYTFTDYYMNWVRQAPGQSLEWMGIINPYTGDTSYNQKFRGRVTLTVDKSSSTAYMELSSLTSEDTAVYYCARYHYGPHLEGYFDVWGQGTTVTVSS

SEQ ID NO: 116 - protein, 122 aa, synthetic construct
  REGION 1..122: note = hu30VH12
QVQLVQSGAEVKKPGASVKVSCKASGYTFTDYYMNWVRQAPGQRLEWMGIINPYNGDTSYNQKFRGRVTITRDTSASTAYMELSSLRSEDTAVYYCARYHYGPHLEGYFDVWGQGTTVTVSS

SEQ ID NO: 117 - protein, 17 aa, synthetic construct
  REGION 1..17: note = hu30VH3, HCDR2
IINPYQGDTSYNQKFRG

SEQ ID NO: 118 - protein, 17 aa, synthetic construct
  REGION 1..17: note = hu30VH4, HCDR2
IINPYSGDTSYNQKFRG

SEQ ID NO: 119 - protein, 17 aa, synthetic construct
  REGION 1..17: note = hu30VH5/VH8/VH9/VH10/VH11 HCDR2
IINPYTGDTSYNQKFRG

SEQ ID NO: 120 - protein, 17 aa, synthetic construct
  REGION 1..17: note = hu30VH6, HCDR2
IINPYNVDTSYNQKFRG

SEQ ID NO: 121 - protein, 450 aa, synthetic construct
  REGION 1..450: note = Hu6 hu6L4H12
EVQLVQSGSELKKPGASVKVSCKASGYTFTDYYMNWVRQAPGQGLEWMGIINPYSGVTIYNHKFKGRFALSVDKSSNTAYLQISSLKAEDTAVYYCARQDSLLDYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 214 aa, synthetic construct
  REGION 1..214: note = Hu6 hu6L4H12
DIQMTQSPSSLSASVGDRVTITCHASQGISGNMGWLQQKPGKAPKGLIYHGANLEDGVPSRFSGSGSGADYTLTISSLQPEDFADYYCVQYAQFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 451 aa, synthetic construct
  REGION 1..451: note = Hu98 hu98L3H4
EVQLVESGGGLVQPGGSLRLSCAASGFTFSDYGIHWVRQAPGKGLEWVAYISSGGYTSYYADTVKGRFTISRDNAKNSLYLQMNSLRAEDTAVYYCARGDYEDIFYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 214 aa, synthetic construct
  REGION 1..214: note = Hu98 hu98L3H4
DIQMTQSPSSLSASVGDRVTITCRASRDITKFLNWYQQKPGGAVKLLIYYTSRLHSGVPSRFSGSGSGTDYTLTISSLQPEDFATYYCQQGNTLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 454 aa, synthetic construct
  REGION 1..454: note = Hu110 hu110L2H3
EVQLVQSGAEVKKPGASVKVSCKTSGYTFTDYYMNWVRQAPGQSLEWMGIINPYQGGTSYTQKFKGRVTITVDKSASTAYMELSSLRSEDTAVYYCARHFYDSSYLTYSTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 214 aa, synthetic construct
  REGION 1..214: note = Hu110 hu110L2H3
NIVMTQSPKSLAVSLGERATINCKASENVGIFVSWYQQKPGQSPKLLIYGASNRYTGVPDRFSGSGSATDFTLTISSLQAEDVADYYCGQSHSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 452 aa, synthetic construct
  REGION 1..452: note = Hu30 hu30L1H5
EVRLVQSGAEVKKPGASVKVSCKASGYTFTDYYMNWVRQAPGQSLEWMGIINPYTGDTSYNQKFRGRVTLTVDKSASTAYMELSSLRSEDTAVYYCARYHYGPHLEGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 213 aa, synthetic construct
  REGION 1..213: note = Hu30 hu30L1H5
DIVMTQSPDSLAVSLGERATINCKASENVGYVSWYQQKPGQPPKLLIYGASNRYTGVPDRFSGSGSGTDFTLTISSLQAEDVAVYYCGQSYSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 5 aa, synthetic construct
  REGION 1..5: note = S107E I2C HCDR1
KYAMN

SEQ ID NO: 130 - protein, 19 aa, synthetic construct
  REGION 1..19: note = S107E I2C HCDR2
RIRSKYNNYATYYADSVKD

SEQ ID NO: 131 - protein, 14 aa, synthetic construct
  REGION 1..14: note = S107E HCDR3
HGNFGNEYISYWAY

SEQ ID NO: 132 - protein, 14 aa, synthetic construct
  REGION 1..14: note = S107E I2C LCDR1
GSSTGAVTSGNYPN

SEQ ID NO: 133 - protein, 7 aa, synthetic construct
  REGION 1..7: note = S107E I2C LCDR2
GTKFLAP

SEQ ID NO: 134 - protein, 9 aa, synthetic construct
  REGION 1..9: note = S107E I2C LCDR3
VLWYSNRWV

SEQ ID NO: 135 - protein, 14 aa, synthetic construct
  REGION 1..14: note = I2C HCDR3
HGNFGNSYISYWAY

SEQ ID NO: 136 - protein, 125 aa, synthetic construct
  REGION 1..125: note = S107E-VH
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSS

SEQ ID NO: 137 - protein, 109 aa, synthetic construct
  REGION 1..109: note = S107E IC2-VL
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 138 - protein, 125 aa, synthetic construct
  REGION 1..125: note = I2C-VH
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 139 - protein, 249 aa, synthetic construct
  REGION 1..249: note = S107E-scFv
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 140 - protein, 249 aa, synthetic construct
  REGION 1..249: note = I2C-scFv
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 141 - protein, 103 aa, synthetic construct
  REGION 1..103: note = IgG1(CH1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 142 - protein, 105 aa, synthetic construct
  REGION 1..105: note = h
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 143 - protein, 227 aa, synthetic construct
  REGION 1..227: note = IgG1Fc knob
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 227 aa, synthetic construct
  REGION 1..227: note = IgG1Fc hole
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 145 - protein, 222 aa, synthetic construct
  REGION 1..222: note = Fc knob
CPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 222 aa, synthetic construct
  REGION 1..222: note = Fc hole
CPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 147 - protein, 5 aa, synthetic construct
  REGION 1..5: note = Linker1
GGGGS

SEQ ID NO: 148 - protein, 15 aa, synthetic construct
  REGION 1..15: note = Linker2
GGGGSGGGGSGGGGS

SEQ ID NO: 149 - protein, 8 aa, synthetic construct
  REGION 1..8: note = Linke3
GGGSGGGG

SEQ ID NO: 150 - skipped

SEQ ID NO: 151 - protein, 9 aa, synthetic construct
  REGION 1..9: note = Linke5
GGGGSGGGG

SEQ ID NO: 152 - protein, 209 aa, synthetic construct
  REGION 1..209: note = hu6VL-G4S-Obscurin
DIQMTQSPSSLSASVGDRVTITCHASQGISGNMGWLQQKPGKAPKGLIYHGANLEDGVPSRFSGSGSGADYTLTISSLQPEDFADYYCVQYAQFPYTFGGGTKVEIKGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 153 - protein, 450 aa, synthetic construct
  REGION 1..450: note = hu6VH-G4S-Titin-hole
EVQLVQSGSELKKPGASVKVSCKASGYTFTDYYMNWVRQAPGQGLEWMGIINPYSGVTIYNHKFKGRFALSVDKSSNTAYLQISSLKAEDTAVYYCARQDSLLDYAMDYWGQGTTVTVSSGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSIDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 154 - protein, 455 aa, synthetic construct
  REGION 1..455: note = S107EVH-CH1-knob
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 214 aa, synthetic construct
  REGION 1..214: note = CD3VL-h
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 156 - protein, 455 aa, synthetic construct
  REGION 1..455: note = I2CVH-CH1-knob
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 214 aa, synthetic construct; identical to SEQ ID NO: 122
  REGION 1..214: note = hu6VL-h

SEQ ID NO: 158 - protein, 450 aa, synthetic construct
  REGION 1..450: note = hu6VH-CH1-hole
EVQLVQSGSELKKPGASVKVSCKASGYTFTDYYMNWVRQAPGQGLEWMGIINPYSGVTIYNHKFKGRFALSVDKSSNTAYLQISSLKAEDTAVYYCARQDSLLDYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 159 - protein, 481 aa, synthetic construct
  REGION 1..481: note = S107E_scFv-knob
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 481 aa, synthetic construct
  REGION 1..481: note = I2C_scFv-knob
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 463 aa, synthetic construct
  REGION 1..463: note = hu6VL-S107EVH-knob
DIQMTQSPSSLSASVGDRVTITCHASQGISGNMGWLQQKPGKAPKGLIYHGANLEDGVPSRFSGSGSGADYTLTISSLQPEDFADYYCVQYAQFPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 461 aa, synthetic construct
  REGION 1..461: note = CD3VL-hu6VH-hole
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGEVQLVQSGSELKKPGASVKVSCKASGYTFTDYYMNWVRQAPGQGLEWMGIINPYSGVTIYNHKFKGRFALSVDKSSNTAYLQISSLKAEDTAVYYCARQDSLLDYAMDYWGQGTTVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 163 - protein, 463 aa, synthetic construct
  REGION 1..463: note = hu6VL-I2CVH-knob
DIQMTQSPSSLSASVGDRVTITCHASQGISGNMGWLQQKPGKAPKGLIYHGANLEDGVPSRFSGSGSGADYTLTISSLQPEDFADYYCVQYAQFPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 209 aa, synthetic construct
  REGION 1..209: note = hu98VL-G4S-Obscurin
DIQMTQSPSSLSASVGDRVTITCRASRDITKFLNWYQQKPGGAVKLLIYYTSRLHSGVPSRFSGSGSGTDYTLTISSLQPEDFATYYCQQGNTLPWTFGGGTKVEIKGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 165 - protein, 451 aa, synthetic construct
  REGION 1..451: note = hu98VH-G4S-Titin-hole
EVQLVESGGGLVQPGGSLRLSCAASGFTFSDYGIHWVRQAPGKGLEWVAYISSGGYTSYYADTVKGRFTISRDNAKNSLYLQMNSLRAEDTAVYYCARGDYEDIFYTMDYWGQGTTVTVSSGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSIDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 166 - protein, 214 aa, synthetic construct; identical to SEQ ID NO: 124
  REGION 1..214: note = hu98VL-h

SEQ ID NO: 167 - protein, 451 aa, synthetic construct
  REGION 1..451: note = hu98VH-CH1-hole
EVQLVESGGGLVQPGGSLRLSCAASGFTFSDYGIHWVRQAPGKGLEWVAYISSGGYTSYYADTVKGRFTISRDNAKNSLYLQMNSLRAEDTAVYYCARGDYEDIFYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 168 - protein, 463 aa, synthetic construct
  REGION 1..463: note = hu98VL-S107EVH-knob
DIQMTQSPSSLSASVGDRVTITCRASRDITKFLNWYQQKPGGAVKLLIYYTSRLHSGVPSRFSGSGSGTDYTLTISSLQPEDFATYYCQQGNTLPWTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 462 aa, synthetic construct
  REGION 1..462: note = CD3VL-hu98VH-hole
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGEVQLVESGGGLVQPGGSLRLSCAASGFTFSDYGIHWVRQAPGKGLEWVAYISSGGYTSYYADTVKGRFTISRDNAKNSLYLQMNSLRAEDTAVYYCARGDYEDIFYTMDYWGQGTTVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 170 - protein, 463 aa, synthetic construct
  REGION 1..463: note = hu98VL-I2CVH-knob
DIQMTQSPSSLSASVGDRVTITCRASRDITKFLNWYQQKPGGAVKLLIYYTSRLHSGVPSRFSGSGSGTDYTLTISSLQPEDFATYYCQQGNTLPWTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 209 aa, synthetic construct
  REGION 1..209: note = hu110VL-G4S-Obscurin
NIVMTQSPKSLAVSLGERATINCKASENVGIFVSWYQQKPGQSPKLLIYGASNRYTGVPDRFSGSGSATDFTLTISSLQAEDVADYYCGQSHSYPLTFGQGTKLEIKGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 172 - protein, 454 aa, synthetic construct
  REGION 1..454: note = hu110VH-G4S-Titin-hole
EVQLVQSGAEVKKPGASVKVSCKTSGYTFTDYYMNWVRQAPGQSLEWMGIINPYQGGTSYTQKFKGRVTITVDKSASTAYMELSSLRSEDTAVYYCARHFYDSSYLTYSTMDYWGQGTTVTVSSGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSIDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 173 - protein, 214 aa, synthetic construct; identical to SEQ ID NO: 126
  REGION 1..214: note = hu110VL-h

SEQ ID NO: 174 - protein, 454 aa, synthetic construct
  REGION 1..454: note = hu110VH-CH1-hole
EVQLVQSGAEVKKPGASVKVSCKTSGYTFTDYYMNWVRQAPGQSLEWMGIINPYQGGTSYTQKFKGRVTITVDKSASTAYMELSSLRSEDTAVYYCARHFYDSSYLTYSTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 175 - protein, 463 aa, synthetic construct
  REGION 1..463: note = hu110VL-S107EVH-knob
NIVMTQSPKSLAVSLGERATINCKASENVGIFVSWYQQKPGQSPKLLIYGASNRYTGVPDRFSGSGSATDFTLTISSLQAEDVADYYCGQSHSYPLTFGQGTKLEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 465 aa, synthetic construct
  REGION 1..465: note = CD3VL-hu110VH-hole
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGEVQLVQSGAEVKKPGASVKVSCKTSGYTFTDYYMNWVRQAPGQSLEWMGIINPYQGGTSYTQKFKGRVTITVDKSASTAYMELSSLRSEDTAVYYCARHFYDSSYLTYSTMDYWGQGTTVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 177 - protein, 463 aa, synthetic construct
  REGION 1..463: note = hu110VL-I2CVH-knob
NIVMTQSPKSLAVSLGERATINCKASENVGIFVSWYQQKPGQSPKLLIYGASNRYTGVPDRFSGSGSATDFTLTISSLQAEDVADYYCGQSHSYPLTFGQGTKLEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 208 aa, synthetic construct
  REGION 1..208: note = hu30VL-G4S-Obscurin
DIVMTQSPDSLAVSLGERATINCKASENVGYVSWYQQKPGQPPKLLIYGASNRYTGVPDRFSGSGSGTDFTLTISSLQAEDVAVYYCGQSYSYPLTFGQGTKLEIKGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 179 - protein, 452 aa, synthetic construct
  REGION 1..452: note = hu30VH-G4S-Titin-hole
EVRLVQSGAEVKKPGASVKVSCKASGYTFTDYYMNWVRQAPGQSLEWMGIINPYTGDTSYNQKFRGRVTLTVDKSASTAYMELSSLRSEDTAVYYCARYHYGPHLEGYFDVWGQGTTVTVSSGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSIDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 180 - protein, 213 aa, synthetic construct; identical to SEQ ID NO: 128
  REGION 1..213: note = hu30VL-h

SEQ ID NO: 181 - protein, 452 aa, synthetic construct
  REGION 1..452: note = hu30VH-CH1-hole
EVRLVQSGAEVKKPGASVKVSCKASGYTFTDYYMNWVRQAPGQSLEWMGIINPYTGDTSYNQKFRGRVTLTVDKSASTAYMELSSLRSEDTAVYYCARYHYGPHLEGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 182 - protein, 462 aa, synthetic construct
  REGION 1..462: note = hu30VL-S107EVH-knob
DIVMTQSPDSLAVSLGERATINCKASENVGYVSWYQQKPGQPPKLLIYGASNRYTGVPDRFSGSGSGTDFTLTISSLQAEDVAVYYCGQSYSYPLTFGQGTKLEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 463 aa, synthetic construct
  REGION 1..463: note = CD3VL-hu30VH-hole
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGEVRLVQSGAEVKKPGASVKVSCKASGYTFTDYYMNWVRQAPGQSLEWMGIINPYTGDTSYNQKFRGRVTLTVDKSASTAYMELSSLRSEDTAVYYCARYHYGPHLEGYFDVWGQGTTVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 184 - protein, 462 aa, synthetic construct
  REGION 1..462: note = hu30VL-I2CVH-knob
DIVMTQSPDSLAVSLGERATINCKASENVGYVSWYQQKPGQPPKLLIYGASNRYTGVPDRFSGSGSGTDFTLTISSLQAEDVAVYYCGQSYSYPLTFGQGTKLEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 463 aa, synthetic construct
  REGION 1..463: note = hu6VL (D56E) -S107EVH-knob
DIQMTQSPSSLSASVGDRVTITCHASQGISGNMGWLQQKPGKAPKGLIYHGANLEEGVPSRFSGSGSGADYTLTISSLQPEDFADYYCVQYAQFPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 463 aa, synthetic construct
  REGION 1..463: note = hu6VL(D56S) -S107EVH-knob
DIQMTQSPSSLSASVGDRVTITCHASQGISGNMGWLQQKPGKAPKGLIYHGANLESGVPSRFSGSGSGADYTLTISSLQPEDFADYYCVQYAQFPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 463 aa, synthetic construct
  REGION 1..463: note = hu6VL8 -S107EVH-knob
DIQMTQSPSSLSASVGDRVTITCHASQGISGNMGWLQQKPGKAPKGLIYHGANLEEGVPSRFSGSGSGADYTLTISSLQPEDFADYYCVQYAQYPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 461 aa, synthetic construct
  REGION 1..461: note = CD3VL-hu6VH14-hole
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGEVQLVQSGSELKKPGASVKVSCKASGYTFTDYYMNWVRQAPGQGLEWMGIINPYSGVTTYNHKFKGRFALSVDKSSNTAYLQISSLKAEDTAVYYCARQDSLLDYAMDYWGQGTTVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 189 - protein, 461 aa, synthetic construct
  REGION 1..461: note = CD3VL-hu6VH15-hole
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGEVQLQQSGSELKKPGASVKVSCKASGYTFTDYYMNWVRQAPGQGLEWMGIINPYSGSTIYNHKFKGRFALSVDKSSNTAYLQISSLKAEDTAVYYCARQDSLLDYAMDYWGQGTTVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 190 - protein, 463 aa, synthetic construct
  REGION 1..463: note = hu6VL9-S107EVH-knob
NIQMTQSPSSLSASVGDRVTITCHASQGISGNMGWLQQKPGKAPKGLIYHGRNLEEGVPSRFSGSGSGADYTLTISSLQPEDFADYYCVQYAQYPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 463 aa, synthetic construct
  REGION 1..463: note = hu6VL10-S107EVH-knob
EIVLTQSPATLSLSPGERATLSCHASQGISGNMGWLQQKPGKAPRGLIYHGANLEEGIPARFSGSGSGADYTLTISRLQPEDFADYYCVQYAQFPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 98 aa, synthetic construct
  REGION 1..98: note = T.0 192
GIPPKIEALPSDISIDEGKVLTVACAFTGEPTPEVTWSCGGRKIHSQEQGRFHIENTDDLTTLIIMDVQKQDGGLYTLSLGNEFGSDSATVNIHIRSI

SEQ ID NO: 193 - protein, 98 aa, synthetic construct
  REGION 1..98: note = T.1 193
GIPPKIECLPSDISIDEGKVLTVASAFTGEPTPEVTWSTGGRKIHSQEQGRFHIENTDDLTTLIIMDVQKQDGGLYTLSLGNEFGSDSATVNIHIRSI

SEQ ID NO: 194 - protein, 98 aa, synthetic construct
  REGION 1..98: note = T.2 194
GIPPKIEALPSDISIDEGKCLTVASAFTGEPTPEVTWSTGGRKIHSQEQGRFHIENTDDLTTLIIMDVQKQDGGLYTLSLGNEFGSDSATVNIHIRSI

SEQ ID NO: 195 - protein, 98 aa, synthetic construct
  REGION 1..98: note = T.3 195
GIPPKIEALPSDISIDEGKVLTVASCFTGEPTPEVTWSTGGRKIHSQEQGRFHIENTDDLTTLIIMDVQKQDGGLYTLSLGNEFGSDSATVNIHIRSI

SEQ ID NO: 196 - protein, 98 aa, synthetic construct
  REGION 1..98: note = T.4 196
GIPPKIEALPSDISIDEGKVLCVASAFTGEPTPEVTWSTGGRKIHSQEQGRFHIENTDDLTTLIIMDVQKQDGGLYTLSLGNEFGSDSATVNIHIRSI

SEQ ID NO: 197 - protein, 103 aa, synthetic construct
  REGION 1..103: note = T.5 197
KAGIRGIPPKIECLPSDISIDEGKVLTVASAFTGEPTPEVTWSTGGRKIHSQEQGRFHIENTDDLTTLIIMDVQKQDGGLYTLSLGNEFGSDSATVNIHIRSI

SEQ ID NO: 198 - protein, 103 aa, synthetic construct
  REGION 1..103: note = T.6 198
KAGIRGIPPKIEALPSDISIDEGKVLTVACAFTGEPTPEVTWSCGGRKIHSQEQGRFHIENTDDLTTLIIMDVQKQDGGLYTLSLGNEFGSDSATVNIHIRSI

SEQ ID NO: 199 - protein, 98 aa, synthetic construct
  REGION 1..98: note = T.7 199
GIPPKIECLPSDISIDEGKVLTVASAFTGEPTPEVTWSTGGRKIHSQEQGRFHIENTDDLTTLIISDVQKQDGGLYSLSLGNEFGSDSATVNIHIRSI

SEQ ID NO: 200 - protein, 98 aa, synthetic construct
  REGION 1..98: note = T.8 200
GIPPKIECLPSDISIDEGKVLTVASAFTGEPTPEVTWSTGGRKIHSQEQGRFHIENTDDLTTLIIKDVQRQDGGLYTLTLRNEFGSDSATVNIHIRSI

SEQ ID NO: 201 - protein, 98 aa, synthetic construct
  REGION 1..98: note = T.9 201
GIWPKIECLPIDLSIDEGKVLMVASAFTGEPTPEVTWSTGGRKIHSQEQGRFHIENTDDLTTLIIMDVQKQDGGLYTLSLGMEFGSDSATVNIHIRSI

SEQ ID NO: 202 - protein, 98 aa, synthetic construct
  REGION 1..98: note = T.10 202
GIPPKIECLPIDISIDEGKVLTVASAFTGEPTPEVTWSTGGRKIHSQEQGRFHIENTDDLTTLIIKDVQKQDGGLYTLTLRNEFGSDSATVNIHIRSI

SEQ ID NO: 203 - protein, 98 aa, synthetic construct
  REGION 1..98: note = T.11 203
GIPPKIECLPSDISIDEGKVLTVASAFTGEPTPEVTWSTSGKKISSEEGGRFHIESTEDLTTLIIMDVQKQDGGVYTLSLGNDLGSDSATVNIHIRSI

SEQ ID NO: 204 - protein, 98 aa, synthetic construct
  REGION 1..98: note = T.12 204
GIPPKIECLPSDISIDEGKVLTVASAFTGEPTPEVTWSTGGRKIHSEEGGRFHIESTEDLTTLIIMDVQKQDGGVYTLSLGNEFGSDSATVNIHIRSI

SEQ ID NO: 205 - protein, 98 aa, synthetic construct
  REGION 1..98: note = T.13 205
GIPPKIECLPSDISIDEGKVLTVASAFTGEPTPEVTWSTGGRKIHSQEQGRFHIESTEDLTTLIIMDVQKQDGGVYTLSLGNEFGSDSATVNIHIRSI

SEQ ID NO: 206 - protein, 98 aa, synthetic construct
  REGION 1..98: note = T.14 206
GIPPKIECLPSDISIDEGKVLTVASAFTGEPTPEVTWSTGGRKIHSQEQGRFHIESTEDLTTLIISDVQKQDGGLYSLSLGNEFGSDSATVNIHIRSI

SEQ ID NO: 207 - protein, 103 aa, synthetic construct
  REGION 1..103: note = T.15 207
KAGIRGIPPKIECLPSDISIDEGKVLTVASAFTGEPTPEVTWSTGGRKIHSQEQGRFHIESTEDLTTLIISDVQKQDGGLYSLSLGNEFGSDSATVNIHIRSI

SEQ ID NO: 208 - protein, 98 aa, synthetic construct
  REGION 1..98: note = T.16 208
GIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 209 - protein, 98 aa, synthetic construct
  REGION 1..98: note = T.17 209
GIPPKIECLPIDISIDEGKCLTVASAFTGEPTPEVTWSTGGRKIHSQEQGRFHIENTDDSTTLTIKDVQKQDGGLYTLTLRNEFGSDSATVNIHIRSI

SEQ ID NO: 210 - protein, 98 aa, synthetic construct
  REGION 1..98: note = T.18 210
GIPPKIECLPIDISIDEGKVLTVASCFTGEPTPEVTWSTGGRKIHSQEQGRFHIENTDDSTTLTIKDVQKQDGGLYTLTLRNEFGSDSATVNIHIRSI

SEQ ID NO: 211 - protein, 97 aa, synthetic construct
  REGION 1..97: note = O.0 211
SGAPRFLTRPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 212 - protein, 98 aa, synthetic construct
  REGION 1..98: note = OL.0 212
QGSPPCFLRFPRPVRVVSGAEAELKCVVLGEPPPVVVWEKGGQQLAASERLSFPADGAEHGLLLTAALPTDAGVYVCRARNAAGEAYAAAAVTVLEPP

SEQ ID NO: 213 - protein, 97 aa, synthetic construct
  REGION 1..97: note = O.1 213
SGAPRFLTRPKAFVVSVGKDATLSCQIVGNPTPQVSWEKDQQPVAAGARFRLAQDGDLYRLTILDLALGDSGQYVCRARNAIGEAFACVGLQVDAEA

SEQ ID NO: 214 - protein, 97 aa, synthetic construct
  REGION 1..97: note = O.2 214
SGCPRFLTRPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 215 - protein, 97 aa, synthetic construct
  REGION 1..97: note = O.3 215
SGAPRFLTCPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 216 - protein, 97 aa, synthetic construct
  REGION 1..97: note = O.4 216
SGAPRFLTRPKAFVVSVGKDATLSSQIVGNPTPQVSWEKDQQPVAAGARFRLAQDGDLYRLTILDLALGDSGQYVSRARNAIGEAFACVGLQVDAEA

SEQ ID NO: 217 - protein, 97 aa, synthetic construct
  REGION 1..97: note = O.5 217
SGCPRFLTRPKAFVVSVGKDATLSSQIVGNPTPQVSWEKDQQPVAAGARFRLAQDGDLYRLTILDLALGDSGQYVSRARNAIGEAFAAVGLQVDAEA

SEQ ID NO: 218 - protein, 97 aa, synthetic construct
  REGION 1..97: note = O.6 218
SGAPRFLTCPKAFVVSVGKDATLSSQIVGNPTPQVSWEKDQQPVAAGARFRLAQDGDLYRLTILDLALGDSGQYVSRARNAIGEAFAAVGLQVDAEA

SEQ ID NO: 219 - protein, 97 aa, synthetic construct
  REGION 1..97: note = O.7 219
SGAPRFKTRPKAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 220 - protein, 97 aa, synthetic construct
  REGION 1..97: note = O.8 220
SGAPRFKTRPKAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 221 - protein, 97 aa, synthetic construct
  REGION 1..97: note = O.9 221
SGAPRFLTRPLAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 222 - protein, 97 aa, synthetic construct
  REGION 1..97: note = O.10 222
SGAPRFLTRPLAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 223 - protein, 102 aa, synthetic construct
  REGION 1..102: note = O.11 223
DQPQFSGAPRFLTRPKAFVVSVGKDATLSSQIVGNPTPQVSWEKDQQPVAAGARFRLAQDGDLYRLTILDLALGDSGQYVSRARNAIGEAFACVGLQVDAEA

SEQ ID NO: 224 - protein, 102 aa, synthetic construct
  REGION 1..102: note = O.12 224
DQPQFSGAPRFKTRPKAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 225 - protein, 102 aa, synthetic construct
  REGION 1..102: note = O.13 225
DQPQFSGAPRFKTRPKAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 226 - protein, 102 aa, synthetic construct
  REGION 1..102: note = O.14 226
DQPQFSGAPRFRTRPKAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 227 - protein, 102 aa, synthetic construct
  REGION 1..102: note = O.15 227
DQPQFSGAPRFRTRPKAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 228 - protein, 102 aa, synthetic construct
  REGION 1..102: note = O.16 228
DQPQFSGAPRFLTRPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 229 - protein, 98 aa, synthetic construct
  REGION 1..98: note = OL.1 229
QGSPPEFLRFPRPVRVVSGAEAELKCVVLGEPPPVVVWEKGGQQLAASERLSFPADGAEHGLLLTAALPTDAGCYVCRARNAAGEAYAAAAVTVLEPP

SEQ ID NO: 230 - protein, 98 aa, synthetic construct
  REGION 1..98: note = OL.2 230
QGSPPEFLRFPRPVRVVSGAEAELKCVVLGEPPPVVVWEKGGQQLAASERLSFPADGAEHGLLLTAALPTDAGVYVCRARNAACEAYAAAAVTVLEPP

SEQ ID NO: 231 - protein, 98 aa, synthetic construct
  REGION 1..98: note = OL.3 231
QGSPPEFLRFPRPVRVVSGAEAELKCVVLGEPPPVVVWEKGGQQLAASERLSFPADGAEHGLLLTAALPTDAGVYVCRARNAAGECYAAAAVTVLEPP

SEQ ID NO: 232 - protein, 98 aa, synthetic construct
  REGION 1..98: note = OL.4 232
QGSPPEFLRFPRPVRVVSGAEAELKSVVLGEPPPVVVWEKGGQQLAASERLSFPADGAEHGLLLTAALPTDAGCYVSRARNAAGEAYAAAAVTVLEPP

SEQ ID NO: 233 - protein, 98 aa, synthetic construct
  REGION 1..98: note = OL.5 233
QGSPPEFLRFPRPVRVVSGAEAELKSVVLGEPPPVVVWEKGGQQLAASERLSFPADGAEHGLLLTAALPTDAGVYVSRARNAACEAYAAAAVTVLEPP

SEQ ID NO: 234 - protein, 98 aa, synthetic construct
  REGION 1..98: note = OL.6 234
QGSPPEFLRFPRPVRVVSGAEAELKSVVLGEPPPVVVWEKGGQQLAASERLSFPADGAEHGLLLTAALPTDAGVYVSRARNAAGECYAAAAVTVLEPP

SEQ ID NO: 235 - protein, 97 aa, synthetic construct
  REGION 1..97: note = O.17 235
SGAPRFLTRPKSYTVSVGKDASLSSQIVGNPTPQVSWEKDQQPVAAGARFRLAQDGDLYRLKILDLALGDSGQYVSRARNAIGEAFACVGLQVDAEA

SEQ ID NO: 236 - protein, 97 aa, synthetic construct
  REGION 1..97: note = O.18 236
SEAPRFLTRPLAFVVSEGKDATLSSQIVGDPPPEVTWEKDQQPITAGARFRLAQDGDVYRLEILDLQLSDSGQYVSRARNAIGEAFACVGLQVDAEG

SEQ ID NO: 237 - protein, 97 aa, synthetic construct
  REGION 1..97: note = O.19 237
SGAPRFLTRPLAFVVSVGKLAMLSSQIVGNPTPQVSWEKDQQPVAAGARFRLLQDGDLYRLKILDLALGDSGQYVSRARNAIGEAFACVGLQVDAEA

SEQ ID NO: 238 - protein, 97 aa, synthetic construct
  REGION 1..97: note = O.20 238
SGAPRFLTRPLAFVVSVGKDATLSSQIVGNPTPQVSWEKDKQPVTAGARFRLAQDGDLYRLKILDLQLSDSGQYVSRARNAIGEAFACLGLQVDAEA

SEQ ID NO: 239 - protein, 97 aa, synthetic construct
  REGION 1..97: note = O.21 239
SGAPRFLTRPLAFVVSVGKDATLSSQIVGNPTPQVSWEKDQLPVTAGARFRLAQDGDLYRLKILDLTLSDSGQYVSRARNAIGEAFACVGLEVGAEA

SEQ ID NO: 240 - protein, 97 aa, synthetic construct
  REGION 1..97: note = O.22 240
SGAPRFLTRPLSYVVSVGKDASLSSQIVGNPTPQVSWEKDQLPVTAGARFRLAQDGDLYRLKILDLQLSDSGQYVSRARNAIGEAFACVGLEVGAEA

SEQ ID NO: 241 - protein, 102 aa, synthetic construct
  REGION 1..102: note = O.23 241
DQPQFSGAPRFLTRPLSYVVSVGKDASLSSQIVGNPTPQVSWEKDQLPVTAGARFRLAQDGDLYRLKILDLQLSDSGQYVSRARNAIGEAFACVGLEVGAEA

SEQ ID NO: 242 - protein, 97 aa, synthetic construct
  REGION 1..97: note = O.24 242
SGAPRFLTRPKAFVVSVGKDATLSSQIVGNPFPQVSWEKDKQPVTAGVRFRLAQDGDLYRLKILDLQLSDSGQYVSRARNAIGEAFACLGLQVDAEA

SEQ ID NO: 243 - protein, 97 aa, synthetic construct
  REGION 1..97: note = O.25 243
SGAPRFLTRPKASVVSVGKDATLSSQIVGNPFPQVSWEKDKQPVTAGVRFRLAQDGDLYRLKILDLQLSDSGQYVSRARNAHGEAFACLGLQVDAEA

SEQ ID NO: 244 - protein, 97 aa, synthetic construct
  REGION 1..97: note = O.26 244
SGCPRFLTRPKASVVSVGKDATLSSQIVGNPFPQVSWEKDKQPVTAGVRFRLAQDGDLYRLKILDLQLSDSGQYVSRARNAHGEAFACLGLQVDAEA

SEQ ID NO: 245 - protein, 97 aa, synthetic construct
  REGION 1..97: note = O.27 245
SGAPRFLTCPKASVVSVGKDATLSSQIVGNPFPQVSWEKDKQPVTAGVRFRLAQDGDLYRLKILDLQLSDSGQYVSRARNAHGEAFACLGLQVDAEA

SEQ ID NO: 246 - protein, 97 aa, synthetic construct
  REGION 1..97: note = O.28 246
SGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 247 - protein, 97 aa, synthetic construct
  REGION 1..97: note = O.29 247
SGCPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 248 - protein, 97 aa, synthetic construct
  REGION 1..97: note = O.30 248
SGAPRFLTCPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 249 - protein, 97 aa, synthetic construct
  REGION 1..97: note = O.31 249
SGAPRFLTRPKASVVSVGKDATLSSQIVGNPFPQVSWEKDKQPVTAGVRFRLAQDGDLYRLKILDCQLSDSGQYVSRARNAHGEAFACLGLQCDAEA

SEQ ID NO: 250 - protein, 97 aa, synthetic construct
  REGION 1..97: note = O.32 250
SGCPRFLTRPKASVVSVGKDATLSSQIVGNPFPQVSWEKDKQPVTAGVRFRLAQDGDLYRLKILDCQLSDSGQYVSRARNAHGEAFACLGLQCDAEA

SEQ ID NO: 251 - protein, 97 aa, synthetic construct
  REGION 1..97: note = O.33 251
SGAPRFLTCPKASVVSVGKDATLSSQIVGNPFPQVSWEKDKQPVTAGVRFRLAQDGDLYRLKILDCQLSDSGQYVSRARNAHGEAFACLGLQCDAEA

SEQ ID NO: 252 - protein, 982 aa, synthetic construct
  REGION 1..982: note = AMG-757
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - protein, 705 aa, synthetic construct
  REGION 1..705: note = BI-764532 1
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 254 - protein, 707 aa, synthetic construct
  REGION 1..707: note = BI-764532 2
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 255 - protein, 5 aa, synthetic construct
  source 1..5: note = linker
ASTKG

SEQ ID NO: 256 - protein, 5 aa, synthetic construct
  source 1..5: note = linker
RTVAS

SEQ ID NO: 257 - protein, 118 aa, synthetic construct
  source 1..118: note = VH2-L
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 258 - protein, 108 aa, synthetic construct
  source 1..108: note = VL2-L
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 259 - protein, 463 aa, synthetic construct
  source 1..463: note = hu6VL (D56E) -I2CVH-knob
DIQMTQSPSSLSASVGDRVTITCHASQGISGNMGWLQQKPGKAPKGLIYHGANLEEGVPSRFSGSGSGADYTLTISSLQPEDFADYYCVQYAQFPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 463 aa, synthetic construct
  source 1..463: note = hu6VL (D56S) -I2CVH-knob
DIQMTQSPSSLSASVGDRVTITCHASQGISGNMGWLQQKPGKAPKGLIYHGANLESGVPSRFSGSGSGADYTLTISSLQPEDFADYYCVQYAQFPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK